(19) United States
(12) Patent Application Publication
(10) Pub. No.: US 2024/0376100 A1
(43) Pub. Date: Nov. 14, 2024

(54) PAPD5 AND/OR PAPD7 INHIBITING 4-OXO-1, 4-DIHYDROQUINOLINE-3-CARBOXYLIC ACID DERIVATIVES

(71) Applicant: Redona Therapeutics, Inc., Watertown, MA (US)

(72) Inventors: Darren Martin Harvey, Acton, MA (US); Shomir Ghosh, Brookline, MA (US)

(21) Appl. No.: 18/690,127

(22) PCT Filed: Sep. 8, 2022
(86) PCT No.: PCT/US2022/042937

§ 371 Date: Mar. 7, 2024

Related U.S. Application Data
(60) Provisional application No. 63/241,798, filed on Sep. 8, 2021.

(51) Int. Cl. C07D 471/04; A61K 31/4375; A61K 31/444; A61K 31/4545; A61K 31/4709; A61K 31/497; C07D 401/04; C07D 401/14; C07D 519/00

(52) U.S. Cl. CPC C07D 471/04; A61K 31/4375; A61K 31/444; A61K 31/4545; A61K 31/4709; A61K 31/497; C07D 401/04; C07D 401/14; C07D 519/00

(57) ABSTRACT

The invention relates to a compound represented by Formula (I) or a pharmaceutically acceptable salt thereof, compositions comprising the same and methods of preparing and using the same.

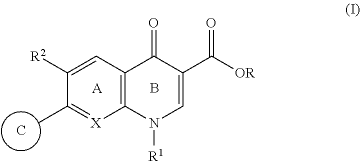

### RELATED APPLICATIONS

This application claims the benefit of U.S. Provisional Application No. 63/241,798, filed Sep. 8, 20021, the entire teaching of which are incorporated herein by reference.

### TECHNICAL FIELD

This disclosure relates to compounds that inhibit PAP Associated Domain Containing 5 (PAPD5, also known as Topoisomerase-related function protein 4-2 (TRF4-2) and Terminal Nucleotidyltransferase 4B (TENT4B)) and/or PAP Associated Domain Containing 7 (PAPD7, also known as Topoisomerase-related function protein 4-1 (TRF4-1) and Terminal Nucleotidyltransferase 4A (TENT4A)), and to methods of using these compounds to treat conditions such as HBV, telomere diseases, aging-related diseases, and other degenerative disorders.

### BACKGROUND

A telomere is a region of repetitive nucleotide sequences at each end of a chromosome. For vertebrates, the sequence of nucleotides in telomeres is TTAGGG. In humans, this sequence of TTAGGG is repeated hundreds to thousands of times.

Telomerase is a ribonucleoprotein polymerase that maintains telomere ends by addition of the telomere repeat TTAGGG. The enzyme consists of a protein component (TERT) with reverse transcriptase activity, and a non-coding RNA component referred to as Telomerase RNA component (also referred to as “TERC”). TERC serves at least two functions: (1) it encodes the template sequence used by telomerase reverse transcriptase (TERT) for the addition of the hexanucleotide repeats to telomeres, and (2) it is the scaffold that nucleates multiple proteins that target telomerase to the Cajal body, where telomeres are extended.

Various mutations (e.g., TERC, PARN, NOP10, NHP2, NAF1, or DKC1) can result in the accumulation of 3′ oligo-adenylated forms of nascent Telomerase RNA Component (TERC) RNA transcripts, which are targeted for destruction, thus causing telomerase deficiency and telomere associated diseases. PAPD5 and PAPD7 are members of the family of noncanonical poly(A) polymerases in human cells. PAPD5 and PAPD7 have been shown to act as a polyadenylase on abnormal pre-ribosomal RNAs in vivo in a manner analogous to degradation-mediating. PAPD5 is also involved in the 3′-end maturation of nascent TERC. 3′ ends of nascent TERC RNA are subject to PAPD5-mediated oligo-adenylation, which targets transcripts for degradation by the exosome. Diminished TERC levels and the increased oligo(A) forms of TERC can therefore be normalized by inhibiting PAPD5 and/or PAPD7. A PAPD5 and/or PAPD7 inhibitor can therefore increase the level or activity of TERC, thereby increasing telomerase activity and telomere elongation in TERC, PARN, NOP10, NHP2, NAF1, or DCK1 mutant cells.

Telomeres are shortened when a cell divides via mitosis. Telomerase restores the short bits of DNA which are otherwise shortened as a consequence of cellular mitosis. Cells with impaired telomerase function often have limited capacity for self-renewal, i.e., an abnormal state or condition characterized by an inability of cells (e.g., stem cells) to divide sufficiently. Accordingly, shortening of telomeres is believed to be a factor in aging. Moreover, this deficiency can lead to a broad range of diseases characterized by shortened telomeres, telomere dysfunction or telomerase dysfunction (Blackburn, et al., Science 350.6265 (2015): 1193-1198), e.g., dysteratosis congenital, Hoyeraal-Hreidarsson syndrome, aplastic anemia, pulmonary fibrosis, hepatic cirrhosis, bone marrow failure, and the like.

Given that diminished telomerase activity is associated with inadequate levels of TERC, and that PAPD5 and/or PAPD7 inhibition can restore TERC levels, PAPD5 and PAPD7 offer an attractive target for treating these diseases. New and improved inhibitors of PAPD5 and PAPD7 could, therefore, provide means for treating these diseases.

PAPD5 and PAPD7 inhibitors have also been disclosed as useful for treating Hepatitis B virus (HBV) infection (WO 2017/216391).

### SUMMARY

The disclosure provides compounds that modulate the level or activity of PAPD5 and/or PAPD7 and therefore modulate TERC levels.

In one aspect, the present disclosure is directed to a compound represented by Formula

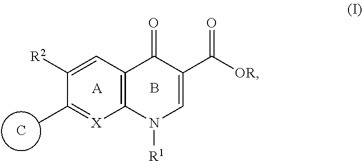

or a pharmaceutically acceptable salt thereof, wherein:
R¹ is C₁₋₆ alkyl, C₃₋₇ cycloaliphatic, phenyl, 5 to 6-membered heteroaryl or 5 to 12-membered heterocyclyl, each optionally substituted with one or more R¹⁰;
   each R¹⁰ is independently halo, —OR^{1a}, —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, —SO₂N(R^{1a})₂, C₁₋₄ alkyl, C₃₋₆ cycloaliphatic, phenyl, 5 to 6-membered heteroaryl or 3 to 6-membered heterocyclyl, wherein the C₁₋₄ alkyl, C₃₋₆ cycloaliphatic, phenyl, 5 to 6-membered heteroaryl and 3 to 6-membered heterocyclyl are each optionally substituted by one or more R¹⁵;
   each R¹⁵ is independently halo, —OR^{1a}, —N(R^{1a})₂, —N(R^{1a})C(O)OR^{1a}, —C(O)R^{1a}, —N(R^{1a})C(O)OR^{1a}, —N(R^{1a})C(O)N(R^{1a})₂, —N(R^{1a})SO₂R^{1a}, —C(O)N(R^{1a})₂, C₁₋₄ alkyl or C₁₋₄ haloalkyl, wherein the C₁₋₄ alkyl is optionally substituted by OR^{1a};
   each R^{1a} is independently H, C₁₋₄ alkyl, phenyl, C₃₋₇ cycloaliphatic or 6 to 7-membered heterocyclyl, wherein the C₁₋₄ alkyl, phenyl, C₃₋₇ cycloaliphatic and 6 to 7-membered heterocyclyl are each optionally substituted with one or more R^{1b} or two R^{1a} taken together with the nitrogen atom to which they are bonded form a 5-6 membered heterocyclyl optionally substituted with one or more R⁵;
   each R^{1b} is independently halo, —OH, —OCH₃, halomethoxy, methyl, halomethyl, —NH₂, —N(H)CH₃, —N(CH₃)₂, phenyl or 4 to 6-membered heterocyclyl;
R² is H, halo, CN, —OR^{2a} or C₁₋₆ alkyl optionally substituted with one or more selected from halo, —OR^{2a}, —NHR^{2a} or —N(R^{2a})₂;
   each R^{2a} is independently H or C₁₋₆ alkyl optionally substituted with one or more R^{2b};
   each R^{2b} is independently halo, —OH, —O—C₁₋₄alkyl, —O—C₁₋₄haloalkyl, —NH₂, —N(H)—C₁₋₄alkyl or —N(C₁₋₄alkyl)₂;
ring C is indazolyl, isoindolinyl, pyrazolyl, dihydro-pyrrolopyrazinyl, dihydro-pyrrolopyridinyl, dihydro-pyrrolopyridazinyl, tetrahydronaphthyridinyl (e.g., tetrahydro-1,7-naphthyridinyl, tetrahydro-2,7-naphthyridinyl, tetrahydro-1,6-naphthyridinyl, tetrahydro-2,6-naphthyridinyl), tetrahydroisoquinolinyl, tetrahydropyrido[4,3-d]pyrimidinyl, or dihydro-pyrrolopyrimidinyl, each optionally substituted with one or more R³;
each R³ is independently H, halo, —OR^{3a}, —N(R^{3a})₂, —N(R^{3a})C(O)R^{3a}, —N(R^{3a})C(O)OR^{3a}, —N(R^{3a})C(O)N(R^{3a})₂, —N(R^{3a})SO₂R^{3a}, —C(O)R^{3a}, —C(O)N(R^{3a})₂, oxo, C₁₋₄ alkyl, phenyl or 5 to 6-membered heteroaryl, wherein the C₁₋₄ alkyl, phenyl and 5 to 6-membered heteroaryl are each optionally substituted by one or more R³⁰;
   each R³⁰ is independently halo, —OR^{3a}, C₁₋₄ alkyl, C₁₋₄ haloalkyl, phenyl or 5 to 6-membered heteroaryl;
   each R^{3a} is independently H, C₁₋₄ alkyl, phenyl or 5 to 6-membered heteroaryl, wherein the C₁₋₄ alkyl, phenyl and 5 to 6-membered heteroaryl are each optionally substituted with one or more R^{3b};
   each R^{3b} is independently Br, Cl, F, —OH, —OCH₃, —OCH₂F, —OCH₂CH₃, —OCH₂CF₃, —OCH₂CH₂F, —NH₂, —N(H)CH₃, —N(CH₃)₂, —CH₃, —CH₂CH₃, —CH₂F, —CHF₂, —CF₃, phenyl or 4 to 6-membered heterocyclyl; and
X is N or CR⁴;
R is H or C₁-C₄ alkyl;
R⁴ is H or halo, and
   with the provisos that:
when R² is halo and ring C is an isoindolinyl, then R¹ is: i) C₁₋₆ alkyl substituted with C₃₋₆ cycloaliphatic, 5 to 6-membered heteroaryl or 3 to 6-membered heterocyclyl, wherein the C₃₋₆ cycloaliphatic, 5 to 6-membered heteroaryl and 3 to 6-membered heterocyclyl are each optionally substituted by one or more R¹⁵; ii) 5 to 6-membered heteroaryl optionally substituted with one or more R¹⁰; or iii) 5 to 12-membered heterocyclyl optionally substituted with one or more R¹⁰;
when R² is halo and ring C is a dihydro-pyrrolopyridinyl, dihydro-pyrrolopyrimidinyl or dihydro-pyrrolopyrazinyl, then R¹ is: i) C₁₋₆ alkyl substituted with C₃₋₆ cycloaliphatic, phenyl, 5 to 6-membered heteroaryl or 3 to 6-membered heterocyclyl, wherein the C₃₋₆ cycloaliphatic, phenyl, 5 to 6-membered heteroaryl and 3 to 6-membered heterocyclyl are each optionally substituted by one or more R¹⁵; ii) 5 to 6-membered heteroaryl optionally substituted with one or more R¹⁰; iii) 5 to 12-membered heterocyclyl optionally substituted with one or more R¹⁰; or iv) phenyl substituted with one or more R¹⁰ provided that at least one R¹⁰ is other than F;
when ring C is pyrazolyl, and R² is halo, then i) R¹ is: C₁₋₆ alkyl substituted with C₃₋₆ cycloaliphatic, phenyl, 5 to 6-membered heteroaryl or 3 to 6-membered heterocyclyl, wherein the C₃₋₆ cycloaliphatic, phenyl, 5 to 6-membered heteroaryl and 3 to 6-membered heterocyclyl are each optionally substituted by one or more R¹⁰; or ii) R¹ is C₄₋₇ cycloaliphatic, phenyl, 5 to 6-membered heteroaryl or 5 to 12-membered heterocyclyl, each optionally substituted with one or more R¹⁰, provided that when R¹ is phenyl substituted by one or more R¹⁰, then R³ is other than pyridyl and at least one R¹⁰ is —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, C₁₋₄ alkyl, C₃₋₆ cycloaliphatic, phenyl, 5 to 6-membered heteroaryl or 3 to 6-membered heterocyclyl, wherein the C₁₋₄ alkyl, C₃₋₆ cycloaliphatic, phenyl, 5 to 6-membered heteroaryl and 3 to 6-membered heterocyclyl are each optionally substituted by one or more R¹⁵; and
when ring C is indazolyl, R¹ is phenyl optionally substituted with one or more R¹⁰ and R² is halo, then the indazolyl is optionally substituted with one or more R³ wherein each R³ is independently H, halo, —N(R^{3a})₂, —N(R^{3a})C(O)R^{3a}, —N(R^{3a})C(O)OR^{3a}, —N(R^{3a})C(O)N(R^{3a})₂, —N(R^{3a})SO₂R^{3a}, —C(O)R^{3a}, —C(O)N(R^{3a})₂, oxo, C₁₋₄ alkyl, phenyl or 5 to 6-membered heteroaryl, wherein the C₁₋₄ alkyl, phenyl and 5 to 6-membered heteroaryl are each optionally substituted by one or more R³⁰.

Alternatively, present disclosure is directed to a compound of Formula (I), where in:
R is H;
   each R¹⁰ is independently halo, —OR^{1a}, —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, C₁₋₄ alkyl, C₃₋₆ cycloaliphatic, phenyl, 5 to 6-membered heteroaryl or 3 to 6-membered heterocyclyl, wherein the C₁₋₄ alkyl, C₃₋₆ cycloaliphatic, phenyl, 5 to 6-membered heteroaryl and 3 to 6-membered heterocyclyl are each optionally substituted by one or more R¹⁵;
   each R¹⁵ is independently halo, —OR^{1a}, —N(R^{1a})₂, —N(R^{1a})C(O)OR^{1a}, —C(O)R^{1a}, —N(R^{1a})C(O)OR^{1a}, —N(R^{1a})C(O)N(R^{1a})₂, —N(R^{1a})SO₂R^{1a}, —C(O)N(R^{1a})₂, C₁₋₄ alkyl or C₁₋₄ haloalkyl;
   each R^{1a} is independently H, C₁₋₄ alkyl, phenyl, C₃₋₇ cycloaliphatic or 6 to 7-membered heterocyclyl, wherein the C₁₋₄ alkyl, phenyl, C₃₋₇ cycloaliphatic and 6 to 7-membered heterocyclyl are each optionally substituted with one or more R^{1b};
ring C is indazolyl, isoindolinyl, pyrazolyl, dihydro-pyrrolopyrazinyl, dihydro-pyrrolopyridinyl, dihydro-pyrrolopyridazinyl or dihydro-pyrrolopyrimidinyl, each optionally substituted with one or more R³, and the remainder of the variables are as described above for Formula (I).

In another aspect, the present disclosure provides a composition (e.g., a pharmaceutical composition) comprising a compound disclosed herein, or a salt (e.g., a pharmaceutically acceptable salt) thereof. The composition may also include a carrier (e.g., a pharmaceutically acceptable carrier).

In another aspect, the disclosure relates to a method of treating a disorder associated with telomere or telomerase dysfunction, the method comprising administering to a subject in need thereof a therapeutically effective amount of a compound disclosed herein, or a pharmaceutically acceptable salt thereof, or a pharmaceutical composition disclosed herein.

In another aspect, the present disclosure also includes a method of treating a telomere disease or disorder associated with telomer dysfunction in a subject comprising administering to the subject a therapeutically effective amount of a compound disclosed herein, or a pharmaceutically acceptable salt thereof, except that the provisos described in the first aspect above do not apply. In yet another aspect, the provisos of formula above do apply.

In another aspect, the present disclosure provides a method of treating a subject with hepatitis B, the method comprising administering to the subject a therapeutically effective amount of a compound disclosed herein, or a pharmaceutically acceptable salt thereof, or a pharmaceutical composition disclosed herein. In yet another aspect, the provisos of formula above apply. In yet another aspect, the provisos of formula above do apply.

Other features or advantages will be apparent from the following detailed description of several embodiments, and also from the appended claims.

### DETAILED DESCRIPTION

The compounds or pharmaceutically acceptable salts thereof, as described herein, have activity as PAPD5 and/or PAPD7 modulators. In particular, compounds or pharmaceutically acceptable salts thereof, as described herein, can be PAPD5 and/or PAPD7 inhibitors.

### I. Compounds of the Disclosure

First embodiment: a compound of the disclosure is represented by Formula (I):

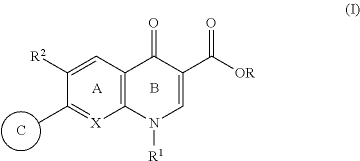

or a pharmaceutically acceptable salt thereof. The variables in Formula (I) are described in the Summary above.

Second embodiment: a compound represented by Formula (I), or a pharmaceutically acceptable salt thereof, wherein R¹ is C₁₋₄ alkyl, C₃₋₆ cycloaliphatic, phenyl, 6-membered heteroaryl or 6-membered heterocyclyl, each optionally substituted with one or more R¹⁰;
each R¹⁰ is independently halo, —OR^{1a}, —N(R^{1a})₂, —N(R^{1a})C(O)R^{1a}, —N(R^{1a})C(O)OR^{1a}, —C(O)R^{1a}, —N(R^{1a})C(O)N(R^{1a})₂, —N(R^{1a})SO₂R^{1a}, —C(O)N(R^{1a})₂, C₁₋₃ alkyl, C₃₋₆ cycloaliphatic, phenyl, 5 to 6-membered heteroaryl or 4 to 6-membered heterocyclyl, wherein the C₁₋₃ alkyl, C₃₋₆ cyclo aliphatic, phenyl, 5 to 6-membered heteroaryl and 4- to 6-membered heterocyclyl are each optionally substituted by one or more R¹⁵; each R¹⁵ is independently halo, —OR^{1a}, —N(R^{1a})₂, —N(R^{1a})C(O)OR^{1a}, —C(O)R^{1a}, —C(O)N(R^{1a})₂ or —CH₃; each R^{1a} is independently H, C₁₋₄ alkyl, phenyl, cyclopropyl or 6-membered heterocyclyl, wherein the C₁₋₄ alkyl, phenyl, cyclopropyl and 6-membered heterocyclyl are each optionally substituted with one or more R^{1b}; R² is H, halo, CN, OR^{2a} or C₁₋₆ alkyl; R^{2a} is H or C₁₋₆ alkyl; ring C is indazolyl, isoindolinyl, pyrazolyl, 6,7-dihydro-5H-pyrrolo[3,4-b]pyrazinyl, 1,3-dihydro-2H-pyrrolo[3,4-c]pyridin-2-yl, 5,7-dihydro-6H-pyrrolo[3,4-b]pyridin-6-yl, 6,7-dihydro-5H-pyrrolo[3,4-c]pyridazinyl or 6,7-dihydro-5H-pyrrolo[3,4-d]pyrimidinyl, each optionally substituted with one or more R³; each R³ is independently H, halo, —OR^{3a}, —N(R^{3a})₂, —N(R^{3a})C(O)R^{3a}, —C(O)N(R₃ₐ)₂, —CH₃, phenyl or 6-membered heteroaryl, wherein the —CH₃, phenyl and 6-membered heteroaryl are each optionally substituted by one or more R³⁰; each R³⁰ is independently halo, —OR^{3a}, —CH₃ or phenyl; each R^{3a} is independently H, —CH₃ or 6-membered heteroaryl, wherein the —CH₃ and 6-membered heteroaryl are each optionally substituted with one or more R^{3b}; and each R^{3b} is independently Cl, —OH, —OCH₃ or —CH₃.

Third embodiment: a compound represented by Formula (II):

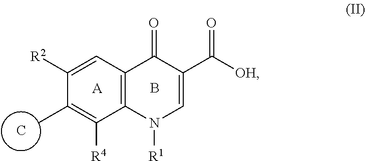

or a pharmaceutically acceptable salt thereof. The variables in Formula (II) are described in the first or second embodiment above.

Fourth embodiment: a compound represented by Formula (III):

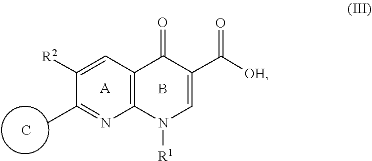

or a pharmaceutically acceptable salt thereof. The variables in Formula (III) are described in the first or second embodiment above.

Fifth embodiment: a compound is represented by Formula (IV), (V) or (VI):

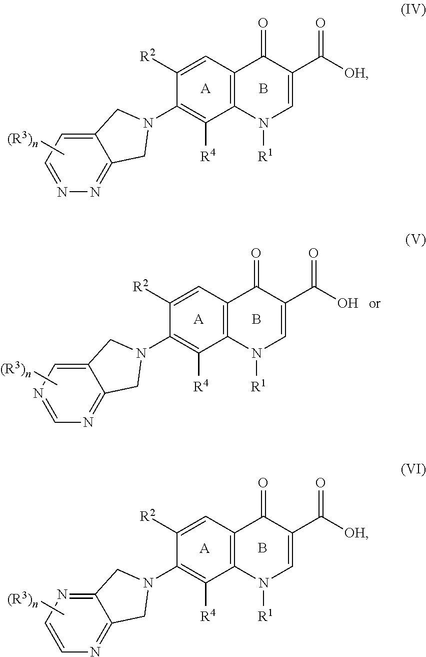

or a pharmaceutically acceptable salt thereof, wherein n is 0, 1, 2, 3, 4 or 5 (alternatively, n is 0 or 1). The variables in Formula (IV), (V) and (VI) are described in the first or second embodiment above.

Sixth embodiment: a compound is represented by Formula (VII), (VIII) or (IX):

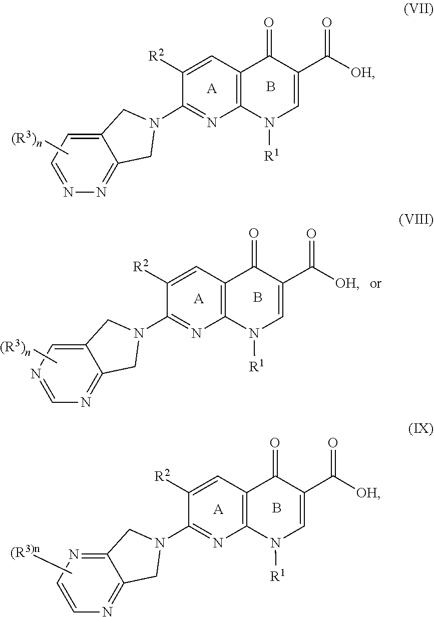

or a pharmaceutically acceptable salt thereof, wherein n is 0, 1, 2, 3, 4 or 5 (alternatively, n is 0 or 1). The variables in Formula (VII), (VIII) and (IX) are described in the first or second embodiment above.

Seventh embodiment: a compound is represented by Formula (X), (XI), (XII), (XIII) or (XIV):

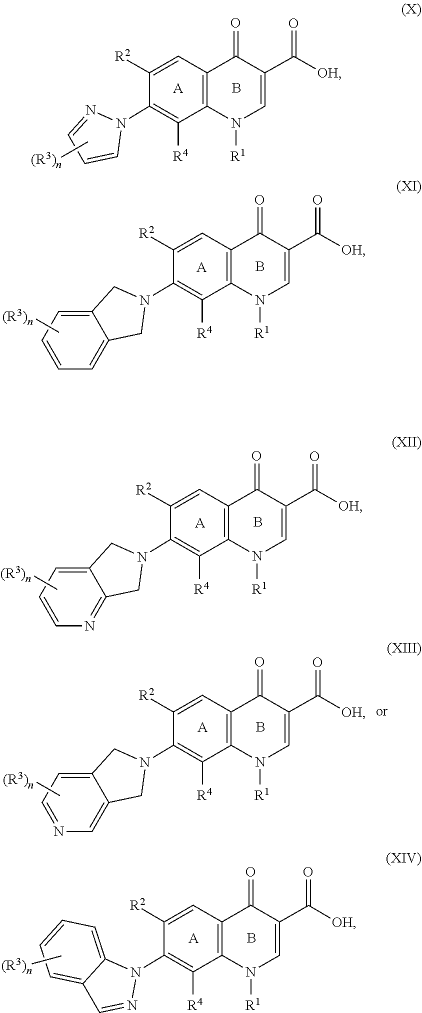

or a pharmaceutically acceptable salt thereof, wherein n is 0, 1, 2, 3, 4 or 5 (alternatively, n is 0 or 1). The variables in Formula (X), (XI), (XII) and (XIII) are described in the first or second embodiment above.

Eighth embodiment: a compound is represented by Formula (XV), (XVI), (XVII), (XVIII) or (XIX):

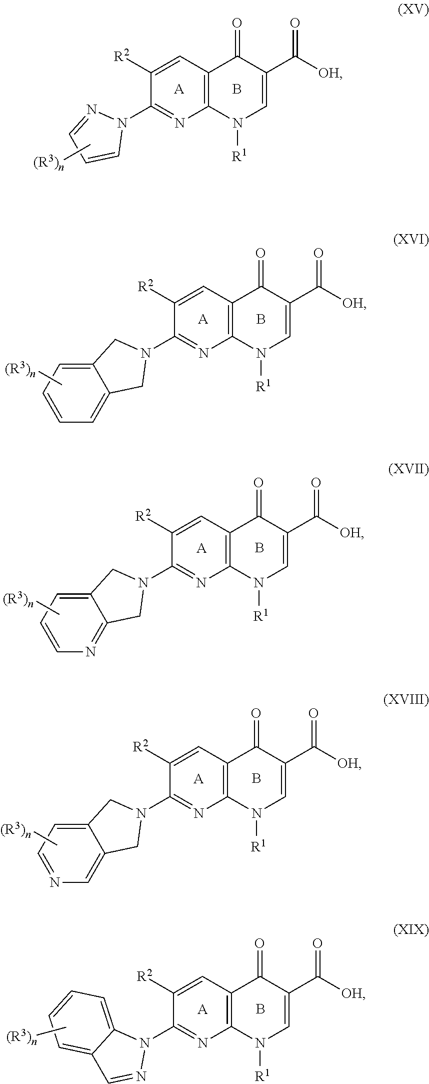

or a pharmaceutically acceptable salt thereof, wherein n is 0, 1, 2, 3, 4 or 5 (alternatively, n is 0 or 1). The variables in Formula (XV), (XVI), (XVII), (XVIII) and (XIX) are described in the first or second embodiment above.

Ninth embodiment: a compound of Formula (X), (XI), (XII), (XIII), (XV), (XVI), (XVII), (XVIII) or (XIX) or a pharmaceutically acceptable salt thereof, wherein wherein R¹ is —CH₃, —CH₂CH₃, —CH(CH₃)₂, cyclohexyl, cyclopropyl, phenyl, piperidinyl, pyridinyl, pyrazinyl, pyrimidinyl or tetrahydropyranyl, each optionally substituted with one or more R¹⁰, for example R¹ is —CH₃, —CH₂CH₃, —CH(CH₃)₂, cyclohexyl, cyclopropyl, phenyl, piperidinyl, pyridinyl, pyrazinyl or tetrahydropyranyl, each optionally substituted with one or more R¹⁰. The remaining variables in Formulae (X), (XI), (XII), (XIII), (XV), (XVI), (XVII), (XVIII) or (XIX) are described in the first or second embodiment above.

Tenth embodiment: a compound of Formula (X), (XI), (XII), (XIII), (XV), (XVI), (XVII), (XVIII) or (XIX), or a pharmaceutically acceptable salt thereof, wherein R¹ is a group of Formula (a) through (g):

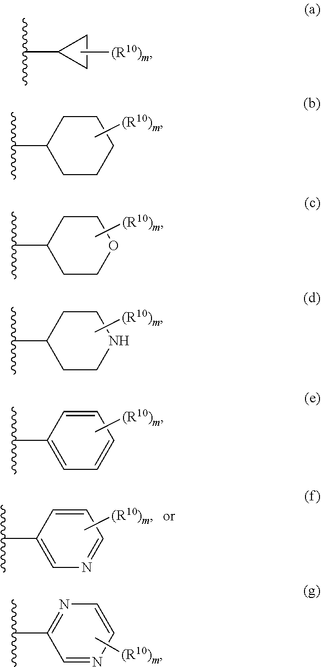

wherein m is 0, 1, 2, 3, 4 or 5, and

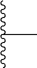

represents a bond to ring B. The remaining variables in Formulae (X), (XI), (XII), (XIII), (XV), (XVI), (XVII), (XVIII) and (XIX) are described in the first or second embodiment above.

Eleventh embodiment: a compound of Formula (X), (XI), (XII), (XIII), (XV), (XVI), (XVII), (XVIII) or (XIX), or a pharmaceutically acceptable salt thereof, wherein R¹ is a group of Formula (h) through (x):

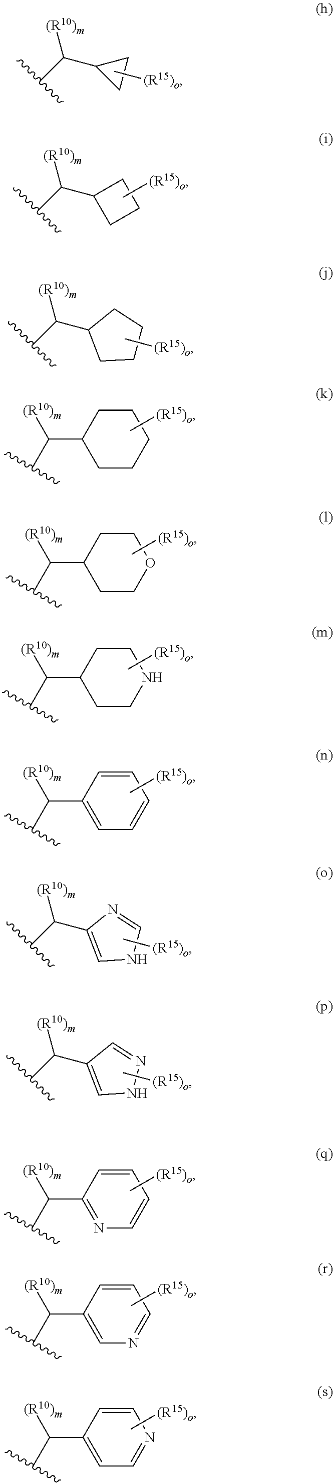

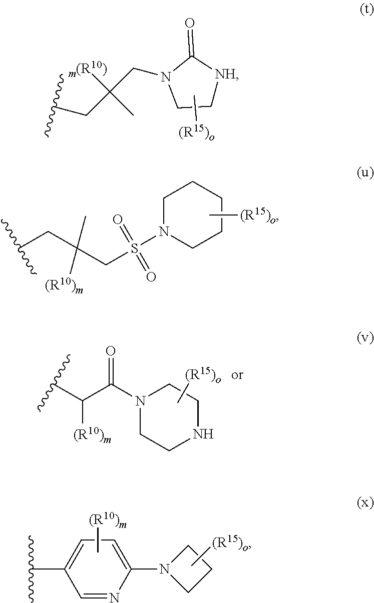

wherein R¹⁰ is —CH₃, —CH₂CH₃ or —CH(CH₃)₂; m is 0, 1 or 2; o is 0, 1, 2, 3, 4 or 5; and

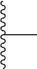

represents a bond to ring B. The remaining variables in Formulae (X), (XI), (XII), (XIII), (XV), (XVI), (XVII), (XVIII) or (XIX) are described in the first or second embodiment above.

Twelfth embodiment: a compound of Formula (X), (XI), (XII), (XIII), (XV), (XVI), (XVII), (XVIII) or (XIX), or a pharmaceutically acceptable salt thereof, wherein each R¹⁰ is independently halo, —OR^{1a}, —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, —CH₃, —CH₂CH₃, —CH₂CH₂CH₃, —CH(CH₃)₂, azetidinyl, cyclobutyl, cyclopentyl, cyclopropyl, cyclohexyl, imidazolyl, 2-oxoimidazolidin-1-yl, phenyl, piperidinyl, pyranyl or pyrazolyl, wherein each of the —CH₃, —CH₂CH₃, —CH₂CH₂CH₃, —CH(CH₃)₂, azetinyl, cyclobutyl, cyclopentyl, cyclopropyl, cyclohexyl, imidazolyl, 2-oxoimidazolidin-1-yl, phenyl, piperidinyl, pyranyl and pyrazolyl are optionally substituted by one or more R¹⁵, for example each R¹⁰ is independently halo, —OR^{1a}, —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, —CH₃, —CH₂CH₃, —CH(CH₃)₂, azetidinyl, cyclobutyl, cyclopentyl, cyclopropyl, cyclohexyl, imidazolyl, 2-oxoimidazolidin-1-yl, phenyl, piperidinyl, pyranyl or pyrazolyl, wherein each of the —CH₃, —CH₂CH₃, —CH(CH₃)₂, azetinyl, cyclobutyl, cyclopentyl, cyclopropyl, cyclohexyl, imidazolyl, 2-oxoimidazolidin-1-yl, phenyl, piperidinyl, pyranyl and pyrazolyl are optionally substituted by one or more R¹⁵. The remaining variables in Formulae (X), (XI), (XII), (XIII), (XV), (XVI), (XVII), (XVIII) and (XIX) are described in the first, second, ninth, tenth or eleventh embodiment above.

Thirteenth embodiment: a compound of Formula (X), (XI), (XII), (XIII), (XV), (XVI), (XVII), (XVIII) or (XIX), or a pharmaceutically acceptable salt thereof, wherein each R¹⁰ is independently a group of Formula (i) through (xvi):

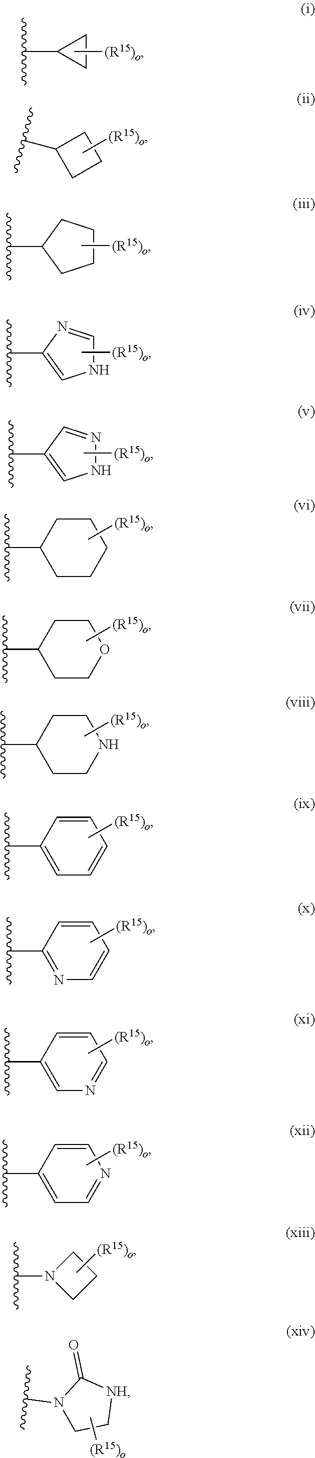

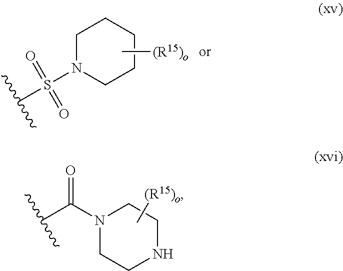

wherein o is 0, 1, 2, 3, 4 or 5, and

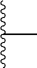

represents a bond to R¹. The remaining variables in Formulae (X), (XI), (XII), (XIII), (XV), (XVI), (XVII), (XVIII) and (XIX) are described in the first, second, ninth, tenth or eleventh embodiment above.

Fourteenth embodiment: a compound of Formula (X), (XI), (XII), (XIII), (XV), (XVI), (XVII), (XVIII) or (XIX), or a pharmaceutically acceptable salt thereof, wherein each R^{1a} is independently H, —CH₃, —CH₂CH₃, —CH(CH₃)₂, —CF₃, tert-butyl, phenyl, cyclopropyl, morpholinyl, piperidin-1-yl or piperazin-1-yl, wherein the —CH₃, —CH₂CH₃, —CH(CH₃)₂, tert-butyl, phenyl, cyclopropyl, morpholinyl, piperidin-1-yl and piperazin-1-yl are each optionally substituted by one or more R^{1b}. The remaining variables in Formulae (X), (XI), (XII), (XIII), (XV), (XVI), (XVII), (XVIII) and (XIX) are described in the first, second, ninth, tenth, eleventh, twelfth or thirteenth embodiment above.

Fifteenth embodiment: a compound of Formula (X), (XI), (XII), (XIII), (XV), (XVI), (XVII), (XVIII) or (XIX), or a pharmaceutically acceptable salt thereof, wherein each R^{1b} is independently piperidinyl, morpholinyl, —OCH₃, —N(H)CH₃ or —N(CH₃)₂, for example each R^{1b} is independently —OCH₃, —N(H)CH₃ or —N(CH₃)₂. The remaining variables in Formulae (X), (XI), (XII), (XIII), (XV), (XVI), (XVII), (XVIII) and (XIX) are described in the first, second, ninth, tenth, eleventh, twelfth, thirteenth or fourteenth embodiment above.

Sixteenth embodiment: a compound of Formula (X), (XI), (XII), (XIII), (XV), (XVI), (XVII), (XVIII) or (XIX), or a pharmaceutically acceptable salt thereof, wherein each R^{1a} is independently H, —CH₃, —CH₂CH₃, —CH₂CH₂OCH₃, —CH₂CH₂N(CH₃)₂, —CH₂CH₂NH(CH₃), —C(CH₃)₂, —CF₃, tert-butyl, phenyl, cyclopropyl, morpholinyl, piperidin-1-yl or piperazin-1-yl. The remaining variables in Formulae (X), (XI), (XII), (XIII), (XV), (XVI), (XVII), (XVIII) and (XIX) are described in the first, second, ninth, tenth, eleventh, twelfth or thirteenth embodiment above.

Seventeenth embodiment: a compound of Formula (X), (XI), (XII), (XIII), (XV), (XVI), (XVII), (XVIII) or (XIX), or a pharmaceutically acceptable salt thereof, wherein each R¹⁵ is independently Cl, F, —OH, —OCH₃, —N(CH₃)₂, —NHC(O)Ot-Bu, —C(O)CH₃, —C(O)N(CH₃)₂, —CH₃ or —CH₂CH₂OCH₃. The remaining variables in Formulae (X), (XI), (XII), (XIII), (XV), (XVI), (XVII), (XVIII) and (XIX) are described in the first, second, ninth, tenth, eleventh, twelfth or thirteenth embodiment above.

Eighteenth embodiment: a compound of Formula (X), (XI), (XII), (XIII), (XV), (XVI), (XVII), (XVIII) or (XIX), or a pharmaceutically acceptable salt thereof, wherein each R¹⁰ is independently selected from the group consisting of F, —OH, —OCH₃, —NH₂, —CH₂OH, —OCH₂CH₂OMe, —OCH₂CH₂N(H)CH₃, —OCH₂CH₂N(CH₃)₂, —N(H)CH₃, —N(CH₃)₂, —NHCH₂CH₂OCH₃, —N(CH₃)CH₂CH₂N(CH₃)₂, —N(H)C(O)CH₃, —N(H)C(O)CH₂CH₃, —N(H)C(O)CH(CH₃)₂, —N(H)C(O)cyclopropyl, (2-(piperidin-1-yl)ethyl)amino, 1-(dimethyl carbamoyl)piperidin-4-yl, methyl(2-(piperidin-1-yl)ethyl)amino, (2-morpholinoethyl)amino, methyl(2-morpholino ethyl)amino, —NHC(O)Ot-Bu, —N(H)C(O)N(CH₃)₂, —N(H)C(O)(N-morpholine), —N(H)SO₂CH₃, —N(H)SO₂CF₃, —C(O)CH₃, —C(O)phenyl, —C(O)N(CH₃)₂, —CH₃, —CH(CH₃)₂, azetidin-1-yl, 3-(dimethylamino)azetidin-1-yl, benzoyl, 1H-imidazol-4-yl, 1-methyl-1H-imidazol-4-yl, 2-oxoimidazolidin-1-yl, pyridin-2-yl, pyridin-3-yl, pyridin-4-yl, 3-(dimethyl amino)azetidin-1-yl)pyridin-3-yl, cyclobutyl, cyclopentyl, cyclopropyl, cyclohenzyl, phenyl, 2-chlorophenyl, 2-fluorophenyl, 4-methoxyphenyl, 2-methylphenyl, 1-methylpiperidin-4-yl, piperidin-1-ylsulfonyl, tetrahydro-2H-pyran-4-yl, pyrazolyl and 1-methyl-1H-pyrazol-4-yl. The remaining variables in Formulae (X), (XI), (XII), (XIII), (XV), (XVI), (XVII), (XVIII) and (XIX) are described in the first, second, ninth, tenth or eleventh embodiment above.

Nineteenth embodiment: a compound of Formula (X), (XI), (XII), (XIII), (XV), (XVI), (XVII), (XVIII) or (XIX), or a pharmaceutically acceptable salt thereof, wherein R¹ is selected from the group consisting of isobutyl, benzyl, 2-chlorobenzyl, 2-fluorobenzyl, 2-methoxybenzyl, 3-methoxybenzyl, 4-methoxybenzyl, 2-methylbenzyl, 1-phenylethyl, (1-methylpiperidin-4-yl)methyl, 1-(dimethylcarbamoyl)piperidin-4-yl, 2-oxo-2-(piperazin-1-yl)ethyl, 2-(methyl sulfonamido)ethyl, 2-(piperidin-1-ylsulfonyl)ethyl, 2-(2-oxoimidazolidin-1-yl)ethyl, cyclobutylmethyl, 1-cyclobutylethyl, 2-cyclobutylpropan-2-yl, cyclopropylmethyl, isobutyl, cyclohexylmethyl, 4-hydroxycyclohexyl, cyclopentylmethyl, 1-methylcyclopropyl, pyridin-2-ylmethyl, pyridin-3-ylmethyl, pyridin-4-ylmethyl, tetrahydro-2H-pyran-4-yl, (tetrahydro-2H-pyran-4-yl)methyl, 4-hydroxyphenyl, 2-fluoro-4-hydroxyphenyl, 3-fluoro-4-hydroxyphenyl, 2-fluoro-4-(methylsulfonamido)phenyl, 2-fluoro-4-(hydroxymethyl) phenyl, piperidin-4-yl, piperidin-4-ylmethyl, 1-acetylpiperidin-4-yl, 1-methylpiperidin-4-yl, 1-methylpiperidin-4-yl)methyl, 1-(2-methoxyethyl)piperidin-4-yl, 1-benzoylpiperidin-4-yl), (1-(tert-butoxy carbonyl)piperidin-4-yl)methyl, (1-acetylpiperidin-4-yl)methyl, (2-methoxypyridin-3-yl)methyl, 1-(dimethylcarbamoyl)piperidin-4-yl)methyl, (1H-pyrazol-4-yl)methyl, (1-methyl-1H-pyrazol-4-yl)methyl, (1H-imidazol-4-yl)methyl, (1-methyl-1H-imidazol-4-yl)methyl, pyrazin-2-yl, pyridin-2-yl, pyridin-3-yl, 6-hydroxypyridin-3-yl, 6-hydroxy-4-methylpyridin-3-yl, 4-methyl-6-(methyl amino)pyridin-3-yl, 4-methyl-6-((2-(methylamino)ethyl)amino)pyridin-3-yl, 4-methyl-6-(methylsulfonamido)pyridin-3-yl, 4-methyl-6-((trifluoromethyl)sulfonamido)pyridin-3-yl, 5-aminopyrazin-2-yl, 5-amino-3-methylpyrazin-2-yl, 5-methoxy-3-methylpyrazin-2-yl, 6-methyl pyrazin-2-yl, 5-methoxypyrazin-2-yl, 6-hydroxy-2-methylpyridin-3-yl, 6-methoxypyridin-3-yl, 6-(dimethylamino)pyridin-3-yl, 6-(dimethylamino)-4-methylpyridin-3-yl, 4-methyl-6-(methyl(2-(piperidin-1-yl)ethyl)amino)pyridin-3-yl, 4-methyl-6-((2-(piperidin-1-yl)ethyl)amino) pyridin-3-yl, 6-((tert-butoxycarbonyl)amino)-4-methylpyridin-yl, 6-aminopyridin-3-yl, 6-amino-5-fluoropyridin-3-yl, 6-amino-2-methylpyridin-3-yl, 6-amino-4-methylpyridin-3-yl, 6-(azetidin-1-yl)-4-methylpyridin-3-yl, 6-amino-2,4-dimethylpyridin-3-yl, 6-acetamidopyridin-3-yl, 6-amino-4-ethylpyridin-3-yl, 6-((2-(dimethylamino)ethyl)(methyl)amino)-4-methylpyridin-3-yl, 6-amino-4-cyclopropylpyridin-3-yl, 6-acetamido-4-methylpyridin-3-yl, 4-methyl-6-propionamidopyridin-3-yl, 6-isobutyramido-4-methylpyridin-3-yl, 6-(cyclopropane carboxamido)-4-methylpyridin-3-yl, 6-((2-methoxyethyl)amino)-4-methylpyridin-3-yl, 6-(azetidin-1-yl)pyridin-3-yl, 6-(3-(dimethylamino)azetidin-1-yl)pyridin-3-yl, 6-(3-(dimethyl amino)azetidin-1-yl)-4-methyl pyridin-3-yl, 6-((tert-butoxycarbonyl)amino)-2-methylpyridin-3-yl, 6-((tert-butoxy carbonyl)amino)pyridin-3-yl, 6-(2-methoxyethoxy)pyridin-3-yl, 6-(2-(dimethyl amino)ethoxy)pyridin-3-yl, 6-(3-(dimethylamino)azetidin-1-yl)-4-methylpyridin-3-yl, 6-((2-methoxyethyl)amino)pyridin-3-yl, 4-methyl-6-(morpholine-4-carboxamido)pyridin-3-yl, 4-methyl-6-(methyl(2-morpholinoethyl)amino)pyridin-3-yl, and 6-(3,3-dimethylureido)-4-methylpyridin-3-yl. The remaining variables in Formulae (X), (XI), (XII), (XIII), (XV), (XVI), (XVII), (XVIII) and (XIX) are described in the first or second embodiment above.

Twentieth embodiment: a compound of Formula (X), (XI), (XII), (XIII), (XV), (XVI), (XVII), (XVIII) or (XIX), or a pharmaceutically acceptable salt thereof, wherein R² is H, Cl, F, —CN, —OCH₃, —OCH₂CH₃ or —CH₃. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth or nineteenth embodiment above.

Twenty-first embodiment: a compound of Formula (X), (XI), (XII), (XIII), (XV), (XVI), (XVII), (XVIII) or (XIX), or a pharmaceutically acceptable salt thereof, wherein each R³ is independently H, halo, —OR^{3a}, —N(R^{3a})₂, —N(R^{3a})C(O)R^{3a}, —C(O)N(R₃ₐ)₂, —CH₃, —CH₂F, —CF₃, phenyl or 6-membered heteroaryl, wherein the methyl, phenyl and 6-membered heteroaryl are each optionally substituted by one or more R³⁰. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth or twentieth embodiment above.

Twenty-second embodiment: a compound of Formula (X), (XI), (XII), (XIII), (XV), (XVI), (XVII), (XVIII) or (XIX), or a pharmaceutically acceptable salt thereof, wherein each R³ is independently H, Br, Cl, F, —OH, —OCH₃, —NH₂, —N(H)CH₃, —N(H)CH₂CH₃, —N(CH₃)₂, —N(CH₃)CH₂CH₃, —N(H)CH₂CH₂OH, —N(H)CH₂CH₂OCH₃, —N(CH₃)CH₂CH₂OCH₃, —C(O)NH₂, —N(CH₃)CH₂CH₂OH, —N(H)C(O)CH₃, —C(O)N(H)CH₃, —CH₃, —CF₃, benzyl, phenyl, pyrazin-2-yl, pyridin-2-yl, pyridin-3-yl or pyridin-4-yl, wherein the —CH₃, benzyl, phenyl, pyrazin-2-yl, pyridin-2-yl, pyridin-3-yl and pyridin-4-yl are each optionally substituted by one or more R³⁰. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth or twentieth embodiment above.

Twenty-third embodiment: a compound of Formula (X), (XI), (XII), (XIII), (XV), (XVI), (XVII), (XVIII) or (XIX), or a pharmaceutically acceptable salt thereof, wherein each R³⁰ is independently halo, —OR^{3a}, —CH₃ or phenyl. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth, twentieth, twenty-first or twenty-second embodiment above.

Twenty-fourth embodiment: a compound of Formula (X), (XI), (XII), (XIII), (XV), (XVI), (XVII), (XVIII) or (XIX), or a pharmaceutically acceptable salt thereof, wherein each R^{3a} is independently H, —CH₃, —CH₂CH₃ or 6-membered heteroaryl, wherein the —CH₃, —CH₂CH₃ and 6-membered heteroaryl are each optionally substituted with one or more R^{3b}, for example each R^{3a} is independently H, —CH₃ or 6-membered heteroaryl, wherein the —CH₃ and 6-membered heteroaryl are each optionally substituted with one or more R^{3b}. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth, twentieth, twenty-first, twenty-second or twenty-third embodiment above.

Twenty-fifth embodiment: a compound of Formula (X), (XI), (XII), (XIII), (XV), (XVI), (XVII), (XVIII) or (XIX), or a pharmaceutically acceptable salt thereof, wherein each R^{3b} is independently Cl, —OH, —OCH₃ or —CH₃. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth, twentieth, twenty-first, twenty-second, twenty-third or twenty-fourth embodiment above.

Twenty-sixth embodiment: a compound of Formula (X), (XI), (XII), (XIII), (XV), (XVI), (XVII), (XVIII) or (XIX), or a pharmaceutically acceptable salt thereof, wherein each R^{3a} is independently H, —CH₃, —CH₂CH₃, —CH₂CH₂OH, —CH₂CH₂OCH₃, pyridin-2-yl, 3-methyl pyridin-2-yl or 3-chloro-6-methoxypyridin-2-yl. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth, twentieth, twenty-first, twenty-second or twenty-third embodiment above.

Twenty-seventh embodiment: a compound of (X), (XI), (XII), (XIII), (XV), (XVI), (XVII), (XVIII) or (XIX), or a pharmaceutically acceptable salt thereof, wherein each R³⁰ is independently Cl, F, —OCH₃, pyridin-2-yloxy, (3-chloro-6-methoxypyridin-2-yl)oxy, —CH₃ or phenyl. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth, twentieth, twenty-first or twenty-second embodiment above.

Twenty-eighth embodiment: a compound of Formula (X), (XI), (XII), (XIII), (XV), (XVI), (XVII), (XVIII) or (XIX), or a pharmaceutically acceptable salt thereof, wherein each R³ is independently H, Br, Cl, F, —OH, —OCH₃, —NH₂, —N(CH₃)₂, —N(H)CH₃, —C(O)NH₂, —N(H)CH₂CH₂OH, —N(H)CH₂CH₂OCH₃, —N(CH₃)CH₂CH₂OCH₃, —N(CH₃)CH₂CH₂OH, —N(H)C(O)CH₃, —C(O)N(H)CH₃, —CH₃, —CF₃, benzyl, phenyl, 3-chlorophenyl, 3-fluorophenyl, 3-methylphenyl, 3-methoxyphenyl, 4-methoxyphenyl, pyrazin-2-yl, pyridin-2-yl, pyridin-3-yl, pyridin-4-yl, 3-chloropyridin-4-yl, 3-methylpyridin-4-yl, (pyridin-2-yloxy)methyl, 3-(((3-methyl pyridin-2-yl)oxy)methyl or ((3-chloro-6-methoxypyridin-2-yl)oxy)methyl. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth or twentieth embodiment above.

Twenty-ninth embodiment: a compound of Formula (X), (XI), (XII), (XIII), (XV), (XVI), (XVII), (XVIII) or (XIX), or a pharmaceutically acceptable salt thereof, wherein ring C is selected from the group consisting of isoindolin-2-yl, 4-chloroindolin-1-yl, 4-fluoro isoindolin-2-yl, 4-methoxyisoindolin-2-yl, 5-fluoroisoindolin-2-yl, 5-methoxyisoindolin-2-yl, 7-bromo-1H-indazol-1-yl, 5,7-dichloro-1H-indazol-1-yl, 6-fluoro-1H-indazol-1-yl, 5,6-difluoro-1H-indazol-1-yl, 7-methyl-1H-indazol-1-yl, 1H-pyrazol-1-yl, 3-carbamoyl-1H-pyrazol-1-yl, 4-carbamoyl-1H-pyrazol-1-yl, 3-(3-chloropyridin-4-yl)-1H-pyrazol-1-yl, 4-(methyl carbamoyl)-1H-pyrazol-1-yl, 3-fluoro-1H-pyrazol-1-yl, 4-fluoro-1H-pyrazol-1-yl, 3-(trifluoro methyl)-1H-pyrazol-1-yl, 3-amino-1H-pyrazol-1-yl, 3-(methylamino)-1H-pyrazol-1-yl, 3-bromo-1H-pyrazol-1-yl, 3-chloro-1H-pyrazol-1-yl, 5-methyl-3-(pyridin-2-yl)-1H-pyrazol-1-yl, 3-(methylamino)-1H-pyrazol-1-yl, 3-(dimethylamino)-1H-pyrazol-1-yl, 4-bromo-3-methyl-1H-pyrazol-1-yl, 3-acetamido-1H-pyrazol-1-yl, 4-acetamido-1H-pyrazol-1-yl, 3-(4-methoxyphenyl)-1H-pyrazol-1-yl, 3-((2-hydroxyethyl)amino)-1H-pyrazol-1-yl, 3-((2-hydroxyethyl)(methyl) amino)-1H-pyrazol-1-yl, 3-((2-methoxyethyl)amino)-1H-pyrazol-1-yl, 3-(3-methylpyridin-4-yl)-1H-pyrazol-1-yl, 3-(((3-methylpyridin-2-yl)oxy)methyl)-1H-pyrazol-1-yl, 3-((2-methoxyethyl) (methyl)amino)-1H-pyrazol-1-yl, 3-(pyrazin-2-yl)-1H-pyrazol-1-yl, 3-((pyridin-2-yloxy)methyl)-1H-pyrazol-1-yl, 3-phenyl-1H-pyrazol-1-yl, 3-(pyridin-3-yl)-1H-pyrazol-1-yl, 3-(pyridin-4-yl)-1H-pyrazol-1-yl, 5,7-dihydro-6H-pyrrolo[3,4-b]pyridin-6-yl, 4-fluoro-5,7-dihydro-6H-pyrrolo [3,4-b]pyridin-6-yl, 3-fluoro-5,7-dihydro-6H-pyrrolo[3,4-b]pyridin-6-yl, 4-methyl-5,7-dihydro-6H-pyrrolo[3,4-b]pyridin-6-yl, 3-methyl-5,7-dihydro-6H-pyrrolo[3,4-b]pyridin-6-yl, 2-methyl-5,7-dihydro-6H-pyrrolo[3,4-b]pyridin-6-yl, 3-methyl-5,7-dihydro-6H-pyrrolo[3,4-b]pyridin-6-yl, 1,3-dihydro-2H-pyrrolo[3,4-c]pyridin-2-yl, 5-oxo-5,7-dihydro-6H-pyrrolo[3,4-b]pyridin-6-yl, 7-methyl-5,7-dihydro-6H-pyrrolo[3,4-b]pyridin-6-yl, 5-methyl-5,7-dihydro-6H-pyrrolo[3,4-b] pyridin-6-yl, 4-methyl-4,5,6,7-tetrahydro-2H-pyrazolo[3,4-b]pyrazin-2-yl, 7-methyl-4,5,6,7-tetrahydro-2H-pyrazolo[3,4-b]pyrazin-2-yl and 7-methyl-6,7-dihydropyrazolo[4,3-b][1,4]oxazin-2(5H)-yl. The remaining variables in Formulae (X), (XI), (XII), (XIII), (XV), (XVI), (XVII), (XVIII) and (XIX) are described in the first, second, ninth, tenth, eleventh, twelfth, thirteenth, fourteenth, fifteenth, sixteenth, seventeenth, eighteenth, nineteenth or twentieth embodiment above.

Thirtieth embodiment: a compound of Formula (X), (XI), (XII) or (XIII), or a pharmaceutically acceptable salt thereof, wherein R⁴ is H or F. The remaining variables in Formulae (X), (XI), (XII) and (XIII) are described in the first, second, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-eighth or twenty-ninth embodiment above.

Thirty-first embodiment: a compound is represented by Formula (XX), (XXI), (XXII), (XXIII), (XXIV) or (XXV):

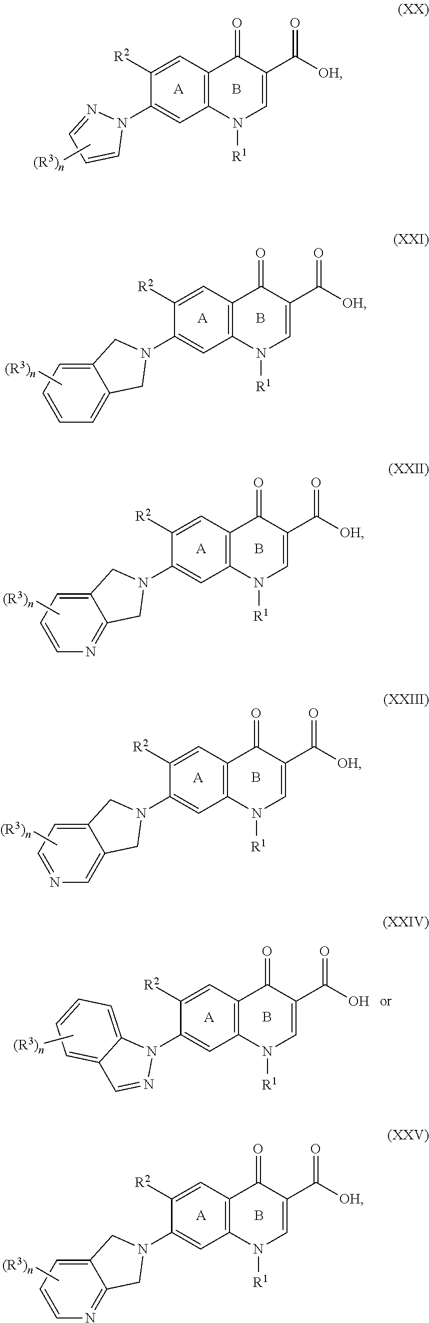

or a pharmaceutically acceptable salt thereof, wherein: R¹ is —CH₃, —CH₂CH(CH₃)₂, phenyl, piperidin-4-yl, pyridin-3-yl or pyrazin-2-yl, wherein the —CH₃, phenyl, piperidin-4-yl, pyridin-3-yl and pyrazin-2-yl are each each optionally substituted with one or more R¹⁰; R¹⁰

is independently F, —OH, —OCH₃, —OCH₂CH₂OCH₃, —NH₂, —N(CH₃)₂, —OCH₂CH₂N(CH₃)₂, —NHCH₂CH₂OCH₃, —NHC(O)CH₃, —NHSO₂CH₃, —NHC(O)OC(CH₃)₃, —CH₃, —C(O)OC(CH₃)₃, benzoyl, —CH₂OH, phenyl, cyclobutyl, cyclohexyl, cyclopentyl, azetidin-1-yl, piperidin-4-yl, pyridin-2-yl, pyridin-3-yl or pyridin-4-yl, wherein the —CH₃, cyclobutyl, azetidin-1-yl, piperidin-4-yl, phenyl and pyridin-3-yl are each optionally substituted by one or more R¹⁵; R¹⁵ is independently Cl, F, —OH, —OCH₃, —N(CH₃)₂, —C(O)OC(CH₃)₃ or —CH₃; R² is Cl, F or —CN; R³ is independently Br, Cl, F, —OCH₃, —NHCOCH₃, —CH₃ or 4-methoxyphenyl; and n is 0, 1 or 2.

Thirty-second embodiment: a compound is represented by Formula (XXVI) or (XXVII):

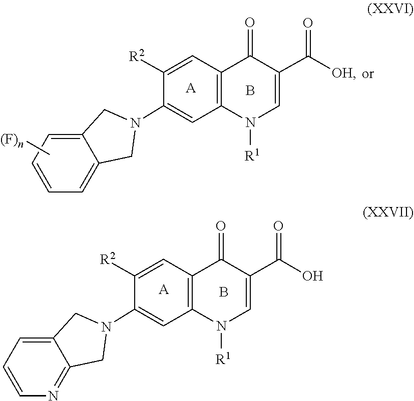

or a pharmaceutically acceptable salt thereof, wherein: R¹ is —CH₃, phenyl, pyridin-3-yl or pyrazin-2-yl, each optionally substituted with F, —OH, —OCH₃, —NH₂, —NHC(O)OC(CH₃)₃, —NHSO₂CH₃, —CH₃ or 2-chlorophenyl; R² is Cl, F or —CN; and n is 0 or 1.

In one embodiment, the disclosure provides a compound selected from those described in Exemplification section, e.g., Examples 1-87, including pharmaceutically acceptable salts thereof and the neutral form.

### II. Definitions

As used herein, the term “alkyl” refers to a fully saturated branched or unbranched hydrocarbon moiety having 1 to 20 carbon atoms. Preferably, the alkyl comprises 1 to 6 carbon atoms, or 1 to 4 carbon atoms. Representative examples of alkyl include, but are not limited to, methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like.

The terms “cycloaliphatic”, “cycloaliphatic group” or “cycloaliphatic ring” are used interchangeably to refer to a saturated (i.e., a cycloalkyl that is also defined below) or unsaturated non-aromatic, monocyclic or bicyclic carbon ring system (including fused and bridged bicyclic) which has 3- to 12-ring members, alternatively 3 to 7 members. The term “cycloaliphatic” also includes ring systems in which a carbocyclic (hydrocarbon) aromatic ring is fused to a saturated or partially unsaturated (non-aromatic) hydrocarbon ring. Examples of monocyclic cycloaliphatic ring systems include, but are not limited to, cyclopropyl, cyclobutyl, cyclopentyl, cyclohexyl, cycloheptyl, cyclopropenyl, cyclobutenyl, cyclopenentyl, cyclohexenyl, cycloheptenyl and the like. Examples of bicyclic cycloaliphatic ring systems include, but are not limited to octahydronapthalenyl, decalinyl, and the like.

The term “cycloalkyl” refers to completely saturated monocyclic or bicyclic hydrocarbon rings (including fused and bridged bicyclic) having 3-12 ring carbon atoms, alternatively 3-7 ring carbon atoms. Exemplary bicyclic cycloalkyl groups include bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl and decalinyl. Examples of monocyclic cycloalkyl groups include, but are not limited to, cyclopropyl, cyclobutyl, cyclopentyl, cyclohexyl, cycloheptyl, cyclooctyl, cyclononyl, cyclodecyl, cycloundecyl, cyclododecyl, and the like.

The term “alkoxy” as used herein refers to the group —OR, in which R is an alkyl as defined above.

The terms “heterocyclyl,” “heterocycle,” and “heterocyclic ring” refer to a 3 to 12-membered (alternatively 3-7 membered), saturated or partially unsaturated (non-aromatic), monocyclic or bicyclic ring system (including fused and bridged bicyclic) having at least one or more heteroatom selected from O, S and N as a ring member, and wherein C can be oxidized (e.g., C(O)), N can be oxidized (e.g., N(O)) or quaternized, and S can be optionally oxidized to sulfoxide and sulfone. The term “heterocyclyl” includes ring systems in which an aryl or heteroaryl ring is fused to a saturated or partially unsaturated (non-aromatic) ring having at least one heteroatom as a ring member.

Examples of heterocyclyls include, but are not limited to, aziridinyl, oxiranyl, thiiranyl, oxaziridinyl, azetidinyl, oxetanyl, tetrahydropyranyl, thietanyl, pyrrolidinyl, tetrahydrofuranyl, thiolanyl, imidazolidinyl, pyrazolidinyl, oxazolidinyl, 2-oxoimidazolidin-1-yl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, dihydropyranyl, 2,3-dihydrobenzofuranyl, indolinyl (or 2,3-dihydroindolyl), isoindolinyl, dihydro-pyrrolo pyridinyl, 1,3-dihydro-2H-pyrrolo[3,4-c]pyridin-2-yl, 5,7-dihydro-6H-pyrrolo[3,4-b]pyridin-6-yl, 2,3-dihydrobenzothiophenyl, 2,3-dihydrobenzothiazolyl, 1,2,3,4-tetrahydroquinolinyl, 1,2,3,4-tetrahydroisoquinolinyl, 5,6,7,8-tetrahydropyrido[3,4-b]pyrazinyl, 1,3-dihydroisobenzofuranyl, 6,7-dihydro-5H-pyrrolo[3,4-b]pyrazinyl, 1,3-dihydro-2H-pyrrolo[3,4-c]pyridin-2-yl, 5,7-dihydro-6H-pyrrolo[3,4-b]pyridin-6-yl, 6,7-dihydro-5H-pyrrolo[3,4-c]pyridazinyl, 6,7-dihydro-5H-pyrrolo[3,4-d]pyrimidinyl, and the like. Examples of bridged bicyclics include azabicyclo[2.2.1]hepantyl, azabicyclo[3.2.1]octanyl, azabicyclo [3.3.1]nonanyl, diazabicyclo[2.2.1]hepantyl, diazabicyclo[3.2.1]octanyl and diazabicyclo [3.3.1]nonanyl. Examples of oxygen containing bridged bicyclics include oxobicyclo[2.2.1]hepantyl, oxobicyclo[3.2.1]octanyl, oxobicyclo [3.3.1]nonanyl, oxa-azabicyclo[2.2.1]hepantyl, oxa-azabicyclo[3.2.1]octanyl and oxa-azabicyclo [3.3.1]nonanyl.

As used herein, “heteroaryl” can be used interchangeably with “heteroaromatic,” “heteroaryl ring,” “heteroaryl group,” “heteroaromatic ring,” and “heteroaromatic group”. It refers to a 5 to 10-membered, fully aromatic, monocyclic or fused bicyclic ring system having at least one to four heteroatoms selected from O, N and S. Rings containing oxidized forms of N (e.g., N(O)) or S (e.g., sulfoxide and sulfone) are not encompassed within the meaning of “heteroaryl”. “Heteroaryl” includes monocyclic rings and bicyclic rings in which a monocyclic heteroaromatic ring is fused to a carbocyclic aromatic hydrocarbon or heteroaromatic ring.

Examples of heteroaryls include, but are not limited to, furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridinyl (or pyridyl, e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), isothiazolyl, triazolyl (e.g., 2-triazolyl, 5-triazolyl), tetrazolyl (e.g., tetrazolyl), thienyl (e.g., 2-thienyl, 3-thienyl), pyranyl, thiopyranyl, pyrazinyl, oxazinyl, thiazinyl, dioxinyl, dithiinyl, oxathianyl, triazinyl, tetrazinyl, azaindolyl, benzimidazolyl, benzofuryl, benzoisoxazolyl, benzoisothiazolyl, benzothiadiazolyl, benzothiazolyl, benzothienyl, benzotriazolyl, benzoxadiazolyl, benzoxazolyl, furopyridinyl, imidazopyridyl, imidazopyrimidinyl, indazolyl, indolizinyl, indolyl, isoquinolinyl, oxazolo pyridinyl, purinyl, pyrazolopyrimidinyl, pyrazolopyrazinyl, pyridopyazinyl, pyridopyrimidinyl, pyrrolo[2,3]pyrimidinyl, pyrrolopyrazolyl, pyrroloimidazolyl, pyrrolotriazolyl, quinazolinyl, quinoxalinyl, quinolinyl, isoquinolinyl, thiazolopyridinyl, thienopyridinyl, thienopyrimidinyl, thienopyrazinyl, napthyridyl, and the like.

The number of carbon atoms in a group is specified herein by the prefix “Cₓ₋ₓₓ”, wherein x and xx are integers. For example, “C₁₋₄alkyl” is an alkyl group which has from 1 to 4 carbon atoms.

The term “fused ring system”, as used herein, is a ring system that has two rings each of which are independently selected from a carbocyclyl or a heterocyclyl, wherein the two ring structures share two adjacent ring atoms. In one embodiment, a fused ring system have from 9 to 12 ring members.

The term “bridged ring system”, as used herein, is a ring system that has a carbocyclyl or heterocyclyl ring wherein two non-adjacent atoms of the ring are connected (bridged) by one or more (preferably from one to three) atoms selected from C, N, O, and S. In one embodiment, a bridged ring system have from 6 to 12 ring members.

The term “Halogen” or “halo” as used herein refers to F, Cl, Br or I. Preferably, halo is F, Cl, or Br.

The term “haloalkyl” refers to an alkyl group having at least one halogen substitution. “Haloalkoxy” is a haloalkyl group which is attached to another moiety via an oxygen

The term “oxo” refers to the diradical ═O. Oxo groups are not substituents on non-aromatic rings.

If a group is described as being “optionally substituted,” the group may be either (1) not substituted, or (2) substituted. If a carbon of a group is described as being optionally substituted with one or more of a list of substituents, one or more of the hydrogen atoms on the carbon (to the extent there are any) may separately and/or together be replaced with an independently selected optional substituent. If a group is substituted with one or more substituents, it can be substituted with 1, 2, 3, 4, 5, 6, or more independently selected substituents. In certain embodiments, it can be substituted with 1, 2, 3, 4, 5 or 6 independently selected substituents. In certain embodiments, it can be substituted with 1, 2 or 3 independently selected substituents.

The term “pharmaceutically acceptable salt” as used herein refers to pharmaceutically acceptable organic or inorganic salts of a bifunctional compound of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the disclosure contains one or more basic moieties, desired salts (e.g., pharmaceutically acceptable salts) may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the disclosure contains one or more acidic moieties, desired salts (e.g., pharmaceutically acceptable salts) may be prepared by any suitable method, for example, treatment of the free acid with an inorganic, such as an alkali metal hydroxide or alkaline earth metal hydroxide, organic base, such as an amine (primary, secondary or tertiary), or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

### III. Pharmaceutical Compositions and Methods of Uses

### Pharmaceutical Compositions

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with several of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

### Therapeutic Methods

The PAPD5 and PAPD7 inhibitors disclosed herein are potential therapeutics for the treatment of telomere associated diseases or disorders. A “telomere associated disease or disorder” refers to a disease or disorder that is caused, at least in part, by an alteration in a gene associated with the telomere or the telomere pathway. A telomere associated disease or disorder can also refer to any disease or disorder that is caused, at least in part, by shortening of the telomere. Telomere associated diseases or disorders include those affecting the blood and immune systems, lungs, liver, skin, mucosal surfaces, bones, cardiovascular system, endocrine system, and/or gastrointestinal system, as cells with the impaired self-renewal capacity can affect the normal function of organs or systems. A “telomere associated disease or disorder” is often associated with a cellular state marked with decreased self-renewal capacity that can be attributed to an alteration in telomere length. Telomere disease also includes tissue failure and organ failure. The tissue failure that relates to telomere disease can have various causes, e.g., infection, inflammation, environmental (radiation, chemical, physical insults) causes, medications and chemotherapy, among others. These various causes can all contribute to telomere deficiency.

“Telomere deficiency” refers to a cellular state in the body, including stem cells, induced pluripotent cells and fibroblasts, and is often marked by a perturbation in expression or activity of an enzyme that is involved in regulating telomere size. “Telomerase dysfunction” refers to abnormal levels or fabrication of telomerase in a cell or patient. For example, telomerase dysfunction can include telomerase deficiency, such as where telomerase levels are lower than normal due to excess or unwanted telomerase degradation, and telomerase over-activity, such as where telomerase levels are higher than normal due to deficient telomerase degradation.

Dysteratosis congenita is a telomere associated disease predominantly caused by impairment of TERC levels and function. Reduced TERC levels in dysteratosis congenita patients leads to loss of telomerase function and premature shortening of telomeres, leading to bone marrow failure, widespread tissue dysfunction and mortality. Reduction in telomerase RNA degradation by inhibiting either the poly(A) polymerase PAPD5, PAPD7 or the 3′ to 5′ exonuclease EXOSC10 has been shown to rescue telomerase RNA levels and telomerase activity. More importantly, a partial depletion of PAPD5 and/or PAPD7 is able to improve the hematopoietic potential of dysteratosis congenita cells, thereby rescuing the major phenotype observed in this disease (WO2021/092159).

Other telomere associated diseases treatable by the disclosed PAPD5 and/or PAPD7 inhibitors are aplastic anemia, myelodysplastic syndrome, or familial pulmonary fibrosis. These diseases are characterized by reduced or disrupted TERC processing. Some examples of mutations in telomere maintenance genes that lead to these diseases include mutations in TERC, PARN and ZCCHC8. Inhibition of PAPD5 and/or PAPD7 can restore TERC processing in subjects with these diseases (WO2021/092159).

Hoyeraal-Hreidarsson syndrome is a multisystem genetic disorder characterized by very short telomeres (less than first percentile for age) and is considered a clinically severe variant of dysteratosis congenital. Patients with Hoyeraal-Hreidarsson syndrome present in early childhood with cerebellar hypoplasia, microcephaly, immunodeficiency, bone marrow failure, and intrauterine growth retardation. (Glousker et al., Br. J. Haematol. 170:457-471.2015). One embodiment of the invention is a method of treating a subject with Hoyeraal-Hreidarsson syndrome with the disclosed PAPD5 and/or PAPD7 inhibitors.

Idiopathic pulmonary fibrosis is another telomere associated disease characterized by telomere shortening and mutations in TERC and TERT (Armanios, M. Mutation Research/Fundamental and Molecular Mechanisms of Mutagensis 730:52 (2012). Idiopathic pulmonary fibrosis is a chronic and ultimately fatal disease characterized by a progressive decline in lung function. Another embodiment of the invention is treating a subject with idiopathic pulmonary fibrosis with a disclosed PAPD5 and/or PAPD7 inhibitor.

Liver disease associated with telomere shortening consists of mainly fibrosis with inflammation and nodular regenerative hyperplasia, a leading cause of noncirrhotic portal hypertension (Calado, et al., PLoS One. 4:e7926 (2009)). Other hepatic diseases associated with telomere shortening include chronic liver disease, non-alcoholic steatohepatitis, and hepatic cirrhosis (WO2020/051375). The invention includes treating subjects suffering from these liver diseases with the disclosed PAPD5 and/or PAPD7 inhibitors.

Certain neurodegenerative disorders can be characterized by shortened telomeres, decreased levels of TERC, and/or decreased levels of telomerase relative to normal tissues. Exemplary of these neurodegenerative disorders include Motor Neuron Disease, Creutzfeldt-Jakob disease, Machado-Joseph disease, Spino-cerebellar ataxia, Multiple sclerosis (MS), Parkinson's disease, Huntington's disease, hearing and balance impairments, ataxias, epilepsy, mood disorders such as schizophrenia, bipolar disorder, and depression, dementia, Pick's Disease, stroke, central nervous system hypoxia, cerebral senility, and neural injury such as head trauma (WO2020/051375). Moreover, recent studies have shown the association between shorter telomeres and Alzheimer's disease Zhan, et al., JAMA neurology 72.10 (2015): 1202-1203. Treatment of these neurodegenerative diseases by the disclosed PAPD5 and/or PAPD7 inhibitors is encompassed by the invention.

There has been reported an association between leucocyte telomere length and risk of coronary heart disease. Haycock, et al., BMJ “2014; 349: g4227; and Codd et al., Nature genetics 45.4 (2013): 422-427. As such, the disclosed PAPD5 and/or PAPD7 inhibitors can be used to treat subjects with cardiovascular disease or coronary artery disease such as atherosclerosis, hypertension, atherosclerosis, coronary artery disease and ischemia/reperfusion injury (WO2020/051375).

Another telomeres associated disease is type 2 diabetes. Zhao et al., PLoS One 8.11 (2013): e79993. As such, a method of treating a subject with type 2 diabetes with the disclosed PAPD5 inhibitors is another embodiment of the invention.

WO 2017/216391 discloses the use of PAPD5 and/or PAPD7 inhibitors for the treatment and prevention of hepatitis B infection. Accordingly, another embodiment of the invention is a method of treating a subject with hepatitis B infection or reducing the likelihood of a subject contracting hepatitis B infection when the subject is at risk of developing hepatitis B infection.

When used to treat hepatitis B infection, the disclosed compounds can be co-administered with a therapeutically effective amount of an additional therapeutic agent effective against hepatitis B. The additional therapeutic agent is selected from core inhibitor, which includes GLS4, GLS4JHS, JNJ-379, ABI-H0731, ABI-H2158, AB-423, AB-506, WX-066, and QL-0A6A; immune modulator or immune stimulator therapies, which includes T-cell response activator AIC649 and biological agents belonging to the interferon class, such as interferon alpha 2a or 2b or modified interferons such as pegylated interferon, alpha 2a, alpha 2b, lamda; or STING (stimulator of interferon genes) modulator; or TLR modulators such as TLR-7 agonists, TLR-8 agonists or TLR-9 agonists; or therapeutic vaccines to stimulate an HBV-specific immune response such as virus-like particles composed of HBcAg and HBsAg, immune complexes of HBsAg and HBsAb, or recombinant proteins comprising HBx, HBsAg and HBcAg in the context of a yeast vector; or immunity activator such as SB-9200 of certain cellular viral RNA sensors such as RIG-I, NOD2, and MDA5 protein, or RNA inference (RNAi) or small interfering RNA (siRNA) such as ARC-520, ARC-521, ARB-1467, and ALN-HBV RNAi, or antiviral agents that block viral entry or maturation or target the HBV polymerase such as nucleoside or nucleotide or non-nucleos(t)ide polymerase inhibitors, and agents of distinct or unknown mechanism including agents that disrupt the function of other essential viral protein(s) or host proteins required for HBV replication or persistence such as REP 2139 and AB-452. In an embodiment of the combination therapy, the reverse transcriptase inhibitor is at least one of Zidovudine, Didanosine, Zalcitabine, ddA, Stavudine, Lamivudine, Aba-cavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine. In another embodiment of the combination therapy, the TLR-7 agonist is selected from the group consisting of SM360320 (12-benzyl-8-hydroxy-2-(2-methoxy-ethoxy)adenine), AZD 8848 (methyl [3-({[3-(6-amino-2-butoxy-8-oxo-7,8-dihydro-9H-purin-12-yl)propyl][3-(4-morpholinyl) propyl] amino Imethyl)phenyl] acetate), GS-9620 (4-Amino-2-butoxy-8-[3-(2-pyrrolidinylmethyl)benzyl]-7,8-dihydro-6(5H)-pteridinone), AL-034 (TQ-A3334), and RO6864018. In another embodiment of the combination therapy, the TLR-8 agonist is GS-9688.

Additional therapeutics that can be co-administered to treat hepatitis B include immunomodulators. For example, the immunomodulator is an anti-PD-1 antibody chosen 30 from MDX-1 106, Merck 3475 or CT-01 1. Alternatively, the immunomodulator is a PD-1 inhibitor such as AMP-224, anti-PD-LI antibody, an anti-PD-L1 binding antagonist chosen from YW243.55.570, MPDL3280A, MEDI-4736, MSB-0010718C, or MDX-1 105. MDX-1 105, also known as BMS-936559, is an anti-PD-LI antibody described in WO2007/005874. Antibody YW243.55.570 is an anti-PD-LI described in WO 2010/077634. Alternatively, the immunomodulator is nivolumab (CAS Registry Number: 946414-94-4). Alternative names for nivolumab include MDX-1 106, MDX-1 106-04, ONO-4538, or BMS-936558. Nivolumab is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449, EP2161336 and WO2006/121 168. In another alternative, the immunomodulator is an anti-PD-1 antibody Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509, WO2009/1 14335, and WO2013/079174.

In yet another alternative, the immunomodulator is Pidilizumab (CT-01 1; Cure Tech), a humanized IgG1 k monoclonal antibody that binds to PD1. Pidilizumab and other humanized anti-PD-1 monoclonal antibodies are disclosed in WO2009/10161 1. Other anti-PD1 antibodies useful as immunomodulators for use in the methods disclosed herein include AMP 514 (Amplimmune), and anti-PD1 antibodies disclosed in U.S. Pat. No. 8,609,089, US 2010028330, and/or US 201201 14649. In some embodiments, the anti-PD-L1 antibody is MSB0010718C. MSB0010718C (also referred to as A09-246-2; Merck Serono) is a monoclonal antibody that binds to PD-L1.

In yet another alternative, the immunomodulator is MDPL3280A (Genentech/Roche), a human Fc optimized IgG1 monoclonal antibody that binds to PD-L1. MDPL3280A and other human monoclonal antibodies to PD-L1 are disclosed in U.S. Pat. No. 7,943,743 and U.S. Publication No.: 20120039906. Other anti-PD-L1 binding agents useful as immunomodulators for methods of the invention include YW243.55.S70 (see WO2010/077634), MDX-1 105 (also referred to as BMS-936559), and anti-PD-L1 binding agents disclosed in WO2007/005874.

In some embodiments, the immunomodulator is AMP-224 (B7-DCIg;

Amplimmune; e.g., disclosed in WO2010/027827 and WO201 1/066342), is a PD-L2 Fc fusion soluble receptor that blocks the interaction between PD1 and B7-H1. In some embodiments, the immunomodulator is an anti-LAG-3 antibody such as BMS-986016. BMS-986016 (also referred to as BMS986016) is a monoclonal antibody that binds to LAG-3. BMS-986016 and other humanized anti-LAG-3 antibodies are disclosed in US 201 1/0150892, WO2010/019570, and WO2014/008218

In certain embodiments, the combination therapies disclosed herein include a modulator of a costimulatory molecule or an inhibitory molecule, e.g., a co-inhibitory ligand or receptor. In one embodiment, the costimulatory modulator, e.g., agonist, of a costimulatory molecule is chosen from an agonist (e.g., an agonistic antibody or antigen-binding fragment thereof, or soluble fusion) of 0X40, CD2, CD27, CDS, ICAM-1, LFA-1 (CD1 1 a/CD18), ICOS (CD278), 4-1 BB (CD137), GITR, CD30, CD40, BAFFR, HVEM, CD7, LIGHT, NKG2C, SLAMF7, NKrdO, CD160, B7-H3 or CD83 ligand.

In another embodiment, the combination therapies disclosed herein include an immunomodulator that is a costimulatory molecule, e.g., an agonist associated with a positive signal that includes a costimulatory domain of CD28, CD27, ICOS and/or GITR.

Exemplary GITR agonists include, e.g., GITR fusion proteins and anti-GITR antibodies (e.g., bivalent anti-GITR antibodies), such as, a GITR fusion protein described in U.S. Pat. No. 6,111,090, European Patent No.: 090505B1, U.S. Pat. No. 8,586,023, PCT Publication Nos.: WO 2010/0031 18 and 201 1/090754, or an anti-GITR antibody described, e.g., in U.S. Pat. No. 7,025,962, European Patent No.: 1947183B1, U.S. Pat. Nos.

U.S. Pat. Nos. 7,812,135, 8,388,967, 8,591,886, European Patent No.: EP 1866339, PCT Publication No.: WO 201 1/028683, PCT Publication No.: WO 2013/039954, PCT Publication No.: WO2005/007190, PCT Publication No.: WO 2007/133822, PCT

Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/1 15451, U.S. Pat. No. 7,618,632, and PCT Publication No.: WO 2011/051726.

In one embodiment, the immunomodulator used is a soluble ligand (e.g., a CTLA-4-Ig), or an antibody or antibody fragment that binds to PD-L1, PD-L2 or CTLA4. For example, the anti-PD-1 antibody molecule can be administered in combination with an anti-CTLA-4 antibody, e.g., ipilimumab, for example. Exemplary anti-CTLA4 antibodies include Tremelimumab (IgG2 monoclonal antibody available from Pfizer, formerly known as ticilimumab, CP-675,206); and Ipilimumab (CTLA-4 antibody, also known as MDX-010, CAS No. 477202-00-9).

When administered as a combination therapy, the agents can be administered at different times or simultaneously as part of the same formulation or separately as different formulations.

“Aging” refers to degeneration of organs and tissues over time, in part due to inadequate replicative capacity in stem cells that regenerate tissues over time. Aging may be due to natural disease processes that occur over time, or those that are driven by cell intrinsic or extrinsic pressures that accelerate cellular replication and repair. Such pressures include natural chemical, mechanical, and radiation exposure; biological agents such as bacteria, viruses, fungus, and toxins; autoimmunity, medications, chemotherapy, therapeutic radiation, cellular therapy. As the telomere is an important factor in aging and disease development, the compounds described herein can be used for treating, mitigating, or minimizing the risk of, a disorder associated with aging (and/or one or more symptoms of a disorder associated with aging) in a subject.

“Disorders associated with aging” or “age-related diseases” refer to disorders that are associated with the ageing process. Exemplary age related diseases include, e.g., Other telomere associated diseases or disorders include glaucoma, cataracts, diabetes mellitus (e.g., type 2 diabetes) osteoarthritis, macular degeneration, rheumatoid arthritis, sarcopenia, cardiovascular diseases such as hypertension, atherosclerosis, coronary artery disease and ischemia/reperfusion injury, osteoporosis, osteonecrosis, inflammatory bowel disease, as well as age-related decline in cognitive function, cardiopulmonary function, muscle strength, vision, and hearing. Another embodiment of the invention is a method of treating a subject with one of the aforementioned diseases by administering an effective amount of one of the disclosed PAPD5 and/or PAPD7 inhibitors (WO2020/051375).

Yet another embodiment of the invention is a method of treating a subject with Revesz syndrome, Coats plus syndrome by administering an effective amount of one of the disclosed PAPD5 and/or PAPD7 inhibitors (WO2020/051375).

PAPD7/TENT4A, has been demonstrated to regulate translesion DNA synthesis in tumor cells (osteosarcoma, breast cancer), in which error-prone DNA polymerases bypass unrepaired DNA lesions (Swain et al., Int. J. Mol. Sci. 22:6957 [2021]). The authors showed that TENT4A regulates mRNA stability and/or translation of DNA polymerase η and RAD18 E3 ligase, which guides the polymerase to replication stalling sites and mono-ubiquitinates PCNA, thereby enabling recruitment of error-prone DNA polymerases to damaged DNA sites. As such, PAPD7 inhibitors may have therapeutic benefit in treating various cancers, sensitizing tumor cells to DNA damaging chemotherapy and/or irradiation.

The disclosed PAPD7 inhibitions can be used for treating pre-leukemic conditions, pre-cancerous conditions, dysplasia and/or cancers. Pre-leukemic conditions include, e.g., Myelodysplastic syndrome, and smoldering leukemia. Dysplasia refers to an abnormality of development or an epithelial anomaly of growth and differentiation, including e.g., hip dysplasia, fibrous dysplasia, and renal dysplasia, Myelodysplastic syndromes, and dysplasia of blood-forming cells.

A precancerous condition or premalignant condition is a state of disordered morphology of cells that is associated with an increased risk of cancer. If left untreated, these conditions may lead to cancer. Such conditions can be dysplasia or benign neoplasia.

Cancers that can be treated using the compounds disclosed herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, endometrial cancer, prostate cancer and/or testicular tumors, non-small cell carcinoma of the lung, cancer of the small intestine and cancer of the esophagus.

In some embodiments, the compounds disclosed herein are used for treating a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. Cancers treatable using the methods described herein are cancers that have increased levels of TERC, an increased expression of genes such as TERC and/or TERT, or increased activity of a telomerase relative to normal tissues or to other cancers of the same tissues.

In some embodiments, agents that decrease the level or activity of TERC (e.g., PAPD5/7 inhibitors) are used to treat cancer. In some embodiments, these agents are used in combination

with other cancer treatments, e.g., chemotherapies, surgery, or radiotherapy.

### Administration

The compositions described herein may be administered systemically or locally, e.g. orally (including, but not limited to solid dosage forms including hard or soft capsules (e.g. gelatin capsules), tablets, pills, powders, sublingual tablets, troches, lozenges, and granules; and liquid dosage forms including, but are not limited to, pharmaceutically acceptable emulsions, microemulsions, aqueous or oil solutions, suspensions, syrups and elixirs, by inhalation (e.g. with an aerosol, gas, inhaler, nebulizer or the like), to the ear (e.g. using ear drops), topically (e.g. using creams, gels, inhalants, liniments, lotions, ointments, patches, pastes, powders, solutions, sprays, transdermal patches, etc.), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc.), via ear drops, via an implanted reservoir or the like, or parenterally depending on the severity and type of the disease being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The terms “patient”, “subject” and “individual” are used interchangeably herein, and refer to an animal, particularly a human, to whom treatment is provided. The term “subject” as used herein refers to human and non-human animals, such as apes, monkeys, horses, cattle, sheep, goats, dogs, cats, rabbits, guinea pigs, rats, and mice. In one embodiment, the subject is human.

The terms “administer”, “administering” or “administration” in reference to a compound, composition or dosage form of the disclosure means introducing the compound into the system of the subject or patient in need of treatment. When a compound of the disclosure is provided in combination with one or more other active agents, “administration” and its variants are each understood to include concurrent and/or sequential introduction of the compound and the other active agents.

The term “treat”, “treatment” or “treating” refers to alleviation of the symptoms of the disease or disorder being treated, inhibition or a delay in the recurrence of symptoms of the disease or of the disease itself or an increase in the longevity of the subject compared with the absence of the treatment, i.e., therapeutic treatment. “Treat”, “treatment” or “treating” also refers to reducing the likelihood of developing a disease or disorder in a subject known to be at risk of developing the disease or disorder, e.g., a subject with a gene mutation which predisposes the subject to the disease.

The term “therapeutically effective amount” means that an amount of an active compound that elicits the desired biological response in a subject, e.g., “treating” a disease or disorder in a subject. Determination of the therapeutically effective amount is well within the capability of those skilled in the art, especially in light of the detailed disclosure provided herein. Toxicity and therapeutic efficacy of a compound of the present disclosure can be determined by standard pharmaceutical procedures in cell cultures and in experimental animals. The therapeutically effective amount of a compound of the present disclosure or other therapeutic agent to be administered to a subject will depend on the stage, category and status of the disease and characteristics of the subject, such as general health, age, sex, body weight and drug tolerance. The therapeutically effective amount of the compound of the present disclosure or other therapeutic agent to be administered will also depend on administration route and dosage form. Dosage amount and interval can be adjusted individually to provide plasma levels of the active compound that are sufficient to maintain desired therapeutic effects. A therapeutically

effective amount typically ranges from 1 μg to 1000 mg.

### IV. Exemplification

The invention is illustrated by the following examples, which are not intended to be limiting.

### A. Abbreviations and Acronyms

Abbreviations and acronyms used herein include the following:
d: doublet
dd: doublet of doublet
m: multiplet
s: singlet
t: triplet
q: quartet
brs/bs: broad singlet
dd: doublet of doublet
td: triplet of doublet
dt: doublet of triplet
DEAD: diethyl azadicarboxylate
DCM: dichloromethane
DIPEA: N,N-diisopropylethylamine
DMF: N, N-dimethyl formamide
DMSO: dimethyl sulfoxide
eq: equivalent (s)
g: gram (s)
h: hour(s)
NaH: sodium hydride
THF: tetrahydrofuran
BBr₃: boron tribromide
CDI: carbonyl diimidazole
EtOAc: ethyl acetate
ACN: acetonitrile
TLC: thin layer chromatography
RT: room temperature
° C.: degree Celsius
mmol: millimoles
μmol: micromoles
ESI: electrospray ionization
EtOAc: ethyl acetate
HCl: hydrochloric acid
IPA: isopropyl alcohol
K₂CO₃: potassium carbonate
LiOH: lithium hydroxide
MeOH: methanol
MS: mass spectrometry
RBF: round bottomed flask
TEA: triethyl amine

### B. General Analytical Methods

TABLE-1
LC-MS conditions

| Cond. # | Conditions |
|---|---|
| 1 | Column: Luna omega polar c18 (2.1*50 mm*1.8μ) • Mobile phase-A: 0.05% |
| | Formic acid in Aqueous, Mobile phase-B: 0.05% Formic acid in |
| | Acetonitrile:Methanol (1:1), Wavelength PDA, Sample concentration: 0.5 mg/mL • Gradient |
| | (T/% B): 0/2, 2/50, 2.8/80, 4/80, 4.2/2, Run time: 5.3 minutes, Diluent: A:B (9:1) |
| 2 | Column: Waters BEH C18(50*2.1 mm*1.7μ) • Mobile phase-A: 0.01% Formic |
| | acid in Aqueous, Mobile phase-B: Acetonitrile, Wavelength: PDA, Sample |
| | concentration: 0.5 mg/mL • Gradient (T/% B): 0/10, 3.0/90, 4.5/90, 4.6/10 • Run |
| | time: 5.6 minutes. Diluent: A:B (7:3) |
| 3 | Column: Luna C8(50*4.6 mm*3.0μ) • Mobile phase-A: 0.01% TFA in Aqueous, |
| | Mobile phase-B: Acetonitrile, Wavelength: PDA, Sample concentration: 0.5 |
| | mg/mL, Gradient (T/% B): 0/50, 1.8/95, 8.0/95, 8.1/50 • Run time: 9.5 minutes. |
| | Diluent: A:B (1:1) |

TABLE-2
Prep. HPLC conditions

| Cond. # | Conditions |
|---|---|
| 1 | Column: X bridge Amide (250*19.0 mm*5.0μ) • Mobile phase A: 5 mM |
| | Ammonium bi carbonate in Aqueous • Mobile phase-B: Acetonitrile:Methanol |
| | (1:1) • Wavelength: PDA • Gradient (T/% B): 0/10, 10/50, 15/80, 20/95 • Flow: |
| | 16.0 mL/min. |
| 2 | Column: kinetix-C18 (250*21.2 mm*5.0μ), Mobile phase-A: 0.1% TFA in |
| | Aqueous, Mobile phase-B: Acetonitrile, Wavelength: PDA, Gradient (T/% B): |
| | 0/30, 12/50, 15/95, 20/95, 20.1/30, Flow: 16.0 mL/min; diluent: ACN/THF |
| 3 | Column: X bridge-C18 (250*19.0 mm*3μ), Mobile phase-A: 0.01% TFA in |
| | Aqueous, Mobile phase-B: Acetonitrile, Wavelength: PDA, Sample |
| | concentration: 30 mg/mL, Gradient (T/% B): 0/20, 10/35, 25/66, Flow: 20.0 |
| | mL/min; diluent: Acetonitrile/TFA/THF |
| 4 | Column: Gemini-C18 (250*19.0 mm*3μ), Mobile phase-A: 0.01% Formic acid |
| | in Aqueous, Mobile phase-B: Acetonitrile, Wavelength: PDA, Sample |
| | concentration: 30 mg/mL, Gradient (T/% B): 0/20, 8/60, 15/68, 35/95, Flow: 20.0 |
| | mL/min; diluent: Acetonitrile/Water/THF |
| 5 | Column: Kinetex, C-18, Mobile phase-A: 0.01% Formic acid in Aqueous, |
| | Mobile phase-B: B:ACN + MeOH (50:50), Wavelength: PDA, Sample |
| | concentration: 30 mg/mL, Gradient (T/% B): 0/40, 14/75, 25/95, Flow: 20.0 |
| | mL/min; diluent: Acetonitrile/MeOH/THF. |
| 6 | Column: YMC Triart, C-18, Mobile phase-A: 0.01% TFA in Aqueous, Mobile |
| | phase-B: ACN, Wavelength: PDA, Sample concentration: 30 mg/mL, Gradient |
| | (T/% B): 0/30, 15/75, 25/85 @ 14.0 ml/min; diluent: Acetonitrile/H2O. |
| 7 | Column: Gemini-C18 (250*19.0 mm*3μ), Mobile phase-A: 0.01% TFA in |
| | Aqueous, Mobile phase-B: Acetonitrile, (T/% B): 0/20, 15/40, 25/95, Flow: 20.0 |
| | mL/min; diluent: Acetonitrile/Water |
| 8 | Column: Luna-C18 (250*21.2 mm*5μ), Mobile phase-A: 0.01% FA in Aqueous, |
| | Mobile phase-B: Acetonitrile, (T/% B):, 0/25, 10/50, 20/60, 25/95, Flow: 20.0 |
| | mL/min; diluent: Acetonitrile/Water/THA/TFA |
| 9 | Column: Gemini-C18 (250*19.0 mm*3μ), Mobile phase-A: 100% water, Mobile |
| | phase-B: Acetonitrile, Wavelength: PDA, Sample concentration: 30 mg/mL, |
| | Gradient (T/% B): 0/20, 8/60, 15/68, 35/95, Flow: 20.0 mL/min; diluent: |
| | Acetonitrile/Water/THF |
| 10 | Column: POROSHELL C-18, 3.0*50 mm, 2.7 μm; flow: 0.6 ml/min Mobile |
| | Phase-A: 0.01% FA IN Water Mobile Phase-B: 100% ACN; Program (Time/% B): |
| | 0.01/5, 6.00/90, 8.00/90, 10.01; diluent: Acetonitrile/Water/THF |
| 11 | Column: ACQUITY BEH C-18, 250 × 212 mm, 5 mm; flow rate: 0.4 mL/min; |
| | Program (Time/% of B); 0/3, 3/95, 6/95, 6.01/3, 6.50/3, Mobile phase-A: 0.1% |
| | Formic acid in water, mobile phase-B: ACN; Solubility: ACN + H2O |
| 12 | Column: AtlantisT3 C-18; (250 × 19 mm) 7 μm, Mobile phase A: H2O, Mobile |
| | phase B: 100% ACN; Program (time/% of B) 0/65, 10/80, 15/25, 20/95@16 ml/min; |
| | flow rate: 0.4 mL/min; Solubility: ACN, THF, H2O. |
| 13 | Column: Agilent C18; Solubility: ACN, water, THF, FA; Program/method: 0/5, |
| | 15/45, 25/85 @12 ml, Mobile phase-A: 0.1% of FA in water, mobile phase-B: |
| | ACN; loading: 8 mg; flow rate: 0.4 mL/min; |

### C. Synthesis of Exemplified Compounds

### Example 1: Synthesis of 1-(6-aminopyridin-3-yl)-6-chloro-7-(5,7-dihydro-6H-pyrrolo[3,4-b]pyridin-6-yl)-4-oxo-1,4-dihydroquinoline-3-carboxylic acid

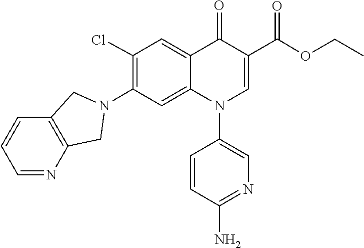 [SMILES: CCOC(=O)c1cn(-c2ccc(N)nc2)c2cc(N3Cc4cccnc4C3)c(Cl)cc2c1=O]

### Step-1. Synthesis of Ethyl 6-chloro-1-(6-acetamidopyridin-3-yl)-7-fluoro-4-oxoquinoline-3-carboxylate

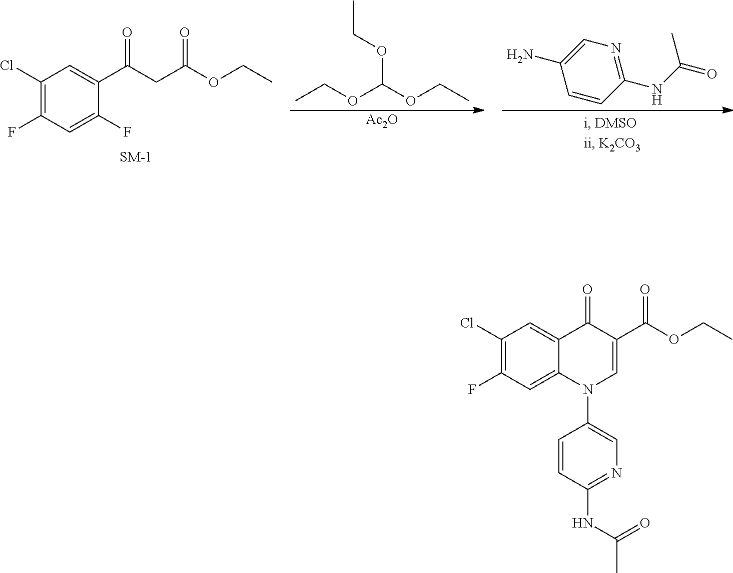

To a mixture of ethyl 3-(5-chloro-2,4-difluorophenyl)-3-oxopropanoate (1.0 g, 3.81 mmol, 1.0 equiv) in acetic anhydride (1.2 g, 11.42 mmol, 3.0 equiv) was added triethyl orthoformate (846 mg, 5.71 mmol, 1.5 equiv). The resulting mixture was stirred at 100° C. for 2 h. The resulting mixture was concentrated under vacuum. To the resulting mixture in DMSO (25 mL) was added N-(5-aminopyridin-2-yl)acetamide (576 mg, 3.81 mmol, 1.0 equiv). The resulting mixture was stirred at 25° C. for 2 h. To the resulting mixture was added K₂CO₃ (263 mg, 1.90 mmol, 0.5 equiv). The resulting mixture was stirred at 100° C. for 1 h. The reaction was quenched by the addition of 35 mL water. The precipitated solids were collected by filtration. This resulted in ethyl 6-chloro-1-(6-acetamidopyridin-3-yl)-7-fluoro-4-oxoquinoline-3-carboxylate (800 mg, 52%) as a yellow solid. LCMS (ESI) [M+H]⁺: 404.1.

### Step-2. Synthesis of Ethyl 6-chloro-1-(6-acetamidopyridin-3-yl)-4-oxo-7-[5H,7H-pyrrolo[3,4-b]pyridin-6-yl]quinoline-3-carboxylate

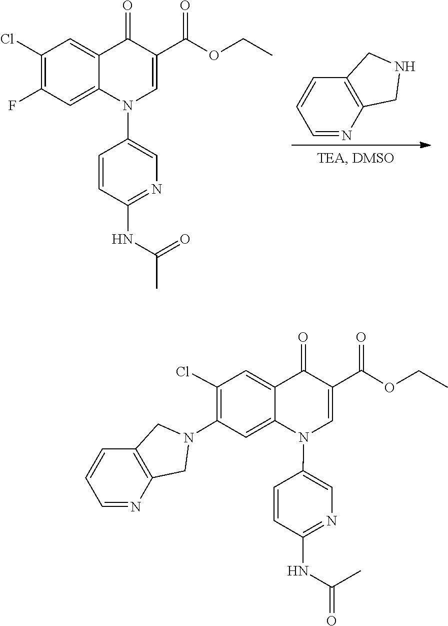 [SMILES: CCOC(=O)c1cn(-c2ccc(NC(C)=O)nc2)c2cc(F)c(Cl)cc2c1=O ; c1cnc2c(c1)CNC2 ; CCOC(=O)c1cn(-c2ccc(NC(C)=O)nc2)c2cc(N3Cc4cccnc4C3)c(Cl)cc2c1=O]

To a mixture of ethyl 6-chloro-1-(6-acetamidopyridin-3-yl)-7-fluoro-4-oxoquinoline-3-carboxylate (200 mg, 0.50 mmol, 1.0 equiv) in DMSO (5 mL) was added Et₃N (150 mg, 1.49 mmol, 3.0 equiv) and 5H,6H,7H-pyrrolo[3,4-b]pyridine (89 mg, 0.74 mmol, 1.5 equiv). The resulting mixture was stirred for 1.5 h at 80° C. under nitrogen atmosphere. The mixture was allowed to cool down to room temperature. The resulting mixture was concentrated under vacuum. The crude product was purified by Prep-HPLC with the following conditions (Column: XBridge Shield RP18 OBD Column, 5 um, 19*150 mm; Mobile Phase A:Water (10 MMOL/L NH₄HCO₃), Mobile Phase B: ACN; Flow rate: 25 mL/min; Gradient: 50% B to 80% B in 8 min; 220 nm nm; Rt: 8.2 min). This resulted in ethyl 6-chloro-1-(6-acetamidopyridin-3-yl)-4-oxo-7-[5H,7H-pyrrolo[3,4-b]pyridin-6-yl]quinoline-3-carboxylate (55 mg, 22%) as a yellow solid. LCMS (ESI) [M+H]⁺: 504.1.

### Step-3. Synthesis of 1-(6-Aminopyridin-3-yl)-6-chloro-4-oxo-7-[5H,7H-pyrrolo[3,4-b]pyridin-6-yl]quinoline-3-carboxylic acid

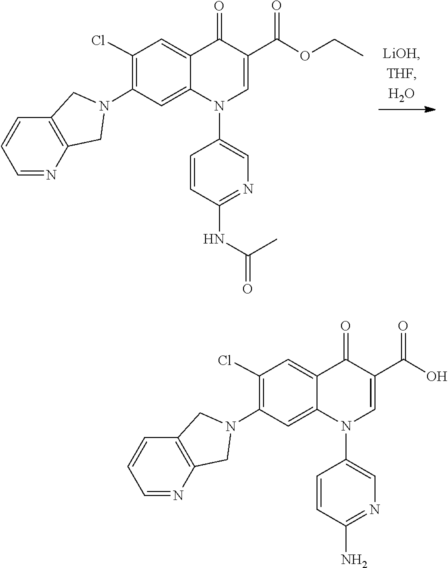 [SMILES: CCOC(=O)c1cn(-c2ccc(NC(C)=O)nc2)c2cc(N3Cc4cccnc4C3)c(Cl)cc2c1=O ; Nc1ccc(-n2cc(C(=O)O)c(=O)c3cc(Cl)c(N4Cc5cccnc5C4)cc32)cn1]

To a mixture of ethyl 6-chloro-1-(6-acetamidopyridin-3-yl)-4-oxo-7-[5H,7H-pyrrolo[3,4-b]pyridin-6-yl]quinoline-3-carboxylate (50 mg, 0.10 mmol, 1.0 equiv) in THF (2 mL) was added LiOH (19 mg, 0.79 mmol, 8.0 equiv) and H₂O (1 mL). The resulting mixture was stirred for 1.5 h at 70° C. under nitrogen atmosphere. The mixture was allowed to cool down to room temperature. The resulting mixture was concentrated under vacuum. The mixture was neutralized to pH 5 with HCl (aq.). The crude product was purified by Prep-HPLC with the following conditions (Column: SunFire Prep C₁₈ OBD Column, 19*150 mm 5 um 10 nm; Mobile Phase A:Water (0.1% FA), Mobile Phase B:ACN; Flow rate:25 mL/min; Gradient:15 B to 42 B in 8 min; 254/220 nm; RT1:9.28; RT2:; Injection Volume: ml; Number Of Runs:;). This resulted in 1-(6-aminopyridin-3-yl)-6-chloro-4-oxo-7-[5H,7H-pyrrolo[3,4-b]pyridin-6-yl]quinoline-3-carboxylic acid (6.3 mg, 14.64%) as a light yellow solid. LCMS (ESI) [M+H]⁺: 433.95. ¹H NMR (300 MHz, DMSO-d6) δ 15.169 (s, 1H), 8.60-8.38 (m, 2H), 8.27-8.16 (m, 2H), 7.85 (d, J=7.6 Hz, 1H), 7.68 (s, 1H), 7.32 (d, J=2.9 Hz, 1H), 6.71-6.60 (m, 3H), 6.41 (s, 1H), 4.94 (d, J=24.6 Hz, 4H), 2.08 (s, 1H).

### Example-2: 6-chloro-4-oxo-1-(pyrazin-2-yl)-7{5H,6H,7H pyrrolo[3,4-b]pyridin-6-yl}-1,4-dihydro-1,8-naphthyridine-3-carboxylic acid

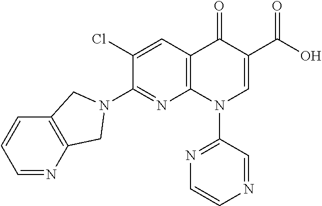 [SMILES: O=C(O)c1cn(-c2cnccn2)c2nc(N3Cc4cccnc4C3)c(Cl)cc2c1=O]

### Step-1: Synthesis of ethyl 3-oxo-3-(2,5,6-trichloropyridin-3-yl)propanoate

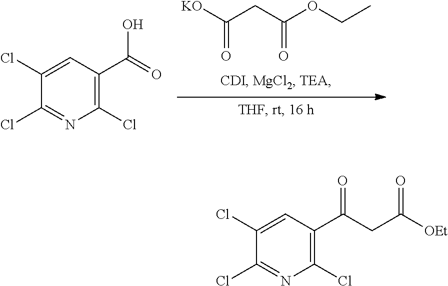

The stirred solution of 2,5,6-trichloropyridine-3-carboxylic acid (20 g, 88.3 mmol) in THF (150 mL), was added carbonyl diimidazole (28.5 g, 176 mmol) in THF (150 mL) and stirred for 4 h, after disappearance of SM on TLC, added triethylamine (36.7 mL, 264 mmol), magnesium chloride (6.72 g, 70.6 mmol) followed by 1-ethyl 3-potassium propanedioate (17.8 g, 105 mmol) and the reaction mixture was stirred at room temperature for 12 h. After completion of the starting material, the reaction mixture was quenched with 1N HCl and pH was adjusted to 2 then extracted with ethyl acetate. The combined organic layers were dried over sodium sulfate and concentrated under reduced pressure, the crude compound was purified by column chromatography using 100-200 silica gel mesh, the desired product was eluted with 5-10% EtOAc in Hexane to afford ethyl 3-oxo-3-(2,5,6-trichloropyridin-3-yl)propanoate (12 g, 40.4 mmol, 39%) as white solid. MS (ESI): m/z 294.0 [M−H]⁻.

### Step-2: Synthesis of ethyl (2Z)-3-ethoxy-2-[(Z)-2,5,6-trichloropyridine-3-carbonyl]prop-2-enoate

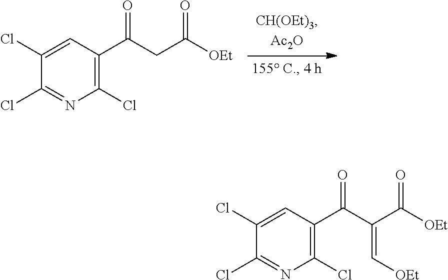 [SMILES: CCOC(=O)CC(=O)c1cc(Cl)c(Cl)nc1Cl ; CCO/C=C(\C(=O)OCC)C(=O)c1cc(Cl)c(Cl)nc1Cl]

To a stirred solution of ethyl 3-oxo-3-(2,5,6-trichloropyridin-3-yl)propanoate (10 g, 33.7 mmol) in acetic anhydride (30.0 mL, 268 mmol) at 25° C., was added triethyl orthoformate (30.0 mL, 180 mmol) and stirred the reaction mixture at 155° C. for 4 h. After completion of reaction, Excess of solvents were removed and the residue was azeotroped with toluene thrice to afford ethyl (2Z)-3-ethoxy-2-[(Z)-2,5,6-trichloropyridine-3-carbonyl]prop-2-enoate (10.0 g, 28.3 mmol, 84%) as pale brown gummy. The material was directly taken for next step without further purification.

### Step-3: Synthesis of ethyl 6,7-dichloro-4-oxo-1-(pyrazin-2-yl)-1,4-dihydro-1,8-naphthyridine3-carboxylate

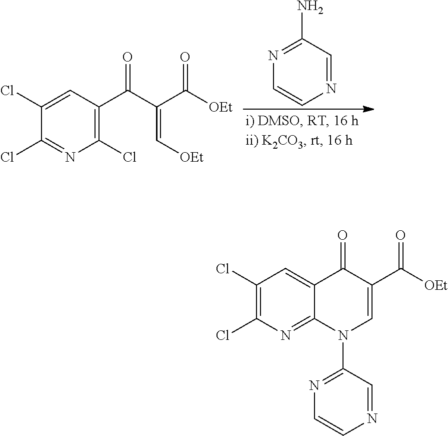 [SMILES: CCO/C=C(\C(=O)OCC)C(=O)c1cc(Cl)c(Cl)nc1Cl ; Nc1cnccn1 ; CCOC(=O)c1cn(-c2cnccn2)c2nc(Cl)c(Cl)cc2c1=O]

To a stirred solution of ethyl (2Z)-3-ethoxy-2-[(Z)-2,5,6-trichloropyridine-3-carbonyl]prop-2-enoate (8.0 g, 22.6 mmol) in DMSO (100 mL), was added pyrazin-2-amine (2.14 g, 22.6 mmol) and stirred the reaction mixture at rt for 16 h under N₂ atmosphere. Then, potassium carbonate (6.24 g, 45.2 mmol) was added and the resulting mixture was stirred at rt for 1 h. After completion of reaction, the reaction mixture was quenched with ice-cold water and precipitated solids were collected by filtration. The solid compound was washed with ice cold water followed by diethyl ether, dried under vacuum to afford ethyl 6,7-dichloro-4-oxo-1-(pyrazin-2-yl)-1,4-dihydro-1,8-naphthyridine3-carboxylate (8.0 g, 21.9 mmol, 97%) as an off white solid. MS (ESI): m/z 365.0 [M+H]⁺.

### Step-4: Synthesis of ethyl 6-chloro-4-oxo-1-(pyrazin-2-yl)-7-{5H,6H,7H-pyrrolo[3,4-b]pyridin-6-yl}-1,4-dihydro-1,8-naphthyridine-3-carboxylate

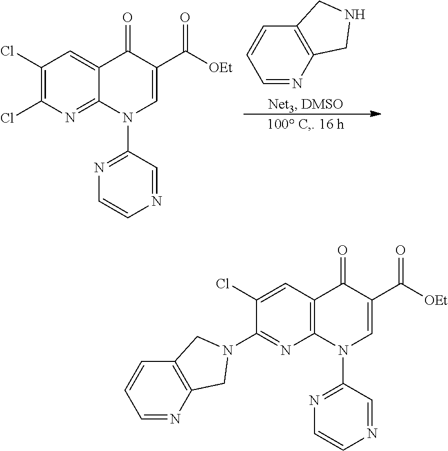

A mixture of ethyl 6,7-dichloro-4-oxo-1-(pyrazin-2-yl)-1,4-dihydro-1,8-naphthyridine-3 carboxylate (10 g, 27.3 mmol) and 5H,6H,7H-pyrrolo[3,4-b]pyridine di HCl (4.25 g, 35.4 mmol) in DMSO (5 mL), was added triethylamine (18.8 mL, 136 mmol) in a sealed tube and heated to 120° C. for 6 h. The reaction mixture was cooled to rt and quenched with ice cold water, stirred for 10 min. Precipitated solid was filtered and washed with diethyl ether, dried under vacuum to afford ethyl 6-chloro-4-oxo-1-(pyrazin-2-yl)-7-{5H,6H,7H-pyrrolo[3,4-b]pyridin-6-yl}-1,4-dihydro1,8-naphthyridine-3-carboxylate (10.0 g, 22.2 mmol, 82%) as an off white solid.

MS (ESI): m/z 448.8 [M+H]⁺.

### Step-5: Synthesis of 6-chloro-4-oxo-1-(pyrazin-2-yl) {5H,6H,7H pyrrolo[3,4-b]pyridin-6-yl}-1,4-dihydro-1,8-naphthyridine-3-carboxylic acid

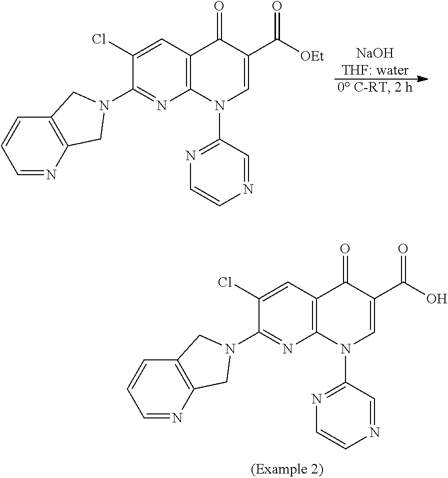 [SMILES: CCOC(=O)c1cn(-c2cnccn2)c2nc(N3Cc4cccnc4C3)c(Cl)cc2c1=O ; O=C(O)c1cn(-c2cnccn2)c2nc(N3Cc4cccnc4C3)c(Cl)cc2c1=O]

To a stirred solution of ethyl 6-chloro-4-oxo-1-(pyrazin-2-yl)-7-{5H,6H,7H pyrrolo[3,4b] pyridin-6-yl}-1,4-dihydro-1,8-naphthyridine-3-carboxylate (3 g, 6.68 mmol) in THF (120 mL) & Water (60 mL), was added sodium hydroxide (799 mg, 20.0 mmol) at 0° C. Then, reaction mixture was allowed to room temperature for 6 h. The reaction mixture was re-cooled with ice-bath, diluted with water (15 mL), pH was adjusted to 4-5 by using 1N HCl which resulted in precipitation of solids. The precipitated solid was collected by filtration, triturated with 10% MeOH in DCM (2×100 mL), dried under vacuum and lyophilized to afford 6-chloro-4-oxo-1-(pyrazin-2-yl)-7 {5H,6H,7H pyrrolo[3,4-b]pyridin-6-yl}-1,4-dihydro-1,8-naphthyridine-3-carboxylic acid (2.47 g, 5.86 mmol, 88%) as pale brown solid.

TLC System: 10% MeOH in DCM; Rf: 0.2. ¹H-NMR (400 MHz, DMSO-d₆): δ 14.70 (bs, 1H), 9.31 (d, J=1.2 Hz, 1H), 9.06 (s, 1H), 8.91 (d, J=2.8 Hz, 1H), 6 8.83-8.81 (m, 1H), 8.46 (d, J=4.8 Hz, 1H), 8.41 (s, 1H), 7.78 (d, J=7.2 Hz, 1H), 7.34-7.30 (m, 1H), 5.17 (bs, 2H), 4.95 (bs, 2H). MS (ESI): m/z 421.0 [M+H]⁺.

Analogues compounds, examples 103 to 106 were synthesized by following similar procedures using appropriate reagentes.

### Example-3: 6-chloro-1-(3-fluoro-4-hydroxyphenyl)-4-oxo-7-{5H,6H,7H-pyrrolo[3,4-b]pyridin-6-yl}-1,4-dihydroquinoline-3-carboxylic acid

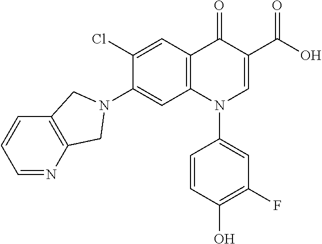 [SMILES: O=C(O)c1cn(-c2ccc(O)c(F)c2)c2cc(N3Cc4cccnc4C3)c(Cl)cc2c1=O]

### Step-1: Synthesis of ethyl 3-(5-chloro-2-fluoro-4-{5H,6H,7H-pyrrolo[3,4-b]pyridin-6-yl}phenyl)-3-oxopropanoate

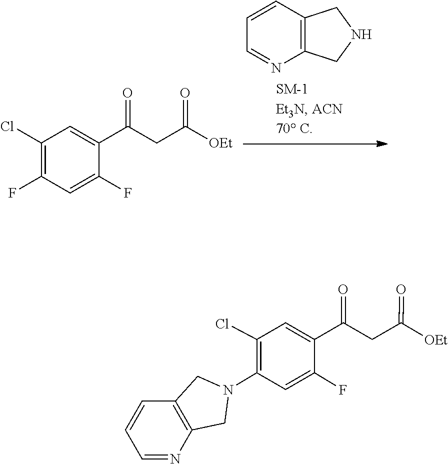 [SMILES: c1cnc2c(c1)CNC2 ; CCOC(=O)CC(=O)c1cc(Cl)c(F)cc1F ; CCOC(=O)CC(=O)c1cc(Cl)c(N2Cc3cccnc3C2)cc1F]

To a stirred solution of ethyl 3-(5-chloro-2,4-difluorophenyl)-3-oxopropanoate (500 mg, 1.90 mmol) in acetonitrile (7 mL) were added triethylamine (528 μL, 3.80 mmol) & 5H,6H,7H-pyrrolo[3,4-b]pyridine (251 mg, 2.09 mmol) at room temperature under inert atmosphere. The resulting mixture was stirred at 70° C. for 16 h. After completion of reaction, it was concentrated under reduced pressure and extracted with EtOAc (2×10 mL). Combined organic fractions were dried over sodium sulfate and concentrated under reduced pressure to get crude product. The crude was purified by column chromatography (100-200 silica gel mesh) eluting at 30% EtOAc in hexanes to afford ethyl 3-(5-chloro-2-fluoro-4-{5H,6H,7H-pyrrolo[3,4-b]pyridin-6-yl}phenyl)-3-oxopropanoate (344 mg, 950 μmol, 50%) as pale brown solid. MS (ESI): m/z 363.1 [M+H]⁺.

### Step-2: Synthesis of ethyl 6-chloro-1-(3-fluoro-4-hydroxyphenyl)-4-oxo-7-{5H,6H,7H-pyrrolo[3,4-b]pyridin-6-yl}-1,4-dihydroquinoline-3-carboxylate

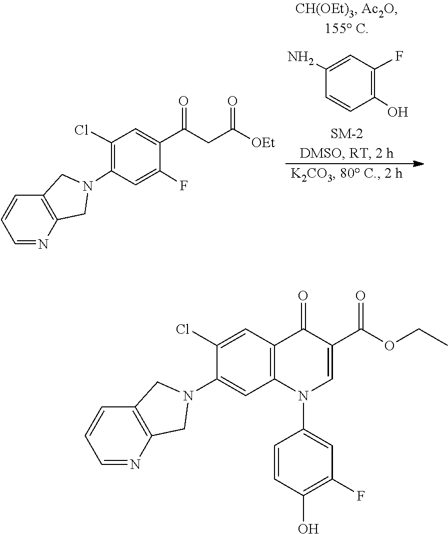 [SMILES: CCOC(=O)CC(=O)c1cc(Cl)c(N2Cc3cccnc3C2)cc1F ; Nc1ccc(O)c(F)c1 ; CCOC(=O)c1cn(-c2ccc(O)c(F)c2)c2cc(N3Cc4cccnc4C3)c(Cl)cc2c1=O]

To a stirred solution of ethyl 3-(5-chloro-2-fluoro-4-{5H,6H,7H-pyrrolo[3,4-b]pyridin-6-yl}phenyl)-3-oxopropanoate (400 mg, 1.10 mmol) in acetic anhydride (103 p L, 1.10 mmol) was added triethyl orthoformate (182 p L, 1.10 mmol) and stirred at 150° C. for 4 h under N₂ atmosphere. The progress of the reaction was monitored by TLC, it indicated consumption of SM. After completion of SM, the reaction mixture was cooled to room temperature, concentrated and codistilled with toluene to get residue. The residue was dissolved in dimethyl sulfoxide (10 mL), was added 4-amino-2-fluorophenol (139 mg, 1.10 mmol) and stirred the reaction mixture at room temperature for 2 h under inert atmosphere. Then added potassium carbonate (152 mg, 1.10 mmol) to the reaction mixture. The resulting mixture was heated to 80° C. and stirred for 2 h. After completion of the reaction (Monitored by TLC), the reaction mixture was cooled to room temperature, quenched with ice-cold water (5 mL), which results in solid precipitatation. The precipitated solid was collected by filtration and washed with water& hexane to afford ethyl 6-chloro-1-(3-fluoro-4-hydroxyphenyl)-4-oxo-7-{5H,6H,7H-pyrrolo[3,4-b]pyridin-6-yl}-1,4-dihydroquinoline-3-carboxylate (142 mg, 296 μmol, 27%) as pale brown solid.

MS (ESI): m/z 480.3 [M+H]⁺.

### Step-3: Synthesis of 6-chloro-1-(3-fluoro-4-hydroxyphenyl)-4-oxo-7-{5H,6H,7H-pyrrolo[3,4-b]pyridin-6-yl}-1,4-dihydroquinoline-3-carboxylic acid

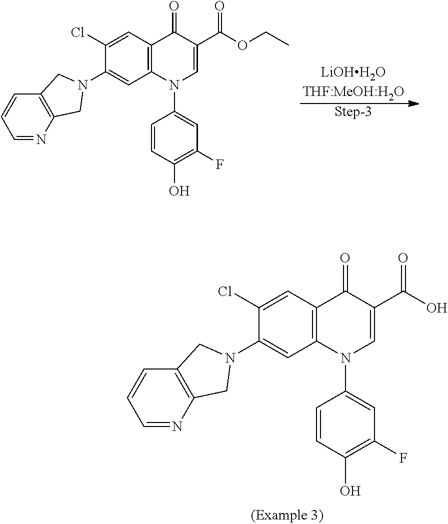 [SMILES: CCOC(=O)c1cn(-c2ccc(O)c(F)c2)c2cc(N3Cc4cccnc4C3)c(Cl)cc2c1=O ; O=C(O)c1cn(-c2ccc(O)c(F)c2)c2cc(N3Cc4cccnc4C3)c(Cl)cc2c1=O]

To a stirred solution of ethyl 6-chloro-1-(3-fluoro-4-hydroxyphenyl)-4-oxo-7-{5H,6H,7H-pyrrolo[3,4-b]pyridin-6-yl}-1,4-dihydroquinoline-3-carboxylate (120 mg, 250 μmol) in a mixture of THF (2 mL): MeOH (2 mL), was added a solution of lithium (1+) hydrate hydroxide (66.7 mg, 1.59 mmol) in H₂O (2 mL). The resulting mixture was stirred at room temperature for 6 h. After completion of the reaction (Monitored by TLC), the reaction mixture was concentrated under reduced pressure until to remove THF and MeOH. The aqueous layer was washed with EtOAc to remove impurities then acidified with 1N HCl (pH>2). The precipitated solid was collected by filtration and washed with H2O & hexane to afford 6-chloro-1-(3-fluoro-4-hydroxyphenyl)-4-oxo-7-{5H,6H,7H-pyrrolo[3,4-b]pyridin-6-yl}-1,4-dihydroquinoline-3-carboxylic acid (38.8 mg, 85.9 μmol, 35%) as grey solid.

¹H NMR (400 MHz, DMSO-d₆) δ 15.11 (brs, 1H), 10.68 (s, 1H), 8.57 (s, 1H), 8.46 (d, J=4.4 Hz, 1H), 8.24 (s, 1H), 7.83 (d, J=7.2 Hz, 1H), 7.67 (dd, J=8.0, 2.8 Hz, 1H), 7.38-7.36 (m, 2H), 7.31 (t, J=2.8 Hz, 1H), 6.38 (s, 1H), 4.99-4.83 (m, 4H); MS (ESI: m/z 452.3[M+H]⁺.

### Examples 4 and 5

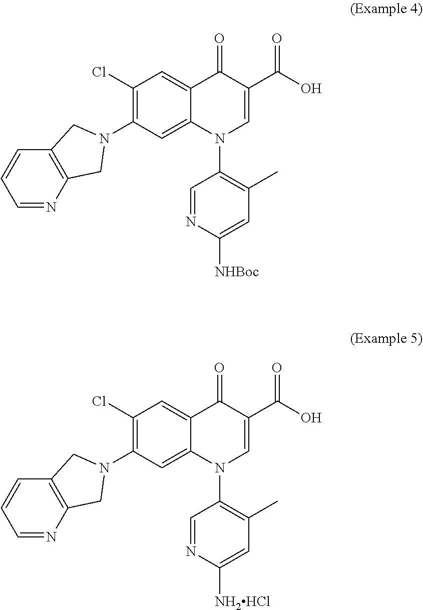

### Step-1: Synthesis of tert-butyl N-(4-methyl-5-nitropyridin-2-yl)carbamate

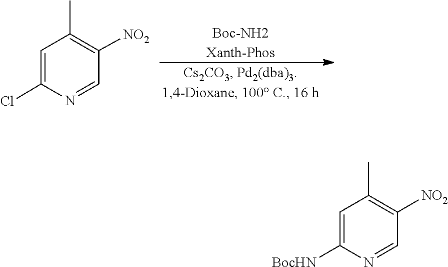 [SMILES: Cc1cc(Cl)ncc1[N+](=O)[O-] ; Cc1cc(NC(=O)OC(C)(C)C)ncc1[N+](=O)[O-]]

To a stirred solution of 2-chloro-4-methyl-5-nitropyridine (1.0 g, 5.79 mmol) in 1,4-Dioxane (30 mL), were added tert-butyl carbamate (1.01 g, 8.68 mmol), Xantphos (335 mg, 579 μmol), cesium carbonate (4.69 g, 14.4 mmol) at rt and the mixture was degassed with N₂ gas for 10 minutes. Then, tris(benzylideneacetone) dipalladium (264 mg, 289 μmol) was added to reaction mixture and again degassed with N₂ gas for 5 minutes. The reaction mixture was heated to 100° C. and stirred for 16 h. The reaction mixture was cooled to rt, filtered through a celite pad, filterate was concentrated to get crude compound which was purified by silica gel (100-200 mesh) column chromatography by eluting with 5-10% of EtOAc in hexane to afford tert-butyl N-(4-methyl-5-nitropyridin-2-yl) carbamate (800 mg, 3.15 mmol, 54%) as an off white solid.

MS (ESI): m/z 254 [M+H]⁺.

### Step-2: Synthesis of tert-butyl N-(5-amino-4-methylpyridin-2-yl)carbamate

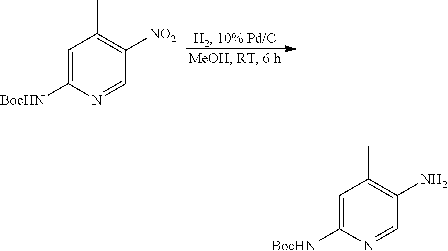 [SMILES: Cc1cc(NC(=O)OC(C)(C)C)ncc1[N+](=O)[O-] ; Cc1cc(NC(=O)OC(C)(C)C)ncc1N]

To a stirred solution of tert-butyl N-(4-methyl-5-nitropyridin-2-yl) carbamate (600 mg, 2.36 mmol) in MeOH (20 mL), was added 10% Pd/C (377 mg, 2.36 mmol) at inert atmosphere, reaction was maintained at rt for 3 h under H2(g) atmosphere. After completion of the reaction (monitored by TLC), Pd/C was filtered through a celite pad, filterate was concentrated to afford tert-butyl N-(5-amino-4-methylpyridin-2-yl) carbamate (500 mg, 2.23 mmol, 92%) as an off white solid. MS (ESI): m/z 224 [M+H]⁺.

### Step-3: Synthesis of ethyl 3-(5-chloro-2,4-difluorophenyl)-3-oxopropanoate

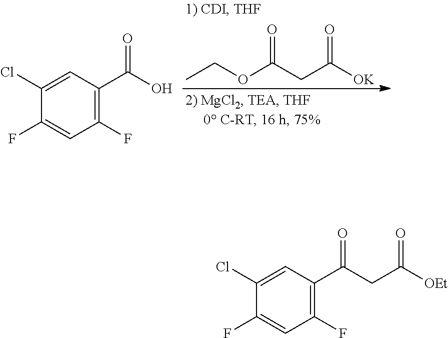

To a stirred solution of 5-chloro-2,4-difluorobenzoic acid (100 g, 503 mmol) in THF (1000 mL), was added CDI (165 g, 1.00 mol) in THF (1000 mL) drop wise at 0° C. The reaction mixture was slowly warmed to room temperature and stirred at same temperature for 1 h. After completion of starting material by TLC, potassium mono ethyl malonate (130 g, 746 mmol), magnesium chloride (38.5 g, 402 mmol) followed by triethylamine (220 mL, 1.57 mol) were added at room temperature and continued the stirring for a period of 16 h. After completion of the reaction by TLC, the reaction mixture was concentrated under reduced pressure. The resulting residue was diluted with water (4000 mL) and extracted with EtOAc (3×3000 mL). The combined organic layers were washed with brine solution (2000 mL), dried over anhydrous sodium sulphate and evaporated to dryness to afford a sticky mass. Further it was triturated with methanol (400 mL) and the precipitated solid was collected by filtration and dried under vacuum to afford ethyl 3-(5-chloro-2,4-difluorophenyl)-3-oxopropanoate (95 g, 75%) as an off white solid. 1H NMR (400 MHz, DMSO-d₆) δ 7.86 (dd, J=8.8, 1.2 Hz, 1H), 7.47 (t, J=10.4 Hz, 1H), 5.13 (s, 1H), 4.01 (q, J=7.2 Hz, 2H), 1.16 (t, J=7.2 Hz, 3H)

Note: Compound generally equilibrates in keto enol form

### Step-4: Synthesis of ethyl (2Z)-2-[(Z)-5-chloro-2,4-difluorobenzoyl]-3-ethoxyprop-2-enoate

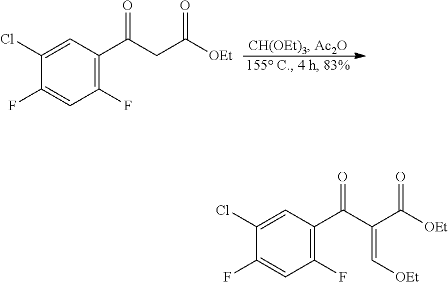 [SMILES: CCOC(=O)CC(=O)c1cc(Cl)c(F)cc1F ; CCO/C=C(\C(=O)OCC)C(=O)c1cc(Cl)c(F)cc1F]

To a stirred solution of ethyl 3-(5-chloro-2,4-difluorophenyl)-3-oxopropanoate (5 g, 19.0 mmol) in acetic anhydride (5.80 g, 56.9 mmol) at 27° C., was added triethyl orthoformate (4.20 g, 28.4 mmol) and stirred the reaction mixture at 155° C. for 4 h. The progress of reaction was monitored by TLC. After completion of reaction, Excess of solvents were removed and azeotrope with toluene to afford ethyl (2Z)-2-[(Z)-5-chloro-2,4-difluorobenzoyl]-3-ethoxyprop-2-enoate (5.00 g, 83%) as pale orange solid. This material was used as such for next step without further purification.

### Step-5: Synthesis of ethyl 1-(6-{[(tert-butoxy)carbonyl]amino}-4-methylpyridin-3-yl)-6-chloro-7-fluoro-4-oxo-1,4-dihydroquinoline-3-carboxylate

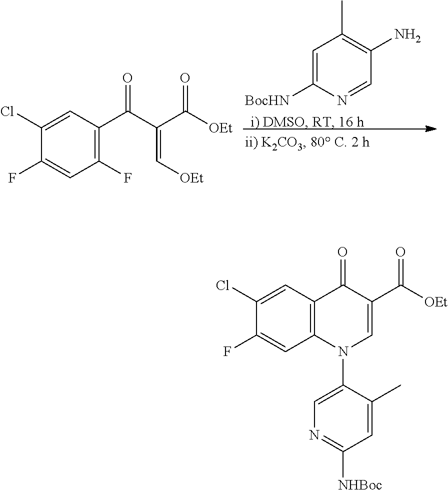 [SMILES: CCO/C=C(\C(=O)OCC)C(=O)c1cc(Cl)c(F)cc1F ; Cc1cc(NC(=O)OC(C)(C)C)ncc1N ; CCOC(=O)c1cn(-c2cnc(NC(=O)OC(C)(C)C)cc2C)c2cc(F)c(Cl)cc2c1=O]

To a stirred solution of ethyl (2Z)-2-[(Z)-3-chloro-4-fluorobenzoyl]-3-ethoxyprop-2-enoate (500 mg, 1.66 mmol) in DMSO (5 mL) was added tert-butyl N-(5-amino-4-methylpyridin-2-yl) carbamate (553 mg, 2.48 mmol) at rt. Reaction was maintained at rt for 16 h. After Imine formation potassium carbonate (342 mg, 2.48 mmol) was added to a reaction mixture, Reaction was maintained at 80° C. and stirred for 16 h. after completion of the reaction (Monitored by TLC), reaction mixture was diluted with water (10 mL) and extracted with 10% MeOH in DCM (2×30 mL). combined organic layers were dried over Na2SO4, filtered and concentrated to get crude compound, Crude was purified by silica gel (100-200 mesh) column chromatography by eluting with 2-5% of MeOH in DCM to afford ethyl 1-(6-{[(tert-butoxy) carbonyl] amino}-4-methylpyridin-3-yl)-6-chloro-7-fluoro-4-oxo-1,4-dihydroquinoline-3-carboxylate (380 mg, 798 μmol, 48%) as an off-white solid. MS (ESI): m/z 476 [M+H]⁺.

### Step-6: Synthesis of ethyl 1-(6-{[(tert-butoxy)carbonyl]amino}-4-methylpyridin-3-yl)-6-chloro-4-oxo-7-{5H,6H,7H-pyrrolo[3,4-b]pyridin-6-yl}-1,4-dihydroquinoline-3-carboxylate

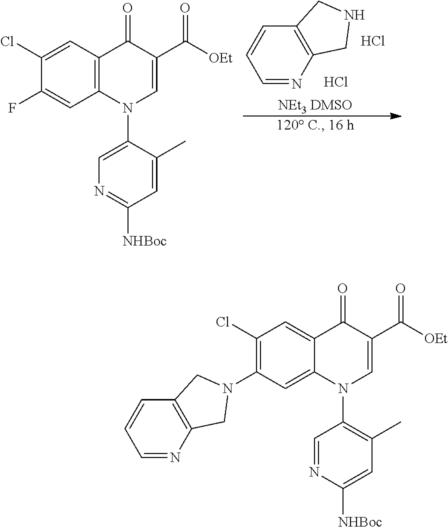

To a stirred solution of ethyl 1-(6-{[(tert-butoxy)carbonyl]amino}-4-methylpyridin-3-yl)-6-chloro-7-fluoro-4-oxo-1,4-dihydroquinoline-3-carboxylate ethyl 1-(6-{[(tert-butoxy)carbonyl]amino}-4-methylpyridin-3-yl)-6-chloro-7-fluoro-4-oxo-1,4-dihydroquinoline-3-carboxylate (350 mg, 735 μmol) 735 μmol) in DMSO (2 mL), were added triethylamine (501 μL, 3.67 mmol) followed by 5H,6H,7H-pyrrolo[3,4-b]pyridine di HCl (176 mg, 1.47 mmol) at rt. The reaction mixture was heated to 120° C. and stirred for 16 h. Then, reaction mixture was cooled to rt, diluted with water (10 mL) and extracted with 10% MeOH in DCM (2×30 mL). The combined organic layers were dried over Na2SO4, filtered and concentrated to get crude compound, crude was purified by silica gel (100-200 mesh) column chromatography by eluting with 5-8% of MeOH in DCM to afford ethyl 1-(6-{[(tert-butoxy)carbonyl]amino}-4-methylpyridin-3-yl)-6-chloro-4-oxo-7-{5H,6H,7H-pyrrolo[3,4-b]pyridin-6-yl}-1,4-dihydroquinoline-3-carboxylate (220 mg, 381 μmol, 52%) as an off white solid.

MS (ESI): m/z 576 [M+H]⁺.

### Step-7: Example 4: Synthesis of 1-(6-{[(tert-butoxy)carbonyl]amino}-4-methylpyridin-3-yl)-6-chloro-4-oxo-7-{5H,6H,7H-pyrrolo[3,4-b]pyridin-6-yl}-1,4-dihydroquinoline-3-carboxylic acid

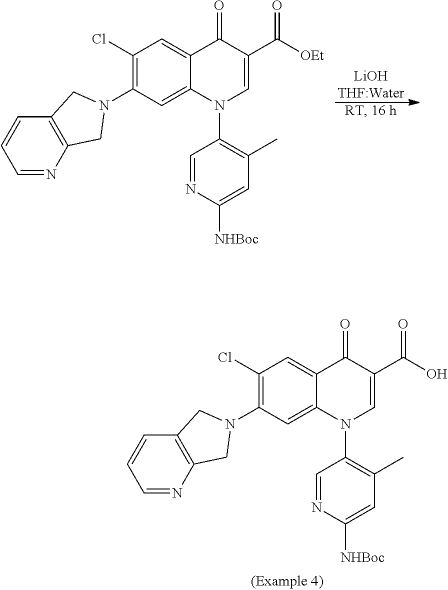 [SMILES: CCOC(=O)c1cn(-c2cnc(NC(=O)OC(C)(C)C)cc2C)c2cc(N3Cc4cccnc4C3)c(Cl)cc2c1=O ; Cc1cc(NC(=O)OC(C)(C)C)ncc1-n1cc(C(=O)O)c(=O)c2cc(Cl)c(N3Cc4cccnc4C3)cc21]

To a stirred solution of ethyl 1-(6-{[(tert-butoxy) carbonyl] amino}-4-methylpyridin-3-yl)-6-chloro-4-oxo-7-{5H,6H,7H-pyrrolo[3,4-b] pyridin-6-yl}-1,4-dihydroquinoline-3-carboxylate (50 mg, 86.7 μmol) in THF (1 mL) and Water (1 mL), was added lithium hydroxide (10.3 mg, 433 μmol) at rt and the mixture was stirred at rt for 16 h. After completion of the reaction (monitored by TLC), solvents were evaporated from reaction mixture, the residue was acidified with 1N HCl, precipitated solid was filtered and dried which was further triturated with MeOH and diethyl ether then, subjected to lyophilization to afford 1-(6-{[(tert-butoxy)carbonyl]amino}-4-methylpyridin-3-yl)-6-chloro-4-oxo-7-{5H,6H,7H-pyrrolo[3,4-b]pyridin-6-yl}-1,4-dihydroquinoline-3-carboxylic acid (13.0 mg, 23.7 μmol, 27%) as an off-white solid. TLC System: 5% MeOH in DCM; Rf: 0.3. ¹H NMR (400 MHz, DMSO-d₆) δ 10.23 (s, 1H), 8.60 (bs, 1H), 8.48-8.37 (m, 2H), 8.24 (s, 1H), 8.03 (s, 1H), 7.82 (d, J=7.2 Hz, 1H), 7.33-7.27 (m, 1H), 6.11 (s, 1H), 4.94-4.80 (m, 4H), 2.08 (s, 3H), 1.52 (s, 9H) (—COOH peak was not observed); MS (ESI): m/z 548 [M+H]⁺.

### Step-8: Example 5: Synthesis of 1-(6-amino-4-methylpyridin-3-yl)-6-chloro-4-oxo-7-{5H,6H,7H-pyrrolo[3,4-b]pyridin-6-yl}-1,4-dihydroquinoline-3-carboxylicacid hydrochloride

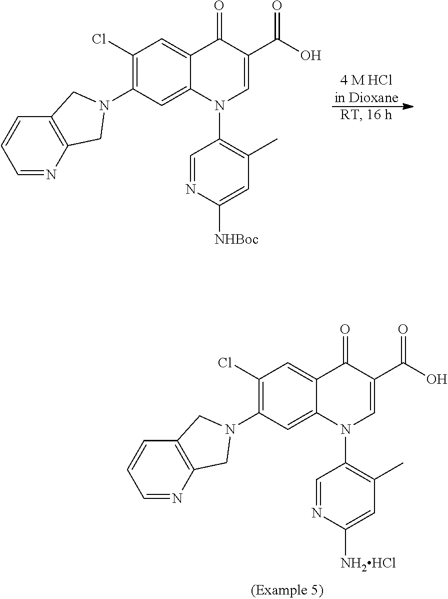

4M HCl in Dioxane (2 mL) was added drop wise to 1-(6-{[(tert-butoxy)carbonyl]amino}-4-methylpyridin-3-yl)-6-chloro-4-oxo-7-{5H,6H,7H-pyrrolo[3,4-b]pyridin-6-yl}-1,4-dihydroquinoline-3-carboxylic acid (100 mg, 182 μmol) at rt and the mixture was stirred for 16 h. After completion of the reaction (monitored by TLC), solvents were evaporated from reaction mixture, triturated with diethyl ether and followed by lyophilization to afford 1-(6-amino-4-methylpyridin-3-yl)-6-chloro-4-oxo-7-{5H,6H,7H-pyrrolo[3,4-b]pyridin-6-yl}-1,4-dihydroquinoline-3-carboxylic acid hydrochloride (16.0 mg, 33.0 μmol, 18%) as an off-white solid. TLC System: 10% MeOH in DCM; Rf-0.2. ¹H NMR (400 MHz, DMSO-d₆): δ 15.09 (bs, 1H), 8.65 (s, 1H), 8.47 (d, J=4.4 Hz, 1H), 8.24 (s, 1H), 8.18 (s, 1H), 7.83 (d, J=6.8 Hz, 1H), 7.35-7.30 (m, 1H), 7.23 (bs, 1H), 6.70 (s, 1H), 6.26 (m, 1H), 5.08-4.82 (m, 4H), 1.97 (s, 3H) (couple of exchangeable protons were not observed); MS (ESI): m/z 448 [M+H]⁺.

Analogues compounds, examples 96, 97, 107 to 118, 132 to 136, 138 to 146, 149 to 151, 154 to 161, 165 to 182, 184 to 187, 191 to 200 and 203 were synthesized by following similar procedures using appropriate reagents.

### Examples 6 and 7

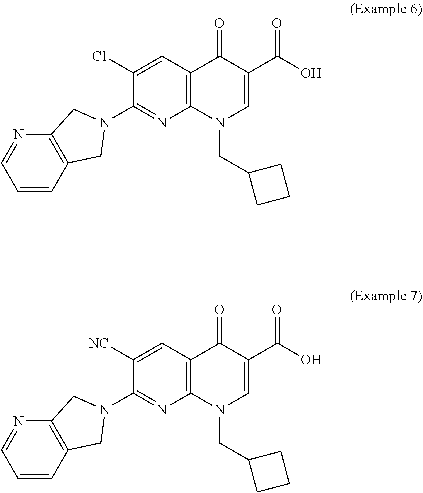 [SMILES: O=C(O)c1cn(CC2CCC2)c2nc(N3Cc4cccnc4C3)c(Cl)cc2c1=O ; N#Cc1cc2c(=O)c(C(=O)O)cn(CC3CCC3)c2nc1N1Cc2cccnc2C1]

### Step-1: Synthesis of ethyl 6,7-dichloro-1-(cyclobutylmethyl)-4-oxo-1,4-dihydro-1,8-naphthyridine-3-carboxylate

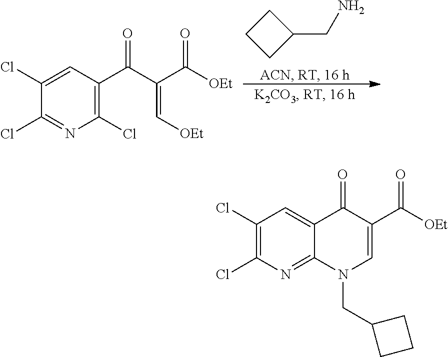 [SMILES: CCO/C=C(\C(=O)OCC)C(=O)c1cc(Cl)c(Cl)nc1Cl ; NCC1CCC1 ; CCOC(=O)c1cn(CC2CCC2)c2nc(Cl)c(Cl)cc2c1=O]

To a stirred solution of ethyl (2Z)-3-ethoxy-2-[(Z)-2,5,6-trichloropyridine-3-carbonyl]prop-2-enoate (450 mg, 1.27 mmol) in ACN (10 mL), 1-cyclobutylmethanamine (97.0 mg, 1.14 mmol) was added and stirred at rt for 16 h. Reaction was monitored by LC-MS, potassium carbonate (351 mg, 2.54 mmol) was added and stirred at rt for 16 h. The reaction mixture was diluted with water then extracted with EtOAc (2×20 mL). Combined organic layer was washed with cold brine solution and dried over anhydrous sodium sulphate, evaporated to afford crude. The crude solid was dissolved in 10% MeOH in DCM and treated with charcoal, filtered through celite bed. The celite pad was washed with 10% MeOH in DCM several times. The filtrate was concentrated under reduced pressure to get crude compound, which was purified by 100-200 mesh silica gel column chromatography using 30% EtOAc/hexanes as an eluent. Collected pure fractions were evaporated to dryness to afford ethyl 6,7-dichloro-1-(cyclobutylmethyl)-4-oxo-1,4-dihydro-1,8-naphthyridine-3-carboxylate (185 mg) as a yellow solid. MS (ESI): m/z 355.0 [M+H]⁺.

### Step-2: Synthesis of ethyl 6-chloro-1-(cyclobutylmethyl)-4-oxo-7-{5H,6H,7H-pyrrolo[3,4-b]pyridin-6-yl}-1,4-dihydro-1,8-naphthyridine-3-carboxylate

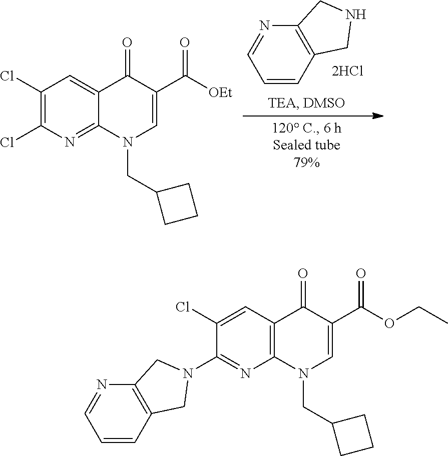 [SMILES: CCOC(=O)c1cn(CC2CCC2)c2nc(Cl)c(Cl)cc2c1=O ; c1cnc2c(c1)CNC2 ; CCOC(=O)c1cn(CC2CCC2)c2nc(N3Cc4cccnc4C3)c(Cl)cc2c1=O]

A mixture of ethyl 6,7-dichloro-1-(cyclobutylmethyl)-4-oxo-1,4-dihydro-1,8-naphthyridine-3-carboxylate (200 mg, 563 μmol) and 5H,6H,7H-pyrrolo[3,4-b]pyridine (81.1 mg, 675 μmol) di HCl in DMSO (1 mL) was added triethylamine (391 μL, 2.81 mmol) in a sealed tube and heated to 120° C. for 6 h. The reaction mixture was cooled to rt and quenched with ice cold water, stirred for 10 min. Precipitated solid was filtered and washed with diethyl ether, dried under vacuum to afford ethyl 6-chloro-1-(cyclobutylmethyl)-4-oxo-7-{5H,6H,7H-pyrrolo[3,4-b]pyridin-6-yl}-1,4-dihydro-1,8-naphthyridine-3-carboxylate (195 mg) as a pale brown solid. MS (ESI): m/z 439.0 [M+H]⁺.

### Step-3: Example 6: Synthesis of 6-chloro-1-(cyclobutylmethyl)-4-oxo-7-{5H,6H,7H-pyrrolo[3,4-b]pyridin-6-yl}-1,4-dihydro-1,8-naphthyridine-3-carboxylic acid

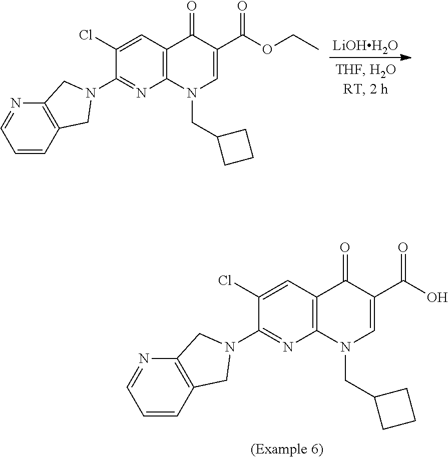

To a stirred solution of ethyl 6-chloro-1-(cyclobutylmethyl)-4-oxo-7-{5H,6H,7H-pyrrolo[3,4-b]pyridin-6-yl}-1,4-dihydro-1,8-naphthyridine-3-carboxylate (150 mg, 341 μmol) in THF (10 mL) and water (5 mL) was added LiOH·H₂O (42.7 mg, 1.02 mmol) and stirred at rt for 2 h. Excess THF was evaporated and the resulting residue was diluted with water and adjusted pH-7 using 1N HCl solution. Precipitated solid was filtered and washed with diethyl ether, dried under vacuum to afford 6-chloro-1-(cyclobutylmethyl)-4-oxo-7-{5H,6H,7H-pyrrolo[3,4-b]pyridin-6-yl}-1,4-dihydro-1,8-naphthyridine-3-carboxylic acid (98.5 mg) as a pale brown solid. TLC System: 5% MeOH/DCM, Rf: 0.3. MS (ESI): m/z 411.0 [M+H]⁺; ¹H NMR (400 MHz, DMSO-d₆) δ 15.19 (s, 1H), 8.99 (s, 1H), 8.53 (d, J=5.2 Hz, 1H), 8.36 (s, 1H), 7.90 (d, J=6.8 Hz, 1H), 7.39 (q, J=4.8 Hz, 1H), 5.38 (s, 2H), 5.30 (s, 2H), 4.59 (d, J=7.2 Hz, 2H), 2.96-2.95 (m, 1H), 1.98-1.94 (m, 2H), 1.91-1.84 (m, 4H).

### Step-4: Example 7: Synthesis of 6-cyano-1-(cyclobutylmethyl)-4-oxo-7-{5H,6H,7H-pyrrolo[3,4-b]pyridin-6-yl}-1,4-dihydro-1,8-naphthyridine-3-carboxylic acid

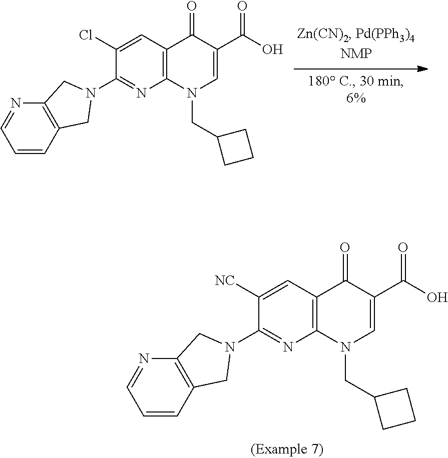 [SMILES: O=C(O)c1cn(CC2CCC2)c2nc(N3Cc4cccnc4C3)c(Cl)cc2c1=O ; N#Cc1cc2c(=O)c(C(=O)O)cn(CC3CCC3)c2nc1N1Cc2cccnc2C1]

In a microwave vial, to a stirred solution of 6-chloro-1-(cyclobutylmethyl)-4-oxo-7-{5H,6H,7H-pyrrolo[3,4-b]pyridin-6-yl}-1,4-dihydro-1,8-naphthyridine-3-carboxylic acid (200 mg, 486 μmol) in NMP (5 mL) was added bis(cyano radical) zinc (182 mg, 1.55 mmol) and degassed with argon for 10 min. To this degassed reaction mixture, tetrakis(triphenylphosphine) palladium (117 mg, 102 μmol) was added and again degassed for 10 min. The reaction mixture was irradiated to 180° C. for 30 min in microwave. Reaction was monitored by LCMS. The reaction mixture was cooled to rt and diluted with ice cold water. Precipitated solid was filtered and dried under vacuum. The solid was dissolved in 10% MeOH/DCM and undissolved material was filtered off. The organic layer was dried over anhydrous sodium sulphate, evaporated to afford crude compound. The crude compound was washed with IPA/diethyl ether and dried under vacuum to afford semi pure compound. The crude product was purified by prep HPLC (Method-7 of Table-2) and collected pure fractions were lyophilized to afford 6-cyano-1-(cyclobutylmethyl)-4-oxo-7-{5H,6H,7H-pyrrolo[3,4-b]pyridin-6-yl}-1,4-dihydro-1,8-naphthyridine-3-carboxylic acid (11.6 mg, 6%) as an off white solid. TLC System: 5% MeOH/DCM, Rf: 0.4 MS (ESI): m/z 402.3 [M+H]⁺; ¹H NMR (400 MHz, DMSO-d₆): δ 14.75 (s, 1H), 9.03 (s, 1H), 8.83 (s, 1H), 8.55 (d, J=4.8 Hz, 1H), 7.95 (d, J=7.6 Hz, 1H), 7.38 (q, J=4.8 Hz, 1H), 5.48-5.12 (brs, 4H), 4.57 (d, J=7.2 Hz, 2H), 2.98-2.92 (m, 1H), 1.98-1.95 (m, 2H), 1.89-1.85 (m, 4H).

Analogues compounds, examples 98 to 102, 119 to 131 and 137 were synthesized by following similar procedures using appropriate reagents

### Examples 8-87: General Procedure for the Synthesis of Examples 8-87

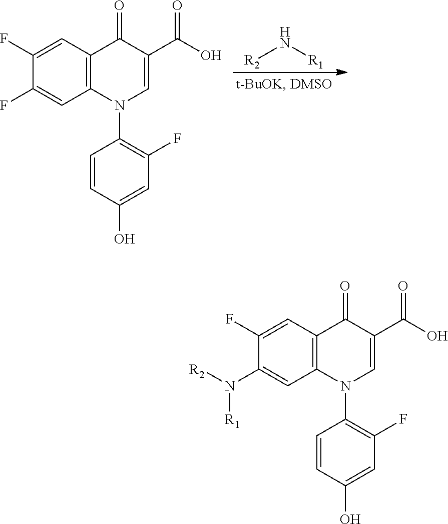

To a mixture of 6,7-difluoro-1-(2-fluoro-4-hydroxyphenyl)-4-oxo-1,4-dihydroquinoline-3-carboxylic acid (100 mg, 0.30 mmol, 1.0 equiv) and the corresponding amine (0.36 mmol, 1.2 equiv) in DMSO (2 mL) was added t-BuOK (336 mg, 3.0 mmol, equiv). The mixture was stirred at 100° C. for 1 h. The mixture was purified by reverse phase flash column with 30-60% acetonitrile in water to afford the corresponding final targets.

### Example 88: Synthesis of Ethyl 6-chloro-1-(6-hydroxypyridin-3-yl)-4-oxo-7-{5H,7H-pyrrolo[3,4-b]pyridin-6-yl}quinoline-3-carboxylate

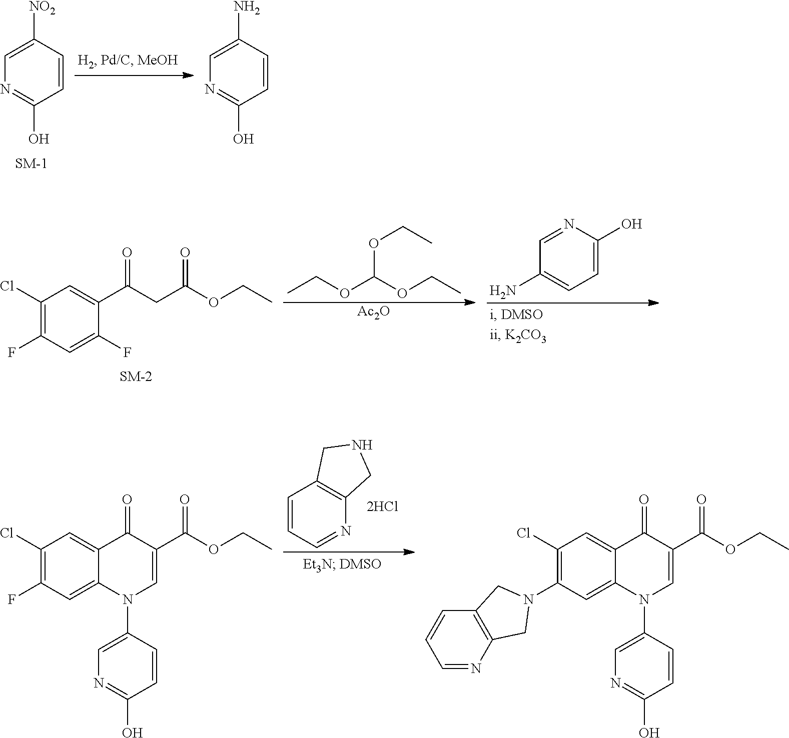

### Step-1. Synthesis of 5-Aminopyridin-2-ol

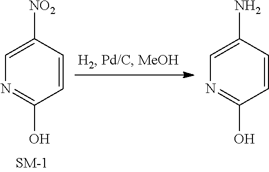 [SMILES: O=[N+]([O-])c1ccc(O)nc1 ; Nc1ccc(O)nc1]

To a mixture of 5-nitropyridin-2-ol (49.0 g, 349.76 mmol, 1.0 equiv) in MeOH (3 L) was added Pd/C (4.9 g) under N₂ atmosphere. After the addition, the N₂ atmosphere was replaced by H₂ atmosphere. The resulting mixture was stirred for overnight at room temperature under H₂ atmosphere. The resulting mixture was filtered. The filter cake was washed with MeOH (3×2 L). The filtrate was concentrated under vacuum. This resulted in 5-aminopyridin-2-ol (48.0 g, crude) as a red oil. LCMS (ESI) [M+H]⁺: 111.1.

### Step-2. Synthesis of Ethyl 6-chloro-7-fluoro-1-(6-hydroxypyridin-3-yl)-4-oxoquinoline-3-carboxylate

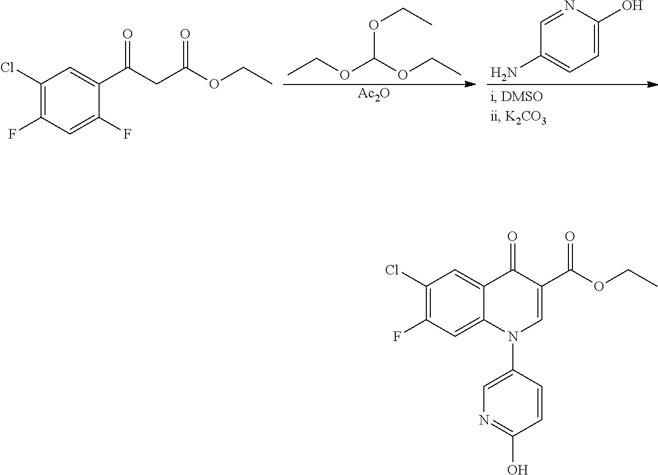

To a mixture of ethyl 3-(5-chloro-2,4-difluorophenyl)-3-oxopropanoate (60.0 g, 228.45 mmol, 1.0 equiv) in acetic anhydride (70.0 g, 685.35 mmol, 3.0 equiv) was added triethyl orthoformate (50.8 g, 342.68 mmol, 1.5 equiv). The resulting mixture was stirred at 100° C. for 2 h. The resulting mixture was concentrated under vacuum. The residue was dissolved in DMSO (3 L) and then 5-aminopyridin-2-ol (30.2 g, 274.14 mmol, 1.2 equiv) was added into the mixture. The resulting mixture was stirred at 25° C. for 2 h. To the resulting mixture was added K₂CO₃ (31.6 g, 228.45 mmol, 1.0 equiv). The resulting mixture was stirred at 100° C. for 1 h. The reaction was quenched by the addition of 3.5 L water. The precipitated solids were collected by filtration. This resulted in ethyl 6-chloro-7-fluoro-1-(6-hydroxypyridin-3-yl)-4-oxoquinoline-3-carboxylate (52.0 g, 63%) as a yellow solid. LCMS (ESI) [M+H]⁺: 363.1.

### Step-3. Synthesis of Ethyl 6-chloro-1-(6-hydroxypyridin-3-yl)-4-oxo-7-{5H,7H-pyrrolo[3,4-b]pyridin-6-yl}quinoline-3-carboxylate

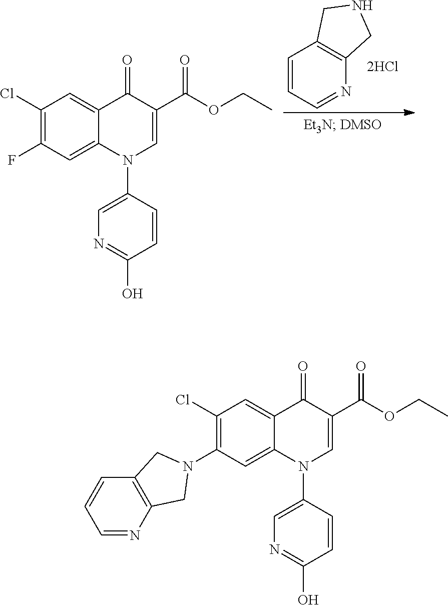

To a mixture of ethyl 6-chloro-7-fluoro-1-(6-hydroxypyridin-3-yl)-4-oxoquinoline-3-carboxylate (10 g, 27.56 mmol, 1.0 equiv) in DMSO (40 mL) was added 6,7-dihydro-5H-pyrrolo[3,4-b]pyridine dihydrochloride (6.387 g, 33.08 mmol, 1.2 equiv) and Et₃N (22 g, 220.54 mmol, 8.0 equiv). The resulting mixture was stirred for 3 h at 110° C. under nitrogen atmosphere. The mixture was allowed to cool down to room temperature. The precipitated solids were collected by filtration to afford the crude product which was recrystallized from acetonitrile and dried to afford ethyl 6-chloro-1-(6-hydroxypyridin-3-yl)-4-oxo-7-{5H,7H-pyrrolo[3,4-b]pyridin-6-yl}quinoline-3-carboxylate (5.4 g, 42.32%) as a purple solid. LCMS (ESI) [M+H]⁺: 463.1. ¹H NMR (400 MHz, DMSO-d₆) δ 12.10 (s, 1H), 8.47 (d, J=4.9 Hz, 1H), 8.40 (s, 1H), 8.08 (s, 1H), 7.97 (d, J=3.0 Hz, 1H), 7.85 (d, J=7.7 Hz, 1H), 7.69 (d, J=9.7, 3.0 Hz, 1H), 7.32 (d, J=7.6, 4.9 Hz, 1H), 6.53 (d, J=9.6 Hz, 1H), 6.42 (s, 1H), 5.14-4.77 (m, 4H), 4.20 (m, J=7.0 Hz, 2H), 1.27 (s, J=7.1 Hz, 3H).

### Example 89: Synthesis of Ethyl 1-(6-aminopyridin-3-yl)-6-chloro-4-oxo-7-{1H,3H-pyrrolo[3,4-c]pyridin-2-yl}quinoline-3-carboxylate

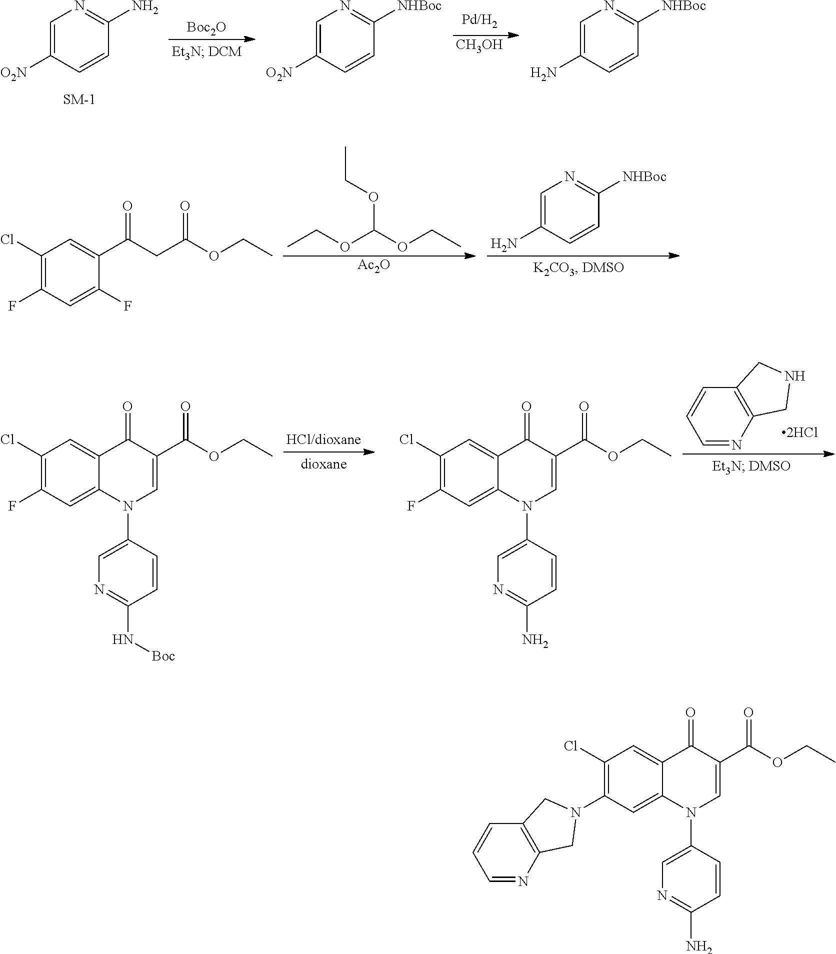

### Step-1. Synthesis of tert-butyl N-(5-nitropyridin-2-yl)carbamate

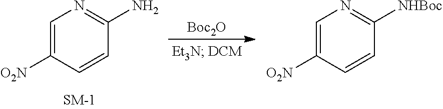 [SMILES: "Boc2O" ; Nc1ccc([N+](=O)[O-])cn1 ; CC(C)(C)OC(=O)Nc1ccc([N+](=O)[O-])cn1]

To a stirred solution of 5-nitropyridin-2-amine (20 g, 143.76 mmol, 1 equiv) and Boc₂O (37.65 g, 172.52 mmol, 1.2 equiv) in DCM (200 mL) was added Et₃N (43.64 g, 431.30 mmol, 3 equiv) at room temperature. The resulting mixture was stirred for 12 h at room temperature under nitrogen atmosphere. The reaction was quenched by the addition of water (300 mL). The resulting mixture was extracted with CH₂Cl₂ (500 ml). The combined organic layers were washed with water (500 ml), dried over anhydrous Na₂SO₄. After filtration, the filtrate was concentrated under reduced pressure. The residue was purified by silica gel column chromatography, eluted with Petroleum ether/EtOAc (1:1) to afford tert-butyl N-(5-nitropyridin-2-yl)carbamate (17 g, 49.43%) as a yellow solid. LCMS (ESI) [M+H]⁺: 240.

### Step-2. Synthesis of Tert-butyl N-(5-aminopyridin-2-yl)carbamate

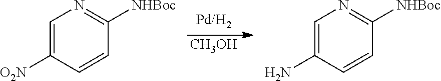 [SMILES: CC(C)(C)OC(=O)Nc1ccc([N+](=O)[O-])cn1 ; "CH3OH" ; CC(C)(C)OC(=O)Nc1ccc(N)cn1]

To a stirred solution of tert-butyl N-(5-nitropyridin-2-yl)carbamate (17 g, 71.06 mmol, 1 equiv) in methanol (400 mL) was added Pd/C (1.7 g) under N₂ atmosphere. After the addition, the N₂ atmosphere was replaced by H₂ atmosphere. The resulting mixture was stirred for 2 h at room temperature under H₂ atmosphere. The resulting mixture was filtered, the filter cake was washed with MeOH (200 ml). The filtrate was concentrated under reduced pressure to afford tert-butyl N-(5-aminopyridin-2-yl)carbamate (14 g, 94.15%) as a yellow solid. The crude product mixture was used in the next step directly without further purification. LCMS (ESI) [M+H]⁺: 210.

### Step-3. Synthesis of Ethyl 1-{6-[(tert-butoxycarbonyl)amino]pyridin-3-yl}-6-chloro-7-fluoro-4-oxoquinoline-3-carboxylate

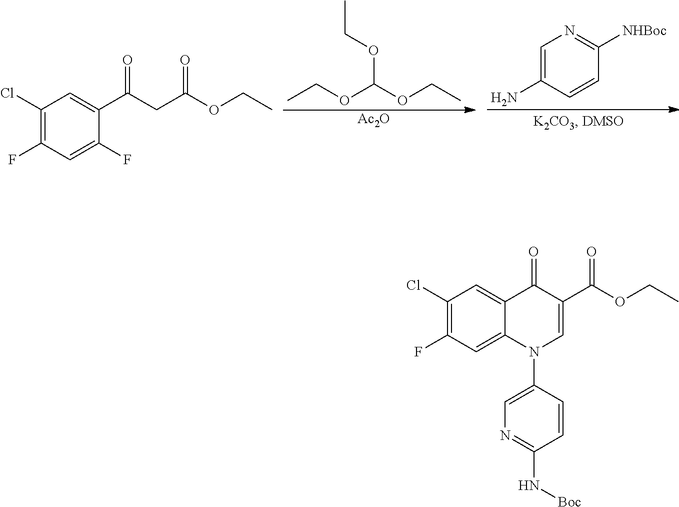

To a stirred solution of ethyl 3-(5-chloro-2,4-difluorophenyl)-3-oxopropanoate (15.06 g, 57.34 mmol, 1.2 equiv) in propionic anhydride (18.66 g, 143.37 mmol, 3 equiv) was added triethyl orthoformate (10.62 g, 71.68 mmol, 1.5 equiv) at room temperature. The resulting mixture was stirred for 2 h at 100° C. under nitrogen atmosphere. The residue was concentrated under reduced pressure to afford 15 g yellow oil. To a solution of the 15 g yellow oil in DMSO (200 mL) was added tert-butyl N-(5-aminopyridin-2-yl)carbamate (10 g, 47.79 mmol, 1 equiv) at room temperature under nitrogen atmosphere, the resulting mixture was stirred for 2 h at room temperature under nitrogen atmosphere. K₂CO₃ (19.81 g, 143.37 mmol, 3 equiv) was then added and the mixture was stirred for 12 h. The mixture was poured into water. The precipitated solids were collected by filtration, washed with water (200 ml) and dried to afford ethyl 1-{6-[(tert-butoxycarbonyl)amino]pyridin-3-yl}-6-chloro-7-fluoro-4-oxoquinoline-3-carboxylate (12 g, 54.37%) as a yellow solid. The crude product mixture was used in the next step directly without further purification. LCMS (ESI) [M+H]⁺: 462.

### Step-4. Synthesis of Ethyl 1-(6-aminopyridin-3-yl)-6-chloro-7-fluoro-4-oxoquinoline-3-carboxylate

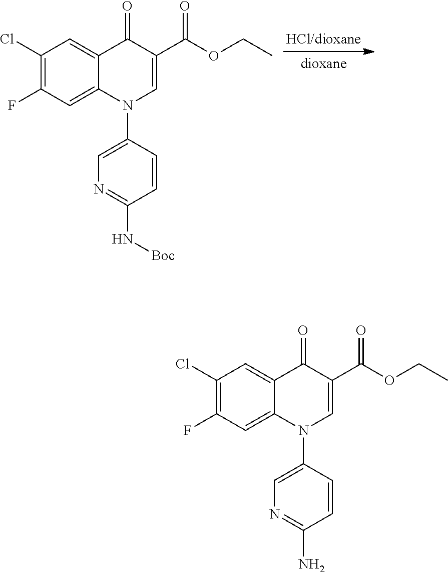 [SMILES: CCOC(=O)c1cn(-c2ccc(NC(=O)OC(C)(C)C)nc2)c2cc(F)c(Cl)cc2c1=O ; CCOC(=O)c1cn(-c2ccc(N)nc2)c2cc(F)c(Cl)cc2c1=O]

To a stirred solution of ethyl 1-{6-[(tert-butoxycarbonyl)amino]pyridin-3-yl}-6-chloro-7-fluoro-4-oxoquinoline-3-carboxylate (10.00 g, 21.65 mmol, 1 equiv) in 1,4-dioxane (200 mL) was added HCl (gas) in 1,4-dioxane (200.00 mL, 4 M) at 0° C. under nitrogen atmosphere. The resulting mixture was stirred for 12 h at room temperature under nitrogen atmosphere. The resulting mixture was concentrated under reduced pressure to afford ethyl 1-(6-aminopyridin-3-yl)-6-chloro-7-fluoro-4-oxoquinoline-3-carboxylate (10.00 g, crude) as a yellow solid. The crude product mixture was used in the next step directly without further purification. LCMS (ESI) [M+H]⁺: 362.

### Step-5. Synthesis of Ethyl 1-(6-aminopyridin-3-yl)-6-chloro-4-oxo-7-{1H,3H-pyrrolo[3,4-c]pyridin-2-yl}quinoline-3-carboxylate

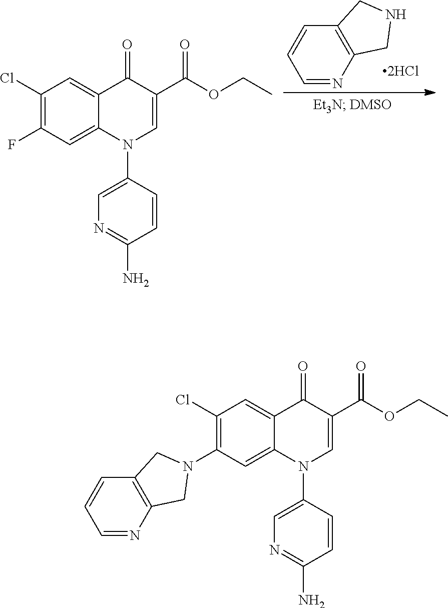

To a stirred solution of ethyl 1-(6-aminopyridin-3-yl)-6-chloro-7-fluoro-4-oxoquinoline-3-carboxylate (10 g, 27.64 mmol, 1 equiv) and 6,7-dihydro-5H-pyrrolo[3,4-b]pyridine dihydrochloride (10.673 g, 55.28 mmol, 2 equiv) in DMSO (100 mL) was added Et₃N (16.78 g, 165.84 mmol, 6 equiv) at room temperature. The resulting mixture was stirred for 4 h at 100° C. under nitrogen atmosphere. The reaction was quenched by the addition of 200 mL water. The resulting mixture was extracted with EtOAc (200 mL×3). The combined organic layers were washed with water (500 mL), dried over anhydrous Na₂SO₄. After filtration, the filtrate was concentrated under reduced pressure. The residue was purified by reverse phase flash chromatography with the following conditions: column, silica gel; mobile phase, MeCN in water, 5% to 100% gradient in 40 min; detector, UV 254 nm to afford ethyl 1-(6-aminopyridin-3-yl)-6-chloro-4-oxo-7-{1H,3H-pyrrolo[3,4-c]pyridin-2-yl}quinoline-3-carboxylate (4.5033 g, 35.27%) as a yellow solid. LCMS (ESI) [M+H]⁺: 462.25. ¹H NMR (400 MHz, DMSO-d₆) δ 8.45-8.44 (m, 1H), 8.31 (s, 1H), 8.17-8.03 (m, 2H), 7.92-7.82 (m, 1H), 7.75-7.65 (m, 1H), 7.38-7.30 (m, 1H), 6.65 (d, J=8.8 Hz, 1H), 6.56 (s, 2H), 6.31 (s, 1H), 4.87-4.80 (d, J=30.1 Hz, 4H), 4.27-4.05 (m, 2H), 1.5-1.20 (m, 3H).

### Example 90: Synthesis of Ethyl 6-chloro-1-(6-chloropyridin-3-yl)-7-(5,7-dihydro-6H-pyrrolo[3,4-b]pyridin-6-yl)-4-oxo-1,4-dihydroquinoline-3-carboxylate

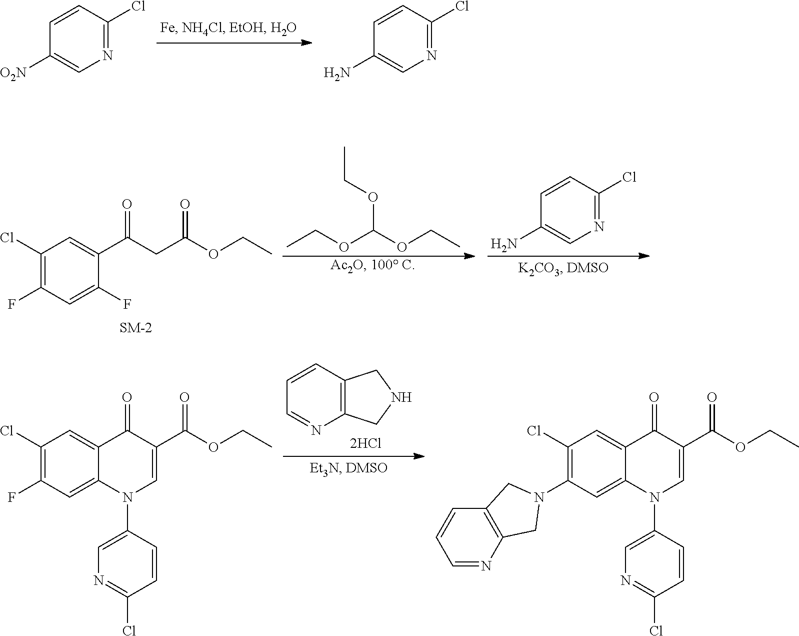

### Step-1. Synthesis of 6-Chloropyridin-3-amine

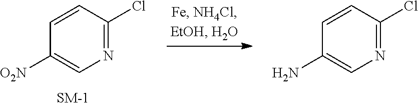 [SMILES: O=[N+]([O-])c1ccc(Cl)nc1 ; Nc1ccc(Cl)nc1]

To a stirred mixture of 2-chloro-5-nitropyridine (50.0 g, 315.38 mmol, 1.0 equiv) in EtOH (1.0 L) and H₂O (200 ml) was added Fe (176.1 g, 3.15 mol, 10.0 equiv) and NH₄Cl (84.3 g, 1.57 mol, 5.0 equiv). The resulting mixture was stirred for 1 h at 85° C. under nitrogen atmosphere. The resulting mixture was filtered. The filter cake was washed with EtOH (500 mL). The filtrate was concentrated under reduced pressure to afford the crude product which was purified by silica gel column chromatography, eluted with Petroleum ether/EtOAc (1:1) to afford 6-chloropyridin-3-amine (30.0 g, 73.99%) as a yellow solid. LCMS (ESI) [M+H]⁺:129.0.

### Step-2. Synthesis of Ethyl 6-chloro-1-(6-chloropyridin-3-yl)-7-fluoro-4-oxo-1,4-dihydroquinoline-3-carboxylate

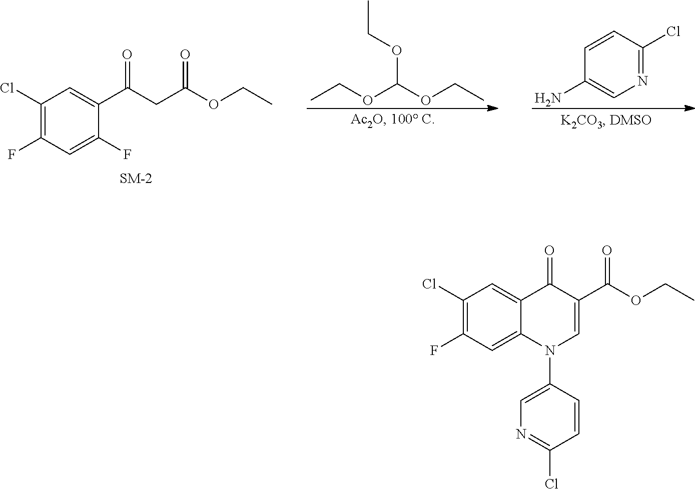

To a mixture of ethyl 3-(5-chloro-2,4-difluorophenyl)-3-oxopropanoate (20.0 g, 76.33 mmol, 1.0 equiv) and triethyl orthoformate (16.9 g, 114.50 mmol, 1.5 equiv) was added acetic anhydride (23.3 g, 229.0 mmol, 3.0 equiv). The resulting mixture was stirred for 1 h at 100° C. under nitrogen atmosphere. The mixture was concentrated under vacuum. To the above mixture was added 6-chloropyridin-3-amine (11.7 g, 91.59 mmol, 1.2 equiv) in DMSO (500 ml). The mixture was stirred for 1 h. K₂CO₃ (21.1 g, 152.66 mmol, 2.0 equiv) was then added to the mixture at room temperature. The resulting mixture was stirred for overnight at room temperature under nitrogen atmosphere. The reaction was quenched with water (2 L) at 0° C. The precipitated solids were collected by filtration, washed with water and dried to afford ethyl 6-chloro-1-(6-chloropyridin-3-yl)-7-fluoro-4-oxo-1,4-dihydroquinoline-3-carboxylate (16.0 g, 55.17%) as a yellow solid. LCMS (ESI) [M+H]⁺:381.0.

### Step-3. Synthesis of Ethyl 6-chloro-1-(6-chloropyridin-3-yl)-7-(5,7-dihydro-6H-pyrrolo [3,4-b]pyridin-6-yl)-4-oxo-1,4-dihydroquinoline-3-carboxylate

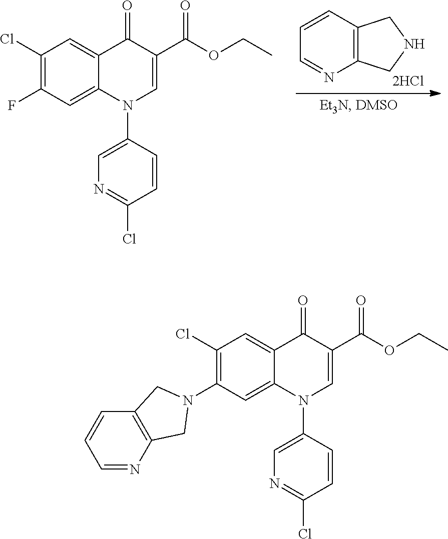

To a mixture of ethyl 6-chloro-1-(6-chloropyridin-3-yl)-7-fluoro-4-oxoquinoline-3-carboxylate (6.0 g, 15.74 mmol, 1.0 equiv) and 6,7-dihydro-5H-pyrrolo[3,4-b]pyridine dihydrochloride (3.646 g, 18.89 mmol, 1.2 equiv) in DMSO (60 mL) was added Et₃N (9.6 g, 94.45 mmol, 6.0 equiv). The resulting mixture was stirred for 16 h at 70° C. under nitrogen atmosphere. Acetonitrile (100 mL) was added. The solid was collected by filtration to afford the crude product which was recrystallized by toluene and HOAc (1:1) to afford ethyl 6-chloro-1-(6-chloropyridin-3-yl)-4-oxo-7-{5H,7H-pyrrolo[3,4-b]pyridin-6-yl}quinoline-3-carboxylate (2.3800 g, 31.41%) as yellow solid. LCMS (ESI) [M+H]⁺: 480.95.

¹H NMR (400 MHz, Chloroform-d) δ 8.58 (d, J=2.5 Hz, 1H), 8.51 (d, J=4.8 Hz, 1H), 8.40 (s, 1H), 8.29 (s, 1H), 7.95-7.84 (m, 1H), 7.67 (d, J=8.2, 2.8 Hz, 2H), 7.29-7.26 (m, 1H), 6.06 (s, 1H), 5.04 (s, 2H), 4.83 (s, 2H), 4.37 (m, 2H), 1.39 (m, 3H).

### Example 91: 1-(6-(azetidin-1-yl)-4-methylpyridin-3-yl)-6-chloro-7-(5,7-dihydro-6H-pyrrolo[3,4-b]pyridin-6-yl)-4-oxo-1,4-dihydro-1,8-naphthyridine-3-carboxylic acid

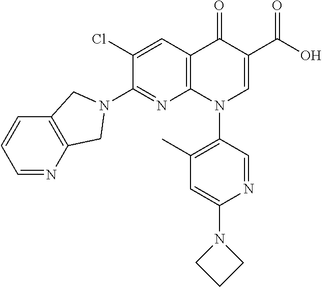 [SMILES: Cc1cc(N2CCC2)ncc1-n1cc(C(=O)O)c(=O)c2cc(Cl)c(N3Cc4cccnc4C3)nc21]

### Step-1: Synthesis of 2-(azetidin-1-yl)-4-methyl-5-nitropyridine

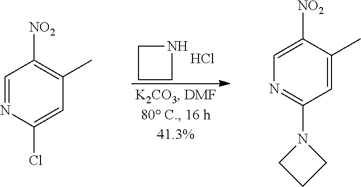

To a stirred solution of azetidine hydrochloride (16.0 g, 172 mmol) in DMF (250 mL) was added potassium carbonate (49.7 g, 360 mmol) and stirred for 10 min. Then, was added 2-chloro-4-methyl-5-nitropyridine (25 g, 144 mmol) to the reaction mixture. The reaction mixture was turned to dark green colour while adding SM and heated to 80° C. for 16 h. After completion of reaction, the reaction mixture was cooled to rt and diluted with ice cold water (1 litre) then stirred for 30 min. Precipitated solid was filtered and dried under vacuum to afford crude, which was dissolved in EtOAc (1 litre) and washed with brine solution (2×500 mL). The organic layer was dried over anhydrous sodium sulphate, evaporated to dryness to afford crude black solid. The crude compound was purified by 100-200 mesh silica gel column chromatography using 10-15% EtOAc/hexanes as an eluent. Collected pure fractions were evaporated to dryness to afford 2-(azetidin-1-yl)-4-methyl-5-nitropyridine (11.5 g, 59.5 mmol) as a pale brown solid.

MS (ESI): m/z 194 [M+H]⁺.

### Step-2: Synthesis of 6-(azetidin-1-yl)-4-methylpyridin-3-amine

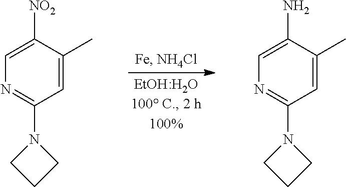

To a stirred solution of 2-(azetidin-1-yl)-4-methyl-5-nitropyridine (5.0 g, 25.8 mmol) in EtOH (50 mL) and H2O (50 mL) was added ammonium chloride (6.90 g, 129 mmol). To this reaction mixture, iron (7.20 g, 129 mmol) powder was added portion wise at RT and heated to 80° C. for 2 h. The reaction mixture was filtered through celite pad and celite pad was washed with 10% MeOH/DCM. The organic layer was dried over anhydrous sodium sulphate and evaporated to dryness to afford crude 6-(azetidin-1-yl)-4-methylpyridin-3-amine (4.60 g, 28.1 mmol) as a dark brown solid. ¹H NMR (400 MHz, DMSO-d₆): δ 7.41-7.16 (m, 3H), 6.43 (s, 1H), 3.94 (s, 4H), 2.31 (s, 2H), 2.15 (s, 3H).

### Step-3: Synthesis of ethyl 1-(6-(azetidin-1-yl)-4-methylpyridin-3-yl)-6,7-dichloro-4-oxo-1,4-dihydro-1,8-naphthyridine-3-carboxylate

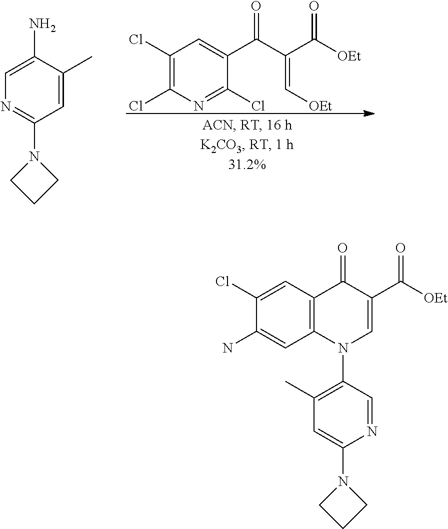 [SMILES: Cc1cc(N2CCC2)ncc1N ; CCO/C=C(\C(=O)OCC)C(=O)c1cc(Cl)c(Cl)nc1Cl ; CCOC(=O)c1cn(-c2cnc(N3CCC3)cc2C)c2cc(N)c(Cl)cc2c1=O]

To a stirred solution of ethyl (2Z)-3-ethoxy-2-[(Z)-2,5,6-trichloropyridine-3-carbonyl]prop-2-enoate (6 g, 17.0 mmol) in ACN (120 mL) was added 6-(azetidin-1-yl)-4-methylpyridin-3-amine (2.77 g, 17.0 mmol) and stirred at rt for 16 h. After 16 h, potassium carbonate (4.69 g, 34.0 mmol) was added to the reaction mixture and stirred at rt for another 16 h. After completion of reaction, excess ACN was evaporated and resulting residue was diluted with water then stirred for 10 min. Precipitated solid was filtered through Buchner funnel and dried under vacuum to afford crude. The crude compound was purified by 100-200 mesh silica gel column chromatography using 50% EtOAc/Hexanes to 100% EtOAc as an eluent. Collected pure fractions were evaporated to dryness to afford ethyl 1-[6-(azetidin-1-yl)-4-methylpyridin-3-yl]-6,7-dichloro-4-oxo-1,4-dihydro-1,8-naphthyridine-3-carboxylate (2.30 g, 5.30 mmol) as an off white solid. MS (ESI): m/z 434.1 [M+H]⁺.

### Step-4: Synthesis of ethyl 1-(6-(azetidin-1-yl)-4-methylpyridin-3-yl)-6-chloro-7-(5,7-dihydro-6H-pyrrolo[3,4-b]pyridin-6-yl)-4-oxo-1,4-dihydro-1,8-naphthyridine-3-carboxylate

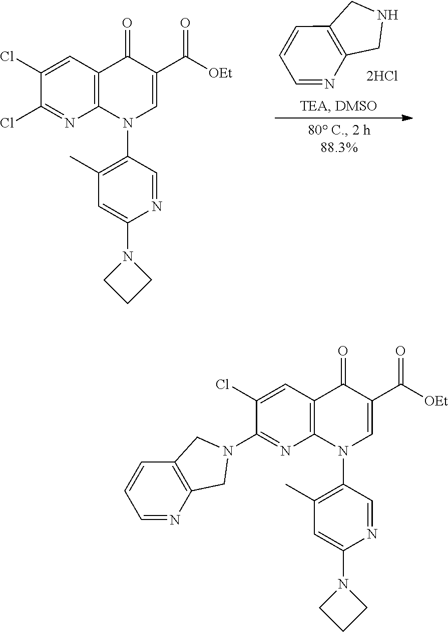 [SMILES: CCOC(=O)c1cn(-c2cnc(N3CCC3)cc2C)c2nc(Cl)c(Cl)cc2c1=O ; c1cnc2c(c1)CNC2 ; CCOC(=O)c1cn(-c2cnc(N3CCC3)cc2C)c2nc(N3Cc4cccnc4C3)c(Cl)cc2c1=O]

A mixture of ethyl 1-[6-(azetidin-1-yl)-4-methylpyridin-3-yl]-6,7-dichloro-4-oxo-1,4-dihydro-1,8-naphthyridine-3-carboxylate (2.3 g, 5.30 mmol) and 5H,6H,7H-pyrrolo[3,4-b]pyridine diHCl (764 mg, 6.36 mmol) in DMSO (5 mL) was added triethylamine (2.20 mL, 15.9 mmol) and heated to 80° C. for 2 h. The reaction mixture was cooled to rt and quenched with ice cold water, stirred for 10 min. Precipitated solid was filtered and dried under vacuum, triturated with diethyl ether to afford ethyl 1-[6-(azetidin-1-yl)-4-methylpyridin-3-yl]-6-chloro-4-oxo-7-{5H,6H,7H-pyrrolo[3,4-b]pyridin-6-yl}-1,4-dihydro-1,8-naphthyridine-3-carboxylate (2.42 g, 4.69 mmol) as a pale brown solid. MS (ESI): m/z 517.6 [M+H]⁺.

### Step-5: Synthesis of 1-(6-(azetidin-1-yl)-4-methylpyridin-3-yl)-6-chloro-7-(5,7-dihydro-6H-pyrrolo[3,4-b]pyridin-6-yl)-4-oxo-1,4-dihydro-1,8-naphthyridine-3-carboxylic acid

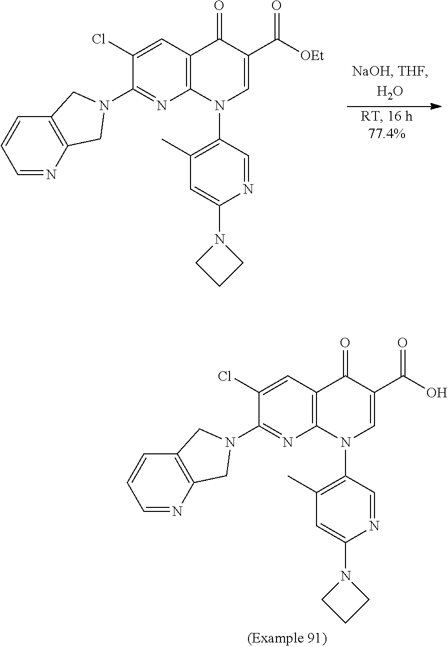 [SMILES: CCOC(=O)c1cn(-c2cnc(N3CCC3)cc2C)c2nc(N3Cc4cccnc4C3)c(Cl)cc2c1=O ; Cc1cc(N2CCC2)ncc1-n1cc(C(=O)O)c(=O)c2cc(Cl)c(N3Cc4cccnc4C3)nc21]

To a stirred solution of ethyl 1-[6-(azetidin-1-yl)-4-methylpyridin-3-yl]-6-chloro-4-oxo-7-{5H,6H,7H-pyrrolo[3,4-b]pyridin-6-yl}-1,4-dihydro-1,8-naphthyridine-3-carboxylate (3.2 g, 6.18 mmol) in THF (160 mL) was added a solution of sodium hydroxide (739 mg, 18.5 mmol) in water (160 mL) and stirred at rt for 16 h. After completion of reaction, excess THF was evaporated and the resulting mixture was adjusted to pH-7 by using aqueous 1N hydrochloric acid. Precipitate solid was filtered and washed with water, dried under vacuum. Obtained solid was triturated with Methanol followed by lyophilization to afford 1-[6-(azetidin-1-yl)-4-methylpyridin-3-yl]-6-chloro-4-oxo-7-{5H,6H,7H-pyrrolo[3,4-b]pyridin-6-yl}-1,4-dihydro-1,8-naphthyridine-3-carboxylic acid (2.34 g, 4.79 mmol) as a pale yellow solid. ¹H NMR (400 MHz, DMSO-d₆): δ 15.01 (s, 1H), 8.64 (s, 1H), 8.46 (d, J=4.0 Hz, 1H), 8.40 (s, 1H), 8.08 (s, 1H), 7.78 (d, J=7.6 Hz, 1H), 7.32 (q, J=4.8 Hz, 1H), 6.44 (s, 1H), 5.15 (s, 2H), 4.81 (s, 2H), 4.09-4.00 (m, 4H), 2.41-2.33 (m, 2H), 1.99 (s, 3H); MS (ESI): m/z 489 [M+H]⁺.

Analogues compounds, examples 183, 188 to 190 and 201 were synthesized by following similar procedures using appropriate reagents.

### Example 92: 1-(6-aminopyridin-3-yl)-6-chloro-7-(3-((2-hydroxyethyl)(methyl)amino)-1H-pyrazol-1-yl)-4-oxo-1,4-dihydroquinoline-3-carboxylic acid

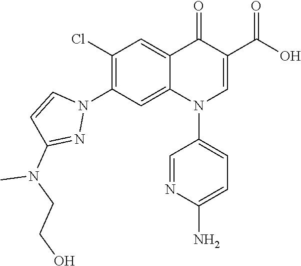 [SMILES: CN(CCO)c1ccn(-c2cc3c(cc2Cl)c(=O)c(C(=O)O)cn3-c2ccc(N)nc2)n1]

### Step-1: Synthesis of tert-butyl (1-(4-methoxybenzyl)-1H-pyrazol-3-yl)(methyl)carbamate

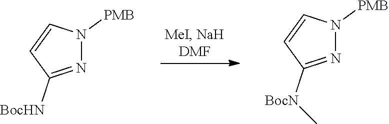

To a stirred solution of tert-butyl N-{1-[(4-methoxyphenyl)methyl]-1H-pyrazol-3-yl}carbamate (3 g, 9.88 mmol) in DMF (10 mL) and was added sodium hydride (591 mg, 14.8 mmol) at 0° C. then stirred at rt for 10 min. Methyl iodide (1.22 mL, 19.7 mmol) was added to the reaction mixture at 0° C. then stirred at rt for 16 h. After Completion of the reaction, the reaction mixture was quenched with ice cold water and extracted with EtOAc (2×100 mL). The combined organic layer was washed with water (20 mL) and brine (20 mL), dried over anhydrous sodium sulphate, concentrated under reduced pressure to get crude compound. which was purified by silica gel column chromatography to afford tert-butyl N-{1-[(4-methoxyphenyl)methyl]-1H-pyrazol-3-yl}-N-methylcarbamate (2.15 g, 6.77 mmol) as colourless oil compound. MS (ESI): m/z 318.1 [M+H]⁺.

### Step-2: Synthesis of 1-(4-methoxybenzyl)-N-methyl-1H-pyrazol-3-amine (TFA salt)

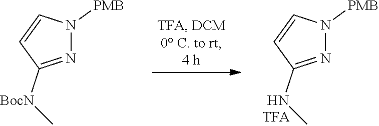

To a stirred solution of tert-butyl N-{1-[(4-methoxyphenyl)methyl]-1H-pyrazol-3-yl}-N-methylcarbamate (2 g, 6.11 mmol) in DCM (20 mL) was added trifluoroacetic acid (4.67 mL, 61.1 mmol) at 0° C. under N₂ atmosphere. The resulting mixture was stirred at room temperature for 4 h. The progress of the reaction was (Monitored by TLC). After completion 10 of the reaction, the reaction mixture was concentrated under reduced pressure until to remove DCM to afforded 1-[(4-methoxyphenyl)methyl]-N-methyl-1H-pyrazol-3-amine (1.33 g, 6.14 mmol) as brown oil compound (as TFA salt). MS (ESI): m/z 218.1 [M+H]⁺.

### Step-3: Synthesis of 1-(4-methoxybenzyl)-N-(2-methoxyethyl)-N-methyl-1H-pyrazol-3-amine

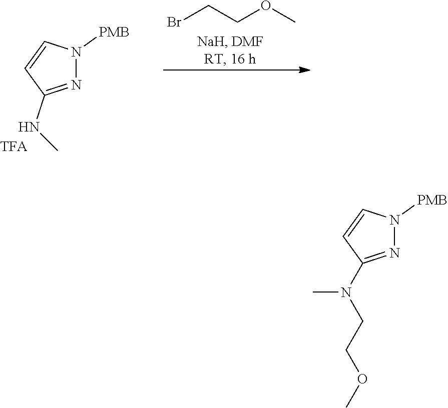

To a stirred solution of 1-[(4-methoxyphenyl)methyl]-N-methyl-1H-pyrazol-3-amine (500 mg, 2.30 mmol) in DMF (5 mL) and was added sodium hydride (183 mg, 4.60 mmol) at 0° C. and stirred at rt for 15 min, then 1-bromo-2-methoxyethane (325 PL, 3.44 mmol) was added slowly to the reaction mixture and stirred at RT for 16 h. After completion of the reaction (monitored by TLC), the reaction mixture was quenched with ice cold water and extracted with ethyl acetate (2×150 mL). The combined organic layer was washed with water (20 mL) and brine (20 mL), dried over sodium sulfate then concentrated under reduced pressure to get the crude compound. It was purified by silica gel column chromatography using 20% EtOAc in hexane as eluent to get the semi pure product N-(2-methoxyethyl)-1-[(4-methoxyphenyl)methyl]-N-methyl-1H-pyrazol-3-amine (243 mg, 882 μmol) as a pale yellow colour oil. MS (ESI): m/z 276.2 [M+H]⁺.

### Step-4: Synthesis of N-(2-methoxyethyl)-N-methyl-1H-pyrazol-3-amine

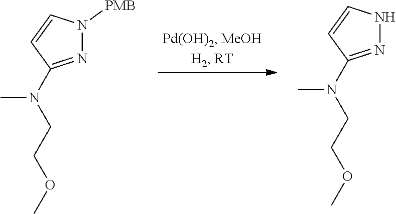

To a stirred solution of N-(2-methoxyethyl)-1-[(4-methoxyphenyl)methyl]-N-methyl-1H-pyrazol-3-amine (200 mg, 602 μmol) in MeOH (5 mL) was added Palladium hydroxide (211 mg, 301 μmol) at inert atmosphere, Reaction was maintained at rt and stirred for 16 h under H2(g) pressure. after completed the reaction, the reaction mixture was filtered through a celite pad, filterate was concentrated to afford N-(2-methoxyethyl)-N-methyl-1H-pyrazol-3-amine (64.0 mg, 412 μmol) as brown viscous liquid. MS (ESI): m/z 156.1 [M+H]⁺.

### Step-5: Synthesis of ethyl 1-(6-((tert-butoxycarbonyl)amino)pyridin-3-yl)-6-chloro-7-(3-((2-methoxyethyl)(methyl)amino)-1H-pyrazol-1-yl)-4-oxo-1,4-dihydroquinoline-3-carboxylate

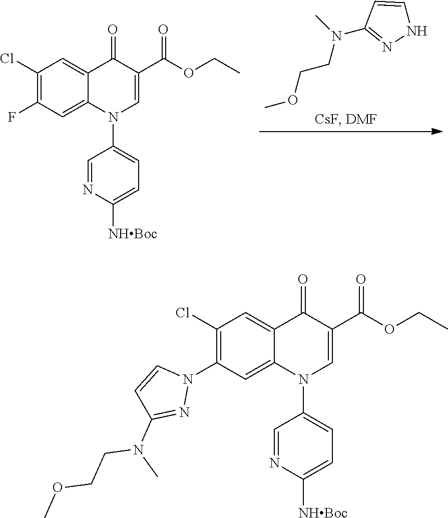

In 10 mL sealed tube a solution of N-(2-methoxyethyl)-N-methyl-1H-pyrazol-3-amine (67.2 mg, 433 μmol) in DMF (2 mL) was added caesium fluoride (131 mg, 866 μmol) followed by ethyl 1-(6-{[(tert-butoxy)carbonyl]amino}pyridin-3-yl)-6-chloro-7-fluoro-4-oxo-1,4-dihydroquinoline-3-carboxylate (200 mg, 433 μmol) at room temperature. The reaction was then heated to 100° C. and stirred for 16 h. After completed the reaction, the reaction mixture was quenched with ice-cold water (10 mL), which resulted in precipitation. Precipitated solid was collected by filtration and washed with water followed by diethyl ether and pentane then dried under vacuum, filtration to afford ethyl 1-(6-{[(tert-butoxy)carbonyl]amino}pyridin-3-yl)-6-chloro-7-13-[(2-methoxyethyl)(methyl)amino]-1H-pyrazol-1-ylI-4-oxo-1,4-dihydroquinoline-3-carboxylate (64.6 mg, 108 μmol) as yellow solid. MS (ESI): m/z 597.3 [M+H]⁺.

### Step-6: Synthesis of 1-(6-aminopyridin-3-yl)-6-chloro-7-(3-((2-hydroxyethyl)(methyl)amino)-1H-pyrazol-1-yl)-4-oxo-1,4-dihydroquinoline-3-carboxylic acid

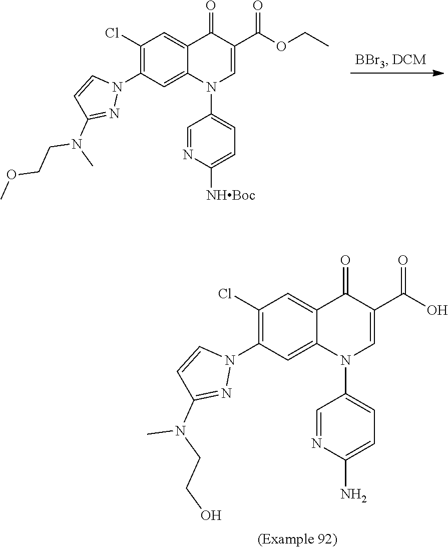

Boron tribromide in DCM (251 μL, 251 μmol) was added to ethyl 1-(6-{[(tert-butoxy)carbonyl]amino}pyridin-3-yl)-6-chloro-7-{3-[(2-methoxyethyl)(methyl)amino]-1H-pyrazol-1-yl}-4-oxo-1,4-dihydroquinoline-3-carboxylate (150 mg, 251 μmol) in DCM (5 mL) at 0° C. under N₂ atmosphere. The resulting mixture was stirred at room temperature for 16 hr. The progress of the reaction was (Monitored by TLC). After completed the reaction, the reaction mixture was concentrated under reduced pressure until to remove solvents. Then the residue was triturated with diethyl ether and purified by prep HPLC followed by lyophilization to afforded 1-(6-aminopyridin-3-yl)-6-chloro-7-13-[(2-hydroxyethyl)(methyl)amino]-1H-pyrazol-1-yl}-4-oxo-1,4-dihydroquinoline-3-carboxylic acid (29.4 mg, 64.6 μmol) as yellow solid ¹H NMR (400 MHz, DMSO-d₆) δ 14.75 (brs, 1H), 8.73 (s, 1H), 8.44 (s, 1H), 8.29 (d, J=2.8 Hz, 1H), 8.16 (d, J=2.4 Hz, 1H), 7.66 (dd, J=8.8, 2.8 Hz, 1H), 7.49 (s, 1H), 6.64 (brs, 2H), 6.61 (d, J=9.2 Hz, 1H), 6.13 (d, J=2.8 Hz, 1H), 4.62 (brs, 1H), 3.52 (t, J=5.2 Hz, 2H), 3.25 (t, J=5.6 Hz, 2H), 2.87 (s, 3H); LC-MS (ESI): m/z 455.2 [M+H]⁺.

Analogues compounds, examples 93 to 95, 147, 148, 152, 153 and 162 to 164 were synthesized by following similar procedures using appropriate reagents.

### Example 202: sodium 1-(6-(azetidin-1-yl)-4-methylpyridin-3-yl)-6-chloro-7-(5,7-dihydro-6H-pyrrolo[3,4-b]pyridin-6-yl)-4-oxo-1,4-dihydro-1,8-naphthyridine-3-carboxylate

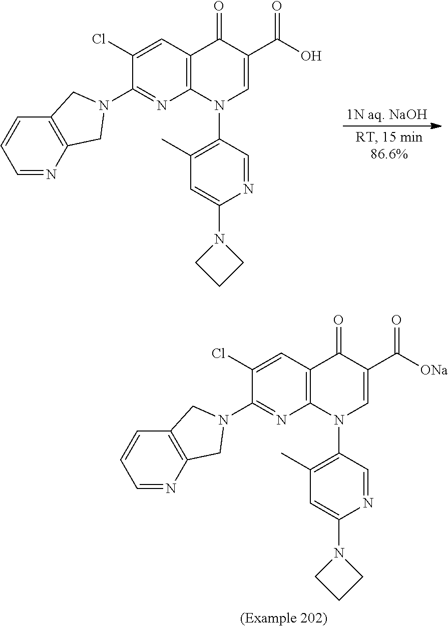

To a stirred suspension of 1-[6-(azetidin-1-yl)-4-methylpyridin-3-yl]-6-chloro-4-oxo-7-{5H,6H,7H-pyrrolo[3,4-b]pyridin-6-yl}-1,4-dihydro-1,8-naphthyridine-3-carboxylic acid (100 mg, 204 μmol) in water (2 mL), was added aqueous 1N sodium hydroxide (204 μL, 204 μmol) solution and stirred for 15 min. After 15 min, clear solution was observed and reaction mixture was lyophilized to afford sodium 1-[6-(azetidin-1-yl)-4-methylpyridin-3-yl]-6-chloro-4-oxo-7-{5H,6H,7H-pyrrolo[3,4-b]pyridin-6-yl}-1,4-dihydro-1,8-naphthyridine-3-carboxylate (90.1 mg, 176 μmol) as an off white solid. ¹H NMR (400 MHz, DMSO-d6): δ 8.44 (d, J=4.0 Hz, 1H), 8.25 (s, 1H), 8.15 (s, 1H), 7.98 (s, 1H), 7.76 (d, J=6.8 Hz, 1H), 7.29 (q, J=5.2 Hz, 1H), 6.43 (s, 1H), 5.08 (s, 2H), 4.77 (s, 2H), 4.06-4.01 (m, 4H), 2.41-2.33 (m, 2H), 1.96 (s, 3H); MS (ESI): m/z 489 [M+H−Na]⁺.

TABLE 3
Exemplified Compounds

| Ex. | Product | Prep HPLC Method | IUPAC name | Analytical data |
|---|---|---|---|---|
| 1 | 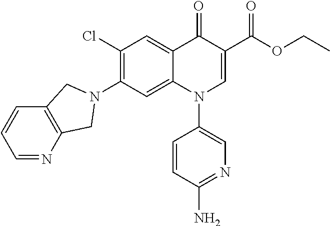 | Column: SunFire Prep C18 OBD Column, 19 * 150 mm 5 um 10 nm; Mobile Phase A: Water (0.1% FA), Mobile Phase B: ACN; Flow rate: 25 mL/min; Gradient: 15 B to 42 B in 8 min; 254/220 nm; RT 1: 9.28 | 1-(6-Aminopyri- din-3-yl)-6-chloro- 4-oxo-7-[5H,7H- pyrrolo[3,4-b]- pyridin-6-yl]quin- oline-3-carboxy- lic acid | LCMS (ESI) [M + H]⁺: 433.95. ¹H NMR (300 MHz, DMSO-d₆) δ 15.169 (s, 1 H), 8.60- 8.38 (m, 2H), 8.27-8.16 (m, 2H), 7.85 (d, J = 7.6 Hz, 1H), 7.68 (s, 1H), 7.32 (d, J = 2.9 Hz, 1H), 6.71-6.60 (m, 3H), 6.41 (s, 1H), 4.94 (d, J = 24.6 Hz, 4H), 2.08 (s, 1H) |
| 2 | 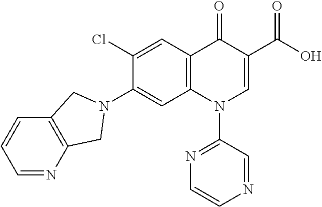 [SMILES: O=C(O)c1cn(-c2cnccn2)c2cc(N3Cc4cccnc4C3)c(Cl)cc2c1=O] | | 6-chloro-4-oxo-1- (pyrazin-2-yl) {5H, 6H,7H pyrrolo- [3,4-b]pyridin-6- yl}-1,4-dihydro- 1,8-naphthyridine- 3-carboxylic acid | ¹H-NMR (400 MHz, DMSO-d₆): δ 14.70 (bs, 1H), 9.31 (d, J = 1.2 Hz, 1H), 9.06 (s, 1H), 8.91 (d, J = 2.8 Hz, 1H), δ 8.83-8.81 (m, 1H), 8.46 (d, J = 4.8 Hz, 1H), 8.41 (s, 1H), 7.78 (d, J = 7.2 Hz, 1H), 7.34-7.30 (m, 1H), 5.17 (bs, 2H), 4.95 (bs, 2H). MS (ESI): m/z 421.0 [M + H]⁺. |
| 3 | 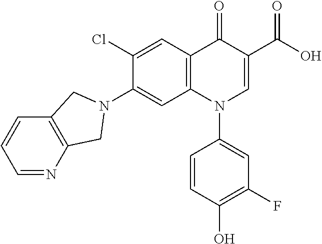 [SMILES: O=C(O)c1cn(-c2ccc(O)c(F)c2)c2cc(N3Cc4cccnc4C3)c(Cl)cc2c1=O] | | 6-chloro-1-(3- fluoro-4-hydroxy- phenyl)-4-oxo-7- {5H,6H,7H-pyr- rolo[3,4-b]pyridin- 6-yl}-1,4-dihydro- quinoline-3-car- boxylic acid | ¹H NMR (400 MHz, DMSO-d₆) δ 15.11 (brs, 1H), 10.68 (s, 1H), 8.57 (s, 1H), 8.46 (d, J = 4.4 Hz, 1H), 8.24 (s, 1H), 7.83 (d, J = 7.2 Hz, 1H), 7.67 (dd, J = 8.0, 2.8 Hz, 1H), 7.38-7.36 (m, 2H), 7.31 (t, J = 2.8 Hz, 1H), 6.38 (s, 1H), 4.99-4.83 (m, 4H); MS (ESI: m/z 452.3[M + H]⁺. |
| 4 | 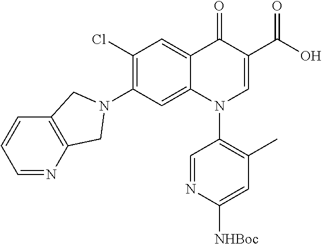 [SMILES: Cc1cc(NC(=O)OC(C)(C)C)ncc1-n1cc(C(=O)O)c(=O)c2cc(Cl)c(N3Cc4cccnc4C3)cc21] | | 1-(6-{[(tert-but- oxy)carbonyl]- amino}-4-methyl- pyridin-3-yl)-6- chloro-4-oxo-7- {5H,6H,7H-pyr- rolo[3,4-b]pyridin- 6-yl}-1,4-dihydro- quinoline-3-car- boxylic acid | ¹H NMR (400 MHz, DMSO-d₆) δ 10.23 (s, 1H), 8.60 (bs, 1H),8.48- 8.37 (m, 2H), 8.24 (s, 1H), 8.03 (s, 1H), 7.82 (d, J = 7.2 Hz, 1H), 7.33-7.27 (m, 1H), 6.11 (s, 1H), 4.94-4.80 (m, 4H), 2.08 (s, 3H), 1.52 (s, 9H) (—COOH peak was not observed); MS (ESI): m/z 548 [M + H]⁺. |
| 5 | 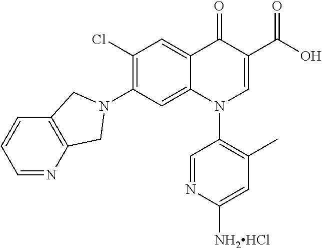 | | 1-(6-amino-4- meth-lpyridin-3- yl)-6-chloro-4- oxo-7-{5H,6H,7H- pyrrolo[3,4-b]pyr- idin-6-yl}-1,4- dihydroquinoline- 3-carboxylicacid hydrochloride | ¹H NMR (400 MHz, DMSO-d₆): δ 15.09 (bs, 1H), 8.65 (s, 1H), 8.47 (d, J = 4.4 Hz, 1H), 8.24 (s, 1H), 8.18 (s, 1H), 7.83 (d, J = 6.8 Hz, 1H), 7.35-7.30 (m, 1H), 7.23 (bs, 1H), 6.70 (s, 1H), 6.26 (m, 1H), 5.08-4.82 (m, 4H), 1.97 (s, 3H) (couple of exchangeable protons were not ob- served); MS (ESI): m/z 448 [M + H]⁺. |
| 6 | 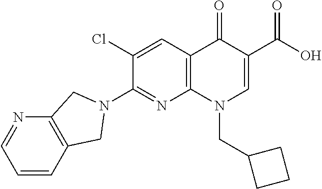 [SMILES: O=C(O)c1cn(CC2CCC2)c2nc(N3Cc4cccnc4C3)c(Cl)cc2c1=O] | | 6-chloro-1-(cyclo- butylmethyl)-4- oxo-7-{5H,6H,7H- pyrrolo[3,4-b]pyr- idin-6-yl}-1,4- dihydro-1,8-naph- thyridine-3-carbox- ylic acid | MS (ESI): m/z 411.0 [M + H]⁺; ¹H NMR (400 MHz, DMSO-d₆) δ 15.19 (s, 1H), 8.99 (s, 1H), 8.53 (d, J = 5.2 Hz, 1H), 8.36 (s, 1H), 7.90 (d, J = 6.8 Hz, 1H), 7.39 (q, J = 4.8 Hz, 1H), 5.38 (s, 2H), 5.30 (s, 2H), 4.59 (d, J = 7.2 Hz, 2H), 2.96-2.95 (m, 1H), 1.98-1.94 (m, 2H), 1.91-1.84 (m, 4H). |
| 7 | 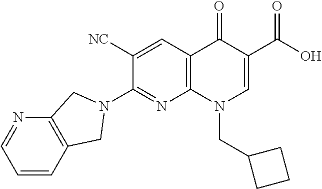 [SMILES: N#Cc1cc2c(=O)c(C(=O)O)cn(CC3CCC3)c2nc1N1Cc2cccnc2C1] | Column: Gemini- C18 (250 * 19.0 mm * 3), Mobile phase-A: 0.01% TFA in Aqueous, Mobile phase-B: Acetonitrile, (T/%B): 0/20, 15/40, 25/95, Flow: 20.0 mL/ min; diluent: Acetonitrile/Water | 6-cyano-1-(cyclo- butylmethyl)-4- oxo-7-{5H,6H,7H- pyrrolo[3,4-b]pyr- idin-6-yl}-1,4- dihydro-1,8-naph- thyridine-3-carbox- ylic acid | MS (ESI): m/z 402.3 [M + H]⁺; ¹H NMR (400 MHz, DMSO-d₆): δ 14.75 (s, 1H), 9.03 (s, 1H), 8.83 (s, 1H), 8.55 (d, J = 4.8 Hz, 1H), 7.95 (d, J = 7.6 Hz, 1H), 7.38 (q, J = 4.8 Hz, 1H), 5.48- 5.12 (brs, 4H), 4.57 (d, J = 7.2 Hz, 2H), 2.98- 2.92 (m, 1H), 1.98-1.95 (m, 2H), 1.89-1.85 (m, |
| | | | | 4H). |
| 8 | 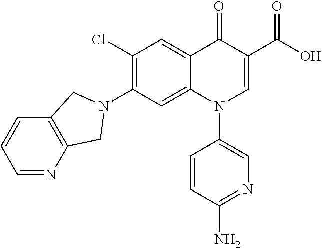 [SMILES: Nc1ccc(-n2cc(C(=O)O)c(=O)c3cc(Cl)c(N4Cc5cccnc5C4)cc32)cn1] | Column: SunFire Prep C18 OBD Column, 19 * 150 mm 5 um 10 nm; Mobile Phase A: Water (0.1% FA), Mobile Phase B: ACN; Flow rate: 25 mL/min; Gradient: 15 B to 42 B in 8 min; 254/220 nm; RT 1: 9.28 | 1-(6-aminopyridin- 3-yl)-6-chloro-7- (5,7-dihydro-6H- pyrrolo[3,4-b]pyr- idin-6-yl)-4-oxo- 1,4-dihydroquin- oline-3-carboxylic acid | LCMS (ESI) [M + H]⁺: 433.95. ¹H NMR (300 MHz, DMSO-d₆) δ 15.169 (s, 1 H), 8.60- 8.38 (m, 2H), 8.27-8.16 (m, 2H), 7.85 (d, J = 7.6 Hz, 1H), 7.68 (s, 1H), 7.32 (d, J = 2.9 Hz, 1H), 6.71-6.60 (m, 3H), 6.41 (s, 1H), 4.94 (d, J = 24.6 Hz, 4H), 2.08 (s, 1H). |
| 9 | 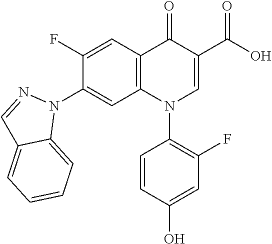 [SMILES: O=C(O)c1cn(-c2ccc(O)cc2F)c2cc(-n3ncc4ccccc43)c(F)cc2c1=O] | Reverse phase flash chromatography | 7-(7-bromo-1H- indazol-1-yl)-6- fluoro-1-(2-fluoro- 4-hydroxyphenyl)- 4-oxo-1,4-dihydro- quinoline-3-carbox- ylic acid | LCMS (ESI) [M + H]⁺: 512. ¹H NMR (300 MHz, DMSO-d₆) δ 10.80 (s, 1H), 9.16 (d, J = 2.4 Hz, 1H), 8.93 (s, 1H), 8.46 (d, J = 11.1 Hz, 1H), 7.90-7.80 (m, 2H), 7.74- 7.63 (m, 2H), 7.10-7.02 (m, 1H), 6.99-6.86 (m, 2H). |
| 10 | 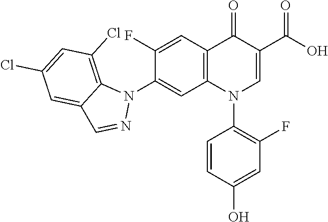 [SMILES: O=C(O)c1cn(-c2ccc(O)cc2F)c2cc(-n3ncc4cc(Cl)cc(Cl)c43)c(F)cc2c1=O] | Reverse phase flash chromatography | 7-(5,7-dichloro- 1H-indazol-1-yl)-6- fluoro-1-(2-fluoro- 4-hydroxyphenyl)- 4-oxo-1,4-dihydro- quinoline-3-carbox- ylic acid | LCMS (ESI) [M + H]⁺: 502.20. ¹H NMR (300 MHz, DMSO-d₆) δ 14.39 (s, 1H), 10.72 (s, 1H), 9.10 (d, J = 2.5 Hz, 1H), 8.93 (s, 1H), 8.47 (d, J = 11.0 Hz, 1H), 7.95 (d, J = 1.7 Hz, 1H), 7.84 (d, J = 6.0 Hz, 1H), 7.69 (t, J = 8.8 Hz, 1H), 7.60 (d, J = 1.7 Hz, 1H), 7.00- 6.85 (m, 2H). |
| 11 | 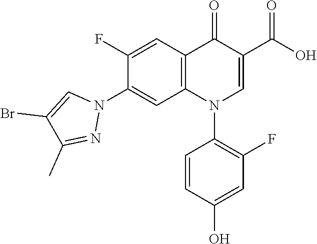 [SMILES: Cc1nn(-c2cc3c(cc2F)c(=O)c(C(=O)O)cn3-c2ccc(O)cc2F)cc1Br] | Reverse phase flash chromatography | 7-(4-bromo-3- methyl-1H-pyr- azol-1-yl)-6-fluoro- 1-(2-fluoro-4- hydroxyphenyl)-4- oxo-1,4-dihydro- quinoline-3-car- boxylic acid | LCMS (ESI) [M + H]⁺: 476. ¹H NMR (300 MHz, DMSO-d₆) δ 14.41 (s, 1H), 10.67 (s, 1H), 8.90 (s, 1H), 8.39 (d, J = 10.0 Hz, 1H), 7.87 (s, 1H), 7.62 (t, J = 8.8 Hz, 1H), 7.45 (dd, J = 6.0, 1.2 Hz, 1H), 6.95-6.80 (m, 2H), 2.16 (d, J = 1.7 Hz, 3H). |
| 12 | 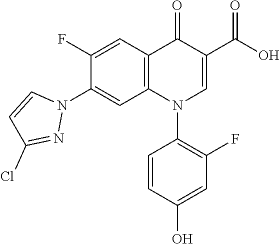 [SMILES: O=C(O)c1cn(-c2ccc(O)cc2F)c2cc(-n3ccc(Cl)n3)c(F)cc2c1=O] | Reverse phase flash chromatography | 7-(3-chloro-1H- pyrazol-1-yl)-6- fluoro-1-(2-fluoro- 4-hydroxyphenyl)- 4-oxo-1,4-dihydro- quinoline-3-carbox- ylic acid | LCMS (ESI) [M + H]⁺: 418.00. ¹H NMR (400 MHz, DMSO-d₆) 14.47 (s, 1H), 10.77 (s, 1H), 8.89 (s, 1H), 8.46 (t, J = 2.8 Hz, 1H), 8.38 (d, J = 11.6 Hz, 1H), 7.66 (t, J = 8.9 Hz, 1H), 7.53 (dd, J = 6.3, 1.2 Hz, 1H), 7.00-6.87 (m, 2H), 6.78 (d, J = 2.7 Hz, 1H). |
| 13 | 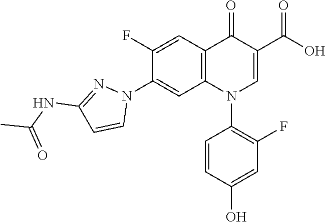 [SMILES: CC(=O)Nc1ccn(-c2cc3c(cc2F)c(=O)c(C(=O)O)cn3-c2ccc(O)cc2F)n1] | Reverse phase flash chromatography | 7-(N-(1H-pyrazol- 3-yl)acetamido)-6- fluoro-1-(2-fluoro- 4-hydroxyphenyl)- 4-oxo-1,4-dihydro- quinoline-3-carbox- ylic acid | LCMS (ESI) [M + H]⁺: 441.10. ¹H NMR (400 MHz, DMSO-d₆) δ 14.54 (s, 1H), 10.77 (s, 2H), 8.82 (s, 1H), 8.38-8.26 (m, 2H), 7.66 (t, J = 8.9 Hz, 1H), 7.54 (dd, J = 6.3, 1.2 Hz, 1H), 7.04- 6.82 (m, 3H), 2.03 (s, 3H). |
| 14 | 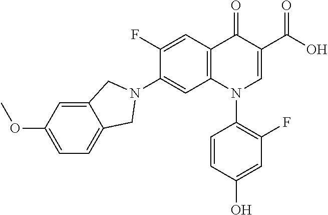 [SMILES: COc1ccc2c(c1)CN(c1cc3c(cc1F)c(=O)c(C(=O)O)cn3-c1ccc(O)cc1F)C2] | Reverse phase flash chromatography | 6-fluoro-1-(2- fluoro-4-hydroxy- phenyl)-7-(5- methoxyisoindo- lin-2-yl)-4-oxo- 1,4-dihydroquin- oline-3-carboxy- lic acid | LCMS (ESI) [M + H]⁺: 465.15. ¹H NMR (300 MHz, DMSO-d₆) δ 8.60 (s, 1H), 7.94 (d, J = 14.3 Hz, 1H), 7.61 (t, J = 8.9 Hz, 1H), 7.28 (d, J = 8.3 Hz, 1H), 7.03-6.80 (m, 4H), 6.06 (d, J = 7.4 Hz, 1H), 4.72 (d, J = 8.9 Hz, 4H), 3.74 (s, 3H). |
| 15 | 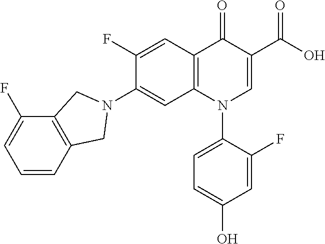 [SMILES: O=C(O)c1cn(-c2ccc(O)cc2F)c2cc(N3Cc4cccc(F)c4C3)c(F)cc2c1=O] | Normal phase silica gel column chromatography | 6-fluoro-1-(2- fluoro-4-hydroxy- phenyl)-7-(4- fluoroisoindolin- 2-yl)-4-oxo-1,4- dihydroquinoline- 3-carboxylic acid | MS (ESI): m/z 453.2 [M + H]⁺; ¹H NMR (400 MHz, DMSO-d₆): δ 15.25 (s, 1H), 10.70 (s, 1H), 8.62 (s, 1H), 7.97 (d, J = 13.2 Hz, 1H), 7.62 (t, J = 9.2 Hz, 1H), 7.40- 7.34 (m, 1H), 7.26 (d, J = 7.2 Hz, 1H), 7.14 (t, J = 8.8 Hz, 1H), 6.94-6.86 (m, 2H), 6.10 (d, J = 7.2 Hz, 1H), 4.88 (s, 2H), 4.82 (s, 2H). |
| 16 | 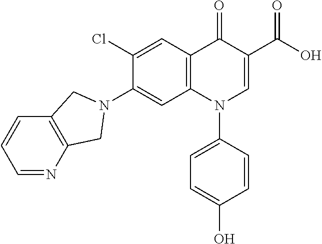 [SMILES: O=C(O)c1cn(-c2ccc(O)cc2)c2cc(N3Cc4cccnc4C3)c(Cl)cc2c1=O] | Trituration with diethyl ether and pentane | 6-chloro-7-(5,7- dihydro-6H-pyr- rolo[3,4-b]pyridin- 6-yl)-1-(4-hydroxy- phenyl)-4-oxo-1,4- dihydroquinoline- 3-carboxylic acid | MS (ESI): m/z 434.3 [M + H]⁺; ¹H NMR (400 MHz, DMSO-d₆): δ 15.02 (s, 1H), 10.22 (s, 1H), 8.53 (s, 1H), 8.46 (d, J = 4.0 Hz, 1H), 8.24 (s, 1H), 7.83 (d, J = 7.2 Hz, 1H), 7.51 (d, J = 8.8 Hz, 2H), 7.34-7.30 (m, 1H), 7.04 (d, J = 8.8 Hz, 2H), 6.38 (s, 1H), 4.94 (s, 2H), 4.84 (s, 2H). |
| 17 | 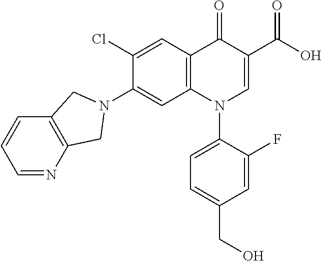 [SMILES: O=C(O)c1cn(-c2ccc(CO)cc2F)c2cc(N3Cc4cccnc4C3)c(Cl)cc2c1=O] | Trituration with diethyl ether and pentane | 6-chloro-7-(5,7- dihydro-6H-pyr- rolo[3,4-b]pyridin- 6-yl)-1-(2-fluoro-4- (hydroxymethyl)- phenyl)-4-oxo-1,4- dihydroquinoline- 3-carboxylic acid | MS (ESI): m/z 466.3 [M + H]⁺; ¹H NMR (400 MHz, DMSO-d₆): δ 14.96 (bs, 1H), 8.72 (s, 1H), 8.45 (d, J = 4.0 Hz, 1H), 8.26 (s, 1H), 7.84- 7.78 (m, 2H), 7.56 (d, J = 11.2 Hz, 1H), 7.48 (d, J = 8.8 Hz, 1H), 7.34-7.29 (m, 1H), 6.25 (s, 1H), 5.52 (bs, 1H), 5.00 (s, 2H), 4.80 (s, 2H), 4.71 (s, 2H). |
| 18 | 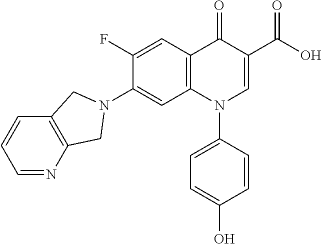 [SMILES: O=C(O)c1cn(-c2ccc(O)cc2)c2cc(N3Cc4cccnc4C3)c(F)cc2c1=O] | Trituration with isopropyl alcohol | 7-(5,7-dihydro-6H- pyrrolo[3,4-b]pyr- idin-6-yl)-6-fluoro- 1-(4-hydroxyphen- yl)-4-oxo-1,4- dihydroquinoline- 3-carboxylic acid | MS (ESI): m/z 418.3 [M + H]⁺; ¹H NMR (400 MHz, DMSO-d₆): δ 15.41 (s, 1H), 10.20 (s, 1H), 8.50-8.45 (m, 2H), 7.98 (d, J = 14.4 Hz, 1H), 7.85 (d, J = 7.2 Hz, 1H), 7.50 (d, J = 8.8 Hz, 2H), 7.34-7.30 (m, 1H), 7.03 (d, J = 8.8 Hz, 2H), 6.20 (d, J = 8.0 Hz, 1H), 4.81 (s, 2H), 4.77 (s, 2H). |
| 19 | 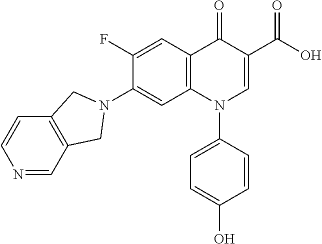 [SMILES: O=C(O)c1cn(-c2ccc(O)cc2)c2cc(N3Cc4ccncc4C3)c(F)cc2c1=O] | Trituration with isopropyl alcohol | 7-(1,3-dihydro-2H- pyrrolo[3,4-c]pyr- idin-2-yl)-6-fluoro- 1-(4-hydroxyphen- yl)-4-oxo-1,4- dihydroquinoline- 3-carboxylic acid | MS (ESI): m/z 418.4 [M + H]⁺; ¹H NMR (400 MHz, DMSO-d₆): δ 15.41 (s, 1H), 10.21 (s, 1H), 8.50-8.45 (m, 2H), 7.97 (d, J = 14.0 Hz, 1H), 7.84 (d, J = 7.2 Hz, 1H), 7.50 (d, J = 8.0 Hz, 2H), 7.34-7.30 (m, 1H), 7.03 (d, J = 8.0 Hz, 2H), 6.20 (d, J = 7.6 Hz, 1H), 4.81 (s, 2H), 4.76 (s, 2H). |
| 20 | 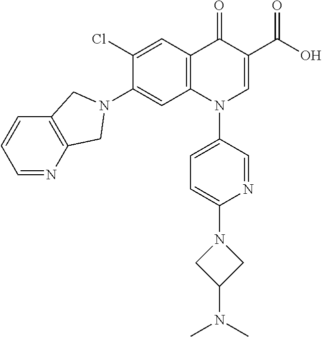 [SMILES: CN(C)C1CN(c2ccc(-n3cc(C(=O)O)c(=O)c4cc(Cl)c(N5Cc6cccnc6C5)cc43)cn2)C1] | Trituration with diethyl ether. | 6-chloro-7-(5,7- dihydro-6H-pyr- rolo[3,4-b]pyridin- 6-yl)-1-(6-(3- (dimethylamino)- azetidin-1-yl)pyri- din-3-yl)-4-oxo- 1,4-dihydroquin- oline-3-carboxylic acid | MS (ESI): m/z 517.3 [M + H]⁺; ¹H NMR (400 MHz, DMSO-d₆): δ 15.11 (s, 1H), 8.55 (s, 1H), 8.47 (d, J = 4.4 Hz, 1H), 8.34 (s, 1H), 8.24 (s, 1H), 7.83 (d, J = 7.2 Hz, 2H), 7.34-7.30 (m, 1H), 6.61 (d, J = 8.8 Hz, 1H), 6.39 (s, 1H), 4.99 (s, 2H), 4.86 (s, 2H), 4.18-4.09 (m, 2H), 3.91-3.85 (m, 2H), 3.32-3.26 (m, 1H), 2.15 (s, 6H). |
| 21 | 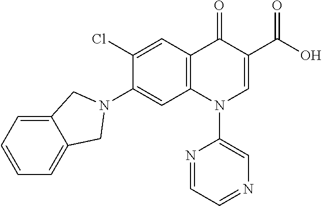 [SMILES: O=C(O)c1cn(-c2cnccn2)c2cc(N3Cc4ccccc4C3)c(Cl)cc2c1=O] | Trituration with isopropanol | 6-chloro-7-(iso- indolin-2-yl)-4- oxo-1-(pyrazin-2- yl)-1,4-dihydro- quinoline-3-carbox- ylic acid | MS (ESI): m/z 419.3 [M + H]⁺; ¹H NMR (400 MHz, DMSO-d₆): δ 14.75 (s, 1H), 8.97 (s, 1H), 8.94 (s, 1H), 8.81 (s, 1H), 8.77 (s, 1H), 8.46 (s, 1H), 7.34-7.27 (m, 4H), 6.38 (s, 2H), 4.97 (s, 4H). |
| 22 | 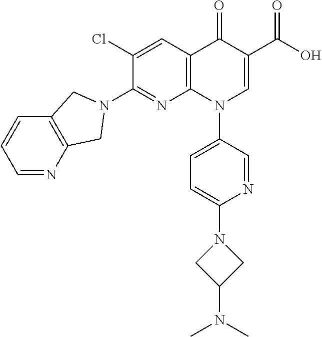 [SMILES: CN(C)C1CN(c2ccc(-n3cc(C(=O)O)c(=O)c4cc(Cl)c(N5Cc6cccnc6C5)nc43)cn2)C1] | Trituration with IPA, diethyl ether and pentane | 6-chloro-7-(5,7- dihydro-6H-pyr- rolo[3,4-b]pyridin- 6-yl)-1-(6-(3- (dimethylamino)- azetidin-1-yl)pyr- idin-3-yl)-4-oxo- 1,4-dihydro-1,8- naphthyridine-3- carboxylic acid | MS (ESI): m/z 518.4 [M + H]⁺; ¹H NMR (400 MHz, DMSO-d₆): δ 15.01 (bs, 1H), 8.66 (s, 1H), 8.47 (d, J = 4.4 Hz, 1H), 8.40 (s, 1H), 8.29 (s, 1H), 7.85-7.77 (m, 2H), 7.35-7.31 (m, 1H), 6.57 (d, J = 9.2 Hz, 1H), 5.16 (s, 2H), 4.92 (s, 2H), 4.20-4.08 (m, 2H), 3.86- 3.81 (m, 2H), 3.26-3.23 (m, 1H), 2.15 (s, 6H). |
| 23 | 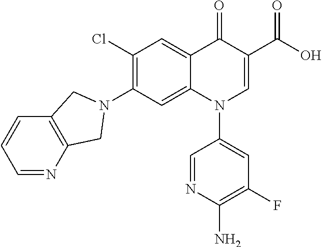 [SMILES: Nc1ncc(-n2cc(C(=O)O)c(=O)c3cc(Cl)c(N4Cc5cccnc5C4)cc32)cc1F] | trituration with diethyl ether and MeOH | 1-(6-amino-5- fluoropyridin-3- yl)-6-chloro-7-(5,7- dihydro-6H-pyr- rolo-[3,4-b]pyridin- 6-yl)-4-oxo-1,4- dihydroquinoline- 3-carboxylic acid | MS (ESI): m/z 452 [M + H]⁺. ¹H NMR (400 MHz, DMSO-d₆): δ 15.09 (bs, 1H), 8.62 (s, 1H), 8.46 (bs, 1H), 8.23 (s, 1H), 8.10 (bs, 1H), 7.90-7.80 (m, 2H), 7.34-7.30 (m, 1H), 6.91 (bs, 2H), 6.43 (s, 1H), 4.98 (s, 2H), 4.94 (s, 2H); |
| 24 | 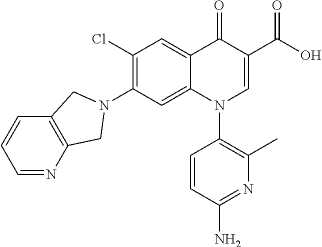 [SMILES: Cc1nc(N)ccc1-n1cc(C(=O)O)c(=O)c2cc(Cl)c(N3Cc4cccnc4C3)cc21] | trituration with diethyl ether and MeOH | 1-(6-amino-2- methylpyridin-3- yl)-6-chloro-7-(5,7- dihydro-6H-pyr- rolo[3,4-b]pyridin- 6-yl)-4-oxo-1,4- dihydroquinoline- 3-carboxylic acid | MS (ESI): m/z 448.1 [M + H]⁺; ¹H NMR (400 MHz, DMSO-d₆): δ 8.69 (bs, 1H), 8.48 (d, J = 4.4 Hz, 1H), 8.24 (s, 1H), 7.90-7.83 (m, 2H), 7.65 (bs, 1H), 7.36-7.31 (m, 1H), 6.82 (d, J = 8.8 Hz, 1H), 6.30 (s, 1H), 5.12- 4.98 (m, 3H), 4.85 (d, J = 14.8 Hz, 1H), 2.13 (s, 3H). (few exchangeable protons were not ob- served by 1HNMR). |
| 25 | 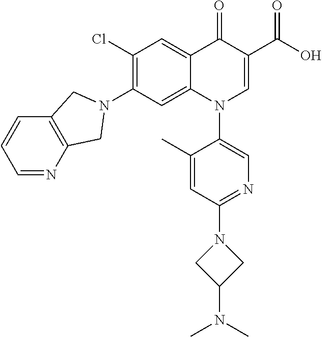 [SMILES: Cc1cc(N2CC(N(C)C)C2)ncc1-n1cc(C(=O)O)c(=O)c2cc(Cl)c(N3Cc4cccnc4C3)cc21] | RP flash column purification | 6-chloro-7-(5,7- dihydro-6H-pyr- rolo[3,4-b]pyridin- 6-yl)-1-(6-(3- (dimethylamino)- azetidin-1-yl)-4- methylpyridin-3- yl)-4-oxo-1,4- dihydroquinoline- 3-carboxylic acid | MS (ESI): m/z 531 [M + H]⁺; ¹H NMR (400 MHz, DMSO-d₆): δ 8.56 (s, 1H), 8.46 (d, J = 4.8 Hz, 1H), 8.26 (s, 1H), 8.21 (s, 1H), 7.83 (d, J = 7.6 Hz, 1H), 7.33-7.30 (m, 1H), 6.53 (s, 1H), 6.18 (s, 1H), 5.00 (s, 2H), 4.82 (s, 2H), 4.15 (bs, 2H), 3.92 (bs, 2H), 3.35-3.30 (m, 1H), 2.26 (bs, 6H), 1.99 (s, 3H) (—COOH peak was not observed); |
| 26 | 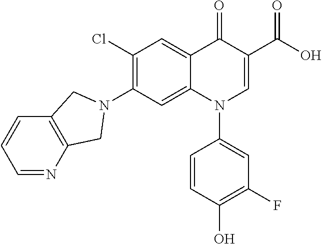 [SMILES: O=C(O)c1cn(-c2ccc(O)c(F)c2)c2cc(N3Cc4cccnc4C3)c(Cl)cc2c1=O] | trituration with IPA & Acetonitrile, diethyl ether, n- pentane | 6-chloro-7-(5,7- dihydro-6H-pyr- rolo[3,4-b]pyridin- 6-yl)-1-(3-fluoro- 4-hydroxyphenyl)- 4-oxo-1,4-dihydro- quinoline-3-carbox- ylic acid | MS (ESI: m/z 452.1 [M + H]⁺; ¹H NMR (400 MHz, DMSO-d₆) δ 15.11 (brs, 1H), 10.68 (s, 1H), 8.57 (s, 1H), 8.46 (d, J = 4.4 Hz, 1H), 8.24 (s, 1H), 7.83 (d, J = 7.2 Hz, 1H), 7.67 (dd, J = 8.0, 2.8 Hz, 1H), 7.40-7.29 (m, 2H), 7.21 (t, J = 2.8 Hz, 1H), 6.38 (s, 1H), 4.96-4.83 (m, 4H); |
| 27 | 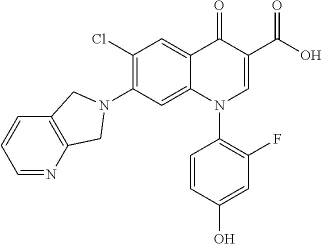 [SMILES: O=C(O)c1cn(-c2ccc(O)cc2F)c2cc(N3Cc4cccnc4C3)c(Cl)cc2c1=O] | Prep-HPLC, Method-4 of Table-2 | 6-chloro-7-(5,7- dihydro-6H-pyr- rolo[3,4-b]pyridin- 6-yl)-1-(2-fluoro- 4-hydroxyphenyl)- 4-oxo-1,4-dihydro- quinoline-3-carbox- ylic acid | MS (ESI: m/z 452.2 [M + H]⁺; ¹H NMR (400 MHz, DMSO-d₆) δ 14.99 (s, 1H), 10.69 (s, 1H), 8.67 (s, 1H), 8.46 (d, J = 4.4 Hz, 1H), 8.24 (s, 1H), 7.83 (d, J = 7.2 Hz, 1H), 7.62 (t, J = 8.8 Hz, 1H), 7.34-7.30 (m, 1H), 6.97- 6.88 (m, 2H), 6.27 (s, 1H), 4.97 (s, 2H), 4.87 (s, 2H); |
| 28 | 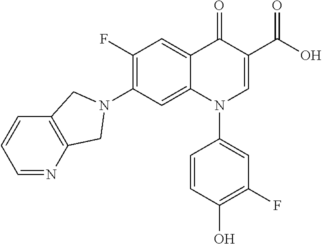 [SMILES: O=C(O)c1cn(-c2ccc(O)c(F)c2)c2cc(N3Cc4cccnc4C3)c(F)cc2c1=O] | trituration with IPA & Acetonitrile, diethyl ether, n- pentane | 7-(5,7-dihydro-6H- pyrrolo[3,4-b]pyr- idin-6-yl)-6-fluoro- 1-(3-fluoro-4- hydroxyphenyl)-4- oxo-1,4-dihydro- quinoline-3-carbox- ylic acid | MS (ESI: m/z 436.2 [M + H]⁺; ¹H NMR (400 MHz, DMSO-d₆) δ 15.37 (s, 1H), 10.67 (bs, 1H), 8.54 (s, 1H), 8.47 (d, J = 4.8 Hz, 1H), 7.98 (d, J = 14.0 Hz, 1H), 7.85 (d, J = 7.6 Hz, 1H), 7.67 (dd, J = 8.0, 2.8 Hz, 1H), 7.40- 7.29 (m, 2H), 7.21 (t, J = 4.8 Hz, 1H), 6.19 (d, J = 7.6 Hz, 1H), 4.90-4.76 (m, 4H); |
| 29 | 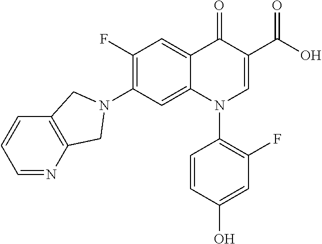 [SMILES: O=C(O)c1cn(-c2ccc(O)cc2F)c2cc(N3Cc4cccnc4C3)c(F)cc2c1=O] | trituration with IPA & Acetonitrile, diethyl ether, n- pentane | 7-(5,7-dihydro-6H- pyrrolo[3,4-b]pyr- idin-6-yl)-6-fluoro- 1-(2-fluoro-4- hydroxyphenyl)-4- oxo-1,4-dihydro- quinoline-3-carbox- ylic acid | MS (ESI: m/z 436.1 [M + H]⁺; ¹H NMR (400 MHz, DMSO-d₆) δ 15.25 (s, 1H), 10.69 (s, 1H), 8.63 (s, 1H), 8.47 (d, J = 4.4 Hz, 1H), 7.98 (d, J = 14.0 Hz, 1H), 7.84 (d, J = 7.2 Hz, 1H), 7.62 (t, J = 8.8 Hz, 1H), 7.34-7.30 (m, 1H), 6.97-6.88 (m, 2H), 6.11 (d, J = 7.2 Hz, 1H), 4.84 (s, 2H), 4.78 (s, 2H); |
| 30 | 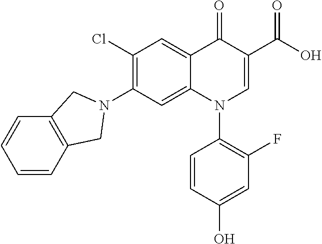 [SMILES: O=C(O)c1cn(-c2ccc(O)cc2F)c2cc(N3Cc4ccccc4C3)c(Cl)cc2c1=O] | trituration with MeOH & n-pentane | 6-chloro-1-(2- fluoro-4-hydroxy- phenyl)-7-(isoindo- lin-2-yl)-4-oxo-1,4- dihydroquinoline- 3-carboxylic acid | MS (ESI): m/z 449 [M − H]; ¹H NMR (400 MHz, DMSO-d₆): δ 15.02 (s, 1H), 10.70 (s, 1H), 8.64 (s, 1H), 8.23 (s, 1H), 7.62 (t, J = 8.8 Hz, 1H), 7.40- 7.37 (m, 2H), 7.31-7.28 (m, 2H), 6.96-6.87 (m, 2H), 6.27 (s, 1H), 4.90 (s, 4H); |
| 31 | 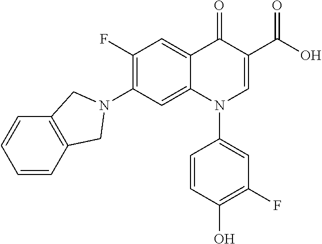 [SMILES: O=C(O)c1cn(-c2ccc(O)c(F)c2)c2cc(N3Cc4ccccc4C3)c(F)cc2c1=O] | trituratation with 30% IPA in diethyl ether | 6-fluoro-1-(3- fluoro-4-hydroxy- phenyl)-7-(isoindo- lin-2-yl)-4-oxo-1,4- dihydroquinoline- 3-carboxylic acid | MS (ESI): m/z 435.3 [M + H]⁺; ¹H NMR (400 MHz, DMSO-d₆): δ 15.39 (s, 1H), 10.67 (s, 1H), 8.51 (s, 1H), 7.96 (d, J = 14.4 Hz, 1H), 7.68-7.65 (m, 1H), 7.40-7.19 (m, 6H), 6.17 (d, J = 7.6 Hz, 1H), 4.86-4.75 (m, 4H). |
| 32 | 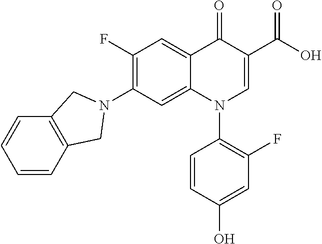 [SMILES: O=C(O)c1cn(-c2ccc(O)cc2F)c2cc(N3Cc4ccccc4C3)c(F)cc2c1=O] | Prep-HPLC, Method-5 of Table-2 | 6-fluoro-1-(2- fluoro-4-hydroxy- phenyl)-7-(isoindol- in-2-yl)-4-oxo-1,4- dihydroquinoline- 3-carboxylic acid | MS (ESI): m/z 435 [M + H]⁺; ¹H NMR (400 MHz, DMSO-d₆): δ 15.29 (s, 1H), 10.69 (s, 1H), 8.60 (s, 1H), 7.96 (d, J = 14.4 Hz, 1H), 7.62 (t, J = 8.8 Hz, 1H), 7.41- 7.28 (m, 4H), 6.96-6.87 (m, 2H), 6.09 (d, J = 7.2 Hz, 1H), 4.80 (s, 4H); |
| 33 | 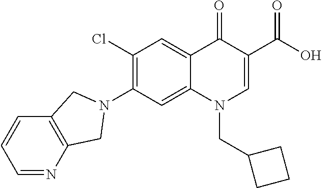 [SMILES: O=C(O)c1cn(CC2CCC2)c2cc(N3Cc4cccnc4C3)c(Cl)cc2c1=O] | Prep-HPLC, Method-6 of Table-2 | 6-chloro-1-(cyclo- butylmethyl)-7- (5,7-dihydro-6H- pyrrolo[3,4-b]pyr- idin-6-yl)-4-oxo- 1,4-dihydroquin- oline-3-carboxylic acid | MS (ESI): m/z 410.0 [M + H]⁺; ¹H NMR (400 MHz, DMSO-d₆): δ 8.92 (s, 1H), 8.52 (d, J = 4.0 Hz, 1H), 8.18 (s, 1H), 7.88 (d, J = 8.0 Hz, 1H), 7.40-7.36 (m, 1H), 7.00 (s, 1H), 5.22 (s, 2H), 5.13 (s, 2H), 4.62 (d, J = 7.6 Hz, 2H), 3.00-2.91 (m, 1H), 2.01-1.80 (m, 6H) (—COOH peak was not |
| | | | | observed). |
| 34 | 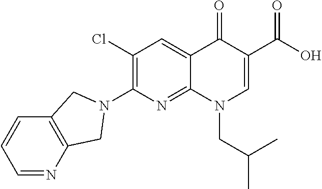 [SMILES: CC(C)Cn1cc(C(=O)O)c(=O)c2cc(Cl)c(N3Cc4cccnc4C3)nc21] | trituration with diethyl ether and IPA (1:1) | 6-chloro-7-(5,7- dihydro-6H-pyr- rolo[3,4-b]pyridin- 6-yl)-1-isobutyl-4- oxo-1,4-dihydro- 1,8-naphthyridine- 3-carboxylic acid | MS (ESI): m/z 399.0 [M + H]⁺; ¹H NMR (400 MHz, DMSO-d₆): δ 15.17 (s, 1H), 8.94 (s, 1H), 8.52 (d, J = 4.4 Hz, 1H), 8.35 (s, 1H), 7.89 (d, J = 7.6 Hz, 1H), 7.39- 7.36 (m, 1H), 5.36 (s, 2H), 5.29 (s, 2H), 4.37 (d, J = 7.6 Hz, 2H), 2.36- 2.30 (m, 1H), 0.95 (d, J = 7.4 Hz, 6H). |
| 35 | 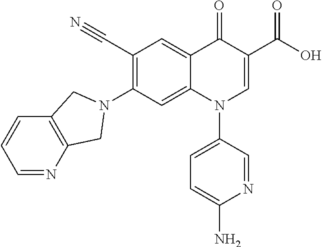 [SMILES: N#Cc1cc2c(=O)c(C(=O)O)cn(-c3ccc(N)nc3)c2cc1N1Cc2cccnc2C1] | trituration with 10% Isopropanol in diethyl ether | 1-(6-aminopyridin- 3-yl)-6-cyano-7- (5,7-dihydro-6H- pyrrolo[3,4-b]pyr- idin-6-yl)-4-oxo- 1,4-dihydroquin- oline-3-carboxylic acid | MS (ESI): m/z 425.0 [M + H]⁺; ¹H NMR (400 MHz, DMSO-d₆): δ 8.77 (s, 1H), 8.56 (s, 1H), 8.52 (d, J = 4.4 Hz, 1H), 8.46 (d, J = 2.0 Hz, 1H), 8.09 (dd, J = 2.4, 9.6 Hz, 1H), 7.92 (d, J = 7.6 Hz, 1H), 7.40-7.36 (m, 1H), 7.13 (d, J = 9.2 Hz, 1H), 6.27 (s, 1H), 5.15-4.96 (m, 4H) (exchangeable protons were not seen); |
| 36 | 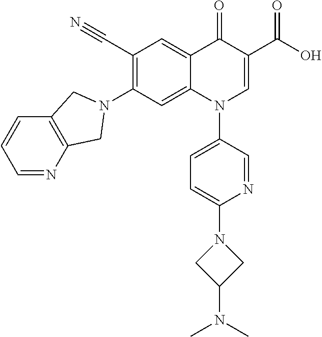 [SMILES: CN(C)C1CN(c2ccc(-n3cc(C(=O)O)c(=O)c4cc(C#N)c(N5Cc6cccnc6C5)cc43)cn2)C1] | trituration with 10% Isopropanol in diethyl ether | 6-cyano-7-(5,7- dihydro-6H-pyr- rolo[3,4-b]pyridin- 6-yl)-1-(6-(3- (dimethylamino)- azetidin-1-yl)pyr- idin-3-yl)-4-oxo- 1,4-dihydroquin- oline-3-carboxylic acid | MS (ESI): m/z 508.5 [M + H]⁺; ¹H NMR (400 MHz, DMSO-d₆): δ 11.80 (brs, 1H), 8.59 (s, 1H), 8.57 (s, 1H), 8.51 (d, J = 4.4 Hz, 1H), 8.42 (d, J = 2.4 Hz, 1H), 8.03 (d, J = 6.8 Hz, 1H), 7.92 (dd, J = 2.8, 5.6 Hz, 1H,), 7.39-7.35 (m, 1H), 6.74 ((d, J = 8.8 Hz, 1H), 6.11 (s, 1H), 5.11 (bs, 2H), 4.91 (bs, 2H), 4.41-4.35 (m, 5H), 2.80 (s, 6H). |
| 37 | 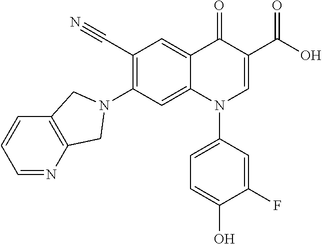 [SMILES: N#Cc1cc2c(=O)c(C(=O)O)cn(-c3ccc(O)c(F)c3)c2cc1N1Cc2cccnc2C1] | trituration with 10% Isopropanol in diethyl ether | 6-cyano-7-(5,7- dihydro-6H-pyr- rolo[3,4-b]pyridin- 6-yl)-1-(3-fluoro- 4-hydroxyphenyl)- 4-oxo-1,4-dihydro- quinoline-3-carbox- ylic acid | MS (ESI): m/z 443 [M + H]⁺; ¹H NMR (400 MHz, DMSO-d₆): δ 14.67 (bs, 1H), 10.69 (s, 1H), 8.57 (s, 1H), 8.55 (s, 1H), 8.49 (d, J = 4.0 Hz, 1H), 7.89 (d, J = 7.6 Hz, 1H), 7.67 (dd, J = 11.2, 2.4 Hz, 1H), 7.40-7.32 (m, 2H), 7.22 (t, J = 9.2 Hz, 1H), 6.17 (s, 1H), 5.00- 4.89 (m, 4H); |
| 38 | 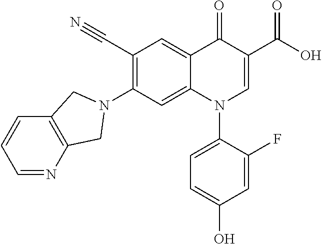 [SMILES: N#Cc1cc2c(=O)c(C(=O)O)cn(-c3ccc(O)cc3F)c2cc1N1Cc2cccnc2C1] | trituration with 10% Isopropanol in diethyl ether | 6-cyano-7-(5,7- dihydro-6H-pyr- rolo[3,4-b]pyridin- 6-yl)-1-(2-fluoro- 4-hydroxyphenyl)- 4-oxo-1,4-dihydro- quinoline-3-carbox- ylic acid | MS (ESI): m/z 443.10 [M + H]⁺; ¹H NMR (400 MHz, DMSO-d₆): δ 10.9 (brs, 1H), 8.67 (s, 1H), 8.55 (s, 1H), 8.51 (d, J = 4.4 Hz, 1H), 7.92 (d, J = 7.6 Hz, 1H), 7.64-7.59 (m, 1H), 7.40-7.36 (m, 1H), 7.01-6.94 (m, 2H), 6.09 (s, 1H), 4.96 (s, 2H), 4.94 (s, 2H) (peak for —COOH proton was not observed); |
| 39 | 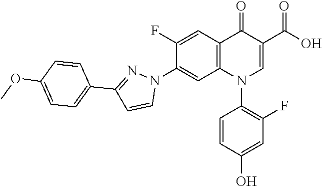 [SMILES: COc1ccc(-c2ccn(-c3cc4c(cc3F)c(=O)c(C(=O)O)cn4-c3ccc(O)cc3F)n2)cc1] | Prep-HPLC, Method-2 of Table-2 | 6-fluoro-1-(2- fluoro-4-hydroxy- phenyl)-7-(3-(4- methoxyphenyl)- 1H-pyrazol-1-yl)- 4-oxo-1,4-dihydro- quinoline-3-carbox- ylic acid | MS (ESI): m/z 490.1 [M + H]⁺; ¹H NMR (400 MHz, DMSO-d₆) δ 14.57 (s, 1H), 10.74 (s, 1H), 8.89 (s, 1H), 8.42 (t, J = 2.8 Hz, 1H), 8.36 (d, J = 11.6 Hz, 1H), 7.76-7.64 (m, 4H), 7.09 (d, J = 2.8 Hz, 1H), 7.02-6.90 (m, 4H), 3.80 (s, 3H). |
| 40 | 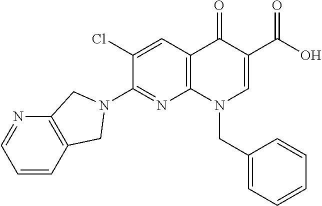 [SMILES: O=C(O)c1cn(Cc2ccccc2)c2nc(N3Cc4cccnc4C3)c(Cl)cc2c1=O] | trituration with 10% Isopropanol in diethyl ether | 1-benzyl-6-chloro- 7-(5,7-dihydro-6H- pyrrolo[3,4-b]pyr- idin-6-yl)-4-oxo- 1,4-dihydro-1,8- naphthyridine-3- carboxylic acid | MS (ESI): m/z 433.0 [M + H]⁺; ¹H NMR (400 MHz, DMSO-d₆): δ 15.14 (bs, 1H), 9.14 (s, 1H), 8.50 (d, J = 4.4 Hz, 1H), 8.35 (s, 1H), 7.86 (d, J = 8.0 Hz, 1H), 7.40- 7.26 (m, 6H), 5.78 (s, 2H), 5.25 (bs, 2H), 5.19 (bs, 2H). |
| 41 | 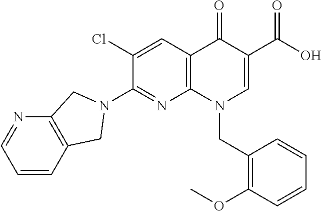 [SMILES: COc1ccccc1Cn1cc(C(=O)O)c(=O)c2cc(Cl)c(N3Cc4cccnc4C3)nc21] | trituration with 10% Isopropanol in diethyl ether | 6-chloro-7-(5,7- dihydro-6H-pyr- rolo[3,4-b]pyridin- 6-yl)-1-(2-meth- oxybenzyl)-4-oxo- 1,4-dihydro-1,8- naphthyridine-3- carboxylic acid | MS (ESI): m/z 463.0 [M + H]⁺; ¹H NMR (400 MHz, DMSO-d₆): δ 15.13 (bs, 1H), 9.07 (s, 1H), 8.51 (d, J = 4.4 Hz, 1H), 8.34 (s, 1H), 7.85 (d, J = 8.0 Hz, 1H), 7.38- 7.26 (m, 2H), 7.14-7.06 (m, 2H), 6.92-6.87 (m, 1H), 5.71 (s, 2H), 5.25 (bs, 2H), 5.13 (bs, 2H), 3.89 (s, 3H). |
| 42 | 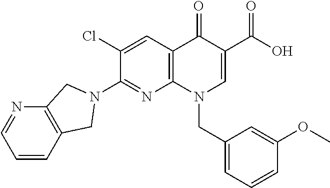 [SMILES: COc1cccc(Cn2cc(C(=O)O)c(=O)c3cc(Cl)c(N4Cc5cccnc5C4)nc32)c1] | trituration with 10% Isopropanol in diethyl ether | 6-chloro-7-(5,7- dihydro-6H-pyr- rolo[3,4-b]pyridin- 6-yl)-1-(3-meth- oxybenzyl)-4-oxo- 1,4-dihydro-1,8- naphthyridine-3- carboxylic acid | MS (ESI): m/z 463.1 [M + H]⁺; ¹H NMR (400 MHz, DMSO-d₆): δ 15.13 (s, 1H), 9.14 (s, 1H), 8.51 (d, J = 4.4 Hz, 1H), 8.34 (s, 1H), 7.84 (d, J = 7.6 Hz, 1H), 7.38- 7.34 (m, 1H), 7.26 (t, J = 8.0 Hz, 1H), 7.01 (s, 1H), 7.14-7.06 (m, 2H), 6.90- 6.84 (m, 2H), 5.74 (s, 2H), 5.25 (bs, 2H), 5.19 |
| | | | | (bs, 2H), 3.74 (s, 3H). |
| 43 | 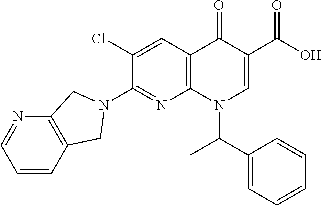 [SMILES: CC(c1ccccc1)n1cc(C(=O)O)c(=O)c2cc(Cl)c(N3Cc4cccnc4C3)nc21] | trituration with 10% Isopropanol in diethyl ether | 6-chloro-7-(5,7- dihydro-6H-pyr- rolo[3,4-b]pyridin- 6-yl)-4-oxo-1-(1- phenylethyl)-1,4- dihydro-1,8-naph- thyridine-3-carbox- ylic acid | MS (ESI): m/z 447.0 [M + H]⁺; ¹H NMR (400 MHz, DMSO-d₆): δ 15.10 (bs, 1H), 8.68 (s, 1H), 8.52 (d, J = 4.0 Hz, 1H), 8.37 (s, 1H), 7.87 (d, J = 8.0 Hz, 1H), 7.49- 7.31 (m, 6H), 6.90-6.76 (s, 1H), 5.35-5.20 (m, 4H), 1.94 (d, J = 6.8 Hz, 3H). |
| 44 | 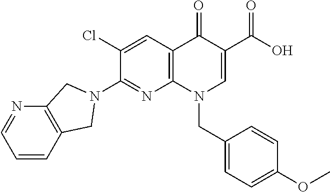 [SMILES: COc1ccc(Cn2cc(C(=O)O)c(=O)c3cc(Cl)c(N4Cc5cccnc5C4)nc32)cc1] | trituration with 10% Isopropanol in diethyl ether | 6-chloro-7-(5,7- dihydro-6H-pyr- rolo[3,4-b]pyridin- 6-yl)-1-(4-meth- oxybenzyl)-4-oxo- 1,4-dihydro-1,8- naphthyridine-3- carboxylic acid | MS (ESI): m/z 463.0 [M + H]⁺; ¹H NMR (400 MHz, DMSO-d₆): δ 15.14 (s, 1H), 9.10 (s, 1H), 8.52 (d, J = 4.4 Hz, 1H), 8.35 (s, 1H), 7.89 (d, J = 8.0 Hz, 1H), 7.40- 7.34 (m, 3H), 6.92 (d, J = 8.8 Hz, 2H), 5.71 (s, 2H), 5.31 (bs, 2H), 5.26 (bs, 2H), 3.70 (s, 3H). |
| 45 | 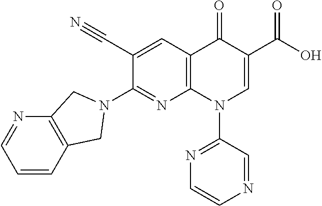 [SMILES: N#Cc1cc2c(=O)c(C(=O)O)cn(-c3cnccn3)c2nc1N1Cc2cccnc2C1] | trituration with diethyl ether | 6-cyano-7-(5,7- dihydro-6H-pyr- rolo[3,4-b]pyridin- 6-yl)-4-oxo-1- (pyrazin-2-yl)-1,4- dihydro-1,8-naph- thyridine-3-carbox- ylic acid | MS (ESI): m/z 412 [M + H]⁺; ¹H NMR (400 MHz, DMSO-d₆): δ 14.30 (bs, 1H), 9.28 (s, 1H), 9.07 (s, 1H), 8.90 (s, 2H), 8.83 (s, 1H), 8.49 (d, J = 4.8 Hz, 1H), 7.86 (bs, 1H), 7.38-7.33 (m, 1H), 5.26 (bs, 2H), 4.75 (bs, 2H). |
| 46 | 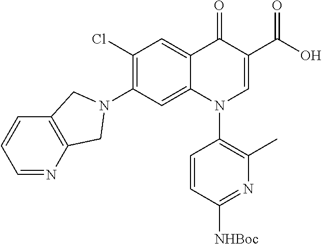 [SMILES: Cc1nc(NC(=O)OC(C)(C)C)ccc1-n1cc(C(=O)O)c(=O)c2cc(Cl)c(N3Cc4cccnc4C3)cc21] | trituration with diethyl ether and MeOH | 1-(6-((tert-butoxy- carbonyl)amino)-2- methylpyridin-3- yl)-6-chloro-7-(5,7- dihydro-6H-pyr- rolo[3,4-b]pyridin- 6-yl)-4-oxo-1,4- dihydroquinoline- 3-carboxylic acid | MS (ESI): m/z 548.0 [M + H]⁺; ¹H NMR (400 MHz, DMSO-d₆): δ 10.22 (s, 1H), 8.46-8.42 (m, 2H), 8.21 (s, 1H), 7.96-7.90 (m, 2H), 7.81 (d, J = 7.6 Hz, 1H), 7.31- 7.27 (m, 1H), 6.12 (s, 1H), 4.90-4.80 (m, 4H), 2.13 (s, 3H), 1.51 (s, 9H) (—COOH peak was not observed); |
| 47 | 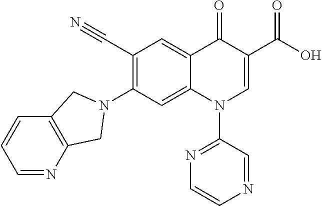 [SMILES: N#Cc1cc2c(=O)c(C(=O)O)cn(-c3cnccn3)c2cc1N1Cc2cccnc2C1] | trituration with 10% Isopropanol in diethyl ether | 6-cyano-7-(5,7- dihydro-6H-pyr- rolo[3,4-b]pyridin- 6-yl)-4-oxo-1-(pyr- azin-2-yl)-1,4- dihydroquinoline- 3-carboxylic acid | MS (ESI): m/z 411.1 [M + H]⁺; ¹H NMR (400 MHz, DMSO-d₆): δ 14.52 (bs, 1H), 9.21 (s, 1H), 9.02 (d, J = 2.8 Hz, 1H), 8.96 (s, 1H), 8.88 (d, J = 4.0 Hz, 1H), 8.58 (s, 1H), 8.49 (d, J = 3.6 Hz, 1H), 7.85 (d, J = 6.8 Hz, 1H), 7.37-7.33 (m, 1H), 6.42 (s, 1H), 5.15- 4.98 (m, 4H). |
| 48 | 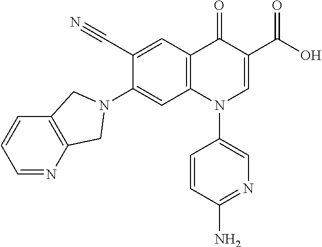 [SMILES: N#Cc1cc2c(=O)c(C(=O)O)cn(-c3ccc(N)nc3)c2cc1N1Cc2cccnc2C1] | trituration with 10% Isopropanol in diethyl ether | 1-(6-aminopyridin- 3-yl)-6-cyano-7- (5,7-dihydro-6H- pyrrolo[3,4-b]pyr- idin-6-yl)-4-oxo- 1,4-dihydroquin- oline-3-carboxylic acid | MS (ESI): m/z 425 [M + H]⁺; ¹H NMR (400 MHz, DMSO-d₆): δ 14.72 (s, 1H), 8.54 (d, J = 6.8 Hz, 2H), 8.48 (d, J = 4.0 Hz, 1H), 8.19 (s, 1H), 7.89 (d, J = 7.2 Hz, 1H), 7.68- 7.66 (m, 1H), 7.36-7.32 (m, 1H), 6.66-6.64 (m, 3H), 6.21 (s, 1H), 4.97 (s, 4H); |
| 49 | 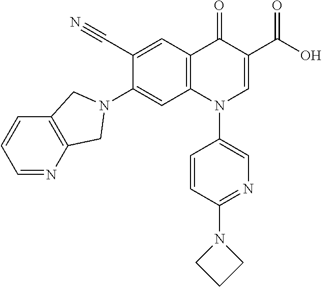 [SMILES: N#Cc1cc2c(=O)c(C(=O)O)cn(-c3ccc(N4CCC4)nc3)c2cc1N1Cc2cccnc2C1] | trituration with diethyl ether and MeOH | 1-(6-(azetidin-1-yl)- pyridin-3-yl)-6- cyano-7-(5,7- dihydro-6H-pyr- rolo-[3,4-b]pyridin- 6-yl)-4-oxo-1,4- dihydroquinoline- 3-carboxylic acid | MS (ESI): m/z 465 [M + H]⁺; ¹H NMR (400 MHz, DMSO-d₆): δ 8.57 (d, J = 7.6 Hz, 2H), 8.51 (d, J = 4.0 Hz, 1H), 8.38 (d, J = 2.4 Hz, 1H), 7.92- 7.87 (m, 1H), 7.39-7.35 (m, 1H), 6.68 (d, J = 13.2 Hz, 1H), 6.20 (s, 1H), 5.07-4.90 (m, 4H), 4.16 (t, J = 8.4 Hz, 4H), 2.42- 2.32 (m, 2H) —COOH proton was not observed; |
| 50 | 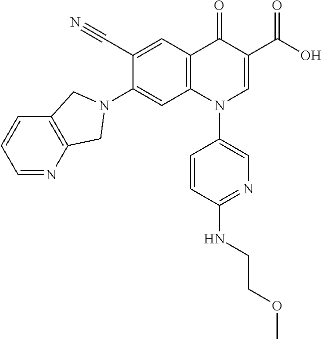 [SMILES: COCCNc1ccc(-n2cc(C(=O)O)c(=O)c3cc(C#N)c(N4Cc5cccnc5C4)cc32)cn1] | trituration with IPA & Acetonitrile, THF and diethyl- ether | 6-cyano-7-(5,7- dihydro-6H-pyr- rolo[3,4-b]pyridin- 6-yl)-1-(6-((2- methoxyethyl)- amino)pyridin-3- yl)-4-oxo-1,4- dihydroquinoline- 3-carboxylic acid | MS (ESI): m/z 483.3 [M + H]⁺; ¹H NMR (400 MHz, DMSO-d₆): δ 8.67 (s, 1H), 8.56 (s, 1H), 8.51 (d, J = 4.4 Hz, 1H), 8.38 (d, J = 2.4 Hz, 1H), 7.92 (d, J = 8.0 Hz, 2H), 7.40- 7.35 (m, 1H), 7.07 (d, J = 10.2 Hz, 1H), 6.25 (s, 1H), 5.12-4.93 (m, 4H), 3.70-3.58 (m, 4H), 3.34 (s, 3H) (—COOH and —NH peaks were not observed); |
| 51 | 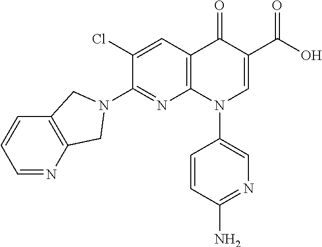 [SMILES: Nc1ccc(-n2cc(C(=O)O)c(=O)c3cc(Cl)c(N4Cc5cccnc5C4)nc32)cn1] | trituration with IPA & Acetonitrile, THF and diethyl- ether | 1-(6-aminopyridin- 3-yl)-6-chloro-7- (5,7-dihydro-6H- pyrrolo[3,4-b]pyr- idin-6-yl)-4-oxo- 1,4-dihydro-1,8- naphthyridine-3- carboxylic acid | MS (ESI): m/z 435.1 [M + H]⁺; ¹H NMR (400 MHz, DMSO-d₆): δ 8.91 (s, 1H), 8.50 (d, J = 3.6 Hz, 1H), 8.42 (s, 2H), 8.20 (d, J = 9.6 Hz, 1H), 7.84 (d, J = 7.6 Hz, 1H), 7.37 (t, J = 5.6 Hz, 1H), 7.15 (d, J = 9.2 Hz, 1H), 5.25 (bs, 2H), 5.00 (bs, 2H) (exchangeable protons were not seen); |
| 52 | 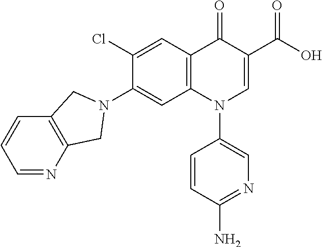 [SMILES: Nc1ccc(-n2cc(C(=O)O)c(=O)c3cc(Cl)c(N4Cc5cccnc5C4)cc32)cn1] | Prep-HPLC, Method-7 of Table-2 | 1-(6-aminopyridin- 3-yl)-6-chloro-7- (5,7-dihydro-6H- pyrrolo[3,4-b]pyr- idin-6-yl)-4-oxo- 1,4-dihydroquin- oline-3-carboxylic acid | MS (ESI): m/z 434.1 [M + H]⁺; ¹H NMR (400 MHz, DMSO-d₆): δ 8.66 (s, 1H), 8.48 (d, J = 3.6 Hz, 1H), 8.34 (d, J = 2.4 Hz, 1H), 8.24 (s, 1H), 7.91 (dd, J = 9.2, 2.4 Hz, 1H), 7.84 (d, J = 7.2 Hz, 1H), 7.35-7.31 (m, 1H), 6.89 (d, J = 9.2 Hz, 1H), 6.44 (s, 1H), 5.10-4.86 (m, 4H) (exchangeable protons were not seen); |
| 53 | 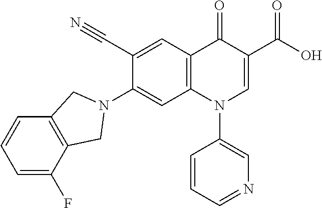 [SMILES: N#Cc1cc2c(=O)c(C(=O)O)cn(-c3cccnc3)c2cc1N1Cc2cccc(F)c2C1] | trituration with 10% Isopropanol in diethyl ether | 6-cyano-7-(4- fluoroisoindolin-2- yl)-4-oxo-1-(pyr- idin-3-yl)-1,4- dihydroquinoline- 3-carboxylic acid | MS (ESI): m/z 427 [M + H]⁺; ¹H NMR (400 MHz, DMSO-d₆): δ 14.64 (bs, 1H), 8.92-8.89 (m, 2H), 8.67 (bs, 1H), 8.56 (s, 1H), 8.23-8.20 (m, 1H), 7.80-7.76 (m, 1H), 7.41- 7.36 (m, 1H), 7.27-7.24 (m, 1H), 7.16 (t, J = 9.2 Hz, 1H), 6.03 (s, 1H), 5.02 (s, 2H), 4.92 (s, 2H); |
| 54 | 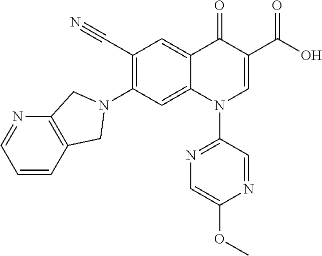 [SMILES: COc1cnc(-n2cc(C(=O)O)c(=O)c3cc(C#N)c(N4Cc5cccnc5C4)cc32)cn1] | trituration with IPA & Acetonitrile, THF and diethyl ether | 6-cyano-7-(5,7- dihydro-6H-pyr- rolo[3,4-b]pyridin- 6-yl)-1-(5-meth- oxypyrazin-2-yl)- 4-oxo-1,4-dihydro quinoline-3-carbox- ylic acid | MS (ESI): m/z 441.1 [M + H]⁺; ¹H NMR (400 MHz, DMSO-d₆): δ 8.56 (s, 1H), 8.27 (s, 1H), 8.55 (s, 1H), 8.49 (d, J = 4.4 Hz, 1H), 8.46 (s, 1H), 7.86 (d, J = 7.6 Hz, 1H), 7.37-7.33 (m, 1H), 6.42 (s, 1H), 5.01 (s, 2H), 4.97 (s, 2H), 4.08 (s, 3H) (—COOH peak was not observed) . |
| 55 | 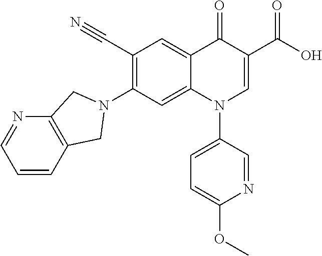 [SMILES: COc1ccc(-n2cc(C(=O)O)c(=O)c3cc(C#N)c(N4Cc5cccnc5C4)cc32)cn1] | trituration with 10% Isopropanol in diethyl ether | 6-cyano-7-(5,7- dihydro-6H-pyr- rolo[3,4-b]pyridin- 6-yl)-1-(6-meth- oxypyridin-3-yl)- 4-oxo-1,4-dihydro- quinoline-3-carbox- ylic acid | MS (ESI): m/z 440 [M + H]⁺; ¹H NMR (400 MHz, DMSO-d₆): δ 14.69 (s, 1H), 8.66 (s, 1H), 8.55 (s, 1H), 8.54 (d, J = 2.4 Hz, 1H), 8.49 (d, J = 4.4 Hz, 1H), 8.07 (dd, J = 8.8, 2.4 Hz, 1H), 7.88 (d, J = 7.6 Hz, 1H), 7.37-7.33 (m, 1H), 7.14 (d, J = 8.8 Hz, 1H), 6.13 (s, 1H), 4.97 (bs, 4H), 4.00 (s, 3H). |
| 56 | 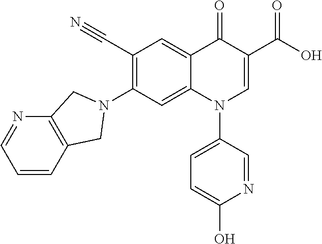 [SMILES: N#Cc1cc2c(=O)c(C(=O)O)cn(-c3ccc(O)nc3)c2cc1N1Cc2cccnc2C1] | trituration with IPA & Acetonitrile, THF and diethyl ether | 6-cyano-7-(5,7- dihydro-6H-pyr- rolo[3,4-b]pyridin- 6-yl)-1-(6-hydroxy- pyridin-3-yl)-4- oxo-1,4-dihydro- quinoline-3-carbox- ylic acid | MS (ESI): m/z 426.1 [M + H]⁺; ¹H NMR (400 MHz, DMSO-d₆): δ 12.13 (bs, 1H), 8.70 (s, 1H), 8.55 (s, 1H), 8.51 (d, J = 3.6 Hz, 1H), 8.00 (d, J = 2.4 Hz, 1H), 7.92 (d, J = 7.6 Hz, 1H), 7.68 (dd, J = 9.6, 2.8 Hz, 1H), 7.39-7.34 (m, 1H), 6.54 (d, J = 9.6 Hz, 1H), 6.30 (s, 1H), 5.14-4.96 (m, 4H), (—COOH peak was not observed). |
| 57 | 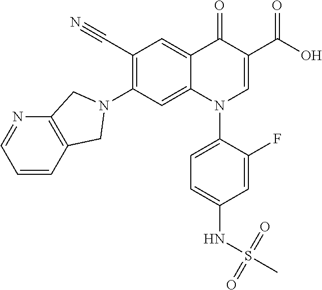 [SMILES: CS(=O)(=O)Nc1ccc(-n2cc(C(=O)O)c(=O)c3cc(C#N)c(N4Cc5cccnc5C4)cc32)c(F)c1] | trituration with 10% MeOH in DCM | 6-cyano-7-(5,7- dihydro-6H-pyr- rolo[3,4-b]pyridin- 6-yl)-1-(2-fluoro- 4-(methylsulfon- amido)phenyl)-4- oxo-1,4-dihydro- quinoline-3-carbox- ylic acid | MS (ESI): m/z 520 [M + H]⁺; ¹H NMR (400 MHz, DMSO-d₆): δ 10.56 (s, 1H), 8.73 (s, 1H), 8.57 (s, 1H), 8.50 (d, J = 2.4 Hz, 1H), 7.88 (d, J = 7.2 Hz, 1H), 7.80 (t, J = 8.8 Hz, 1H), 7.37-7.33 (m, 2H), 7.28 (d, J = 7.2 Hz, 1H), 6.11 (s, 1H), 4.98 (bs, 4H), 3.22 (s, 3H) (—COOH peak was not observed). |
| 58 | 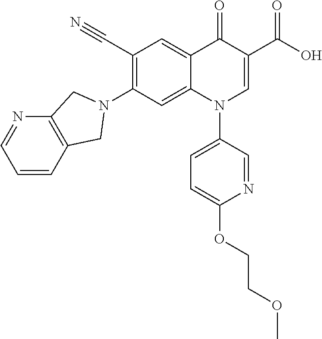 [SMILES: COCCOc1ccc(-n2cc(C(=O)O)c(=O)c3cc(C#N)c(N4Cc5cccnc5C4)cc32)cn1] | trituration with 10% Isopropanol in diethyl ether | 6-cyano-7-(5,7- dihydro-6H-pyr- rolo[3,4-b]pyridin- 6-yl)-1-(6-(2-meth- oxyethoxy)pyridin- 3-yl)-4-oxo-1,4- dihydroquinoline- 3-carboxylic acid | MS (ESI): m/z 484.0 [M + H]⁺; ¹H NMR (400 MHz, DMSO-d₆): δ 14.69 (s, 1H), 8.67 (s, 1H), 8.57 (s, 1H), 8.52 (d, J = 2.4 Hz, 1H), 8.49 (d, J = 2.4 Hz, 1H), 8.08 (dd, J = 8.8, 2.8 Hz, 1H), 7.88 (d, J = 6.8 Hz, 1H), 7.36-7.32 (m, 1H), 7.14 (d, J = 8.8 Hz, 1H), 6.13 (s, 1H), 4.97 (s, 4H), 4.51 (q, J = 4.8 Hz, 2H), 3.74 (t, J = 4.8 Hz, 2H), 3.35 (s, 3H); |
| 59 | 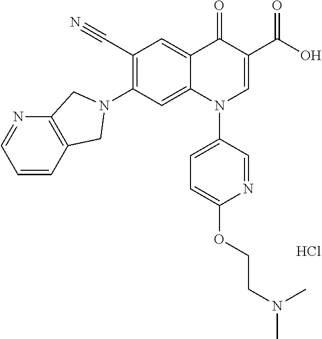 [SMILES: CN(C)CCOc1ccc(-n2cc(C(=O)O)c(=O)c3cc(C#N)c(N4Cc5cccnc5C4)cc32)cn1 ; Cl] | trituration with IPA & Acetonitrile, THF and diethyl ether | 6-cyano-7-(5,7- dihydro-6H-pyr- rolo[3,4-b]pyridin- 6-yl)-1-(6-(2- (dimethylamino)- ethoxy)pyridin-3- yl)-4-oxo-1,4- dihydroquinoline- 3-carboxylic acid hydrochloride | MS (ESI): m/z 497.3 [M + H]⁺; ¹H NMR (400 MHz, DMSO-d₆): δ 10.46 (bs, 1H), 8.64 (s, 1H), 8.58 (d, J = 2.0 Hz, 2H), 8.50 (d, J = 4.0 Hz, 1H), 8.14 (dd, J = 8.8, 2.8 Hz, 1H), 7.89 (d, J = 7.6, Hz, 1H), 7.38-7.34 (m, 1H), 7.20 (d, J = 8.8 Hz, 1H), 6.13 (s, 1H), 5.01 (s, 2H), 4.94 (s, 2H), 4.75 (t, J = 4.8 Hz, 2H), 3.58 (t, J = 4.8 Hz, 2H), 2.90 (s, 3H), 2.89 (s, 3H) (—COOH peak was not observed); |
| 60 | 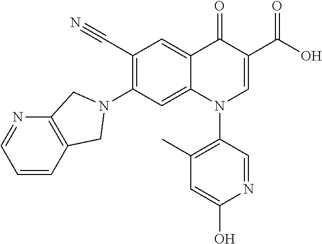 [SMILES: Cc1cc(O)ncc1-n1cc(C(=O)O)c(=O)c2cc(C#N)c(N3Cc4cccnc4C3)cc21] | Prep-HPLC, Method-3 of Table-2 | 6-cyano-7-(5,7- dihydro-6H-pyr- rolo[3,4-b]pyridin- 6-yl)-1-(6-hydroxy- 4-methylpyridin-3- yl)-4-oxo-1,4- dihydroquinoline- 3-carboxylic acid | MS (ESI): m/z 440.1 [M + H]⁺; ¹H NMR (400 MHz, DMSO-d₆): δ 11.97 (bs, 1H), 8.72 (s, 1H), 8.56 (s, 1H), 8.51 (d, J = 3.6 Hz, 1H), 7.93 (d, J = 5.2 Hz, 2H), 7.39- 7.34 (m, 1H), 6.45 (s, 1H), 6.18 (s, 1H), 5.12- 4.96 (m, 4H), 1.88 (s, 3H) (—COOH peak was not observed). |
| 61 | 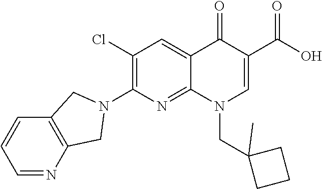 [SMILES: CC1(Cn2cc(C(=O)O)c(=O)c3cc(Cl)c(N4Cc5cccnc5C4)nc32)CCC1] | Trituration with diethyl ether | 6-chloro-7-(5,7- dihydro-6H-pyr- rolo[3,4-b]pyridin- 6-yl)-1-((1-methyl- cyclobutyl)meth- yl)-4-oxo-1,4- dihydro-1,8-naph- thyridine-3-carbox- ylic acid | MS (ESI): m/z 425.0 [M + H]⁺; ¹H NMR (400 MHz, DMSO-d₆): δ 15.15 (bs, 1H), 8.76 (s, 1H), 8.44 (s, 1H), 8.00- 7.77 (m, 2H), 7.28 (s, 1H), 5.20 (bs, 4H), 4.51 (s, 2H), 2.10-1.98 (m, 2H), 1.96-1.83 (m, 1H), 1.80-1.68 (m, 1H), 1.66- 1.57 (m, 2H), 1.14 (bs, 3H). |
| 62 | 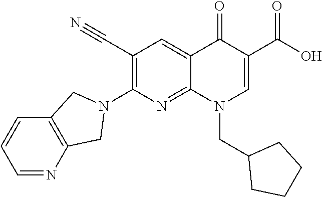 [SMILES: N#Cc1cc2c(=O)c(C(=O)O)cn(CC3CCCC3)c2nc1N1Cc2cccnc2C1] | Normal phase silica gel column chromatography | 6-cyano-1-(cyclo- pentylmethyl)-7- (5,7-dihydro-6H- pyrrolo[3,4-b]pyr- idin-6-yl)-4-oxo- 1,4-dihydro-1,8- naphthyridine-3- carboxylic acid | MS (ESI): m/z 416.0 [M + H]⁺; ¹H NMR (400 MHz, DMSO-d₆): δ 14.78 (bs, 1H), 9.04 (s, 0.5H), 8.92 (s, 0.5H), 8.83 (bs, 0.5H), 8.66 (s, 0.5H), 8.55 (d, J = 5.2 Hz, 1H), 7.96 (d, J = 8.0 Hz, 1H), 7.40-7.37 (m, 1H), 5.24 (bs, 4H), 4.47 (dd, J = 18.0, 8.0 Hz, 2H), 2.46-2.38 (m, 1H), |
| | | | | 1.72-1.60 (m, 4H), 1.59- |
| | | | | 1.49 (m, 2H). 1.40-1.38 |
| | | | | (m, 2H). |
| 63 | 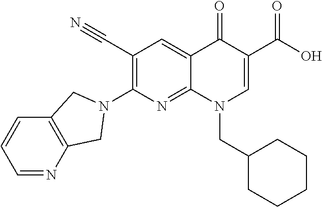 [SMILES: N#Cc1cc2c(=O)c(C(=O)O)cn(CC3CCCCC3)c2nc1N1Cc2cccnc2C1] | Prep-HPLC, Method-7 of Table-2 | 6-cyano-1-(cyclo- hexylmethyl)-7- (5,7-dihydro-6H- pyrrolo[3,4-b]pyr- idin-6-yl)-4-oxo- 1,4-dihydro-1,8- naphthyridine-3- carboxylic acid | MS (ESI): m/z 430.0 [M + H]⁺; ¹H NMR (400 MHz, DMSO-d₆): δ 8.73 (bs, 2H), 8.53 (d, J = 4.0 Hz, 1H), 7.94 (d, J = 7.2 Hz, 1H), 7.40-7.37 (m, 1H), 5.27 (s, 2H), 5.21 (s, 2H), 4.32 (bs, 2H), 1.93 (bs, 1H), 1.72-1.60 (m, 5H) 1.20-1.04 (m, 5H) (—COOH peak was not observed). |
| 64 | 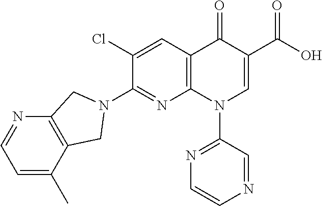 [SMILES: Cc1ccnc2c1CN(c1nc3c(cc1Cl)c(=O)c(C(=O)O)cn3-c1cnccn1)C2] | Trituration with 10% MeOH in DCM followed by IPA | 6-chloro-7-(4-meth- yl-5,7-dihydro-6H- pyrrolo[3,4-b]pyr- idin-6-yl)-4-oxo-1- (pyrazin-2-yl)-1,4- dihydro-1,8-naph- thyridine-3-car- boxylic acid | MS (ESI): m/z 435 [M + H]⁺; 1H NMR (400 MHz, DMSO-d₆): δ 14.72 (s, 1H), 9.33 (s, 1H), 9.07 (s, 1H), 8.89 (s, 1H), 8.82 (s, 1H), 8.39 (s, 1H), 8.33 (d, J = 4.8 Hz, 1H), 7.15 (s, 1H), 5.02 (bs, 4H), 2.24 (s, 3H). |
| 65 | 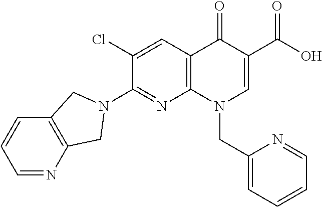 [SMILES: O=C(O)c1cn(Cc2ccccn2)c2nc(N3Cc4cccnc4C3)c(Cl)cc2c1=O] | trituration with 10% Isopropanol in diethyl ether | 6-chloro-7-(5,7- dihydro-6H-pyr- rolo[3,4-b]pyridin- 6-yl)-4-oxo-1- (pyridin-2-ylmeth- yl)-1,4-dihydro- 1,8-naphthyridine- 3-carboxylic acid | MS (ESI): m/z 434.2 [M + H]⁺; ¹H NMR (400 MHz, DMSO-d₆): δ 15.17 (bs, 1H), 9.19 (s, 1H), 8.50 (d, J = 4.8 Hz, 1H), 8.45 (d, J = 4.0 Hz, 1H), 8.33 (s, 1H), 7.86- 7.80 (m, 2H), 7.59 (d, J = 7.6 Hz, 1H), 7.37-7.26 (m, 2H), 5.87 (s, 2H), 5.15 (bs, 2H), 5.07 (bs, 2H). |
| 66 | 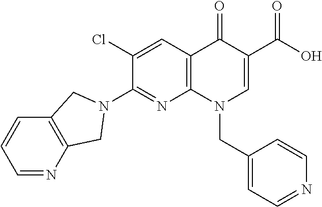 [SMILES: O=C(O)c1cn(Cc2ccncc2)c2nc(N3Cc4cccnc4C3)c(Cl)cc2c1=O] | Rinsed with cold water | 6-chloro-7-(5,7- dihydro-6H-pyr- rolo[3,4-b]pyridin- 6-yl)-4-oxo-1-(pyr- idin-4-ylmethyl)- 1,4-dihydro-1,8- naphthyridine-3- carboxylic acid | MS (ESI): m/z 434.0 [M + H]⁺; ¹H NMR (400 MHz, DMSO-d₆): δ 15.10 (bs, 1H), 9.21 (s, 1H), 8.52 (d, J = 5.6 Hz, 2H), 8.49 (d, J = 4.4 Hz, 1H), 8.34 (s, 1H), 7.82 (d, J = 7.6 Hz, 1H), 7.36- 7.30 (m, 3H), 5.80 (s, 2H), 5.13 (bs, 2H), 5.05 (bs, 2H). |
| 67 | 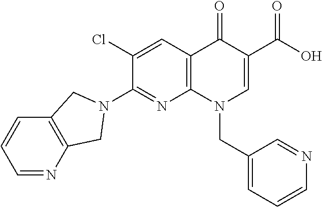 [SMILES: O=C(O)c1cn(Cc2cccnc2)c2nc(N3Cc4cccnc4C3)c(Cl)cc2c1=O] | trituration with 10% Isopropanol in diethyl ether | 6-chloro-7-(5,7- dihydro-6H-pyr- rolo[3,4-b]pyridin- 6-yl)-4-oxo-1-(pyr- idin-3-ylmethyl)- 1,4-dihydro-1,8- naphthyridine-3- carboxylic acid | MS (ESI): m/z 434.0 [M + H]⁺; ¹H NMR (400 MHz, DMSO-d₆): δ 15.11 (bs, 1H), 9.23 (s, 1H), 8.70 (d, J = 1.6 Hz, 1H), 8.52-8.48 (m, 2H), 8.35 (s, 1H), 7.85 (d, J = 7.6 Hz, 1H), 7.76 (d, J = 7.6 Hz, 1H), 7.39-7.34 (m, 2H), 5.82 (s, 2H), 5.24 (bs, 2H), 5.17 (bs, 2H). |
| 68 | 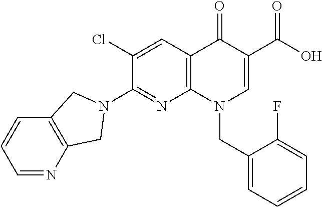 [SMILES: O=C(O)c1cn(Cc2ccccc2F)c2nc(N3Cc4cccnc4C3)c(Cl)cc2c1=O] | trituration with 10% Isopropanol in diethyl ether | 6-chloro-7-(5,7- dihydro-6H-pyr- rolo[3,4-b]pyridin- 6-yl)-1-(2-fluoro- benzyl)-4-oxo-1,4- dihydro-1,8-naph- thyridine-3-carbox- ylic acid | MS (ESI): m/z 451.6 [M + H]⁺; ¹H NMR (400 MHz, DMSO-d₆): δ 15.08 (bs, 1H), 9.15 (s, 1H), 8.50 (d, J = 4.4 Hz, 1H), 8.34 (s, 1H), 7.84 (d, J = 7.2 Hz, 1H), 7.37- 7.14 (m, 5H), 5.83 (s, 2H), 5.21 (bs, 2H), 5.12 (bs, 2H). |
| 69 | 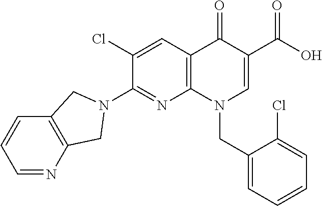 [SMILES: O=C(O)c1cn(Cc2ccccc2Cl)c2nc(N3Cc4cccnc4C3)c(Cl)cc2c1=O] | trituration with 10% Isopropanol in diethyl ether | 6-chloro-1-(2- chlorobenzyl)-7- (5,7-dihydro-6H- pyrrolo[3,4-b]pyr- idin-6-yl)-4-oxo- 1,4-dihydro-1,8- naphthyridine-3- carboxylic acid | MS (ESI): m/z 467.8 [M + H]⁺; 1H NMR (400 MHz, DMSO-d₆): δ 15.11 (bs, 1H), 9.11 (s, 1H), 8.49 (d, J = 4.4 Hz, 1H), 8.36 (s, 1H), 7.89 (d, J = 7.2 Hz, 1H), 7.58 (d, J = 7.2 Hz, 1H), 7.36- 7.24 (m, 3H), 7.00 (d, J = 4.0 Hz, 1H), 5.85 (s, 2H), 5.15 (bs, 2H), 4.99 (bs, 2H). |
| 70 | 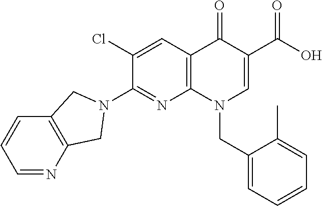 [SMILES: Cc1ccccc1Cn1cc(C(=O)O)c(=O)c2cc(Cl)c(N3Cc4cccnc4C3)nc21] | Trituration with methanol | 6-chloro-7-(5,7- dihydro-6H-pyr- rolo[3,4-b]pyridin- 6-yl)-1-(2-methyl- benzyl)-4-oxo-1,4- dihydro-1,8-naph- thyridine-3-carbox- ylic acid | MS (ESI): m/z 447.7 [M + H]⁺; ¹H NMR (400 MHz, DMSO-d₆): δ 15.13 (bs, 1H), 8.89 (s, 1H), 8.49 (d, J = 4.4 Hz, 1H), 8.38 (s, 1H), 7.81 (d, J = 7.2 Hz, 1H), 7.36- 7.28 (m, 2H), 7.21 (t, J = 7.2 Hz, 1H), 7.12 (d, J = 7.2 Hz, 1H), 6.83 (d, J = 7.6 Hz, 1H), 5.76 (s, 2H), 5.20 (bs, 2H), 5.10 (bs, 2H), 2.39 (s, 3H). |
| 71 | 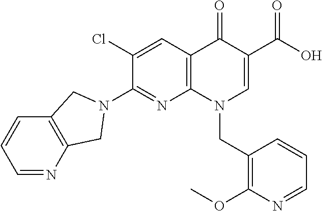 [SMILES: COc1ncccc1Cn1cc(C(=O)O)c(=O)c2cc(Cl)c(N3Cc4cccnc4C3)nc21] | Trituration with 10% MeOH in diethyl ether | 6-chloro-7-(5,7- dihydro-6H-pyr- rolo[3,4-b]pyridin- 6-yl)-1-((2-meth- oxypyridin-3-yl)- methyl)-4-oxo-1,4- dihydro-1,8-naph- thyridine-3-carbox- ylic acid | MS (ESI): m/z 464.0 [M + H]⁺; ¹H NMR (400 MHz, DMSO-d₆): δ 15.11 (bs, 1H), 9.16 (s, 1H), 8.51 (d, J = 4.0 Hz, 1H), 8.33 (s, 1H), 8.09 (dd, J = 5.2, 2.0 Hz, 1H), 7.84 (d, J = 6.8 Hz, 1H), 7.50 (d, J = 6.0 Hz, 1H), 7.38-7.34 (m, 1H), 6.94 (dd, J = 7.2, 4.8 Hz, 1H), 5.69 (s, 2H), 5.20 (bs, 2H), 5.06 (bs, 2H), 4.00 |
| | | | | (s, 3H). |
| 72 | 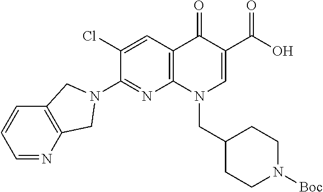 [SMILES: CC(C)(C)OC(=O)N1CCC(Cn2cc(C(=O)O)c(=O)c3cc(Cl)c(N4Cc5cccnc5C4)nc32)CC1] | Rinsed with cold water | 1-((1-(tert-butoxy- carbonyl)piperidin- 4-yl)methyl)-6- chloro-7-(5,7-di- hydro-6H-pyrrolo- [3,4-b]pyridin-6- yl)-4-oxo-1,4-di- hydro-1,8-naph- thyridine-3-carbox- ylic acid | MS (ESI): m/z 540.2 [M + H]⁺; ¹H NMR (400 MHz, DMSO-d₆): δ 8.72 (bs, 1H), 8.51 (d, J = 4.4 Hz, 1H), 8.28 (bs, 1H), 7.89 (d, J = 8.8 Hz, 1H), 7.39-7.34 (m, 1H), 5.32 (s, 2H), 5.26 (s, 2H), 4.40-4.30 (m, 2H), 4.00- 3.90 (m, 2H), 2.70-2.60 (m, 2H), 2.20-2.10 (m, 1H), 1.60-1.50 (m, 2H), |
| | | | | 1.38 (s, 9H), 1.30-1.17 |
| | | | | (m, 2H) (—COOH peak |
| | | | | was not observed). |
| 73 | 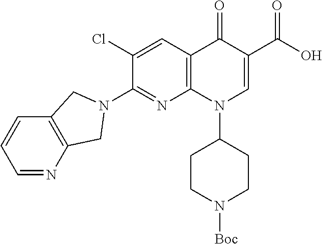 [SMILES: CC(C)(C)OC(=O)N1CCC(n2cc(C(=O)O)c(=O)c3cc(Cl)c(N4Cc5cccnc5C4)nc32)CC1] | Rinsed with cold water | 1-(1-(tert-butoxy- carbonyl)piperidin- 4-yl)-6-chloro-7- (5,7-dihydro-6H- pyrrolo[3,4-b]pyr- idin-6-yl)-4-oxo- 1,4-dihydro-1,8- naphthyridine-3- carboxylic acid | MS (ESI): m/z 526.0 [M + H]⁺; ¹H NMR (400 MHz, DMSO-d₆): δ 8.72 (bs, 1H), 8.50 (d, J = 4.0 Hz, 1H), 8.26 (bs, 1H), 7.88 (d, J = 7.2 Hz, 1H), 7.40-7.37 (m, 1H), 5.36- 5.31 (m, 1H), 5.28 (s, 2H), 5.21 (s, 2H), 4.20 (bs, 2H), 3.16-3.00 (m, 2H), 2.10-2.00 (m, 2H), 1.90-1.78 (m, 2H). 1.45 (s, 9H) (—COOH peak was not observed). |
| 74 | 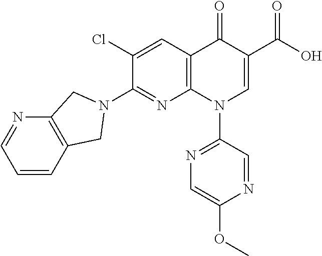 [SMILES: COc1cnc(-n2cc(C(=O)O)c(=O)c3cc(Cl)c(N4Cc5cccnc5C4)nc32)cn1] | trituration with IPA & Acetonitrile, diethyl ether and n-pentane | 6-chloro-7-(5,7- dihydro-6H-pyr- rolo[3,4-b]pyridin- 6-yl)-1-(5-meth- oxypyrazin-2-yl)- 4-oxo-1,4-dihydro- 1,8-naphthyridine- 3-carboxylic acid | MS (ESI): m/z 451.2 [M + H]⁺; ¹H NMR (400 MHz, DMSO-d₆): δ 14.79 (bs, 1H), 8.97 (s, 1H), 8.81 (s, 1H), 8.50- 8.39 (m, 3H), 7.89 (d, J = 8.0 Hz, 1H), 7.34-7.30 (m, 1H), 5.15 (s, 2H), 4.98 (s, 2H), 4.08 (s, 3H). |
| 75 | 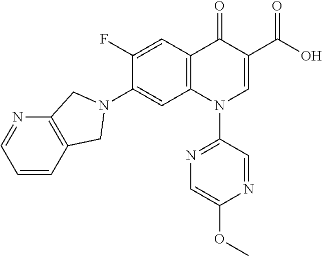 [SMILES: COc1cnc(-n2cc(C(=O)O)c(=O)c3cc(F)c(N4Cc5cccnc5C4)cc32)cn1] | trituration with IPA & Acetonitrile, diethyl ether and n-pentane | 7-(5,7-dihydro-6H- pyrrolo[3,4-b]pyr- idin-6-yl)-6-fluoro- 1-(5-methoxypyr- azin-2-yl)-4-oxo- 1,4-dihydroquin- oline-3-carboxylic acid | MS (ESI): m/z 434.1 [M + H]⁺; ¹H NMR (400 MHz, DMSO-d₆): δ 15.22 (bs, 1H), 8.83 (s, 1H), 8.73 (s, 1H), 8.50- 8.46 (m, 2H), 7.98 (d, J = 14.4 Hz, 1H), 7.81 (d, J = 7.8 Hz, 1H) 7.35-7.31 (m, 1H), 6.36 (d, J = 7.8 Hz, 1H), 4.91 (s, 2H), 4.82 (s, 2H), 4.09 (s, 3H). |
| 76 | 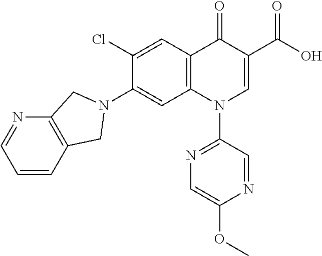 [SMILES: COc1cnc(-n2cc(C(=O)O)c(=O)c3cc(Cl)c(N4Cc5cccnc5C4)cc32)cn1] | trituration with IPA & Acetonitrile, diethyl ether and n-pentane | 6-chloro-7-(5,7- dihydro-6H-pyr- rolo[3,4-b]pyridin- 6-yl)-1-(5-meth- oxypyrazin-2-yl)- 4-oxo-1,4-dihydro- quinoline-3-carbox- ylic acid | MS (ESI): m/z 450.1 [M + H]⁺; ¹H NMR (400 MHz, DMSO-d₆): δ 14.94 (bs, 1H), 8.86 (s, 1H), 8.74 (s, 1H), 8.49- 8.45 (m, 2H), 8.25 (s, 1H), 7.80 (d, J = 7.6 Hz, 1H), 7.34-7.30 (m, 1H), 6.56 (s, 1H), 5.02 (s, 2H), 4.93 (s, 2H), 4.08 (s, 3H). |
| 77 | 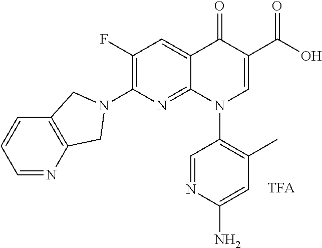 [SMILES: Cc1cc(N)ncc1-n1cc(C(=O)O)c(=O)c2cc(F)c(N3Cc4cccnc4C3)nc21] | Trituration with diethyl ether | 1-(6-amino-4-meth- ylpyridin-3-yl)-7- (5,7-dihydro-6H- pyrrolo[3,4-b]pyr- idin-6-yl)-6-fluoro- 4-oxo-1,4-dihydro- 1,8-naphthyridine- 3-carboxylic acid TFA salt | MS (ESI): m/z 433.1 [M + H]⁺; ¹H NMR (400 MHz, DMSO-d₆): δ 15.18 (bs, 1H), 8.79 (s, 1H), 8.48 (d, J = 4.8 Hz, 1H), 8.21-8.17 (m, 2H), 7.83 (d, J = 7.6 Hz, 1H), 7.69 (bs, 1H), 7.36-7.31 (m, 1H), 6.83 (s, 1H), 5.30 (bs, 2H), 4.54 (bs, 2H), 2.04 (s, 3H) (couple of exchangeable protons were not observed by ¹HNMR). |
| 78 | 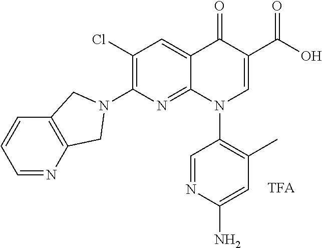 [SMILES: Cc1cc(N)ncc1-n1cc(C(=O)O)c(=O)c2cc(Cl)c(N3Cc4cccnc4C3)nc21] | Trituration with n-pentane | 1-(6-amino-4-meth- ylpyridin-3-yl)-6- chloro-7-(5,7-di- hydro-6H-pyrrolo- [3,4-b]pyridin-6- yl)-4-oxo-1,4-di- hydro-1,8-naph- thyridine-3-carbox- ylic acid TFA salt | MS (ESI): m/z 449 [M + H]⁺; ¹H NMR (400 MHz, DMSO-d₆): δ 14.95 (brs, 1H), 8.82 (s, 1H), 8.47 (d, J = 4.8 Hz, 1H), 8.41 (s, 1H), 8.19 (s, 1H), 7.80-7.68 (m, 3H), 7.33 (q, J = 4.8 Hz, 1H), 6.84 (s, 1H), 5.21 (m, 2H), 4.86 (s, 2H), 2.06 (s, 3H); |
| 79 | 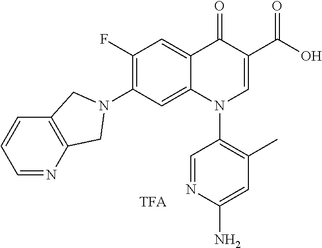 [SMILES: Cc1cc(N)ncc1-n1cc(C(=O)O)c(=O)c2cc(F)c(N3Cc4cccnc4C3)cc21] | trituration with diethyl ether and MeOH | 1-(6-amino-4- methylpyridin-3- yl)-7-(5,7-dihydro- 6H-pyrrolo[3,4-b]- pyridin-6-yl)-6- fluoro-4-oxo-1,4- dihydroquinoline- 3-carboxylic acid TFA salt | MS (ESI): m/z 432 [M + H]⁺; ¹H NMR (400 MHz, DMSO-d₆): δ 15.38 (s, 1H), 8.53 (s, 1H), 8.47 (d, J = 4.8 Hz, 1H), 8.04 (s, 1H), 7.98 (d, J = 14.0 Hz, 1H), 7.86 (d, J = 7.6 Hz, 1H), 7.32 (q, J = 4.8 Hz, 1H), 6.52-6.48 (m, 3H), 6.06 (d, J = 7.6 Hz, 1H), 4.84 (s, 2H), 4.81 (s, 2H), 1.90 (s, 3H); |
| 80 | 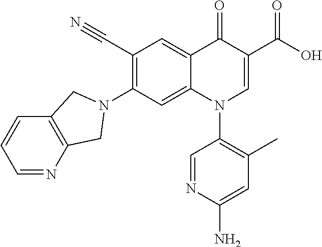 [SMILES: Cc1cc(N)ncc1-n1cc(C(=O)O)c(=O)c2cc(C#N)c(N3Cc4cccnc4C3)cc21] | trituration with 10% Isopropanol in diethyl ether | 1-(6-amino-4- methylpyridin-3- yl)-6-cyano-7-(5,7- dihydro-6H-pyr- rolo[3,4-b]pyridin- 6-yl)-4-oxo-1,4- dihydroquinoline- 3-carboxylic acid | MS (ESI): m/z 439 [M + H]⁺; ¹H NMR (400 MHz, DMSO-d₆): δ 14.72 (s, 1H), 8.57 (d, J = 6.0 Hz, 2H), 8.49 (d, J = 4.4 Hz, 1H), 8.05 (s, 1H), 7.90 (d, J = 7.6 Hz, 1H), 7.37- 7.32 (m, 1H), 6.52 (bs, 3H), 6.04 (s, 1H), 4.96 (s, 4H), 1.93 (s, 3H); |
| 81 | 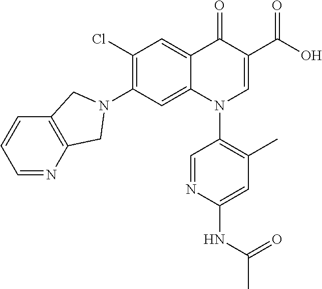 [SMILES: CC(=O)Nc1cc(C)c(-n2cc(C(=O)O)c(=O)c3cc(Cl)c(N4Cc5cccnc5C4)cc32)cn1] | trituration with 10% Isopropanol in diethyl ether | 1-(6-acetamido-4- methylpyridin-3- yl)-6-chloro-7-(5,7- dihydro-6H-pyr- rolo[3,4-b]pyridin- 6-yl)-4-oxo-1,4- dihydroquinoline- 3-carboxylic acid | MS (ESI): m/z 490 [M + H]⁺; ¹H NMR (400 MHz, DMSO-d₆): δ 15.08 (s, 1H), 10.86 (s, 1H), 8.71 (s, 1H), 8.49 (s, 1H), 8.45 (d, J = 4.4 Hz, 1H), 8.29 (s, 1H), 8.25 (s, 1H), 7.81 (d, J = 7.2 Hz, 1H), 7.33- 7.29 (m, 1H), 6.10 (s, 1H), 4.96 (s, 2H), 4.86 (m, 4H), 2.18 (s, 3H), 2.10 (s, 3H); |
| 82 | 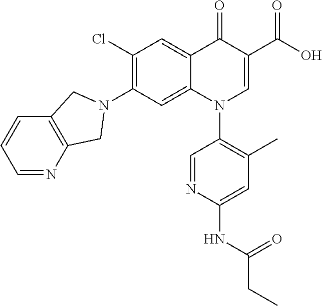 [SMILES: CCC(=O)Nc1cc(C)c(-n2cc(C(=O)O)c(=O)c3cc(Cl)c(N4Cc5cccnc5C4)cc32)cn1] | trituration with diethyl ether and MeOH | 6-chloro-7-(5,7- dihydro-6H-pyr- rolo[3,4-b]pyridin- 6-yl)-1-(4-methyl- 6-propionamido- pyridin-3-yl)-4- oxo-1,4-dihydro- quinoline-3-car- boxylic acid | MS (ESI): m/z 504 [M + H]⁺; ¹H NMR (400 MHz, DMSO-d₆): δ 15.11 (s, 1H), 10.83 (s, 1H), 8.71 (s, 1H), 8.49 (s, 1H), 8.45 (d, J = 4.4 Hz, 1H), 8.33 (s, 1H), 8.26 (s, 1H), 7.81 (d, J = 6.8 Hz, 1H), 7.30 (q, J = 4.8 Hz, 1H), 6.11 (s, 1H), 4.96 (s, 2H), 4.87 (s, 2H), 2.47-2.38 (m, 2H), 2.10 (s, 3H), 1.11 (t, J = 7.6 Hz, 3H); |
| 83 | 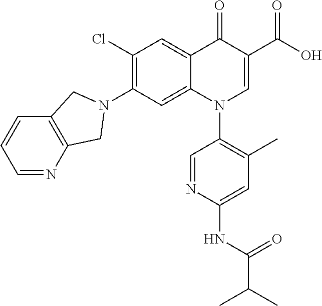 [SMILES: Cc1cc(NC(=O)C(C)C)ncc1-n1cc(C(=O)O)c(=O)c2cc(Cl)c(N3Cc4cccnc4C3)cc21] | Normal phase silica gel column chromatography | 6-chloro-7-(5,7- dihydro-6H-pyr- rolo[3,4-b]pyridin- 6-yl)-1-(6-isobutyr- amido-4-methyl- pyridin-3-yl)-4- oxo-1,4-dihydro- quinoline-3-car- boxylic acid | MS (ESI): m/z 518.2 [M + H]⁺; ¹H NMR (400 MHz, DMSO-d₆): δ 15.08 (s, 1H), 10.81 (s, 1H), 8.70 (s, 1H), 8.49 (s, 1H), 8.45 (d, J = 4.4 Hz, 1H), 8.34 (s, 1H), 8.26 (s, 1H), 7.81 (d, J = 7.6 Hz, 1H), 7.30 (q, J = 4.8 Hz, 1H), 6.13 (s, 1H), 4.96 (s, 2H), 4.87 (s, 2H), 2.88- 2.81 (m, 1H), 2.10 (s, 3H), 1.16-1.13 (m, 6H); |
| 84 | 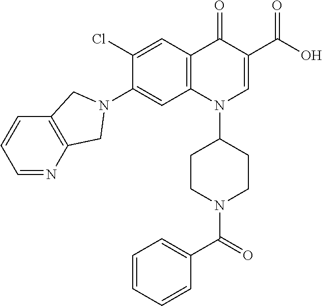 [SMILES: O=C(O)c1cn(C2CCN(C(=O)c3ccccc3)CC2)c2cc(N3Cc4cccnc4C3)c(Cl)cc2c1=O] | Normal phase silica gel column chromatography | 1-(1-benzoylpiper- idin-4-yl)-6-chloro- 7-(5,7-dihydro-6H- pyrrolo[3,4-b]pyr- idin-6-yl)-4-oxo- 1,4-dihydroquin- oline-3-carboxylic acid | MS (ESI): m/z 529.0 [M + H]⁺; ¹H NMR (400 MHz, DMSO-d₆): δ 15.25 (s, 1H), 8.86 (s, 1H), 8.53 (d, J = 4.0 Hz, 1H), 8.24 (s, 1H), 7.87 (d, J = 7.6 Hz, 1H), 7.55- 7.46 (m, 5H), 7.40-7.35 (m, 1H), 7.21 (s, 1H), 5.27 (s, 2H), 5.22 (bs, 1H), 5.16 (s, 2H), 4.74 (bs, 1H), 3.78 (bs, 1H), 3.47 (bs, 1H), 3.21 (bs, 1H), 2.25-2.10 (m, 4H). |
| 85 | 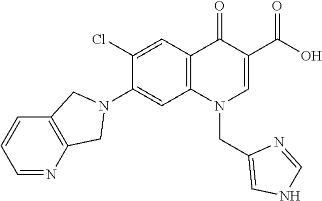 [SMILES: O=C(O)c1cn(Cc2c[nH]cn2)c2cc(N3Cc4cccnc4C3)c(Cl)cc2c1=O] | Prep-HPLC, Method-8 of Table-2 | 1-((1H-imidazol-4- yl)methyl)-6-chloro- 7-(5,7-dihydro-6H- pyrrolo[3,4-b]pyr- idin-6-yl)-4-oxo- 1,4-dihydroquin- oline-3-carboxylic acid | MS (ESI): m/z 422.1 [M + H]⁺; ¹H NMR (400 MHz, DMSO-d₆): δ 15.32 (s, 1H), 12.16 (bs, 1H), 9.07 (s, 1H), 8.53 (d, J = 4.0 Hz, 1H), 8.15 (s, 1H), 7.88 (d, J = 7.6 Hz, 1H), 7.65 (s, 1H), 7.43-7.35 (m, 3H), 5.67 (s, 2H), 5.21 (bs, 2H), 5.10 (bs, 2H). |
| 86 | 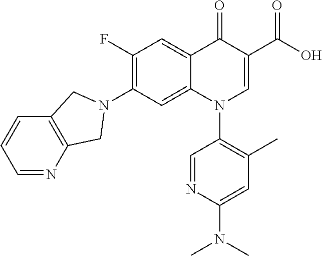 [SMILES: Cc1cc(N(C)C)ncc1-n1cc(C(=O)O)c(=O)c2cc(F)c(N3Cc4cccnc4C3)cc21] | trituration with 10% Isopropanol in diethyl ether | 7-(5,7-dihydro-6H- pyrrolo[3,4-b]pyr- idin-6-yl)-1-(6- (dimethylamino)-4- methylpyridin-3- yl)-6-fluoro-4-oxo- 1,4-dihydroquin- oline-3-carboxylic acid | MS (ESI): m/z 460 [M + H]⁺; ¹H NMR (400 MHz, DMSO-d₆): δ 15.39 (s, 1H), 8.53 (s, 1H), 8.46 (d, J = 3.6 Hz, 1H), 8.21 (s, 1H), 8.00 (d, J = 14.0 Hz, 1H), 7.84 (d, J = 7.6 Hz, 1H), 7.35-7.31 (m, 1H), 6.78 (s, 1H), 6.05 (d, J = 7.6 Hz, 1H), 4.87 (s, 2H), 4.77 (s, 2H), 3.15 (s, 6H), 1.99 (s, 3H). |
| 87 | 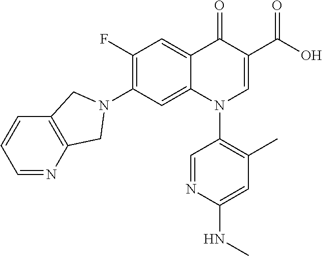 [SMILES: CNc1cc(C)c(-n2cc(C(=O)O)c(=O)c3cc(F)c(N4Cc5cccnc5C4)cc32)cn1] | trituration with 10% Isopropanol in diethyl ether | 7-(5,7-dihydro-6H- pyrrolo[3,4-b]pyr- idin-6-yl)-6-fluoro- 1-(4-methyl-6- (methylamino)pyr- idin-3-yl)-4-oxo- 1,4-dihydroquin- oline-3-carboxylic acid | MS (ESI): m/z 446 [M + H]⁺; ¹H NMR (400 MHz, DMSO-d₆): δ 15.40 (s, 1H), 8.53 (s, 1H), 8.47 (d, J = 3.6 Hz, 1H), 8.12 (s, 1H), 7.98 (d, J = 14.4 Hz, 1H), 7.85 (d, J = 7.6 Hz, 1H), 7.35-7.31 (m, 1H), 7.06 (d, J = 4.8 Hz, 1H), 6.53 (s, 1H), 6.07 (d, J = 7.2 Hz, 1H), 4.85 (s, 2H), 4.79 (s, 2H), 2.87 (d, J = 4.8 Hz, 3H), 1.99 (s, 3H). |
| 88 | 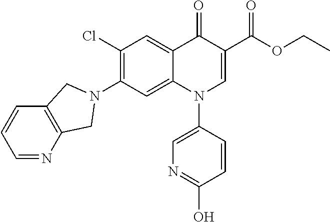 [SMILES: CCOC(=O)c1cn(-c2ccc(O)nc2)c2cc(N3Cc4cccnc4C3)c(Cl)cc2c1=O] | recrystallization | Ethyl 6-chloro-1- (6-hydroxypyridin- 3-yl)-4-oxo-7-{5H, 7H-pyrrolo[3,4-b]- pyridin-6-yl}quin- oline-3-carboxylate | LCMS (ESI) [M + H]⁺: 463.1. ¹H NMR (400 MHz, DMSO-d₆) δ 12.10 (s, 1H), 8.47 (d, J = 4.9 Hz, 1H), 8.40 (s, 1H), 8.08 (s, 1H), 7.97 (d, J = 3.0 Hz, 1H), 7.85 (d, J = 7.7 Hz, 1H), 7.69 (d, J = 9.7, 3.0 Hz, 1H), 7.32 (d, J = 7.6, 4.9 Hz, 1H), 6.53 (d, J = 9.6 Hz, 1H), 6.42 (s, 1H), 5.14-4.77 (m, 4H), 4.20 (m, J = 7.0 Hz, 2H), 1.27 (s, J = 7.1 Hz, |
| | | | | 3H). |
| 89 | 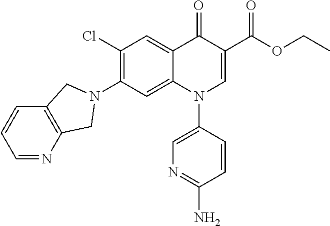 [SMILES: CCOC(=O)c1cn(-c2ccc(N)nc2)c2cc(N3Cc4cccnc4C3)c(Cl)cc2c1=O] | Reverse phase flash chromatography | Ethyl 1-(6-amino- pyridin-3-yl)-6- chloro-4-oxo-7- {1H,3H-pyrrolo- [3,4-c]pyridin-2-yl} quinoline-3-carbox- ylate | LCMS (ESI) [M + H]⁺: 462.25. ¹H NMR (400 MHz, DMSO-d₆) δ 8.45- 8.44 (m, 1H), 8.31 (s, 1H), 8.17-8.03 (m, 2H), 7.92-7.82 (m, 1H), 7.75- 7.65 (m, 1H), 7.38-7.30 (m, 1H), 6.65 (d, J = 8.8 Hz, 1H), 6.56 (s, 2H), 6.31 (s, 1H), 4.87-4.80 (d, J = 30.1 Hz, 4H), 4.27-4.05 (m, 2H), 1.5- 1.20 (m, 3H). |
| 90 | 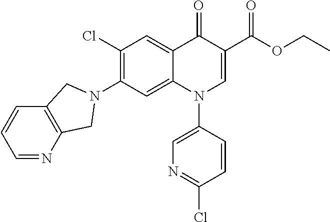 [SMILES: CCOC(=O)c1cn(-c2ccc(Cl)nc2)c2cc(N3Cc4cccnc4C3)c(Cl)cc2c1=O] | recrystallization | Ethyl 6-chloro-1- (6-chloropyridin-3- yl)-7-(5,7-dihydro- 6H-pyrrolo[3,4-b]- pyridin-6-yl)-4- oxo-1,4-dihydro- quinoline-3-carbox- ylate | LCMS (ESI) [M + H]⁺: 480.95. ¹H NMR (400 MHz, Chloroform-d) δ 8.58 (d, J = 2.5 Hz, 1H), 8.51 (d, J = 4.8 Hz, 1H), 8.40 (s, 1H), 8.29 (s, 1H), 7.95-7.84 (m, 1H), 7.67 (d, J = 8.2, 2.8 Hz, 2H), 7.29-7.26 (m, 1H), 6.06 (s, 1H), 5.04 (s, 2H), 4.83 (s, 2H), 4.37 (m, 2H), 1.39 (m, 3H). |
| 93 | 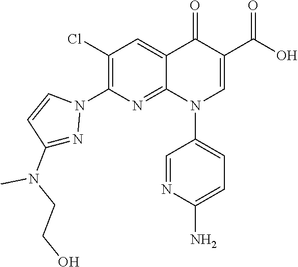 [SMILES: CN(CCO)c1ccn(-c2nc3c(cc2Cl)c(=O)c(C(=O)O)cn3-c2ccc(N)nc2)n1] | | 1-(6-aminopyridin- 3-yl)-6-chloro-7- (3-((2-hydroxyeth- yl)(methyl)amino)- 1H-pyrazol-1-yl)- 4-oxo-1,4-dihydro- 1,8-naphthyridine- 3-carboxylic acid | MS (ESI): m/z 456.1 [M + H]+; 1H NMR (400 MHz, DMSO-d₆) δ 14.48 (brs, 1H), 8.83 (s, 1H), 8.72 (s, 1H), 8.13 (d, J = 2.4 Hz, 1H), 7.83 (d, J = 3.2 Hz, 1H), 7.66 (dd, J = 8.8, 2.4 Hz, 1H), 6.59 (d, J = 8.8 Hz, 1H), 6.48 (brs, 2H), 6.27 (d, J = 2.4 Hz, 1H), 4.64 (t, J = 5.6 Hz, 1H), 3.57 (q, J = 5.6 Hz, 2H), 3.45 (t, J = 5.6 Hz, 2H), 2.97 (s, 3H); |
| 94 | 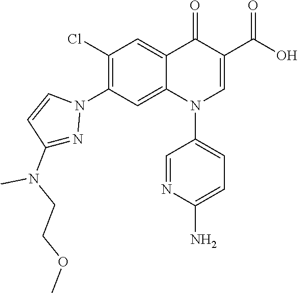 [SMILES: COCCN(C)c1ccn(-c2cc3c(cc2Cl)c(=O)c(C(=O)O)cn3-c2ccc(N)nc2)n1] | | 1-(6-aminopyridin- 3-yl)-6-chloro-7- (3-((2-methoxyeth- yl)(methyl)amino)- 1H-pyrazol-1-yl)- 4-oxo-1,4-dihydro- quinoline-3-carbox- ylic acid | MS (ESI): m/z 469.2 [M + H]+; 1H NMR (400 MHz, DMSO-d₆) δ 14.70 (brs, 1H), 8.77 (s, 1H), 8.45 (s, 1H), 8.29 (d, J = 2.8 Hz, 1H), 8.23 (d, J = 2.4 Hz, 1H), 7.77 (dd, J = 8.8, 2.8 Hz, 1H), 7.50 (s, 1H), 7.00 (brs, 2H), 6.73 (d, J = 9.2 Hz, 1H), 6.14 (d, J = 2.8 Hz, 1H), 3.45 (t, J = 5.2 Hz, 2H), 3.37 (t, J = 5.6 Hz, 2H), 3.22 (s, 3H), 2.87 (s, 3H); |
| 95 | 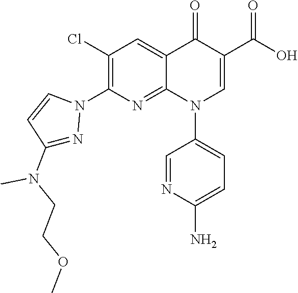 [SMILES: COCCN(C)c1ccn(-c2nc3c(cc2Cl)c(=O)c(C(=O)O)cn3-c2ccc(N)nc2)n1] | | 1-(6-aminopyridin- 3-yl)-6-chloro-7- (3-((2-methoxyeth- yl)(methyl)amino)- 1H-pyrazol-1-yl)- 4-oxo-1,4-dihydro- 1,8-naphthyridine- 3-carboxylic acid | MS (ESI): m/z 470.1 [M + H]+; 1H NMR (400 MHz, DMSO-d₆) δ 14.48 (brs, 1H), 8.83 (s, 1H), 8.72 (s, 1H), 8.13 (d, J = 2.4 Hz, 1H), 7.85 (d, J = 2.8 Hz, 1H), 7.66 (dd, J = 8.8, 2.8 Hz, 1H), 6.59 (d, J = 8.8 Hz, 1H), 6.48 (brs, 2H), 6.29 (d, J = 3.2 Hz, 1H), 3.51 (t, J = 5.2 Hz, 2H), 3.44 (t, J = 5.2 Hz, 2H), 3.23 (s, 3H), 2.95 (s, 3H);. |
| 96 | 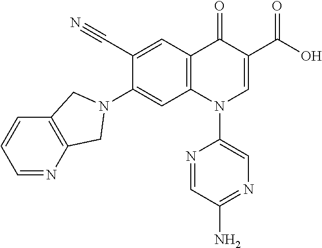 [SMILES: N#Cc1cc2c(=O)c(C(=O)O)cn(-c3cnc(N)cn3)c2cc1N1Cc2cccnc2C1] | | 1-(5-aminopyrazin- 2-yl)-6-cyano-7- (5,7-dihydro-6H- pyrrolo[3,4-b]pyr- idin-6-yl)-4-oxo- 1,4-dihydroquin- oline-3-carboxylic acid | MS (ESI): m/z 426.2 [M + H]⁺; ¹H NMR (400 MHz, DMSO-d₆): δ 14.67 (brs, 1H), 8.76 (s, 1H), 8.55 (s, 1H), 8.50 (d, J = 4.4 Hz, 1H), 8.34 (s, 1H), 7.92 (s, 1H), 7.89 (d, J = 7.8 Hz, 1H), 7.37-7.33 (m, 1H), 7.16 (brs, 2H), 6.36 (s, 1H), 5.00 (s, 4H). |
| 97 | 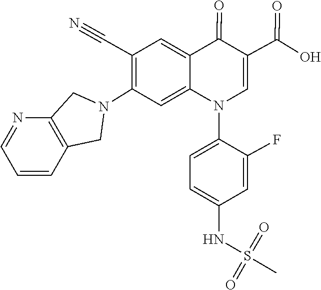 [SMILES: CS(=O)(=O)Nc1ccc(-n2cc(C(=O)O)c(=O)c3cc(C#N)c(N4Cc5cccnc5C4)cc32)c(F)c1] | trituration with 10% MeOH in DCM | 6-cyano-7-(5,7- dihydro-6H-pyr- rolo[3,4-b]pyridin- 6-yl)-1-(2-fluoro- 4-(methylsulfon- amido)phenyl)-4- oxo-1,4-dihydro- quinoline-3-car- boxylic acid | MS (ESI): m/z 520 [M + H]⁺; ¹H NMR (400 MHz, DMSO-d₆): δ 10.56 (s, 1H), 8.73 (s, 1H), 8.57 (s, 1H), 8.50 (d, J = 2.4 Hz, 1H), 7.88 (d, J = 7.2 Hz, 1H), 7.80 (t, J = 8.8 Hz, 1H), 7.37-7.33 (m, 2H), 7.28 (d, J = 7.2 Hz, 1H), 6.11 (s, 1H), 4.98 (bs, 4H), 3.22 (s, 3H) (—COOH peak was not observed). |
| 98 | 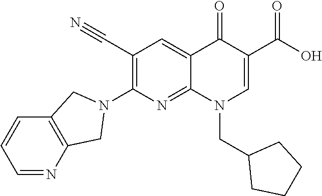 [SMILES: N#Cc1cc2c(=O)c(C(=O)O)cn(CC3CCCC3)c2nc1N1Cc2cccnc2C1] | | 6-cyano-1-(cyclo- pentylmethyl)-7- (5,7-dihydro-6H- pyrrolo[3,4-b]pyr- idin-6-yl)-4-oxo- 1,4-dihydro-1,8- naphthyridine-3- carboxylic acid | MS (ESI): m/z 416 [M + H]⁺; ¹H NMR (400 MHz, DMSO-d₆): δ 14.78 (brs, 1H), 9.03 (s, 0.5H), 8.92 (s, 0.5H), 8.83 (s, 0.5H), 8.66 (s, 0.5H), 8.55 (d, J = 5.2 Hz, 1H), 7.96 (d, J = 8.0 Hz, 1H), 7.43- 7.39 (m, 1H), 5.38-5.22 (m, 4H), 4.51-4.43 (m, 2H), 2.38-2.42 (m, 1H), 1.74-1.62 (m, 4H), 1.58- |
| | | | | 1.49 (m, 2H), 1.40-1.30 |
| | | | | (m, 2H). |
| 99 | 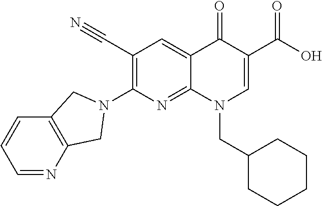 [SMILES: N#Cc1cc2c(=O)c(C(=O)O)cn(CC3CCCCC3)c2nc1N1Cc2cccnc2C1] | Prep-HPLC, Method-7 of Table-2 | 6-cyano-1-(cyclo- hexylmethyl)-7- (5,7-dihydro-6H- pyrrolo[3,4-b]pyr- idin-6-yl)-4-oxo- 1,4-dihydro-1,8- naphthyridine-3- carboxylic acid | MS (ESI): m/z 430.0 [M + H]⁺; ¹H NMR (400 MHz, DMSO-d₆): δ 8.73 (bs, 2H), 8.53 (d, J = 4.0 Hz, 1H), 7.94 (d, J = 7.2 Hz, 1H), 7.40-7.37 (m, 1H), 5.27 (s, 2H), 5.21 (s, 2H), 4.32 (bs, 2H), 1.93 (bs, 1H), 1.72-1.60 (m, 5H). 1.20-1.04 (m, 5H) (—COOH peak was not observed). |
| 100 | 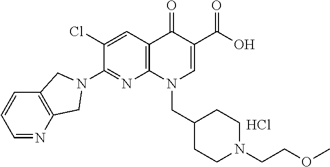 [SMILES: COCCN1CCC(Cn2cc(C(=O)O)c(=O)c3cc(Cl)c(N4Cc5cccnc5C4)nc32)CC1 ; Cl] | | 6-chloro-7-(5,7- dihydro-6H-pyr- rolo[3,4-b]pyridin- 6-yl)-1-((1-(2- methoxyethyl)- piperidin-4-yl)- methyl)-4-oxo- 1,4-dihydro-1,8- naphthyridine-3- carboxylic acid hydro chloride | MS (ESI): m/z 498.4 [M + H]⁺; ¹H NMR (400 MHz, DMSO-d₆): δ 15.26 (brs, 1H), 9.77 (brs, 1H), 9.00 (brs, 1H), 8.53 (d, J = 3.6 Hz, 1H), 8.36 (s, 1H), 7.92 (d, J = 7.2 Hz, 1H), 7.39 (t, J = 6.4 Hz, 1H), 5.40 (s, 2H), 5.31 (s, 2H), 4.50 (d, J = 4.8 Hz, 2H), 3.70- |
| | | | | 3.58 (m, 2H), 3.51-3.48 |
| | | | | (m, 2H), 3.27 (s, 3H), |
| | | | | 3.23-3.16 (m, 2H), 2.92- |
| | | | | 2.78 (m, 2H), 2.30-2.18 |
| | | | | (m, 1H), 1.84-1.60 (m, |
| | | | | 4H). |
| 101 | 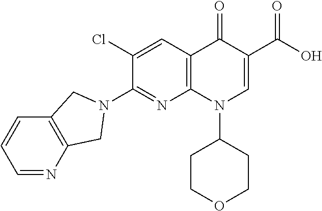 [SMILES: O=C(O)c1cn(C2CCOCC2)c2nc(N3Cc4cccnc4C3)c(Cl)cc2c1=O] | | 6-chloro-7-(5,7- dihydro-6H-pyr- rolo[3,4-b]pyridin- 6-yl)-4-oxo-1- (tetrahydro-2H- pyran-4-yl)-1,4- dihydro-1,8-naph- thyridine-3-car- boxylic acid | MS (ESI): m/z 427 [M + H]⁺; ¹H NMR (400 MHz, CDCl₃): δ 14.87 (s, 1H), 8.79 (s, 1H), 8.60 (d, J = 4.4 Hz, 1H), 8.53 (s, 1H), 7.72 (d, J = 8.0 Hz, 1H), 7.34-7.29 (m, 1H), 5.58- 5.49 (s, 1H), 5.44 (s, 2H), 5.35 (s, 2H), 4.24 (dd, J = 12.0, 4.4 Hz, 2H), 3.68 (td, J = 12.0, 5.6 Hz, 2H), 2.20-2.03 (m, 4H). |
| 102 | 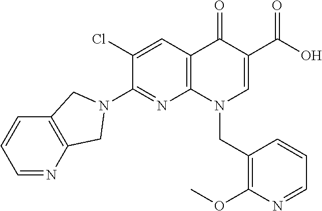 [SMILES: COc1ncccc1Cn1cc(C(=O)O)c(=O)c2cc(Cl)c(N3Cc4cccnc4C3)nc21] | Trituration with 10% MeOH in diethyl ether | 6-chloro-7-(5,7- dihydro-6H-pyr- rolo[3,4-b]pyridin- 6-yl)-1-((2-meth- oxypyridin-3-yl)- methyl)-4-oxo-1,4- dihydro-1,8-naph- thyridine-3-carbox- ylic acid | MS (ESI): m/z 464.0 [M + H]⁺; ¹H NMR (400 MHz, DMSO-d₆): δ 15.11 (bs, 1H), 9.16 (s, 1H), 8.51 (d, J = 4.0 Hz, 1H), 8.33 (s, 1H), 8.09 (dd, J = 5.2, 2.0 Hz, 1H), 7.84 (d, J = 6.8 Hz, 1H), 7.50 (d, J = 6.0 Hz, 1H), 7.38-7.34 (m, 1H), 6.94 (dd, J = 7.2, 4.8 Hz, 1H), 5.69 (s, 2H), 5.20 (bs, 2H), 5.06 (bs, 2H), 4.00 |
| | | | | (s, 3H). |
| 103 | 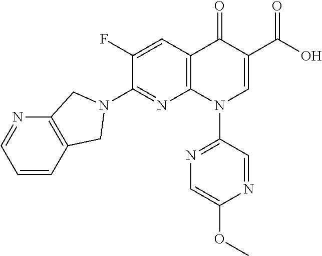 [SMILES: COc1cnc(-n2cc(C(=O)O)c(=O)c3cc(F)c(N4Cc5cccnc5C4)nc32)cn1] | trituration with IPA & Acetonitrile, diethyl ether and n-pentane | 7-(5,7-dihydro-6H- pyrrolo[3,4-b]pyr- idin-6-yl)-6-fluoro- 1-(5-methoxypyr- azin-2-yl)-4-oxo- 1,4-dihydro-1,8- naphthyridine-3- carboxylic acid | MS (ESI): m/z 435.2 [M + H]⁺; ¹H NMR (400 MHz, DMSO-d₆): δ 15.03 (bs, 1H), 8.95 (s, 1H), 8.81 (s, 1H), 8.48 (d, J = 4.0 Hz, 1H), 8.44 (d, J = 1.2 Hz, 1H), 8.20 (d, J = 8.4 Hz, 1H), 7.83 (d, J = 7.6 Hz, 1H), 7.36- 7.32 (m, 1H), 5.24 (bs, 2H), 4.64 (bs, 2H), 4.08 (s, 3H). |
| 104 | 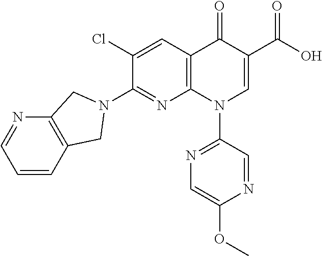 [SMILES: COc1cnc(-n2cc(C(=O)O)c(=O)c3cc(Cl)c(N4Cc5cccnc5C4)nc32)cn1] | trituration with IPA & Acetonitrile, diethyl ether and n-pentane | 6-chloro-7-(5,7- dihydro-6H-pyr- rolo[3,4-b]pyridin- 6-yl)-1-(5-meth- oxypyrazin-2-yl)- 4-oxo-1,4-dihydro- 1,8-naphthyridine- 3-carboxylic acid | MS (ESI): m/z 451.2 [M + H]⁺; ¹H NMR (400 MHz, DMSO-d₆): δ 14.79 (bs, 1H), 8.97 (s, 1H), 8.81 (s, 1H), 8.50- 8.39 (m, 3H), 7.89 (d, J = 8.0 Hz, 1H), 7.34-7.30 (m, 1H), 5.15 (s, 2H), 4.98 (s, 2H), 4.08 (s, 3H). |
| 105 | 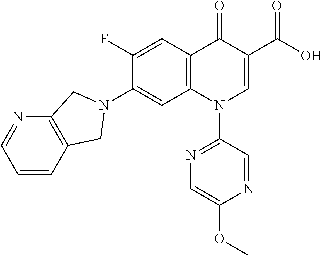 [SMILES: COc1cnc(-n2cc(C(=O)O)c(=O)c3cc(F)c(N4Cc5cccnc5C4)cc32)cn1] | trituration with IPA & Acetonitrile, diethyl ether and n-pentane | 7-(5,7-dihydro-6H- pyrrolo[3,4-b]pyr- idin-6-yl)-6-fluoro- 1-(5-methoxypyr- azin-2-yl)-4-oxo- 1,4-dihydroquin- oline-3-carboxylic acid | MS (ESI): m/z 434.1 [M + H]⁺; ¹H NMR (400 MHz, DMSO-d₆): δ 15.22 (bs, 1H), 8.83 (s, 1H), 8.73 (s, 1H), 8.50- 8.46 (m, 2H), 7.98 (d, J = 14.4 Hz, 1H), 7.81 (d, J = 7.8 Hz, 1H) 7.35-7.31 (m, 1H), 6.36 (d, J = 7.8 Hz, 1H), 4.91 (s, 2H), 4.82 (s, 2H), 4.09 (s, 3H). |
| 106 | 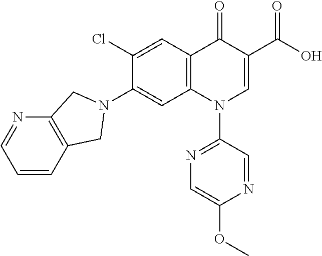 [SMILES: COc1cnc(-n2cc(C(=O)O)c(=O)c3cc(Cl)c(N4Cc5cccnc5C4)cc32)cn1] | trituration with IPA & Acetonitrile, diethyl ether and n-pentane | 6-chloro-7-(5,7- dihydro-6H-pyr- rolo[3,4-b]pyridin- 6-yl)-1-(5-meth- oxypyrazin-2-yl)- 4-oxo-1,4-dihydro- quinoline-3-carbox- ylic acid | MS (ESI): m/z 450.1 [M + H]⁺; ¹H NMR (400 MHz, DMSO-d₆): δ 14.94 (bs, 1H), 8.86 (s, 1H), 8.74 (s, 1H), 8.49- 8.45 (m, 2H), 8.25 (s, 1H), 7.80 (d, J = 7.6 Hz, 1H), 7.34-7.30 (m, 1H), 6.56 (s, 1H), 5.02 (s, 2H), 4.93 (s, 2H), 4.08 (s, 3H). |
| 107 | 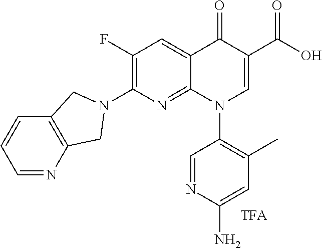 [SMILES: Cc1cc(N)ncc1-n1cc(C(=O)O)c(=O)c2cc(F)c(N3Cc4cccnc4C3)nc21] | Trituration with diethyl ether | 1-(6-amino-4- methylpyridin-3- yl)-7-(5,7-dihydro- 6H-pyrrolo[3,4-b]- pyridin-6-yl)-6- fluoro-4-oxo-1,4- dihydro-1,8-naph- thyridine-3-car- boxylic acid TFA salt | MS (ESI): m/z 433.1 [M + H]⁺; ¹H NMR (400 MHz, DMSO-d₆): δ 15.18 (bs, 1H), 8.79 (s, 1H), 8.48 (d, J = 4.8 Hz, 1H), 8.21-8.17 (m, 2H), 7.83 (d, J = 7.6 Hz, 1H), 7.69 (bs, 1H), 7.36-7.31 (m, 1H), 6.83 (s, 1H), 5.30 (bs, 2H), 4.54 (bs, 2H), 2.04 (s, 3H) (couple of exchangeable protons were not observed by ¹HNMR). |
| 108 | 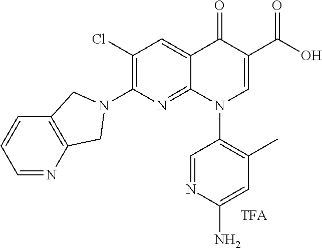 [SMILES: Cc1cc(N)ncc1-n1cc(C(=O)O)c(=O)c2cc(Cl)c(N3Cc4cccnc4C3)nc21] | Trituration with n-pentane | 1-(6-amino-4-meth- ylpyridin-3-yl)-6- chloro-7-(5,7-di- hydro-6H-pyrrolo- [3,4-b]pyridin-6- yl)-4-oxo-1,4- dihydro-1,8-naph- thyridine-3-carbox- ylic acid TFA salt | MS (ESI): m/z 449 [M + H]⁺; ¹H NMR (400 MHz, DMSO-d₆): δ 14.95 (brs, 1H), 8.82 (s, 1H), 8.47 (d, J = 4.8 Hz, 1H), 8.41 (s, 1H), 8.19 (s, 1H), 7.80-7.68 (m, 3H), 7.33 (q, J = 4.8 Hz, 1H), 6.84 (s, 1H), 5.21 (m, 2H), 4.86 (s, 2H), 2.06 (s, 3H); |
| 109 | 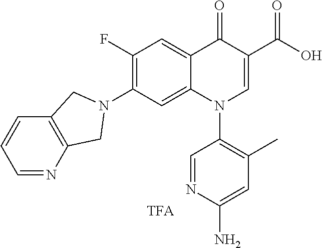 [SMILES: Cc1cc(N)ncc1-n1cc(C(=O)O)c(=O)c2cc(F)c(N3Cc4cccnc4C3)cc21] | trituration with diethyl ether and MeOH | 1-(6-amino-4- methylpyridin-3- yl)-7-(5,7-dihydro- 6H-pyrrolo[3,4-b]- pyridin-6-yl)-6- fluoro-4-oxo-1,4- dihydroquinoline- 3-carboxylic acid TFA salt | MS (ESI): m/z 432 [M + H]⁺; ¹H NMR (400 MHz, DMSO-d₆): δ 15.38 (s, 1H), 8.53 (s, 1H), 8.47 (d, J = 4.8 Hz, 1H), 8.04 (s, 1H), 7.98 (d, J = 14.0 Hz, 1H), 7.86 (d, J = 7.6 Hz, 1H), 7.32 (q, J = 4.8 Hz, 1H), 6.52-6.48 (m, 3H), 6.06 (d, J = 7.6 Hz, 1H), 4.84 (s, 2H), 4.81 (s, 2H), 1.90 (s, 3H); |
| 110 | 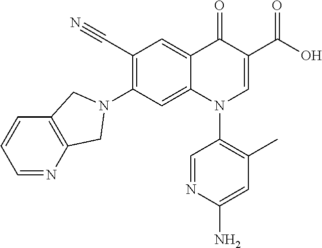 [SMILES: Cc1cc(N)ncc1-n1cc(C(=O)O)c(=O)c2cc(C#N)c(N3Cc4cccnc4C3)cc21] | Trituration with 10% Isopropanol in diethyl ether | 1-(6-amino-4-meth- ylpyridin-3-yl)-6- cyano-7-(5,7- dihydro-6H-pyr- rolo[3,4-b]pyridin- 6-yl)-4-oxo-1,4- dihydroquinoline- 3-carboxylic acid | MS (ESI): m/z 439 [M + H]⁺; ¹H NMR (400 MHz, DMSO-d₆): δ 14.72 (s, 1H), 8.57 (d, J = 6.0 Hz, 2H), 8.49 (d, J = 4.4 Hz, 1H), 8.05 (s, 1H), 7.90 (d, J = 7.6 Hz, 1H), 7.37- 7.32 (m, 1H), 6.52 (bs, 3H), 6.04 (s, 1H), 4.96 (s, 4H), 1.93 (s, 3H); |
| 111 | 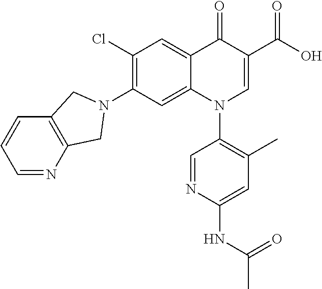 [SMILES: CC(=O)Nc1cc(C)c(-n2cc(C(=O)O)c(=O)c3cc(Cl)c(N4Cc5cccnc5C4)cc32)cn1] | Trituration with 10% Isopropanol in diethyl ether | 1-(6-acetamido-4- methylpyridin-3- yl)-6-chloro-7-(5,7- dihydro-6H-pyr- rolo[3,4-b]pyridin- 6-yl)-4-oxo-1,4- dihydroquinoline- 3-carboxylic acid | MS (ESI): m/z 490 [M + H]⁺; ¹H NMR (400 MHz, DMSO-d₆): δ 15.08 (s, 1H), 10.86 (s, 1H), 8.71 (s, 1H), 8.49 (s, 1H), 8.45 (d, J = 4.4 Hz, 1H), 8.29 (s, 1H), 8.25 (s, 1H), 7.81 (d, J = 7.2 Hz, 1H), 7.33- 7.29 (m, 1H), 6.10 (s, 1H), 4.96 (s, 2H), 4.86 (m, 4H), 2.18 (s, 3H), 2.10 (s, 3H); |
| 112 | 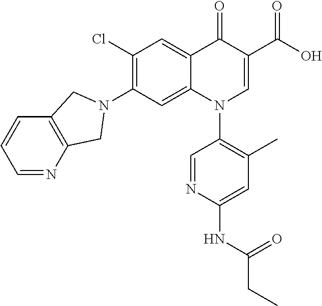 [SMILES: CCC(=O)Nc1cc(C)c(-n2cc(C(=O)O)c(=O)c3cc(Cl)c(N4Cc5cccnc5C4)cc32)cn1] | Trituration with diethyl ether and MeOH | 6-chloro-7-(5,7- dihydro-6H-pyr- rolo[3,4-b]pyridin- 6-yl)-1-(4-methyl- 6-propionamido- pyridin-3-yl)-4- oxo-1,4-dihydro- quinoline-3-car- boxylic acid | MS (ESI): m/z 504 [M + H]⁺; ¹H NMR (400 MHz, DMSO-d₆): δ 15.11 (s, 1H), 10.83 (s, 1H), 8.71 (s, 1H), 8.49 (s, 1H), 8.45 (d, J = 4.4 Hz, 1H), 8.33 (s, 1H), 8.26 (s, 1H), 7.81 (d, J = 6.8 Hz, 1H), 7.30 (q, J = 4.8 Hz, 1H), 6.11 (s, 1H), 4.96 (s, 2H), 4.87 (s, 2H), 2.47-2.38 (m, 2H), 2.10 (s, 3H), 1.11 (t, J = 7.6 Hz, 3H); |
| 113 | 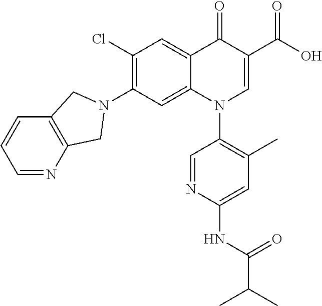 [SMILES: Cc1cc(NC(=O)C(C)C)ncc1-n1cc(C(=O)O)c(=O)c2cc(Cl)c(N3Cc4cccnc4C3)cc21] | Normal phase silica gel column chromatography | 6-chloro-7-(5,7- dihydro-6H-pyr- rolo[3,4-b]pyridin- 6-yl)-1-(6-isobutyr- amido-4-methyl- pyridin-3-yl)-4- oxo-1,4-dihydro- quinoline-3-car- boxylic acid | MS (ESI): m/z 518.2 [M + H]⁺; ¹H NMR (400 MHz, DMSO-d₆): δ 15.08 (s, 1H), 10.81 (s, 1H), 8.70 (s, 1H), 8.49 (s, 1H), 8.45 (d, J = 4.4 Hz, 1H), 8.34 (s, 1H), 8.26 (s, 1H), 7.81 (d, J = 7.6 Hz, 1H), 7.30 (q, J = 4.8 Hz, 1H), 6.13 (s, 1H), 4.96 (s, 2H), 4.87 (s, 2H), 2.88-2.81 (m, 1H), 2.10 (s, 3H), 1.16-1.13 (m, 6H); |
| 114 | 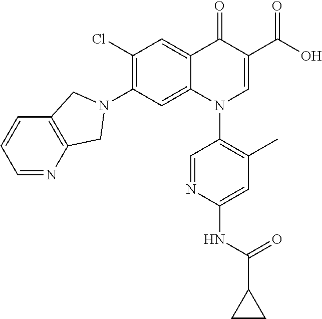 [SMILES: Cc1cc(NC(=O)C2CC2)ncc1-n1cc(C(=O)O)c(=O)c2cc(Cl)c(N3Cc4cccnc4C3)cc21] | Trituration with Et₂O | 6-chloro-1-(6- (cyclopropanecar- boxamido)-4-meth- ylpyridin-3-yl)-7- (5,7-dihydro-6H- pyrrolo[3,4-b]pyr- idin-6-yl)-4-oxo-1, 4-dihydroquin- oline-3-carboxylic acid | MS (ESI): m/z 516 [M + H]⁺; ¹H NMR (400 MHz, DMSO-d₆): δ 15.10 (s, 1H), 11.19 (s, 1H), 8.72 (s, 1H), 8.50 (s, 1H), 8.45 (d, J = 4.0 Hz, 1H), 8.31 (s, 1H), 8.26 (s, 1H), 7.82 (d, J = 7.6 Hz, 1H), 7.31 (q, J = 4.8 Hz, 1H), 6.11 (s, 1H), 4.94 (s, 2H), 4.90 (s, 2H), 2.09 (s, 3H), 2.09-2.07 (m, 1H), 0.90- 0.86 (m, 4H). |
| 115 | 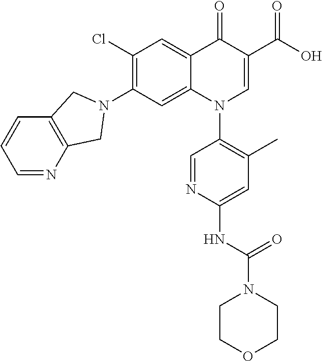 [SMILES: Cc1cc(NC(=O)N2CCOCC2)ncc1-n1cc(C(=O)O)c(=O)c2cc(Cl)c(N3Cc4cccnc4C3)cc21] | Prep-HPLC, Method-4 of Table-2 | 6-chloro-7-(5,7- dihydro-6H-pyr- rolo[3,4-b]pyridin- 6-yl)-1-(4-methyl- 6-(morpholine-4- carboxamido)pyr- idin-3-yl)-4-oxo-1, 4-dihydroquin- oline-3-carboxylic acid | MS (ESI): m/z 561.2 [M + H]⁺; ¹H NMR (400 MHz, DMSO-d₆): δ 14.86 (brs, 1H), 9.48 (bs, 1H), 8.81 (s, 1H), 8.49 (d, J = 6.0 Hz, 1H), 8.46 (s, 1H), 8.35 (s, 1H), 8.15 (s, 1H), 7.82 (d, J = 7.2 Hz, 1H), 7.36-7.32 (m, 1H), 6.44 (s, 1H), 4.88 (brs, 4H), 3.71 (brs, 4H), 3.10-2.97 (m, 4H), 2.06 (s, 3H). |
| 116 | 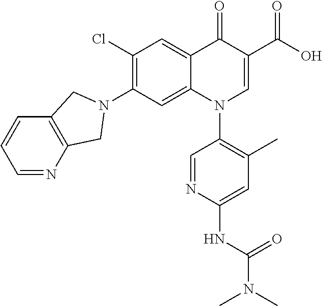 [SMILES: Cc1cc(NC(=O)N(C)C)ncc1-n1cc(C(=O)O)c(=O)c2cc(Cl)c(N3Cc4cccnc4C3)cc21] | Prep-HPLC, Method-4 of Table-2 | 6-chloro-7-(5,7- dihydro-6H-pyr- rolo[3,4-b]pyridin- 6-yl)-1-(6-(3,3- dimethylureido)-4- methylpyridin-3- yl)-4-oxo-1,4-di- hydroquinoline-3- carboxylic acid | MS (ESI): m/z 519.3 [M + H]⁺; ¹H NMR (400 MHz, DMSO-d₆): δ 9.48 (s, 1H), 8.78 (s, 1H), 8.48 (d, J = 4.4 Hz, 1H), 8.47 (s, 1H), 8.29 (s, 1H), 8.14 (s, 1H), 8.07 (s, 1H), 7.83 (d, J = 7.8 Hz, 1H), 7.34 (q, J = 4.8 Hz, 1H), 4.88 (s, 4H), 2.79 (s, 6H), 2.07 (s, 3H), COOH proton was not observed. |
| 117 | 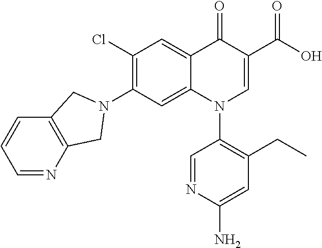 [SMILES: CCc1cc(N)ncc1-n1cc(C(=O)O)c(=O)c2cc(Cl)c(N3Cc4cccnc4C3)cc21] | Trituration with Et₂O | 1-(6-amino-4-ethyl- pyridin-3-yl)-6- chloro-7-(5,7-di- hydro-6H-pyrrolo- [3,4-b]pyridin-6- yl)-4-oxo-1,4-di- hydroquinoline-3- carboxylic acid | MS (ESI): m/z 462 [M + H]⁺; ¹H NMR (400 MHz, DMSO-d₆): δ 15.10 (s, 1H), 8.60 (s, 1H), 8.46 (d, J = 4.4 Hz, 1H), 8.24 (s, 1H), 8.08 (s, 1H), 7.84 (d, J = 7.2 Hz, 1H), 7.34- 7.30 (m, 1H), 6.73 (bs, 2H), 6.62 (s, 1H), 6.22 (s, 1H), 5.00-4.83 (m, 4H), 2.21 (q, J = 7.2 Hz, 2H), 1.01 (t, J = 7.2 Hz, 3H); |
| 118 | 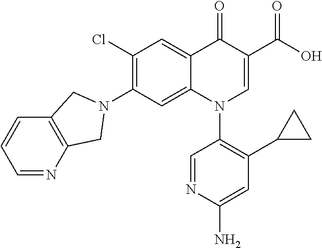 [SMILES: Nc1cc(C2CC2)c(-n2cc(C(=O)O)c(=O)c3cc(Cl)c(N4Cc5cccnc5C4)cc32)cn1] | Prep-HPLC, Method-4 of Table-2 | 1-(6-amino-4- cyclopropylpyridin- 3-yl)-6-chloro-7- (5,7-dihydro-6H- pyrrolo[3,4-b]pyr- idin-6-yl)-4-oxo- 1,4-dihydroquin- oline-3-carboxylic acid | MS (ESI): m/z 474.2 [M + H]⁺; ¹H NMR (400 MHz, DMSO-d₆) δ 8.61 (s, 1H), 8.46 (d, J = 4.4 Hz, 1H), 8.24 (s, 1H), 8.03 (s, 1H), 7.84 (d, J = 8.0 Hz, 1H), 7.32 (q, J = 7.6 Hz, 1H), 6.40 (s, 2H), 6.33 (s, 1H), 6.17 (s, 1H), 5.02-4.82 (m, 4H), 1.29- 1.23 (m, 1H), 0.82-0.68 (m, 4H), COOH proton was not observed. |
| 119 | 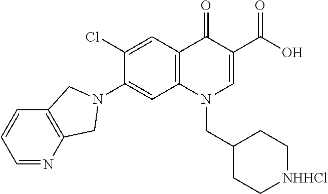 | Trituration with MeOH | 6-chloro-7-(5,7- dihydro-6H-pyr- rolo[3,4-b]pyridin- 6-yl)-4-oxo-1- (piperidin-4-yl- methyl)-1,4- dihydroquinoline- 3-carboxylic acid hydrochloride | MS (ESI): m/z 439.0 [M + H]⁺; ¹H NMR (400 MHz, DMSO-d₆): δ 8.94 (s, 1H), 8.81-8.79 (m, 1H), 8.54 (d, J = 4.4 Hz, 1H), 8.49-8.26 (m, 1H), 8.21 (s, 1H), 7.89 (d, J = 7.6 Hz, 1H), 7.41-7.38 (m, 1H), 7.01 (s, 1H), 5.28 (s, 2H), 5.16 (s, 2H), 4.58 (d, J = 4.4 Hz, 2H), 3.28-3.24 (m, 2H), 2.84- |
| | | | | 2.76 (m, 2H), 2.32-2.29 |
| | | | | (m, 1H), 1.75-1.71 (m, |
| | | | | 2H), 1.54-1.45 (m, 2H) |
| | | | | (—COOH peak was not |
| | | | | observed) |
| 120 | 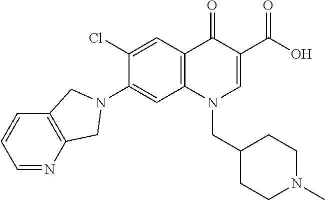 [SMILES: CN1CCC(Cn2cc(C(=O)O)c(=O)c3cc(Cl)c(N4Cc5cccnc5C4)cc32)CC1] | Trituration with MeOH | 6-chloro-7-(5,7- dihydro-6H-pyr- rolo[3,4-b]pyridin- 6-yl)-1-((1-methyl- piperidin-4-yl)- methyl)-4-oxo-1,4- dihydroquinoline- 3-carboxylic acid | MS (ESI): m/z 453.0 [M + H]⁺; ¹H NMR (400 MHz, DMSO-d₆): δ 15.28 (s, 1H), 8.91 (s, 1H), 8.53 (d, J = 4.4 Hz, 1H), 8.20 (s, 1H), 7.88 (d, J = 7.6 Hz, 1H), 7.40- 7.36 (m, 1H), 7.01 (s, 1H), 5.25 (s, 2H), 5.13 (s, 2H), 4.54 (d, J = 4.4 Hz, 2H), 3.12-2.99 (m, 2H), 2.49 (s, 3H), 2.38 (brs, 2H), 2.10-2.00 (m, 1H), |
| | | | | 1.68-1.60 (m, 2H), 1.52- |
| | | | | 1.42 (m, 2H) |
| 121 | 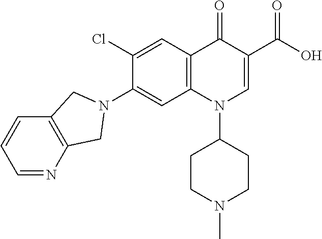 [SMILES: CN1CCC(n2cc(C(=O)O)c(=O)c3cc(Cl)c(N4Cc5cccnc5C4)cc32)CC1] | Trituration with n-Pantane | 6-chloro-7-(5,7- dihydro-6H-pyr- rolo[3,4-b]pyridin- 6-yl)-1-(1-methyl- piperidin-4-yl)-4- oxo-1,4-dihydro- quinoline-3-carbox- ylic acid | MS (ESI): m/z 439 [M + H]⁺; ¹H NMR (400 MHz, DMSO-d₆): δ 15.22 (s, 1H), 8.70 (s, 1H), 8.53 (s, 1H), 8.23 (s, 1H), 7.89 (d, J = 7.6 Hz, 1H), 7.37 (s, 1H), 7.14 (s, 1H), 5.26 (s, 2H), 5.15 (s, 2H), 4.85 (bs, 1H), 2.95 (bs, 2H), 2.40-1.98 (m, 9H). |
| 122 | 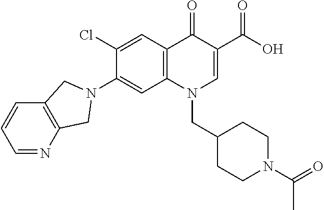 [SMILES: CC(=O)N1CCC(Cn2cc(C(=O)O)c(=O)c3cc(Cl)c(N4Cc5cccnc5C4)cc32)CC1] | Trituration with MeOH | 1-((1-acetylpiper- idin-4-yl)methyl)- 6-chloro-7-(5,7- dihydro-6H-pyr- rolo[3,4-b]pyridin- 6-yl)-4-oxo-1,4- dihydroquinoline- 3-carboxylic acid | MS (ESI): m/z 481.0 [M + H]⁺; ¹H NMR (400 MHz, DMSO-d₆): δ 15.28 (bs, 1H), 8.87 (s, 1H), 8.53 (d, J = 4.4 Hz, 1H), 8.21 (s, 1H), 7.88 (d, J = 8.0 Hz, 1H), 7.40- 7.35 (m, 1H), 7.03 (s, 1H), 5.26 (s, 2H), 5.14 (s, 2H). 4.53-4.37 (m, 3H), 3.83-3.78 (m, 1H), 2.95 (d, J = 11.6 Hz, 2H), 2.32-2.29 (m, 1H), 1.98 |
| | | | | (s, 3H), 1.56-1.52 (m, |
| | | | | 2H), 1.32-1.15 (m, 2H); |
| 123 | 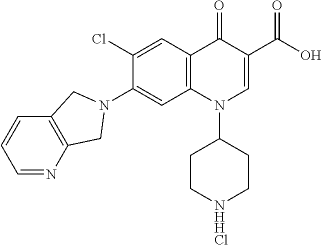 | Trituration with MeOH followed by IPA | 6-chloro-7-(5,7- dihydro-6H-pyr- rolo[3,4-b]pyridin- 6-yl)-4-oxo-1- (piperidin-4-yl)- 1,4-dihydroquin- oline-3-carboxylic acid hydrochloride | MS (ESI): m/z 425 [M + H]⁺; ¹H NMR (400 MHz, DMSO-d₆): δ 9.27 (d, J = 7.2 Hz, 1H), 8.65 (s, 1H), 8.54 (d, J = 4.4 Hz, 1H), 8.23 (s, 1H), 7.89 (d, J = 7.6 Hz, 1H), 7.41-7.37 (m, 1H), 7.19 (s, 1H), 5.29 (s, 3H), 5.17 (s, 2H), 3.58-3.13 (m, 4H), 2.32- 2.25 (m, 4H). |
| 124 | 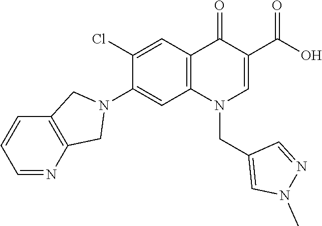 [SMILES: Cn1cc(Cn2cc(C(=O)O)c(=O)c3cc(Cl)c(N4Cc5cccnc5C4)cc32)cn1] | trituration with IPA, ACN, diethyl ether and n-pentane | 6-chloro-7-(5,7- dihydro-6H-pyr- rolo[3,4-b]pyridin- 6-yl)-1-((1-methyl- 1H-pyrazol-4-yl)- methyl)-4-oxo-1,4- dihydroquinoline- 3-carboxylic acid | MS (ESI): m/z 436.1 [M + H]⁺; ¹H NMR (400 MHz, DMSO-d₆): δ 15.27 (s, 1H), 12.16 (bs, 1H), 9.09 (s, 1H), 8.52 (d, J = 4.0 Hz, 1H), 8.17 (s, 1H), 7.89 (d, J = 6.8 Hz, 1H), 7.84 (s, 1H), 7.62 (s, 1H), 7.40-7.35 (m, 1H), 7.12 (s, 1H), 5.66 (s, 2H), 5.20 (s, 2H), 5.10 (s, 2H), 3.77 (s, 3H). |
| 125 | 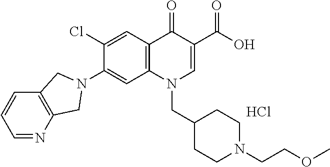 [SMILES: COCCN1CCC(Cn2cc(C(=O)O)c(=O)c3cc(Cl)c(N4Cc5cccnc5C4)cc32)CC1 ; Cl] | Trituration with Et2O and IPA | 6-chloro-7-(5,7- dihydro-6H-pyr- rolo[3,4-b]pyridin- 6-yl)-1-((1-(2- methoxyethyl)- piperidin-4-yl)- methyl)-4-oxo-1,4- dihydroquinoline- 3-carboxylic acid hydrochloride | MS (ESI): m/z 497.0 [M + H]⁺; ¹H NMR (400 MHz, DMSO-d₆): δ 15.26 (brs, 1H), 9.84 (brs, 1H), 8.92 (brs, 1H), 8.53 (d, J = 3.6 Hz, 1H), 8.21 (s, 1H), 7.88 (d, J = 7.6 Hz, 1H), 7.40-7.36 (m, 1H), 7.01 (s, 1H), 5.28 (s, 2H), 5.14 (s, 2H), 4.57 (brs, 2H), 3.70-3.58 |
| | | | | (m, 2H), 3.51-3.48 (m, |
| | | | | 2H), 3.27 (s, 3H), 3.23- |
| | | | | 3.16 (m, 2H), 2.92-2.78 |
| | | | | (m, 2H), 2.30-2.18 (m, |
| | | | | 1H), 1.84-1.58 (m, 4H). |
| 126 | 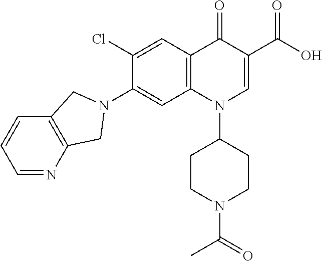 [SMILES: CC(=O)N1CCC(n2cc(C(=O)O)c(=O)c3cc(Cl)c(N4Cc5cccnc5C4)cc32)CC1] | Trituration with MeOH | 1-(1-acetylpiper- idin-4-yl)-6-chloro- 7-(5,7-dihydro-6H- pyrrolo[3,4-b]pyr- idin-6-yl)-4-oxo- 1,4-dihydroquin- oline-3-carboxylic acid | MS (ESI): m/z 467 [M + H]⁺; ¹H NMR (400 MHz, DMSO-d₆): δ 15.23 (s, 1H), 8.77 (s, 1H), 8.53 (d, J = 4.0 Hz, 1H), 8.24 (s, 1H), 7.88 (d, J = 7.2 Hz, 1H), 7.38 (dd, J = 7.6, 4.8 Hz, 1H), 7.22 (s, 1H), 5.27 (s, 2H), 5.23- 5.15 (m, 3H), 4.61 (d, J = 12.4 Hz, 1H), 4.02 (d, J = 12.4 Hz, 1H), 3.50-3.42 (m, 1H), 2.92 (t, J = 12.4 Hz, 1H), 2.20-2.11 (m, 3H), 2.09 (s, 3H), 1.92- 1.82 (m, 1H); |
| 127 | 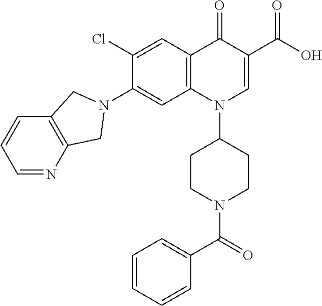 [SMILES: O=C(O)c1cn(C2CCN(C(=O)c3ccccc3)CC2)c2cc(N3Cc4cccnc4C3)c(Cl)cc2c1=O] | Normal phase silica gel column chromatography | 1-(1-benzoylpiper- idin-4-yl)-6-chloro- 7-(5,7-dihydro-6H- pyrrolo[3,4-b]pyr- idin-6-yl)-4-oxo- 1,4-dihydroquin- oline-3-carboxylic acid | MS (ESI): m/z 529.0 [M + H]⁺; ¹H NMR (400 MHz, DMSO-d₆): δ 15.25 (s, 1H), 8.86 (s, 1H), 8.53 (d, J = 4.0 Hz, 1H), 8.24 (s, 1H), 7.87 (d, J = 7.6 Hz, 1H), 7.55- 7.46 (m, 5H), 7.40-7.35 (m, 1H), 7.21 (s, 1H), 5.27 (s, 2H), 5.22 (bs, 1H), 5.16 (s, 2H), 4.74 (bs, 1H), 3.78 (bs, 1H), 3.47 (bs, 1H), 3.21 (bs, 1H), 2.25-2.10 (m, 4H). |
| 128 | 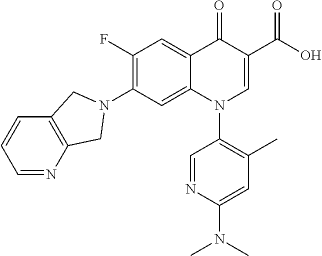 [SMILES: Cc1cc(N(C)C)ncc1-n1cc(C(=O)O)c(=O)c2cc(F)c(N3Cc4cccnc4C3)cc21] | trituration with 10% Isopropanol in diethyl ether | 7-(5,7-dihydro-6H- pyrrolo[3,4-b]pyr- idin-6-yl)-1-(6- (dimethylamino)-4- methylpyridin-3- yl)-6-fluoro-4-oxo- 1,4-dihydroquin- oline-3-carboxylic acid | MS (ESI): m/z 460 [M + H]⁺; ¹H NMR (400 MHz, DMSO-d₆): δ 15.39 (s, 1H), 8.53 (s, 1H), 8.46 (d, J = 3.6 Hz, 1H), 8.21 (s, 1H), 8.00 (d, J = 14.0 Hz, 1H), 7.84 (d, J = 7.6 Hz, 1H), 7.35-7.31 (m, 1H), 6.78 (s, 1H), 6.05 (d, J = 7.6 Hz, 1H), 4.87 (s, 2H), 4.77 (s, 2H), 3.15 (s, 6H), 1.99 (s, 3H). |
| 129 | 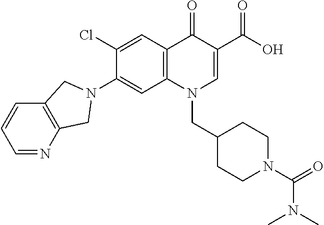 [SMILES: CN(C)C(=O)N1CCC(Cn2cc(C(=O)O)c(=O)c3cc(Cl)c(N4Cc5cccnc5C4)cc32)CC1] | trituration with 10% Isopropanol in diethyl ether | 6-chloro-7-(5,7- dihydro-6H-pyr- rolo[3,4-b]pyridin- 6-yl)-1-((1-(dimeth- ylcarbamoyl)piper- idin-4-yl)methyl)- 4-oxo-1,4-dihydro- quinoline-3-carbox- ylic acid | MS (ESI): m/z 510.3 [M + H]⁺; ¹H NMR (400 MHz, DMSO-d₆): δ 15.27 (s, 1H), 8.88 (s, 1H), 8.52 (d, J = 4.4 Hz, 1H), 8.18 (s, 1H), 7.87 (d, J = 7.2 Hz, 1H), 7.40- 7.35 (m, 1H), 7.01 (s, 1H), 5.25 (s, 2H), 5.13 (s, 2H). 4.56-4.47 (m, 2H), 3.55 (d, J = 12.4 Hz, 2H), 2.71 (s, 6H), 2.67-2.57 (m, 2H), 2.20-2.10 (m, 1H), 1.55-1.50 (m, 2H), 1.40-1.25 (m, 2H); |
| 130 | 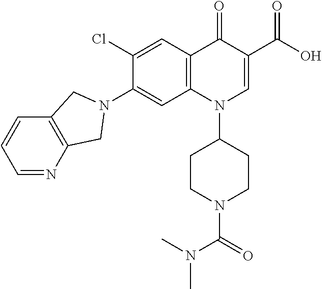 [SMILES: CN(C)C(=O)N1CCC(n2cc(C(=O)O)c(=O)c3cc(Cl)c(N4Cc5cccnc5C4)cc32)CC1] | Trituration with MeOH | 6-chloro-7-(5,7- dihydro-6H-pyr- rolo[3,4-b]pyridin- 6-yl)-1-(1-(dimeth- ylcarbamoyl)piper- idin-4-yl)-4-oxo- 1,4-dihydroquin- oline-3-carboxylic acid | MS (ESI): m/z 496 [M + H]⁺; ¹H NMR (400 MHz, DMSO-d₆): δ 15.24 (s, 1H), 8.73 (s, 1H), 8.53 (d, J = 4.4 Hz, 1H), 8.23 (s, 1H), 7.88 (d, J = 7.2 Hz, 1H), 7.40-7.35 (m, 1H), 7.19 (s, 1H), 5.26 (s, 2H), 5.17 (s, 2H). 5.13- 5.06 (m, 1H), 3.73 (d, J = 13.6 Hz, 2H), 3.15 (t, J = 12.4 Hz, 2H), 2.81 (s, 6H), 2.20-2.00 (m, 4H). |
| 131 | 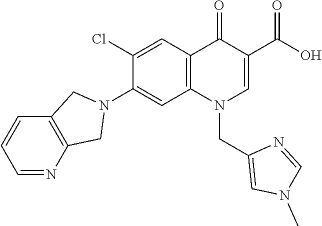 [SMILES: Cn1cnc(Cn2cc(C(=O)O)c(=O)c3cc(Cl)c(N4Cc5cccnc5C4)cc32)c1] | Reverse phase purification; C-18 column, 0.1% FA in ACN | 6-chloro-7-(5,7- dihydro-6H-pyr- rolo[3,4-b]pyridin- 6-yl)-1-((1-methyl- 1H-imidazol-4-yl)- methyl)-4-oxo-1,4- dihydroquinoline- 3-carboxylic acid | MS (ESI): m/z 436.2 [M + H]⁺; ¹H NMR (400 MHz, DMSO-d₆): δ 15.21 (brs, 1H), 9.09 (s, 1H), 8.53 (d, J = 4.0 Hz, 1H), 8.33 (brs, 1H), 8.17 (s, 1H), 7.88 (d, J = 6.4 Hz, 1H), 7.61 (s, 1H), 7.40-7.35 (m, 1H), 7.15 (m, 1H), 5.80 (s, 2H), 5.21 (s, 2H), 5.09 (s, 2H), 3.71 (s, 3H). |
| 132 | 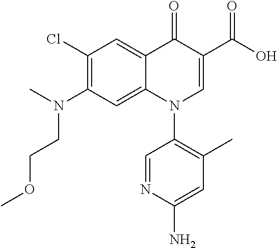 [SMILES: COCCN(C)c1cc2c(cc1Cl)c(=O)c(C(=O)O)cn2-c1cnc(N)cc1C] | trituration with 10% Isopropanol in diethyl ether | 1-(6-amino-4-meth- ylpyridin-3-yl)-6- chloro-7-((2-meth- oxyethyl)(methyl)- amino)-4-oxo-1,4- dihydroquinoline- 3-carboxylic acid | MS (ESI): m/z 417.1 [M + H]⁺; ¹H NMR (400 MHz, DMSO-d₆): δ 8.76 (s, 1H), 8.27 (s, 1H), 8.25 (s, 1H), 7.75 (bs, 2H), 6.81 (s, 1H), 6.56 (s, 1H), 3.58-3.35 (m, 4H), 3.14 (s, 3H), 2.84 (s, 3H), 1.97 (s, 3H) (—COOH proton was not observed). |
| 133 | 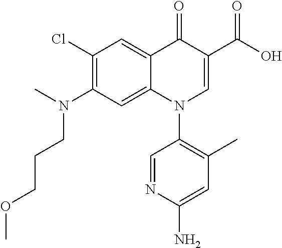 [SMILES: COCCCN(C)c1cc2c(cc1Cl)c(=O)c(C(=O)O)cn2-c1cnc(N)cc1C] | trituration with 10% Isopropanol in diethyl ether | 1-(6-amino-4-meth- ylpyridin-3-yl)-6- chloro-7-((3-meth- oxypropyl)(meth- yl)amino)-4-oxo- 1,4-dihydroquin- oline-3-carboxylic acid | MS (ESI): m/z 431.2 [M + H]⁺; ¹H NMR (400 MHz, DMSO-d₆): δ 8.77 (s, 1H), 8.28 (s, 1H), 8.26 (s, 1H), 7.77 (bs, 2H), 6.82 (s, 1H), 6.48 (s, 1H), 3.28-3.22 (m, 4H), 3.14 (s, 3H), 2.79 (s, 3H), 1.97 (s, 3H), 1.78-1.75 (m, 2H) (—COOH proton was not observed). |
| 134 | 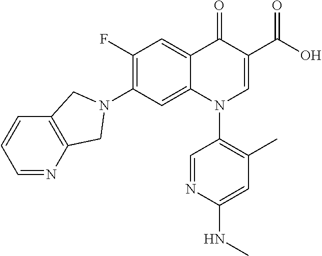 [SMILES: CNc1cc(C)c(-n2cc(C(=O)O)c(=O)c3cc(F)c(N4Cc5cccnc5C4)cc32)cn1] | trituration with 10% Isopropanol in diethyl ether | 7-(5,7-dihydro-6H- pyrrolo[3,4-b]pyr- idin-6-yl)-6-fluoro- 1-(4-methyl-6- (methylamino)pyr- idin-3-yl)-4-oxo- 1,4-dihydroquin- oline-3-carboxylic acid | MS (ESI): m/z 446 [M + H]⁺; ¹H NMR (400 MHz, DMSO-d₆): δ 15.40 (s, 1H), 8.53 (s, 1H), 8.47 (d, J = 3.6 Hz, 1H), 8.12 (s, 1H), 7.98 (d, J = 14.4 Hz, 1H), 7.85 (d, J = 7.6 Hz, 1H), 7.35-7.31 (m, 1H), 7.06 (d, J = 4.8 Hz, 1H), 6.53 (s, 1H), 6.07 (d, J = 7.2 Hz, 1H), 4.85 (s, 2H), 4.79 (s, 2H), 2.87 (d, J = 4.8 Hz, 3H), 1.99 (s, 3H). |
| 135 | 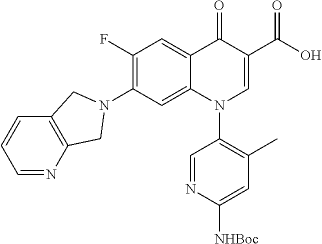 | Prep-HPLC, Method-4 of Table-2 | 1-(6-((tert-butoxy- carbonyl)amino)-4- methylpyridin-3- yl)-7-(5,7-dihydro- 6H-pyrrolo[3,4-b]- pyridin-6-yl)-6- methoxy-4-oxo- 1,4-dihydroquin- oline-3-carboxylic acid | MS (ESI): m/z 544 [M + H]⁺; ¹H NMR (400 MHz, DMSO-d₆): δ 15.86 (brs, 1H), 10.27 (s, 1H), 8.58 (s, 1H), 8.44 (d, J = 3.6 Hz, 1H), 8.41 (s, 1H), 8.03 (s, 1H), 7.81 (d, J = 7.8 Hz, 1H), 7.67 (s, 1H), 7.31-7.27 (m, 1H), 5.88 (s, 1H), 4.82 (s, 4H), 4.00 (s, 3H), 2.06 (s, 3H), 1.53 (s, 9H). |
| 136 | 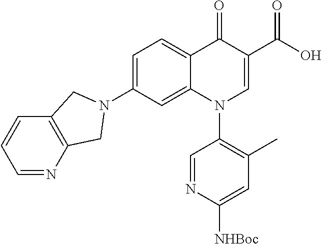 [SMILES: Cc1cc(NC(=O)OC(C)(C)C)ncc1-n1cc(C(=O)O)c(=O)c2ccc(N3Cc4cccnc4C3)cc21] | Prep-HPLC, Method-9 of Table-2 | 1-(6-((tert-butoxy- carbonyl)amino)-4- methylpyridin-3- yl)-7-(5,7-dihydro- 6H-pyrrolo[3,4-b]- pyridin-6-yl)-4- oxo-1,4-dihydro- quinoline-3-carbox- ylic acid | MS (ESI): m/z 514.0 [M + H]+; ¹H NMR (400 MHz, DMSO-d₆): δ 15.64 (s, 1H), 10.25 (s, 1H), 8.62 (s, 1H), 8.47 (d, J = 4.4 Hz, 1H), 8.43 (s, 1H), 8.29 (d, J = 9.2 Hz, 1H), 8.03 (s, 1H), 7.84 (d, J = 8.0 Hz, 1H), 7.32 (q, J = 7.2 Hz, 1H), 7.15 (d, J = 7.2 Hz, 1H), 6.56 (s, 1H), 5.18 (s, 1H), 4.65 (s, 4H), 2.08 (s, 3H), 1.53 (s, 9H) |
| 137 | 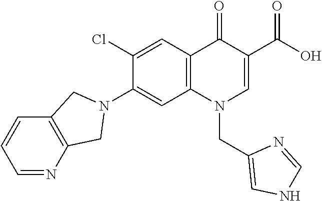 [SMILES: O=C(O)c1cn(Cc2c[nH]cn2)c2cc(N3Cc4cccnc4C3)c(Cl)cc2c1=O] | Prep-HPLC, Method-8 of Table-2 | 1-((1H-imidazol- 4-yl)methyl)-6- chloro-7-(5,7- dihydro-6H-pyr- rolo[3,4-b]pyridin- 6-yl)-4-oxo-1,4- dihydroquinoline- 3-carboxylic acid | MS (ESI): m/z 422.1 [M + H]⁺; ¹H NMR (400 MHz, DMSO-d₆): δ 15.32 (s, 1H), 12.16 (bs, 1H), 9.07 (s, 1H), 8.53 (d, J = 4.0 Hz, 1H), 8.15 (s, 1H), 7.88 (d, J = 7.6 Hz, 1H), 7.65 (s, 1H), 7.43-7.35 (m, 3H), 5.67 (s, 2H), 5.21 (bs, 2H), 5.10 (bs, 2H). |
| 138 | 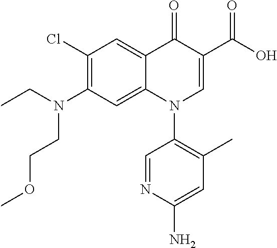 [SMILES: CCN(CCOC)c1cc2c(cc1Cl)c(=O)c(C(=O)O)cn2-c1cnc(N)cc1C] | Prep-HPLC, Method-4 of Table-2 | 1-(6-amino-4-meth- ylpyridin-3-yl)-6- chloro-7-(ethyl(2- methoxyethyl)- amino)-4-oxo-1,4- dihydroquinoline- 3-carboxylic acid | MS (ESI): m/z 431.2 [M + H]⁺; ¹H NMR (400 MHz, DMSO-d₆): δ 15.01 (s, 1H), 8.63 (s, 1H), 8.25 (s, 1H), 8.02 (s, 1H), 6.58 (s, 1H), 6.48 (brs, 3H), 3.42-3.36 (m, 4H), 3.21 (q, J = 7.2 Hz, 2H), 3.09 (s, 3H), 1.86 (s, 3H), 0.99 (t, J = 7.2 Hz, 3H). |
| 139 | 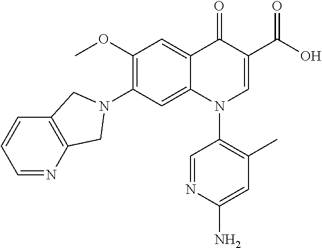 [SMILES: COc1cc2c(=O)c(C(=O)O)cn(-c3cnc(N)cc3C)c2cc1N1Cc2cccnc2C1] | Prep-HPLC, Method-4 of Table-2 | 1-(6-amino-4-meth- ylpyridin-3-yl)-7- (5,7-dihydro-6H- pyrrolo[3,4-b]pyr- idin-6-yl)-6-meth- oxy-4-oxo-1,4- dihydroquinoline- 3-carboxylic acid | MS (ESI): m/z 444 [M + H]⁺; ¹H NMR (400 MHz, DMSO-d₆): δ 15.89 (s, 1H), 8.48 (s, 1H), 8.46 (d, J = 3.6 Hz, 1H), 8.03 (s, 1H), 7.83 (d, J = 7.8 Hz, 1H), 7.65 (s, 1H), 7.34-7.27 (m, 1H), 6.53 (brs, 3H), 6.00 (s, 1H), 4.85 (s, 2H), 4.82 (s, 2H), 4.00 (s, 3H), 1.89 (s, 3H). |
| 140 | 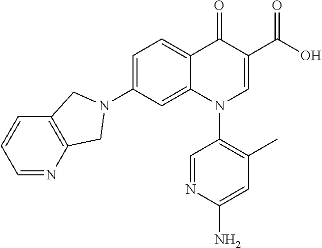 [SMILES: Cc1cc(N)ncc1-n1cc(C(=O)O)c(=O)c2ccc(N3Cc4cccnc4C3)cc21] | Prep-HPLC, Method-4 of Table-2 | 1-(6-amino-4-meth- ylpyridin-3-yl)-7- (5,7-dihydro-6H- pyrrolo[3,4-b]pyr- idin-6-yl)-4-oxo- 1,4-dihydroquin- oline-3-carboxylic acid | MS (ESI): m/z 414.1 [M + H]⁺; ¹H NMR (400 MHz, DMSO-d₆): δ 15.68 (brs, 1H), 8.51 (s, 1H), 8.48 (d, J = 4.4 Hz, 1H), 8.27 (d, J = 8.8 Hz, 1H), 8.04 (s, 1H), 7.87 (d, J = 6.8 Hz, 1H), 7.33 (q, J = 8.0 Hz, 1H), 7.15 (d, J = 9.2 Hz, 1H), 6.52 (s, 1H), 6.48 (s, 2H), 5.92 (s, 1H), 4.67 (s, 4H), 1.91 (s, 3H). |
| 141 | 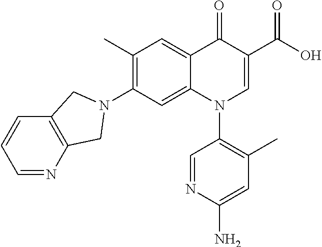 [SMILES: Cc1cc2c(=O)c(C(=O)O)cn(-c3cnc(N)cc3C)c2cc1N1Cc2cccnc2C1] | Prep-HPLC, Method-4 of Table-2 | 1-(6-((tert-butoxy- carbonyl)amino)-4- methylpyridin-3- yl)-7-(5,7-dihydro- 6H-pyrrolo[3,4-b]- pyridin-6-yl)-6- methyl-4-oxo-1,4- dihydroquinoline- 3-carboxylic acid | MS (ESI): m/z 428.2 [M + H]⁺; ¹H NMR (400 MHz, DMSO-d₆): δ 15.68 (s, 1H), 8.51 (s, 1H), 8.45 (d, J = 4.4 Hz, 1H), 8.06 (s, 1H), 8.03 (s, 1H), 7.82 (d, J = 7.6 Hz, 1H), 7.31 (q, J = 5.2 Hz, 1H), 6.55 (s, 1H), 6.51 (s, 1H), 6.48 (s, 2H), 6.09 (s, 1H), 4.83 (s, 2H), 4.78 (s, 2H), 2.73 (s, 3H), 1.90 (s, 3H); |
| 142 | 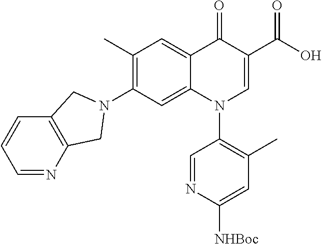 [SMILES: Cc1cc2c(=O)c(C(=O)O)cn(-c3cnc(NC(=O)OC(C)(C)C)cc3C)c2cc1N1Cc2cccnc2C1] | Prep-HPLC, Method-4 of Table-2 | 1-(6-((tert-butoxy- carbonyl)amino)-4- methylpyridin-3- yl)-7-(5,7-dihydro- 6H-pyrrolo[3,4-b]- pyridin-6-yl)-6- methyl-4-oxo-1,4- dihydroquinoline- 3-carboxylic acid | MS (ESI): m/z 528.0 [M + H]⁺; ¹H NMR (400 MHz, DMSO-d₆) δ: 15.65 (s, 1H), 10.25 (s, 1H), 8.61 (s, 1H), 8.45 (d, J = 4.4 Hz, 1H), 8.41 (s, 1H), 8.08 (s, 1H), 8.03 (s, 1H), 7.80 (d, J = 7.6 Hz, 1H), 7.31 (q, J = 5.2 Hz, 1H), 5.97 (s, 1H), 4.81 (s, 2H), 4.78 (s, 2H), 2.74 (s, 3H), 2.07 (s, 3H), 1.53 (s, 9H). |
| 143 | 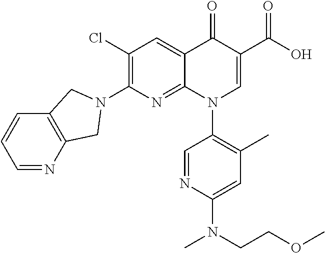 [SMILES: COCCN(C)c1cc(C)c(-n2cc(C(=O)O)c(=O)c3cc(Cl)c(N4Cc5cccnc5C4)nc32)cn1] | trituration with 10% Isopropanol in diethyl ether | 6-chloro-7-(5,7- dihydro-6H-pyr- rolo[3,4-b]pyridin- 6-yl)-1-(6-((2-meth- oxyethyl)(methyl)- amino)-4-methyl- pyridin-3-yl)-4- oxo-1,4-dihydro- 1,8-naphthyridine- 3-carboxylic acid | MS (ESI): m/z 521.2 [M + H]+; 1H NMR (400 MHz, DMSO-d₆): δ 15.04 (brs, 1H), 8.68 (s, 1H), 8.45 (d, J = 4.0 Hz, 1H), 8.41 (s, 1H), 8.10 (s, 1H), 7.75 (d, J = 7.6 Hz, 1H), 7.34-7.30 (m, 1H), 6.76 (s, 1H), 5.14 (brs, 2H), 4.83 (brs, 2H), 3.90- 3.72 (m, 2H), 3.57 (t, J = 5.6 Hz, 2H), 3.30 (s, 3H), 3.13 (s, 3H), 2.02 (s, 3H). |
| 144 | 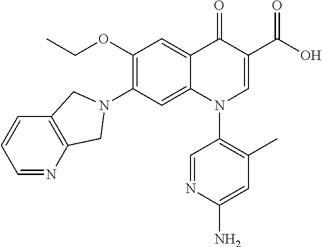 [SMILES: CCOc1cc2c(=O)c(C(=O)O)cn(-c3cnc(N)cc3C)c2cc1N1Cc2cccnc2C1] | Prep-HPLC, Method-4 of Table-2 | 1-(6-amino-4-meth- ylpyridin-3-yl)-7- (5,7-dihydro-6H- pyrrolo[3,4-b]pyr- idin-6-yl)-6-ethoxy- 4-oxo-1,4-dihydro- quinoline-3-car- boxylic acid | MS (ESI): m/z 458 [M + H]⁺; ¹H NMR (400 MHz, DMSO-d₆): δ 15.87 (s, 1H), 8.45 (s, 2H), 8.02 (s, 1H), 7.83 (d, J = 7.6 Hz, 1H), 7.63 (s, 1H), 7.34- 7.27 (m, 1H), 6.52 (s, 1H), 6.46 (brs, 2H), 6.00 (s, 1H), 4.86 (s, 2H), 4.84 (s, 2H), 4.24 (q, J = 6.8 Hz, 2H), 1.88 (s, 3H), 1.50 (t, J = 6.8 Hz, 3H). |
| 145 | 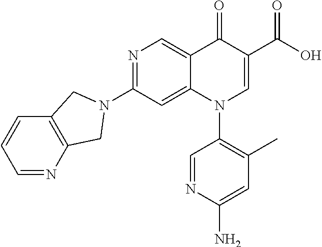 [SMILES: Cc1cc(N)ncc1-n1cc(C(=O)O)c(=O)c2cnc(N3Cc4cccnc4C3)cc21] | Prep-HPLC, Method-10 of Table-2 | 1-(6-amino-4-meth- ylpyridin-3-yl)-7- (5,7-dihydro-6H- pyrrolo[3,4-b]pyr- idin-6-yl)-4-oxo- 1,4-dihydro-1,6- naphthyridine-3- carboxylic acid | MS (ESI): m/z 415 [M + H]⁺; ¹H NMR (400 MHz, DMSO-d₆): δ 14.86 (s, 1H), 9.23 (s, 1H), 8.54 (s, 1H), 8.48 (d, J = 4.4 Hz, 1H), 8.03 (s, 1H), 7.86 (d, J = 6.8 Hz, 1H), 7.35- 7.31 (m, 1H), 6.50 (s, 1H), 6.47 (brs, 2H), 5.68 (s, 1H), 4.89 (brs, 2H), 4.68 (brs, 2H), 1.94 (s, 3H). |
| 146 | 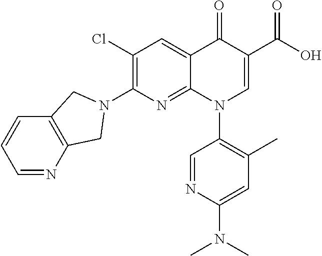 [SMILES: Cc1cc(N(C)C)ncc1-n1cc(C(=O)O)c(=O)c2cc(Cl)c(N3Cc4cccnc4C3)nc21] | Prep-HPLC, Method-11 of Table-2 | 6-chloro-7-(5,7- dihydro-6H-pyr- rolo[3,4-b]pyridin- 6-yl)-1-(6-(dimeth- ylamino)-4-methyl- pyridin-3-yl)-4- oxo-1,4-dihydro- 1,8-naphthyridine- 3-carboxylic acid | MS (ESI): m/z 477.2 [M + H]⁺; ¹H NMR (400 MHz, DMSO-d₆): δ 8.60 (s, 1H), 8.45 (d, J = 3.6 Hz, 1H), 8.40 (s, 1H), 8.37 (s, 1H), 8.09 (s, 1H), 7.78 (d, J = 7.6 Hz, 1H), 7.34-7.30 (m, 1H), 6.74 (s, 1H), 5.16 (s, 2H), 4.81 (s, 2H), 3.13 (s, 6H), 2.00 (s, 3H). |
| 147 | 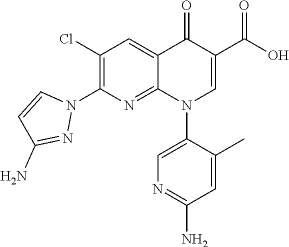 [SMILES: Cc1cc(N)ncc1-n1cc(C(=O)O)c(=O)c2cc(Cl)c(-n3ccc(N)n3)nc21] | Prep-HPLC, Method-4 of Table-2 | 7-(3-amino-1H- pyrazol-1-yl)-1-(6- amino-4-methyl- pyridin-3-yl)-6- chloro-4-oxo-1,4- dihydro-1,8-naph- thyridine-3-carbox- ylic acid | MS (ESI): m/z 412.1 [M + H]⁺; ¹H NMR (400 MHz, DMSO-d₆): δ 14.46 (s, 1H), 8.79 (s, 1H), 8.71 (s, 1H), 7.94 (s, 1H), 7.63 (d, J = 2.8 Hz, 1H), 6.45 (s, 1H), 6.35 (s, 2H), 5.95 (d, J = 2.8 Hz, 1H), 5.61 (s, 2H), 1.90 (s, 3H); |
| 148 | 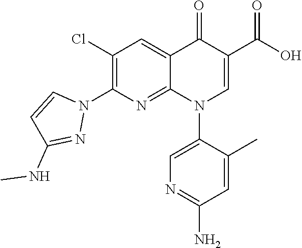 [SMILES: CNc1ccn(-c2nc3c(cc2Cl)c(=O)c(C(=O)O)cn3-c2cnc(N)cc2C)n1] | Prep-HPLC, Method-4 of Table-2 | 1-(6-amino-4-meth- ylpyridin-3-yl)-6- chloro-7-(3-(meth- ylamino)-1H-pyr- azol-1-yl)-4-oxo- 1,4-dihydro-1,8- naphthyridine-3- carboxylic acid | MS (ESI): m/z 426.1 [M + H]⁺; ¹H NMR (400 MHz, DMSO-d₆): δ 14.50 (s, 1H), 8.79 (s, 1H), 8.71 (s, 1H), 7.95 (s, 1H), 7.66 (d, J = 2.8 Hz, 1H), 6.45 (s, 1H), 6.36 (s, 2H), 6.08 (q, J = 5.2 Hz, 1H), 6.00 (d, J = 2.8 Hz, 1H), 2.76 (d, J = 5.2 Hz, 3H), 1.91 (s, 3H); |
| 149 | 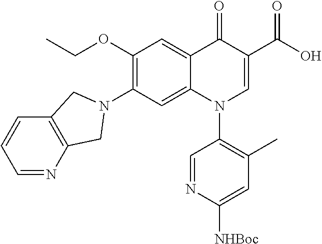 [SMILES: CCOc1cc2c(=O)c(C(=O)O)cn(-c3cnc(NC(=O)OC(C)(C)C)cc3C)c2cc1N1Cc2cccnc2C1] | Prep-HPLC, Method-4 of Table-2 | 1-(6-((tert-butoxy- carbonyl)amino)-4- methylpyridin-3- yl)-7-(5,7-dihydro- 6H-pyrrolo[3,4-b]- pyridin-6-yl)-6- ethoxy-4-oxo-1,4- dihydroquinoline- 3-carboxylic acid | MS (ESI): m/z 558 [M + H]⁺; ¹H NMR (400 MHz, DMSO-d₆): δ 15.86 (s, 1H), 10.27 (s, 1H), 8.57 (s, 1H), 8.43 (d, J = 4.4 Hz, 1H), 8.41 (s, 1H), 8.03 (s, 1H), 7.82 (d, J = 8.0 Hz, 1H), 7.64 (s, 1H), 7.34-7.28 (m, 1H), 5.88 (s, 1H), 4.87 (s, 2H), 4.81 (s, 2H), 4.24 (q, J = 6.8 Hz, 2H), 2.06 (s, 3H), 1.53 (s, 9H), 1.50 (t, J = 6.8 Hz, 3H). |
| 150 | 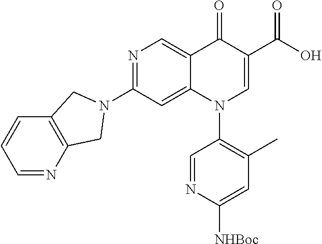 [SMILES: Cc1cc(NC(=O)OC(C)(C)C)ncc1-n1cc(C(=O)O)c(=O)c2cnc(N3Cc4cccnc4C3)cc21] | Prep-HPLC, Method-10 of Table-2 | 1-(6-((tert-butoxy- carbonyl)amino)-4- methylpyridin-3- yl)-7-(5,7-dihydro- 6H-pyrrolo[3,4-b]- pyridin-6-yl)-4-oxo- 1,4-dihydro-1,6- naphthyridine-3- carboxylic acid | MS (ESI): m/z 515 [M + H]⁺; ¹H NMR (400 MHz, DMSO-d₆): δ 14.84 (s, 1H), 10.24 (s, 1H), 9.24 (s, 1H), 8.67 (s, 1H), 8.47 (d, J = 4.0 Hz, 1H), 8.42 (s, 1H), 8.02 (s, 1H), 7.83 (d, J = 6.8 Hz, 1H), 7.34- 7.30 (m, 1H), 5.60 (s, 1H), 4.89 (brs, 2H), 4.61 (brs, 2H), 2.11 (s, 3H), 1.52 (s, 9H). |
| 151 | 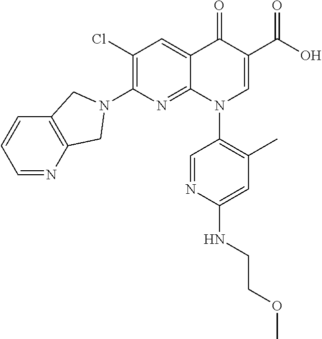 [SMILES: COCCNc1cc(C)c(-n2cc(C(=O)O)c(=O)c3cc(Cl)c(N4Cc5cccnc5C4)nc32)cn1] | Trituration with Et2O followed by n-Pentane | 6-chloro-7-(5,7- dihydro-6H-pyr- rolo[3,4-b]pyridin- 6-yl)-1-(6-((2-meth- oxyethyl)amino)- 4-methylpyridin- 3-yl)-4-oxo-1,4- dihydro-1,8-naph- thyridine-3-carbox- ylic acid | MS (ESI): m/z 507.7 [M + H]⁺; ¹H NMR (400 MHz, DMSO-d₆): δ 15.02 (s, 1H), 8.65 (s, 1H), 8.45 (d, J = 4.4 Hz, 1H), 8.40 (s, 1H), 7.99 (s, 1H), 7.76 (d, J = 7.6 Hz, 1H), 7.34-7.30 (m, 1H), 6.96 (brs, 1H), 6.56 (s, 1H), 5.18 (brs, 2H), 4.82 (brs, 2H), 3.59-3.44 (m, 4H), 3.31 (s, 3H), 1.93 (s, 3H). |
| 152 | 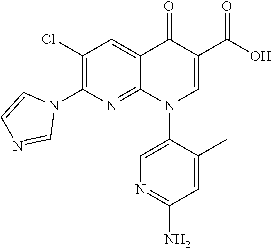 [SMILES: Cc1cc(N)ncc1-n1cc(C(=O)O)c(=O)c2cc(Cl)c(-n3ccnc3)nc21] | Prep-HPLC, Method-4 of Table-2 | 1-(6-amino-4-meth- ylpyridin-3-yl)-6- chloro-7-(1H-imid- azol-1-yl)-4-oxo- 1,4-dihydro-1,8- naphthyridine-3- carboxylic acid | MS (ESI): m/z 397 [M + H]⁺; ¹H NMR (400 MHz, DMSO-d₆): δ 9.00 (s, 1H), 8.91 (s, 1H), 8.19 (s, 1H), 7.97 (s, 1H), 7.62 (t, J = 1.2 Hz, 1H), 7.14 (s, 1H), 6.49 (brs, 3H), 1.93 (s, 3H) (—COOH proton was not observed); |
| 153 | 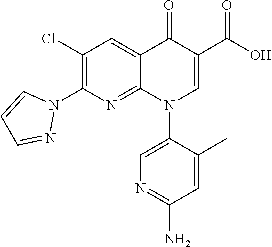 [SMILES: Cc1cc(N)ncc1-n1cc(C(=O)O)c(=O)c2cc(Cl)c(-n3cccn3)nc21] | Prep-HPLC, Method-4 of Table-2 | 1-(6-amino-4-meth- ylpyridin-3-yl)-6- chloro-4-oxo-7- (1H-pyrazol-1-yl)- 1,4-dihydro-1,8- naphthyridine-3- carboxylic acid | MS (ESI): m/z 397.3 [M + H]⁺; ¹H NMR (400 MHz, DMSO-d₆): δ 14.15 (brs, 1H), 8.94 (s, 1H), 8.87 (s, 1H), 7.98 (dd, J = 2.8, 0.4 Hz, 1H), 7.95 (s, 1H), 7.93 (t, J = 1.2 Hz, 1H), 6.64-6.62 (m, 1H), 6.44 (s, 1H), 6.32 (brs, 2H), 1.93 (s, 3H). |
| 154 | 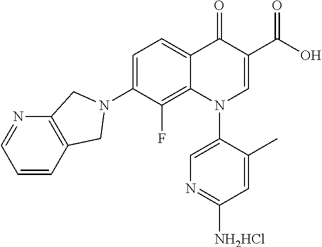 | trituration with 10% Isopropanol in diethyl ether | 1-(6-amino-4-meth- ylpyridin-3-yl)-7- 5,7-dihydro-6H- pyrrolo[3,4-b]pyr- idin-6-yl)-8-fluoro- 4-oxo-1,4-dihydro- quinoline-3-carbox- ylic acid hydro- chloride | MS (ESI): m/z 432 [M + H]⁺; ¹H NMR (400 MHz, DMSO-d₆): δ 8.64 (s, 1H), 8.49 (d, J = 3.6 Hz, 1H), 8.39 (s, 1H), 8.18- 8.06 (m, 3H), 7.82 (d, J = 7.6 Hz, 1H), 7.37-7.28 (m, 2H), 6.90 (s, 1H), 4.98 (s, 2H), 4.89 (s, 2H), 2.15 (s, 3H) (couple of exchangeable protons were not observed). |
| 155 | 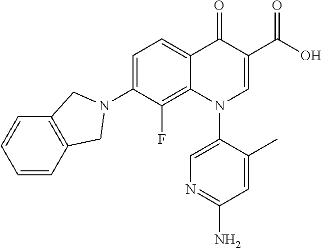 [SMILES: Cc1cc(N)ncc1-n1cc(C(=O)O)c(=O)c2ccc(N3Cc4ccccc4C3)c(F)c21] | Prep-HPLC, Method-4 of Table-2 | 1-(6-amino-4-meth- ylpyridin-3-yl)-8- fluoro-7-(isoindo- lin-2-yl)-4-oxo-1,4- dihydroquinoline- 3-carboxylic acid | MS (ESI): m/z 431 [M + H]⁺; ¹H NMR (400 MHz, DMSO-d₆): δ 15.23 (s, 1H), 8.36 (s, 1H), 8.13- 8.09 (m, 2H), 7.40-7.36 (m, 2H), 7.32-7.21 (m, 3H), 6.47 (m, 3H), 4.89 (s, 4H), 2.00 (s, 3H);. |
| 156 | 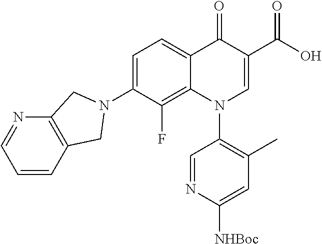 [SMILES: Cc1cc(NC(=O)OC(C)(C)C)ncc1-n1cc(C(=O)O)c(=O)c2ccc(N3Cc4cccnc4C3)c(F)c21] | Prep-HPLC, Method-4 of Table-2 | 1-(6-((tert-butoxy- carbonyl)amino)-4- methylpyridin-3- yl)-7-(5,7-dihydro- 6H-pyrrolo[3,4- b]pyridin-6-yl)-8- fluoro-4-oxo-1,4- dihydroquinoline- 3-carboxylic acid | MS (ESI): m/z 532.2 [M + H]⁺; ¹H NMR (400 MHz, DMSO-d₆): δ 15.17 (s, 1H), 10.12 (s, 1H), 8.48-8.46 (m, 3H), 8.13 (d, J = 8.8 Hz, 1H), 7.91 (s, 1H), 7.79 (d, J = 7.6 Hz, 1H), 7.33-7.27 (m, 2H), 4.94 (s, 2H), 4.84 (s, 2H), 2.15 (s, 3H), 1.52 (s, 9H); |
| 157 | 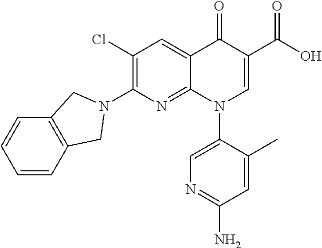 [SMILES: Cc1cc(N)ncc1-n1cc(C(=O)O)c(=O)c2cc(Cl)c(N3Cc4ccccc4C3)nc21] | Trituration with Et2O followed n- Pentane | 1-(6-amino-4-meth- ylpyridin-3-yl)-6- chloro-7-(isoindo- lin-2-yl)-4-oxo-1,4- dihydro-1,8-naph- thyridine-3-carbox- ylic acid | MS (ESI): m/z 448 [M + H]⁺; ¹H NMR (400 MHz, DMSO-d₆) δ 14.94 (s, 1H), 8.84 (s, 1H), 8.39 (s, 1H), 8.22 (s, 1H), 7.95 (brs, 2H), 7.32 (s, 4H), 6.91 (s, 1H), 5.04 (s, 4H), 2.07 (s, 3H). |
| 158 | 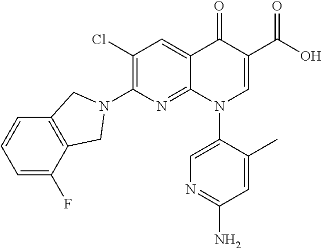 [SMILES: Cc1cc(N)ncc1-n1cc(C(=O)O)c(=O)c2cc(Cl)c(N3Cc4cccc(F)c4C3)nc21] | Trituration with Et2O | 1-(6-amino-4-meth- ylpyridin-3-yl)-6- chloro-7-(4-fluoro- isoindolin-2-yl)-4- oxo-1,4-dihydro- 1,8-naphthyridine- 3-carboxylic acid | MS (ESI): m/z 466.1 [M + H]⁺; ¹H NMR (400 MHz, DMSO-d₆) δ 14.92 (brs, 1H), 8.80 (s, 1H), 8.41 (s, 1H), 8.14 (s, 1H), 7.50 (brs, 2H), 7.41-7.35 (m, 1H), 7.20-7.11 (m, 2H), 6.79 (s, 1H), 5.17 (s, 2H), 4.95 (s, 2H), 2.03 (s, 3H). |
| 159 | 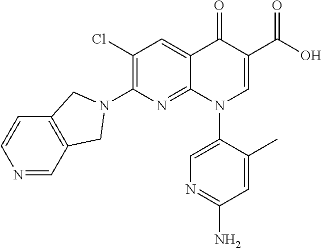 [SMILES: Cc1cc(N)ncc1-n1cc(C(=O)O)c(=O)c2cc(Cl)c(N3Cc4ccncc4C3)nc21] | Trituration with Et2O | 1-(6-amino-4-meth- ylpyridin-3-yl)-6- chloro-7-(1,3- dihydro-2H-pyr- rolo[3,4-c]pyridin- 2-yl)-4-oxo-1,4- dihydro-1,8-naph- thyridine-3-carbox- ylic acid | MS (ESI): m/z 449 [M + H]⁺; ¹H NMR (400 MHz, DMSO-d₆) δ 15.03 (brs, 1H), 8.65 (s, 1H), 8.56 (s, 1H), 8.49 (d, J = 5.2 Hz, 1H), 8.41 (s, 1H), 7.96 (s, 1H), 7.39 (d, J = 5.2 Hz, 1H), 6.54 (s, 1H), 6.49 (brs, 2H), 5.07 (s, 2H), 5.03 (s, 2H), 1.94 (s, 3H). |
| 160 | 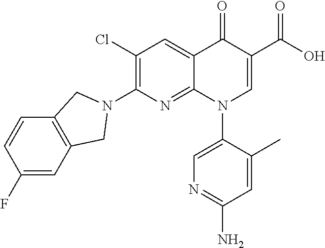 [SMILES: Cc1cc(N)ncc1-n1cc(C(=O)O)c(=O)c2cc(Cl)c(N3Cc4ccc(F)cc4C3)nc21] | Trituration with Et2O | 1-(6-amino-4-meth- ylpyridin-3-yl)-6- chloro-7-(5-fluoro- isoindolin-2-yl)-4- oxo-1,4-dihydro- 1,8-naphthyridine- 3-carboxylic acid | MS (ESI): m/z 466.1 [M + H]⁺; ¹H NMR (400 MHz, DMSO-d₆) δ 14.99 (brs, 1H), 8.78 (s, 1H), 8.40 (s, 1H), 8.15 (s, 1H), 7.55 (brs, 2H), 7.37-7.33 (m, 1H), 7.21-7.12 (m, 2H), 6.80 (s, 1H), 5.03 (s, 2H), 4.98 (s, 2H), 2.03 (s, 3H). |
| 161 | 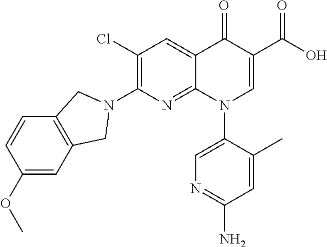 [SMILES: COc1ccc2c(c1)CN(c1nc3c(cc1Cl)c(=O)c(C(=O)O)cn3-c1cnc(N)cc1C)C2] | Trituration with Et2O followed n-Pentane | 1-(6-amino-4-meth- ylpyridin-3-yl)-6- chloro-7-(5-meth- oxyisoindolin-2-yl)- 4-oxo-1,4-dihydro- 1,8-naphthyridine- 3-carboxylic acid | MS (ESI): m/z 478 [M + H]⁺; ¹H NMR (400 MHz, DMSO-d₆): δ 14.97 (s, 1H), 8.77 (s, 1H), 8.38 (s, 1H), 8.14 (s, 1H), 7.46 (s, 1H), 7.21 (d, J = 8.0 Hz, 1H), 6.88-6.85 (m, 2H), 6.79 (s, 1H), 4.99-4.94 (m, 4H), 3.75 (s, 3H), 2.03 (s, 3H). |
| 162 | 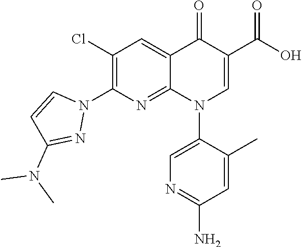 [SMILES: Cc1cc(N)ncc1-n1cc(C(=O)O)c(=O)c2cc(Cl)c(-n3ccc(N(C)C)n3)nc21] | Prep-HPLC, Method-5 of Table-2 | 1-(6-amino-4-meth- ylpyridin-3-yl)-6- chloro-7-(3-(dimeth- ylamino)-1H-pyr- azol-1-yl)-4-oxo- 1,4-dihydro-1,8- naphthyridine-3- carboxylic acid | MS (ESI): m/z 440.2 [M + H]⁺; ¹H NMR (400 MHz, DMSO-d₆): δ 14.42 (s, 1H), 8.81 (s, 1H), 8.73 (s, 1H), 7.95 (s, 1H), 7.74 (s, 1H), 6.45 (s, 1H), 6.33 (s, 2H), 6.29 (s, 1H), 2.87 (s, 6H), 1.92 (s, 3H); |
| 163 | 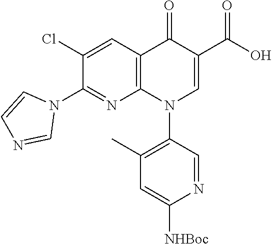 [SMILES: Cc1cc(NC(=O)OC(C)(C)C)ncc1-n1cc(C(=O)O)c(=O)c2cc(Cl)c(-n3ccnc3)nc21] | trituration with 10% Isopropanol in diethyl ether | 1-(6-((tert-butoxy- carbonyl)amino)-4- methylpyridin-3- yl)-6-chloro-7-(1H- imidazol-1-yl)-4- oxo-1,4-dihydro- 1,8-naphthyridine- 3-carboxylic acid | MS (ESI): m/z 497 [M + H]⁺; ¹H NMR (400 MHz, DMSO-d₆): δ 14.02 (brs, 1H), 10.10 (s, 1H), 9.01- 8.76 (m, 1H), 8.33 (s, 1H), 8.14 (s, 1H), 7.93 (s, 1H), 7.58 (s, 1H), 7.11 (s, 1H), 2.09 (s, 3H), 1.50 (s, 9H); |
| 164 | 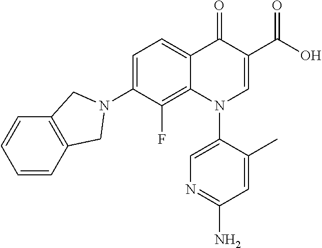 | Prep-HPLC, Method-4 of Table-2 | 1-(6-((tert-butoxy- carbonyl)amino)-4- methylpyridin-3- yl)-6-chloro-4-oxo- 7-(1H-pyrazol-1- yl)-1,4-dihydro- 1,8-naphthyridine- 3-carboxylic acid | MS (ESI): m/z 497.2 [M + H]⁺; ¹H NMR (400 MHz, DMSO-d₆): δ 14.11 (brs, 1H), 10.10 (s, 1H), 8.99 (s, 1H), 8.96 (s, 1H), 8.33 (s, 1H), 7.95- 7.91 (m, 3H), 6.59 (dd, J = 2.8, 0.4 Hz, 1H), 2.10 (s, 3H), 1.51 (s, 9H). |
| 165 | 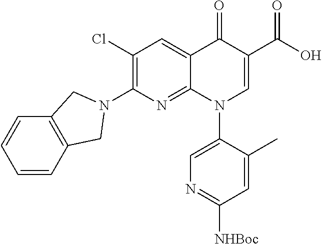 [SMILES: Cc1cc(NC(=O)OC(C)(C)C)ncc1-n1cc(C(=O)O)c(=O)c2cc(Cl)c(N3Cc4ccccc4C3)nc21] | Trituration with Et2O | 1-(6-((tert-butoxy- carbonyl)amino)-4- methylpyridin-3- yl)-6-chloro-7- (isoindolin-2-yl)-4- oxo-1,4-dihydro- 1,8-naphthyridine- 3-carboxylic acid | MS (ESI): m/z 548.2 [M + H]⁺; 1H NMR (400 MHz, DMSO-d₆) δ 10.07 (s, 1H), 8.56 (s, 1H), 8.28 (s, 1H), 8.18 (s, 1H), 7.94 (s, 1H), 7.25 (m, 4H), 4.90 (brs, 4H), 2.04 (s, 3H), 1.53 (s, 9H) (—COOH peak was not observed); |
| 166 | 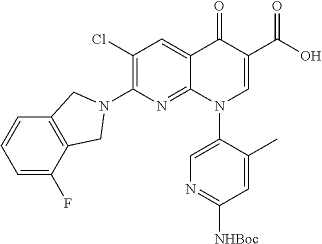 [SMILES: Cc1cc(NC(=O)OC(C)(C)C)ncc1-n1cc(C(=O)O)c(=O)c2cc(Cl)c(N3Cc4cccc(F)c4C3)nc21] | Trituration with Et2O | 1-(6-((tert-butoxy- carbonyl)amino)-4- methylpyridin-3- yl)-6-chloro-7-(4- fluoroisoindolin-2- yl)-4-oxo-1,4- dihydro-1,8-naph- thyridine-3-carbox- ylic acid | MS (ESI): m/z 566 [M + H]⁺; ¹H NMR (400 MHz, DMSO-d₆) δ 14.99 (brs, 1H), 10.07 (s, 1H), 8.80- 8.60 (m, 1H), 8.42-8.18 (m, 2H), 7.95 (s, 1H), 7.34 (brs, 1H), 7.20-7.15 (m, 2H), 5.08 (s, 2H), 4.85 (s, 2H), 2.08 (s, 3H), 1.52 (s, 9H). |
| 167 | 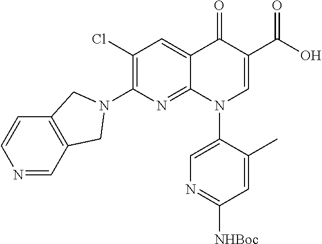 [SMILES: Cc1cc(NC(=O)OC(C)(C)C)ncc1-n1cc(C(=O)O)c(=O)c2cc(Cl)c(N3Cc4ccncc4C3)nc21] | Trituration with Et2O | 1-(6-((tert-butoxy- carbonyl)amino)-4- methylpyridin-3- yl)-6-chloro-7-(1,3- dihydro-2H-pyr- rolo[3,4-c]pyridin- 2-yl)-4-oxo-1,4- dihydro-1,8-naph- thyridine-3-carbox- ylic acid | MS (ESI): m/z 550 [M + H]⁺; ¹H NMR (400 MHz, DMSO-d₆) δ 14.98 (brs, 1H), 10.08 (s, 1H), 8.75 (s, 1H), 8.54 (s, 1H), 8.47 (d, J = 5.2 Hz, 1H), 8.42 (s, 1H), 8.31 (s, 1H), 7.95 (s, 1H), 7.36 (d, J = 5.2 Hz, 1H), 5.01 (s, 2H), 4.97 (s, 2H), 2.09 (s, 3H), 1.53 (s, 9H). |
| 168 | 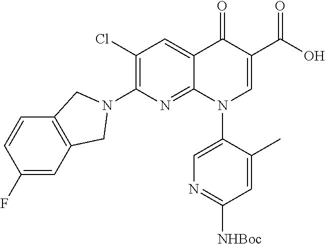 [SMILES: Cc1cc(NC(=O)OC(C)(C)C)ncc1-n1cc(C(=O)O)c(=O)c2cc(Cl)c(N3Cc4ccc(F)cc4C3)nc21] | Trituration with Et2O | 1-(6-((tert-butoxy- carbonyl)amino)- 4-methylpyridin-3- yl)-6-chloro-7-(5- fluoroisoindolin-2- yl)-4-oxo-1,4- dihydro-1,8-naph- thyridine-3-carbox- ylic acid | MS (ESI): m/z 566 [M + H]⁺; 1H NMR (400 MHz, DMSO-d₆) δ 15.00 (s, 1H), 10.06 (s, 1H), 8.75 (s, 1H), 8.40 (s, 1H), 8.31 (s, 1H), 7.93 (s, 1H), 7.34-7.29 (m, 1H), 7.17- 7.09 (m, 2H), 4.98 (s, 2H), 4.82 (s, 2H), 2.09 (s, 3H), 1.53 (s, 9H). |
| 169 | 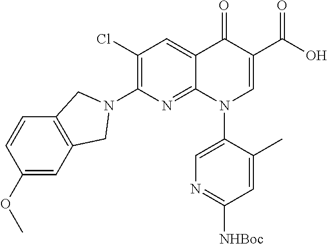 [SMILES: COc1ccc2c(c1)CN(c1nc3c(cc1Cl)c(=O)c(C(=O)O)cn3-c1cnc(NC(=O)OC(C)(C)C)cc1C)C2] | Prep-HPLC, Method-12 of Table-2 | 1-(6-((tert-butoxy- carbonyl)amino)-4- methylpyridin-3- yl)-6-chloro-7-(5- methoxyisoindolin- 2-yl)-4-oxo-1,4- dihydro-1,8-naph- thyridine-3-carbox- ylic acid | MS (ESI): m/z 578.3 [M + H]⁺; 1H NMR (400 MHz, DMSO-d₆): δ 15.02 (s, 1H), 10.08 (s, 1H), 8.75 (s, 1H), 8.39 (s, 1H), 8.31 (s, 1H), 7.94 (s, 1H), 7.17 (d, J = 9.2 Hz, 1H), 6.85-6.82 (m, 2H), 4.92-4.88 (m, 4H), 3.74 (s, 3H), 2.09 (s, 3H). 1.53 (s, 9H); |
| 170 | 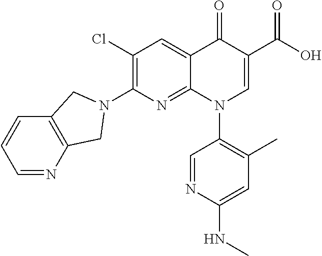 [SMILES: CNc1cc(C)c(-n2cc(C(=O)O)c(=O)c3cc(Cl)c(N4Cc5cccnc5C4)nc32)cn1] | Prep-HPLC, Method-4 of Table-2 | 6-chloro-7-(5,7- dihydro-6H-pyr- rolo[3,4-b]pyridin- 6-yl)-1-(4-methyl- 6-(methylamino)- pyridin-3-yl)-4- oxo-1,4-dihydro- 1,8-naphthyridine- 3-carboxylic acid | MS (ESI): m/z 463.2 [M + H]⁺; ¹H NMR (400 MHz, DMSO-d₆, 1 drop of TFA-d) δ 8.90 (s, 1H), 8.67 (d, J = 5.2 Hz, 1H) 8.47 (s, 1H), 8.37 (s, 1H), 8.13 (d, J = 8.0 Hz, 1H) 7.34-7.30 (m, 1H), 7.12 (brs, 1H), 5.25 (s, 2H), 5.08 (s, 2H), 3.06 (s, 3H), 2.15 (s, 3H). |
| 171 | 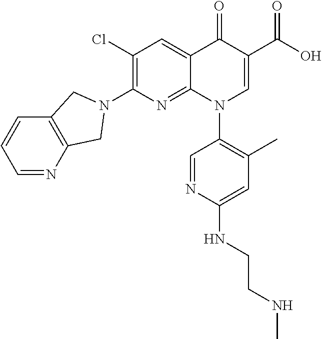 [SMILES: CNCCNc1cc(C)c(-n2cc(C(=O)O)c(=O)c3cc(Cl)c(N4Cc5cccnc5C4)nc32)cn1] | Prep-HPLC, Method-4 of Table-2 | 6-chloro-7-(5,7- dihydro-6H- pyrrolo[3,4-b]pyr- idin-6-yl)-1-(4- methyl-6-((2- (methylamino)- ethyl)amino)pyr- idin-3-yl)-4-oxo- 1,4-dihydro-1,8- naphthyridine-3- carboxylic acid | MS (ESI): m/z 506.3 [M + H]⁺; ¹H NMR (400 MHz, DMSO-d₆): δ 8.62 (s, 1H), 8.46 (d, J = 4.4 Hz, 1H), 8.42 (s, 1H), 8.05 (s, 1H), 7.78 (d, J = 7.6 Hz, 1H), 7.35-7.31 (m, 1H), 7.04 (t, J = 5.6 Hz, 1H), 6.58 (s, 1H), 5.22 (brs, 2H), 4.79 (brs, 2H), 3.62-3.44 (m, 2H), 3.02 (t, J = 6.0 Hz, 2H), 2.55 (s, 3H), 1.97 (s, 3H) (couple of exchangeable protons were not observed). |
| 172 | 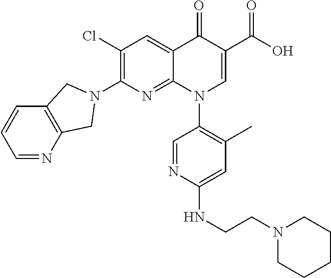 [SMILES: Cc1cc(NCCN2CCCCC2)ncc1-n1cc(C(=O)O)c(=O)c2cc(Cl)c(N3Cc4cccnc4C3)nc21] | Prep-HPLC, Method-4 of Table-2 | 6-chloro-7-(5,7- dihydro-6H-pyr- rolo[3,4-b]pyridin- 6-yl)-1-(4-methyl- 6-((2-(piperidin-1- yl)ethyl)amino)- pyridin-3-yl)-4- oxo-1,4-dihydro- 1,8-naphthyridine- 3-carboxylic acid | MS (ESI): m/z 560.3 [M + H]⁺; ¹H NMR (400 MHz, DMSO-d₆): δ 15.01 (brs, 1H), 9.86 (brs, 1H), 8.64 (s, 1H), 8.45 (d, J = 4.4 Hz, 1H), 8.41 (s, 1H), 8.09 (s, 1H), 7.79 (d, J = 7.2 Hz, 1H), 7.34-7.27 (m, 2H), 6.62 (s, 1H), 5.23 (brs, 2H), 4.77 (brs, 2H), 3.76- 3.68 (m, 2H), 3.63-3.44 (m, 2H), 3.30-3.24 (m, 2H), 3.12-2.88 (m, 2H), 1.98 (s, 3H), 1.82-1.73 (m, 4H), 1.66-1.46 (m, 2H). |
| 173 | 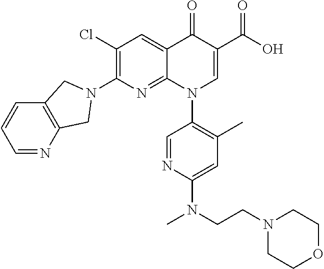 [SMILES: Cc1cc(N(C)CCN2CCOCC2)ncc1-n1cc(C(=O)O)c(=O)c2cc(Cl)c(N3Cc4cccnc4C3)nc21] | Prep-HPLC, Method-4 of Table-2 | 6-chloro-7-(5,7- dihydro-6H-pyr- rolo[3,4-b]pyridin- 6-yl)-1-(4-methyl- 6-(methyl(2-morph- olinoethyl)amino)- pyridin-3-yl)-4- oxo-1,4-dihydro- 1,8-naphthyridine- 3-carboxylic acid | MS (ESI): m/z 576.2 [M + H]⁺; ¹H NMR (400 MHz, DMSO-d₆): δ 15.03 (s, 1H), 8.66 (s, 1H), 8.45 (d, J = 4.4 Hz, 1H), 8.41 (s, 1H), 8.08 (s, 1H), 7.75 (d, J = 8.0 Hz, 1H), 7.34-7.30 (m, 1H), 6.70 (s, 1H), 5.17 (brs, 2H), 4.81 (brs, 2H), 3.75 (t, J = 5.6 Hz, 2H), 3.56 (t, J = 4.4 Hz, 4H), 3.12 (s, 3H), 2.48 (brs, 6H), 2.01 (s, 3H). |
| 174 | 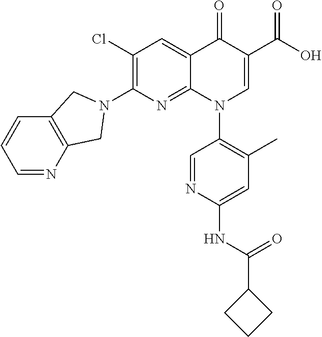 [SMILES: Cc1cc(NC(=O)C2CCC2)ncc1-n1cc(C(=O)O)c(=O)c2cc(Cl)c(N3Cc4cccnc4C3)nc21] | trituration with 10% Isopropanol in diethyl ether | 6-chloro-1-(6- (cyclobutanecar- boxamido)-4-meth- ylpyridin-3-yl)-7- (5,7-dihydro-6H- pyrrolo[3,4-b]pyr- idin-6-yl)-4-oxo- 1,4-dihydro-1,8- naphthyridine-3- carboxylic acid | MS (ESI): m/z 531.2 [M + H]⁺; ¹H NMR (400 MHz, DMSO-d₆): δ 15.02 (s, 1H), 10.58 (s, 1H), 8.42-8.20 (m, 5H), 7.73 (d, J = 6.8, 1H), 7.29 (s, 1H), 5.07 (s, 2H), 4.80 (s, 2H), 3.46-3.37 (m, 1H), 2.33-1.82 (m, 9H); |
| 175 | 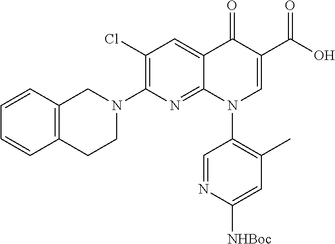 [SMILES: Cc1cc(NC(=O)OC(C)(C)C)ncc1-n1cc(C(=O)O)c(=O)c2cc(Cl)c(N3CCc4ccccc4C3)nc21] | Prep-HPLC, Method-4 of Table-2 | 1-(6-((tert-butoxy- carbonyl)amino)-4- methylpyridin-3- yl)-6-chloro-7-(3,4- dihydroisoquinolin- 2(1H)-yl)-4-oxo- 1,4-dihydro-1,8- naphthyridine-3- carboxylic acid | MS (ESI): m/z 562.3 [M + H]⁺; 1H NMR (400 MHz, DMSO-d₆): δ 14.83 (brs, 1H), 10.10 (s, 1H), 8.80 (s, 1H), 8.46 (s, 1H), 8.27 (s, 1H), 7.91 (s, 1H), 7.17-7.07 (m, 3H), 6.85 (d, J = 6.8 Hz, 1H), 4.58-4.48 (m, 2H), 3.92- 3.85 (m, 1H), 3.78-3.71 (m, 1H), 2.75 (t, J = 6.0 Hz, 2H), 1.94 (s, 3H). 1.53 (s, 9H); |
| 176 | 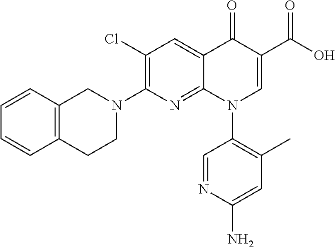 [SMILES: Cc1cc(N)ncc1-n1cc(C(=O)O)c(=O)c2cc(Cl)c(N3CCc4ccccc4C3)nc21] | Trituration with Et2O | 1-(6-amino-4-meth- ylpyridin-3-yl)-6- chloro-7-(3,4-di- hydroisoquinolin- 2(1H)-yl)-4-oxo- 1,4-dihydro-1,8- naphthyridine-3- carboxylic acid | MS (ESI): m/z 462.0 [M + H]⁺; 1H NMR (400 MHz, DMSO-d₆): δ 14.82 (brs, 1H), 8.82 (s, 1H), 8.46 (s, 1H), 8.09 (s, 1H), 7.58 (brs, 2H), 7.20- 7.10 (m, 3H), 6.95-6.91 (m, 1H), 6.76 (s, 1H), 4.60 (s, 2H), 3.95-3.79 (m, 2H), 2.80 (t, J = 6.0 Hz, 2H), 1.88 (s, 3H). |
| 177 | 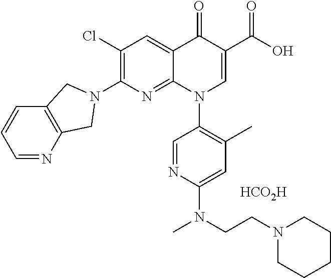 [SMILES: Cc1cc(N(C)CCN2CCCCC2)ncc1-n1cc(C(=O)O)c(=O)c2cc(Cl)c(N3Cc4cccnc4C3)nc21 ; "HCO2H"] | Prep-HPLC, Method-5 of Table-2 | 6-chloro-7-(5,7- dihydro-6H-pyr- rolo[3,4-b]pyridin- 6-yl)-1-(4-methyl- 6-(methyl(2-(piper- idin-1-yl)ethyl)- amino)pyridin-3- yl)-4-oxo-1,4- dihydro-1,8-naph- thyridine-3-carbox- ylic acid | MS (ESI): m/z 574.3 [M + H]⁺; ¹H NMR (400 MHz, DMSO-d₆): δ 8.61 (s, 1H), 8.51 (s, 1H), 8.45 (d, J = 4.4 Hz, 1H), 8.41 (s, 1H), 8.08 (s, 1H), 7.79 (d, J = 7.2 Hz, 1H), 7.35- 7.27 (m, 2H), 6.62 (s, 1H), 5.26 (brs, 2H), 4.73 (brs, 2H), 3.84-3.78 (m, 2H), 3.60-3.42 (m, 5H), 3.14 (s, 3H), 1.97 (s, 3H), 1.83-1.76 (m, 4H), 1.66- 1.46 (m, 2H) (—COOH proton was not observed). |
| 178 | 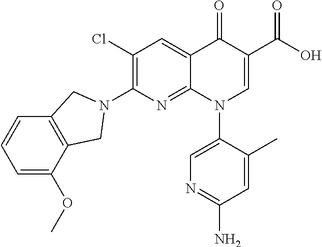 [SMILES: COc1cccc2c1CN(c1nc3c(cc1Cl)c(=O)c(C(=O)O)cn3-c1cnc(N)cc1C)C2] | trituration with 10% Isopropanol in diethyl ether | 1-(6-amino-4-meth- ylpyridin-3-yl)-6- chloro-7-(4-meth- oxyisoindolin-2- yl)-4-oxo-1,4- dihydro-1,8-naph- thyridine-3- carboxylic acid | MS (ESI): m/z 478.2 [M + H]⁺; ¹H NMR (400 MHz, DMSO-d₆): δ 15.07 (s, 1H), 8.64 (s, 1H), 8.37 (s, 1H), 7.91 (s, 1H), 7.29 (t, J = 8.0 Hz, 1H), 6.91-6.85 (m, 2H), 6.48 (s, 1H), 6.30 (s, 2H), 4.97 (s, 2H), 4.91 (s, 2H), 3.81 (s, 3H), 1.91 (s, 3H); |
| 179 | 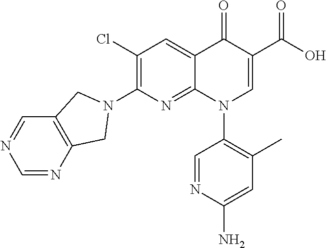 [SMILES: Cc1cc(N)ncc1-n1cc(C(=O)O)c(=O)c2cc(Cl)c(N3Cc4cncnc4C3)nc21] | Prep-HPLC, Method-5 of Table-2 | 1-(6-amino-4-meth- ylpyridin-3-yl)-6- chloro-7-(5,7-di- hydro-6H-pyrrolo- [3,4-d]pyrimidin- 6-yl)-4-oxo-1,4- dihydro-1,8-naph- thyridine-3-carbox- ylic acid | MS (ESI): m/z 450.1 [M + H]⁺; ¹H NMR (400 MHz, DMSO-d₆) δ 9.10 (s, 1H), 8.82 (s, 2H), 8.44 (s, 1H), 8.17 (s, 1H), 7.68 (brs, 2H), 6.83 (s, 1H), 5.23 (s, 2H), 5.00 (s, 2H), 2.05 (s, 3H) (—COOH proton was not observed). |
| 180 | 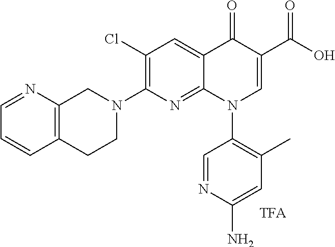 [SMILES: Cc1cc(N)ncc1-n1cc(C(=O)O)c(=O)c2cc(Cl)c(N3CCc4cccnc4C3)nc21 ; "TFA"] | Trituration with Et2O | 1-(6-amino-4-meth- ylpyridin-3-yl)-6- chloro-7-(5,8-di- hydro-1,7-naph- thyridin-7(6H)-yl)- 4-oxo-1,4-dihydro- 1,8-naphthyridine- 3-carboxylic acid | MS (ESI): m/z 463.2 [M + H]⁺; 1H NMR (400 MHz, DMSO-d₆): δ 8.87 (s, 1H), 8.51 (s, 1H), 8.38 (d, J = 4.8 Hz, 1H), 8.15 (s, 1H), 7.93 (brs, 2H), 7.59 (d, J = 6.8 Hz, 1H), 7.27-7.23 (m, 1H), 6.82 (s, 1H), 4.58 (s, 2H), 3.99-3.80 (m, 2H), 2.84 (t, J = 6.0 Hz, 2H), 1.91 (s, 3H) (couple of exchangeable protons were not observed). |
| 181 | 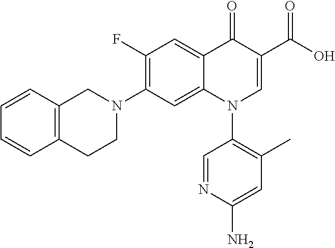 [SMILES: Cc1cc(N)ncc1-n1cc(C(=O)O)c(=O)c2cc(F)c(N3CCc4ccccc4C3)cc21] | Prep-HPLC, Method-11 of Table-2 | 1-(6-amino-4-meth- ylpyridin-3-yl)-7- (3,4-dihydroiso- quinolin-2(1H)-yl)- 6-fluoro-4-oxo-1,4- dihydroquinoline- 3-carboxylic acid | MS (ESI): m/z 445.2 [M + H]⁺; 1H NMR (400 MHz, DMSO-d₆): δ 15.11 (brs, 1H), 8.69 (s, 1H), 8.17 (s, 1H), 8.01 (d, J = 9.6 Hz, 1H), 7.50 (brs, 2H), 7.22-7.03 (m, 4H), 6.68 (s, 1H), 6.39 (d, J = 7.6 Hz, 1H), 4.38 (ABq, J = 16.8 Hz, 2H), 3.72-3.54 (m, 2H), 2.83 (t, J = 6.0 Hz, 2H), 1.75 (s, 3H). |
| 182 | 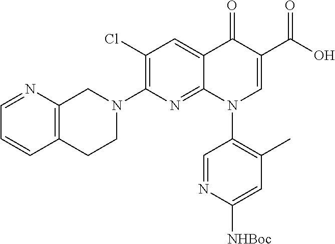 [SMILES: Cc1cc(NC(=O)OC(C)(C)C)ncc1-n1cc(C(=O)O)c(=O)c2cc(Cl)c(N3CCc4cccnc4C3)nc21] | trituration with 10% Isopropanol in diethyl ether | 1-(6-((tert-butoxy- carbonyl)amino)-4- methylpyridin-3- yl)-6-chloro-7-(5,8- dihydro-1,7-naph- thyridin-7(6H)-yl)- 4-oxo-1,4-dihydro- 1,8-naphthyridine- 3-carboxylic acid | MS (ESI): m/z 563.2 [M + H]⁺; 1H NMR (400 MHz, DMSO-d₆): δ 14.86 (brs, 1H), 10.13 (s, 1H), 8.80 (s, 1H), 8.51 (s, 1H), 8.34 (d, J = 4.8 Hz, 1H), 8.29 (s, 1H), 7.92 (s, 1H), 7.55 (d, J = 6.8 Hz, 1H), 7.23-7.19 (m, 1H), 4.54 (ABq, J = 6.8 Hz, 2H), 3.87-3.71 (m, 2H), 2.79 (t, J = 6.0 Hz, 2H), 1.98 (s, 3H), 1.53 (s, 9H). |
| 183 | 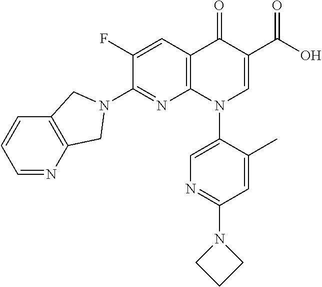 [SMILES: Cc1cc(N2CCC2)ncc1-n1cc(C(=O)O)c(=O)c2cc(F)c(N3Cc4cccnc4C3)nc21] | trituration with 10% Isopropanol in diethyl ether | 1-(6-(azetidin-1- yl)-4-methylpyr- idin-3-yl)-7-(5,7- dihydro-6H-pyr- rolo[3,4-b]pyridin- 6-yl)-6-fluoro-4- oxo-1,4-dihydro- 1,8-naphthyridine- 3-carboxylic acid | MS (ESI): m/z 473.2 [M + H]⁺; 1H NMR (400 MHz, DMSO-d₆): δ 15.24 (s, 1H), 8.61 (s, 1H), 8.46 (d, J = 2.8 Hz, 1H), 8.18 (d, J = 12.4 Hz, 1H), 8.07 (s, 1H), 7.82 (d, J = 8.0 Hz, 1H), 7.33 (q, J = 5.6 Hz, 1H), 6.43 (s, 1H), 5.20 (s, 2H), 4.54 (s, 2H), 4.07-4.02 (m, 6H), 1.98 (s, 3H) |
| 184 | 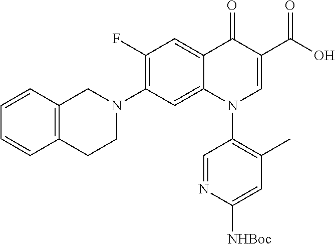 [SMILES: Cc1cc(NC(=O)OC(C)(C)C)ncc1-n1cc(C(=O)O)c(=O)c2cc(F)c(N3CCc4ccccc4C3)cc21] | Prep-HPLC, Method-4 of Table-2 | 1-(6-((tert-butoxy- carbonyl)amino)-4- methylpyridin-3- yl)-7-(3,4-dihydro- isoquinolin-2(1H)- yl)-6-fluoro-4-oxo- 1,4-dihydroquin- oline-3-carboxylic acid | MS (ESI): m/z 545.3 [M + H]⁺; 1H NMR (400 MHz, DMSO-d₆): δ 15.14 (s, 1H), 10.21 (s, 1H), 8.68 (s, 1H), 8.35 (s, 1H), 8.02 (d, J = 13.2 Hz, 1H), 7.91 (s, 1H), 7.20- 7.09 (m, 3H), 6.95 (d, J = 7.6 Hz, 1H), 6.24 (d, J = 7.6 Hz, 1H), 4.29 (ABq, J = 16.8 Hz, 2H), 3.66- 3.53 (m, 2H), 2.81 (t, J = 6.0 Hz, 2H), 1.83 (s, 3H), 1.54 (s, 9H). |
| 185 | 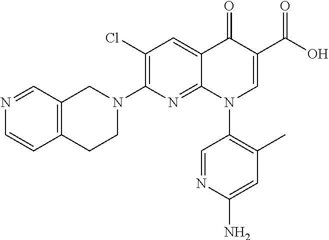 [SMILES: Cc1cc(N)ncc1-n1cc(C(=O)O)c(=O)c2cc(Cl)c(N3CCc4ccncc4C3)nc21] | Prep-HPLC, Method-13 of Table-2 | 1-(6-amino-4-meth- ylpyridin-3-yl)-6- chloro-7-(3,4-di- hydro-2,7-naph- thyridin-2(1H)-yl)- 4-oxo-1,4-dihydro- 1,8-naphthyridine- 3-carboxylic acid | MS (ESI): m/z 463.3 [M + H]⁺; 1H NMR (400 MHz, DMSO-d₆): δ 8.84 (s, 1H), 8.51 (s, 1H), 8.43 (s, 1H), 8.32 (s, 1H), 8.09 (s, 1H), 7.33 (brs, 1H), 6.76 (s, 1H), 4.69 (d, J = 5.6 Hz, 2H), 3.96-3.77 (m, 2H), 2.86 (t, J = 6.0 Hz, 2H), 1.87 (s, 3H) (few exchangeable pro- tons were not observed). |
| 186 | 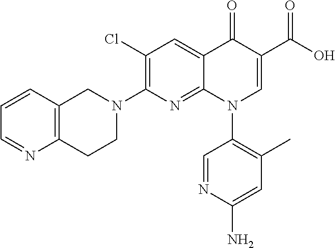 [SMILES: Cc1cc(N)ncc1-n1cc(C(=O)O)c(=O)c2cc(Cl)c(N3CCc4ncccc4C3)nc21] | Trituration with Et2O | 1-(6-amino-4-meth- ylpyridin-3-yl)-6- chloro-7-(7,8-di- hydro-1,6-naph- thyridin-6(5H)-yl)- 4-oxo-1,4-dihydro- 1,8-naphthyridine- 3-carboxylic acid | MS (ESI): m/z 463.3 [M + H]⁺; 1H NMR (400 MHz, DMSO-d₆): δ 14.73 (brs, 1H), 8.82 (s, 1H), 8.50 (s, 1H), 8.41 (d, J = 4.0 Hz, 1H), 8.05 (s, 1H), 7.50 (brs, 2H), 7.45 (d, J = 7.6 Hz, 1H), 7.31-7.26 (m, 1H), 6.71 (s, 1H), 4.67 (d, J = 5.6 Hz, 2H), 3.99-3.89 (m, 2H), 2.86 (t, J = 6.0 Hz, 2H), 1.85 (s, 3H). |
| 187 | 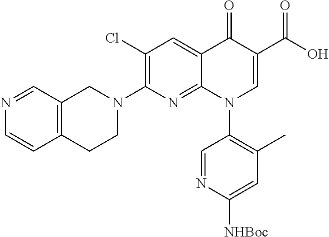 [SMILES: Cc1cc(NC(=O)OC(C)(C)C)ncc1-n1cc(C(=O)O)c(=O)c2cc(Cl)c(N3CCc4ccncc4C3)nc21] | Prep-HPLC, Method-13 of Table-2 | 1-(6-((tert-butoxy- carbonyl)amino)-4- methylpyridin-3- yl)-6-chloro-7-(3,4- dihydro-2,7-naph- thyridin-2(1H)-yl)- 4-oxo-1,4-dihydro- 1,8-naphthyridine- 3-carboxylic acid | MS (ESI): m/z 563.2 [M + H]⁺; 1H NMR (400 MHz, DMSO-d₆): δ 14.81 (brs, 1H), 10.08 (s, 1H), 8.82 (s, 1H), 8.49 (s, 1H), 8.30 (d, J = 4.8 Hz, 1H), 8.28 (s, 1H), 8.14 (s, 1H), 7.92 (s, 1H), 7.12 (d, J = 6.8 Hz, 1H), 4.60 (ABq, J = 6.8 Hz, 2H), 3.86-3.67 (m, 2H), 2.74 (t, J = 6.0 Hz, 2H), 1.97 (s, 3H), 1.53 (s, 9H). |
| 188 | 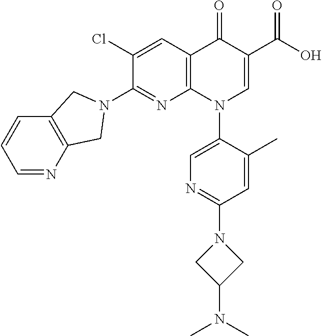 [SMILES: Cc1cc(N2CC(N(C)C)C2)ncc1-n1cc(C(=O)O)c(=O)c2cc(Cl)c(N3Cc4cccnc4C3)nc21] | Prep-HPLC, Method-4 of Table-2 | 6-chloro-7-(5,7- dihydro-6H-pyr- rolo[3,4-b]pyridin- 6-yl)-1-(6-(3- (dimethylamino)- azetidin-1-yl)-4- meth-ylpyridin-3- yl)-4-oxo-1,4- dihydro-1,8-naph- thyridine-3-carbox- ylic acid | MS (ESI): m/z 532.2 [M + H]⁺; ¹H NMR (400 MHz, DMSO-d₆): δ 15.01 (bs, 1H), 8.66 (s, 1H), 8.46 (d, J = 4.4 Hz, 1H), 8.41 (s, 1H), 8.09 (s, 1H), 7.78 (d, J = 8.8 Hz, 1H), 7.34-7.30 (m, 1H), 6.51 (s, 1H), 5.14 (s, 2H), 4.83 (s, 2H), 4.12- 4.06 (m, 2H), 3.85-3.79 (m, 2H), 3.26-3.20 (m, 1H), 2.15 (s, 6H), 1.99 (s, 3H). |
| 189 | 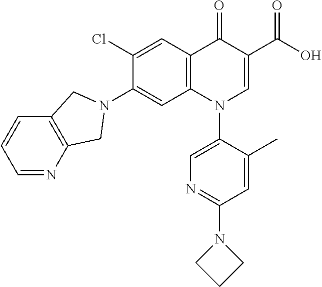 [SMILES: Cc1cc(N2CCC2)ncc1-n1cc(C(=O)O)c(=O)c2cc(Cl)c(N3Cc4cccnc4C3)cc21] | trituration with 10% Isopropanol in diethyl ether | 1-(6-(azetidin-1- yl)-4-methylpyr- idin-3-yl)-6-chloro- 7-(5,7-dihydro-6H- pyrrolo[3,4-b]pyr- idin-6-yl)-4-oxo- 1,4-dihydroquin- oline-3-carboxylic acid | MS (ESI): m/z 488.2 [M + H]⁺; ¹H NMR (400 MHz, DMSO-d₆): δ 8.55 (s, 1H), 8.46 (d, J = 4.4 Hz, 1H), 8.25 (s, 1H), 8.18 (s, 1H), 7.83 (d, J = 7.2 Hz, 1H), 7.31 (q, J = 4.8 Hz, 1H), 6.47 (s, 1H), 6.19 (s, 1H), 5.00 (s, 2H), 4.83 (s, 2H), 4.10-4.05 (m, 4H), 2.32 (m, 2H), 1.98 (s, 3H), COOH proton was not observed; |
| 190 | 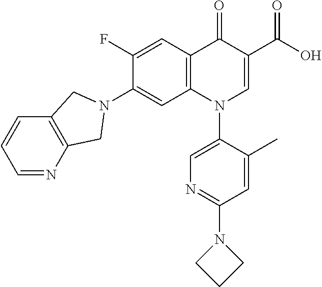 [SMILES: Cc1cc(N2CCC2)ncc1-n1cc(C(=O)O)c(=O)c2cc(F)c(N3Cc4cccnc4C3)cc21] | trituration with 10% Isopropanol in diethyl ether | 1-(6-(azetidin-1- yl)-4-methylpyr- idin-3-yl)-7-(5,7- dihydro-6H-pyr- rolo[3,4-b]pyridin- 6-yl)-6-fluoro-4- oxo-1,4-dihydro- quinoline-3-carbox- ylic acid | MS (ESI): m/z 472.2 [M + H]⁺; ¹H NMR (400 MHz, DMSO-d₆): δ 15.36 (s, 1H), 8.52 (s, 1H), 8.47 (d, J = 4.4 Hz, 1H), 8.18 (s, 1H), 7.97 (d, J = 14.4 Hz, 1H), 7.84 (d, J = 7.2 Hz, 1H), 7.32 (q, J = 4.8 Hz, 1H), 6.47 (s, 1H), 6.01 (d, J = 7.6 Hz, 1H), 4.86 (s, 2H), 4.76 (s, 2H), 4.12-4.03 (m, 4H), 2.43-2.38 (m, 2H), 1.97 (s, 3H); |
| 191 | 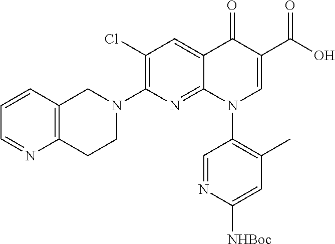 [SMILES: Cc1cc(NC(=O)OC(C)(C)C)ncc1-n1cc(C(=O)O)c(=O)c2cc(Cl)c(N3CCc4ncccc4C3)nc21] | trituration with 10% Isopropanol in diethyl ether | 1-(6-((tert-butoxy- carbonyl)amino)-4- methylpyridin-3- yl)-6-chloro-7-(7,8- dihydro-1,6-naph- thyridin-6(5H)-yl)- 4-oxo-1,4-dihydro- 1,8-naphthyridine- 3-carboxylic acid | MS (ESI): m/z 563.3 [M + H]⁺; 1H NMR (400 MHz, DMSO-d₆): δ 14.47 (brs, 1H), 9.57 (s, 1H), 8.69 (s, 1H), 8.43 (s, 1H), 8.32 (d, J = 4.8 Hz, 1H), 8.20 (s, 1H), 7.83 (s, 1H), 7.22 (d, J = 6.8 Hz, 1H), 7.13-7.09 (m, 1H), 4.55 (s, 2H), 3.89-3.68 (m, 2H), 2.82 (t, J = 6.0 Hz, 2H), 1.91 (s, 3H), 1.54 (s, 9H). |
| 192 | 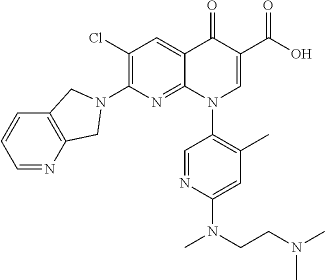 [SMILES: Cc1cc(N(C)CCN(C)C)ncc1-n1cc(C(=O)O)c(=O)c2cc(Cl)c(N3Cc4cccnc4C3)nc21] | Trituration with Et2O followed n-Pentane | 6-chloro-7-(5,7- dihydro-6H-pyr- rolo[3,4-b]pyridin- 6-yl)-1-(6-((2- (dimethylamino)- ethyl)(methyl)- amino)-4-methyl- pyridin-3-yl)-4- oxo-1,4-dihydro- 1,8-naphthyridine- 3-carboxylic acid | MS (ESI): m/z 534.3 [M + H]⁺; ¹H NMR (400 MHz, DMSO-d₆): δ 15.00 (brs, 1H), 8.67 (s, 1H), 8.45 (d, J = 4.4 Hz, 1H), 8.41 (s, 1H), 8.08 (s, 1H), 7.74 (d, J = 7.6 Hz, 1H), 7.34-7.30 (m, 1H), 6.69 (s, 1H), 5.14 (brs, 2H), 4.83 (brs, 2H), 3.79- 3.66 (m, 2H), 3.10 (s, 3H), 2.48 (brs, 2H), 2.24 (s, 6H), 2.00 (s, 3H). |
| 193 | 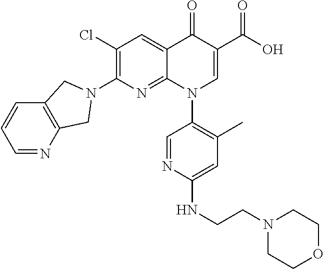 [SMILES: Cc1cc(NCCN2CCOCC2)ncc1-n1cc(C(=O)O)c(=O)c2cc(Cl)c(N3Cc4cccnc4C3)nc21] | Reverse phase purification, C-18 column, water/ACN | 6-chloro-7-(5,7- dihydro-6H-pyr- rolo[3,4-b]pyridin- 6-yl)-1-(4-methyl- 6-((2-morpholino- ethyl)amino)pyr- idin-3-yl)-4-oxo- 1,4-dihydro-1,8- naphthyridine-3- carboxylic acid | MS (ESI): m/z 562.3 [M + H]⁺; ¹H NMR (400 MHz, DMSO-d₆): δ 15.01 (s, 1H), 8.63 (s, 1H), 8.45 (d, J = 4.4 Hz, 1H), 8.41 (s, 1H), 8.10 (s, 1H), 7.78 (d, J = 8.0 Hz, 1H), 7.35-7.31 (m, 1H), 7.22 (brs, 1H), 6.26 (s, 1H), 5.24 (brs, 2H), 4.77 (brs, 2H), 3.85 (s, 4H), 3.80-3.69 (m, 2H), 3.52- 3.48 (m, 2H), 3.40-3.28 (m, 4H), 1.98 (s, 3H). |
| 194 | 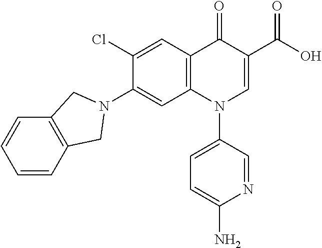 [SMILES: Nc1ccc(-n2cc(C(=O)O)c(=O)c3cc(Cl)c(N4Cc5ccccc5C4)cc32)cn1] | Prep-HPLC, Method-13 of Table-2 | 1-(6-aminopyridin- 3-yl)-6-chloro-7- (isoindolin-2-yl)-4- oxo-1,4-dihydro- quinoline-3-carbox- ylic acid | MS (ESI): m/z 433 [M + H]⁺; 1H NMR (400 MHz, DMSO-d₆) δ 15.15 (s, 1H), 8.53 (s, 1H), 8.22 (s, 1H), 8.18 (d, J = 2.4 Hz, 1H), 7.67 (dd, J = 8.8, 2.8 Hz, 1H), 7.41-7.37 (m, 2H), 7.32-7.28 (m, 2H), 6.64 (d, J = 8.8 Hz, 1H), 6.61 (brs, 2H), 6.40 (s, 1H), 4.92 (s, 4H). |
| 195 | 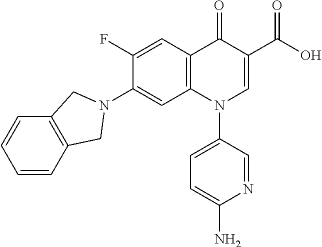 [SMILES: Nc1ccc(-n2cc(C(=O)O)c(=O)c3cc(F)c(N4Cc5ccccc5C4)cc32)cn1] | Prep-HPLC, Method-4 of Table-2 | 1-(6-aminopyr- idin-3-yl)-6-fluoro- 7-(isoindolin-2-yl)- 4-oxo-1,4-dihydro- quinoline-3-carbox- ylic acid | MS (ESI): m/z 417 [M + H]⁺; 1H NMR (400 MHz, DMSO-d₆) δ 15.41 (s, 1H), 8.51 (s, 1H), 8.20 (d, J = 2.4 Hz, 1H), 7.96 (d, J = 14.4 Hz, 1H), 7.69 (dd, J = 8.8, 2.8 Hz, 1H), 7.43-7.39 (m, 2H), 7.32- 7.28 (m, 2H), 6.72 (brs, 2H), 6.67 (d, J = 8.8 Hz, 1H), 6.22 (d, J = 7.6 Hz, 1H), 4.83 (s, 4H). |
| 196 | 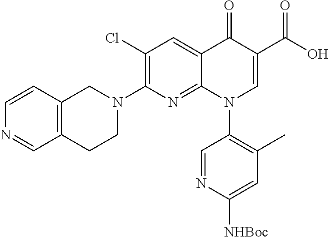 [SMILES: Cc1cc(NC(=O)OC(C)(C)C)ncc1-n1cc(C(=O)O)c(=O)c2cc(Cl)c(N3CCc4cnccc4C3)nc21] | Prep-HPLC, Method-13 of Table-2 | 1-(6-((tert-butoxy- carbonyl)amino)-4- methylpyridin-3- yl)-6-chloro-7-(3,4- dihydro-2,6-naph- thyridin-2(1H)-yl)- 4-oxo-1,4-dihydro- 1,8-naphthyridine- 3-carboxylic acid | MS (ESI): m/z 563.2 [M + H]⁺; 1H NMR (400 MHz, DMSO-d₆): δ 14.78 (brs, 1H), 10.11 (s, 1H), 8.81 (s, 1H), 8.50 (s, 1H), 8.29-8.25 (m, 3H), 7.89 (s, 1H), 6.86 (d, J = 5.2 Hz, 1H), 4.54 (ABq, J = 2.8 Hz, 2H), 3.95- 3.76 (m, 2H), 2.71 (t, J = 6.0 Hz, 2H), 1.92 (s, 3H), 1.54 (s, 9H). |
| 197 | 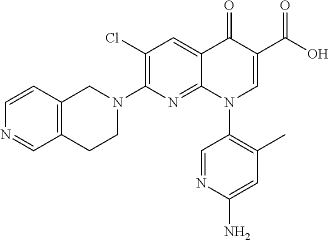 [SMILES: Cc1cc(N)ncc1-n1cc(C(=O)O)c(=O)c2cc(Cl)c(N3CCc4cnccc4C3)nc21] | Trituration with Et2O | 1-(6-amino-4-meth- ylpyridin-3-yl)-6- chloro-7-(3,4-di- hydro-2,6-naph- thyridin-2(1H)-yl)- 4-oxo-1,4-dihydro- 1,8-naphthyridine- 3-carboxylic acid | MS (ESI): m/z 463.2 [M + H]⁺; 1H NMR (400 MHz, DMSO-d₆): δ 14.70 (brs, 1H), 8.85 (s, 1H), 8.53 (s, 1H), 8.49 (s, 1H), 8.47 (d, J = 5.6 Hz, 1H), 8.08 (s, 1H), 7.76 (brs, 2H), 7.24 (d, J = 5.2 Hz, 1H), 6.76 (s, 1H), 4.70 (s, 2H), 3.98-3.84 (m, 2H), 2.82 (t, J = 6.0 Hz, 2H), 1.87 (s, 3H). |
| 198 | 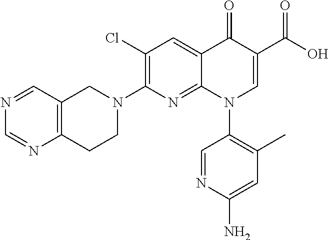 [SMILES: Cc1cc(N)ncc1-n1cc(C(=O)O)c(=O)c2cc(Cl)c(N3CCc4ncncc4C3)nc21] | Trituration with Et2O | 1-(6-amino-4-meth- ylpyridin-3-yl)-6- chloro-7-(7,8-di- hydropyrido[4,3- d]pyrimidin-6(5H)- yl)-4-oxo-1,4- dihydro-1,8-naph- thyridine-3-carbox- ylic acid | MS (ESI): m/z 464.2 [M + H]⁺; 1H NMR (400 MHz, DMSO-d₆): δ 14.72 (brs, 1H), 8.93 (s, 1H), 8.82 (s, 1H), 8.52 (s, 1H), 8.42 (s, 1H), 8.05 (s, 1H), 7.40 (brs, 2H), 6.71 (s, 1H), 4.70 (ABq, J = 8.0 Hz, 2H), 4.01-3.82 (m, 2H), 2.81 (t, J = 6.0 Hz, 2H), 1.84 (s, 3H). |
| 199 | 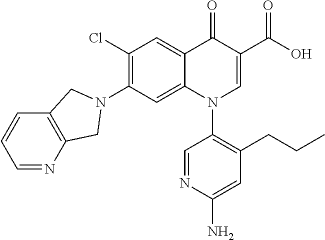 [SMILES: CCCc1cc(N)ncc1-n1cc(C(=O)O)c(=O)c2cc(Cl)c(N3Cc4cccnc4C3)cc21] | Prep-HPLC, Method-13 of Table-2 | 1-(6-amino-4- propylpyridin-3-yl)- 6-chloro-7-(5,7- dihydro-6H-pyr- rolo[3,4-b]pyridin- 6-yl)-4-oxo-1,4- dihydroquinoline- 3-carboxylic acid | MS (ESI): m/z 476.2 [M + H]⁺; ¹H NMR (400 MHz, DMSO-d₆) δ 15.20 (s, 1H), 8.55 (s, 1H), 8.46 (d, J = 4.8 Hz, 1H), 8.24 (s, 1H), 8.04 (s, 1H), 7.84 (d, J = 8.0 Hz, 1H), 7.32 (q, J = 7.6 Hz, 1H), 6.54 (s, 1H), 6.49 (s, 2H), 6.21 (s, 1H), 5.01-4.81 (m, 4H), 2.33-2.08 (m, 2H), 1.44-1.39 (m, 2H), 0.75 (t, J = 7.2 Hz, 3H). |
| 200 | 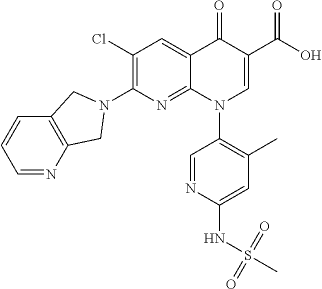 [SMILES: Cc1cc(NS(C)(=O)=O)ncc1-n1cc(C(=O)O)c(=O)c2cc(Cl)c(N3Cc4cccnc4C3)nc21] | Prep-HPLC, Method-4 of Table-2 | 6-chloro-7-(5,7- dihydro-6H-pyr- rolo[3,4-b]pyridin- 6-yl)-1-(4-methyl- 6-(methylsulfon- amido)pyridin-3- yl)-4-oxo-1,4- dihydro-1,8-naph- thyridine-3-carbox- ylic acid | MS (ESI): m/z 527.1 [M + H]⁺; ¹H NMR (400 MHz, DMSO-d₆): δ 14.98 (s, 1H), 10.89 (s, 1H), 8.82 (s, 1H), 8.46 (d, J = 4.4 Hz, 1H), 8.42 (s, 1H), 8.38 (s, 1H), 7.73 (d, J = 7.8 Hz, 1H), 7.32 (q, J = 7.2 Hz, 1H), 7.07 (s, 1H), 5.20 (s, 2H), 4.85 (s, 2H), 3.38 (s, 3H), 2.08 (s, 3H); |
| 201 | 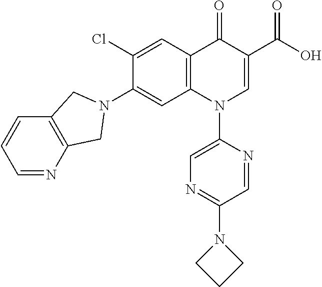 [SMILES: O=C(O)c1cn(-c2cnc(N3CCC3)cn2)c2cc(N3Cc4cccnc4C3)c(Cl)cc2c1=O] | trituration with diethyl ether, n- Pentane and ACN, IPA | 1-(5-(azetidin-1- yl)pyrazin-2-yl)-6- chloro-7-(5,7-di- hydro-6H-pyr- rolo[3,4-b]pyridin- 6-yl)-4-oxo-1,4- dihydroquinoline- 3-carboxylic acid | MS (ESI): m/z 475.1 [M + H]⁺; ¹H NMR (400 MHz, CDCl₃): δ 14.91 (s, 1H), 8.64 (s, 1H), 8.52 (d, J = 3.6 Hz, 1H), 8.45 (s, 1H), 8.16 (d, J = 1.2 Hz, 1H), 7.71 (d, J = 1.2 Hz, 1H), 7.64 (d, J = 7.2 Hz, 1H), 7.24 (d, J = 4.8 Hz, 1H), 6.35 (s, 1H), 5.27 (s, 2H), 4.73 (s, 2H), 4.35 (t, J = 7.6 Hz, 4H), 2.68-2.60 (m, 2H). |
| 203 | 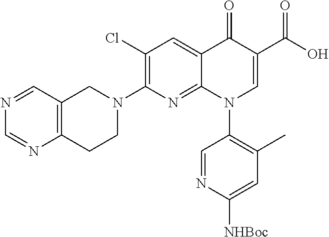 [SMILES: Cc1cc(NC(=O)OC(C)(C)C)ncc1-n1cc(C(=O)O)c(=O)c2cc(Cl)c(N3CCc4ncncc4C3)nc21] | Prep-HPLC, Method-9 of Table-2 | 1-(6-((tert-butoxy- carbonyl)amino)-4- methylpyridin-3- yl)-6-chloro-7-(7,8- dihydropyrido[4,3- d]pyrimidin-6(5H)- yl)-4-oxo-1,4- dihydro-1,8-naph- thyridine-3-carbox- ylic acid | MS (ESI): m/z 564 [M + H]⁺; 1H NMR (400 MHz, DMSO-d₆): δ 14.73 (brs, 1H), 10.07 (s, 1H), 8.91 (s, 1H), 8.78 (s, 1H), 8.50 (s, 1H), 8.37 (s, 1H), 8.26 (s, 1H), 7.91 (s, 1H), 4.64 (ABq, J = 7.6 Hz, 2H), 3.91-3.73 (m, 2H), 2.73 (t, J = 6.0 Hz, 2H), 1.96 (s, 3H), 1.53 (s, 9H). |

### D. Testing of Exemplified Compounds in PAPD5 Assay

The purpose of the PAPD5 assay is to determine compound potency against PAPD5 enzyme through the measurement of IC₅₀. Compound inhibition is measured as a function of AMP incorporation onto the 3′-terminus of an RNA substrate in the presence of active PAPD5, ATP, and inhibitor. The PAPD5 assay was done in a black, non-binding, 384 well plate (Corning #3575). All steps were performed at room temperature. For a typical assay, a 40 ul aliquot of assay buffer (50 mM HEPES pH 7.3, 20 mM KCl, 10 mM MgCl₂, 10 mMM DTT, 0.01% Triton X-100, 40 U/ml RNAsin (Promega)) containing PAPD5 protein (final 10 concentration: 0.3 nM) and ATP (final concentration 50 μM) was added to each well. Next, 1 ul of compound in 100% DMSO is transferred from a compound dilution plate in which the compound is serially diluted. The plate was placed on a plate shaker for 30 seconds to produce mixing, then incubated for 30 minutes. Next, 10 ul of a 5× mix of RNA substrate (5′-fluorescein-AUAGAG-3′ (IDT)) and a DNA oligonucleotide that is complementary to the AMP modified RNA product (5′-TCTCTATT-3′-TAMRA, wherein the first two nucleotides are locked nucleic acids (IDT)) was added (final concentrations: 5 nM and 20 nM, respectively). The plate was placed on a plate shaker for 30 seconds to produce mixing then immediately placed in a Biotek Cytation 5 plate reader, and the time-dependent decrease in fluorescein fluorescence resulting from annealing of the TAMRA labeled quenching probe was recorded, using the kinetics mode of the reader (typical run time is 90 minutes). Reaction rates were obtained using linear fits to the early portion of progress curves. IC₅₀ values were calculated using a four-parameter fit with 100% enzyme activity determined from the DMSO control and 0% activity from control samples lacking UTP.

The potency of the compounds described herein against PAPD5 enzyme is shown in Table 4 below. “+” represents an IC₅₀ value that is greater than 50 PM; “++” represents an IC₅₀ value that is greater than 10 μM and equal to or less than 50 μM; “+++” represents an IC₅₀ value that is greater than 1 μM and equal to or less than 10 μM; and “++++” represents an IC₅₀ value that is equal to or less than 1 μM.

TABLE 4
Potency PAPD5 − IC50 (μm)

| | | Potency |
|---|---|---|
| | | PAPD5 |
| Example | Product | IC50 (μm) |
| 1 | 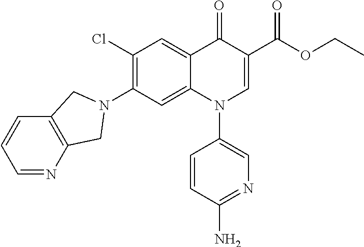 [SMILES: CCOC(=O)c1cn(-c2ccc(N)nc2)c2cc(N3Cc4cccnc4C3)c(Cl)cc2c1=O] | ++++ |
| 2 | 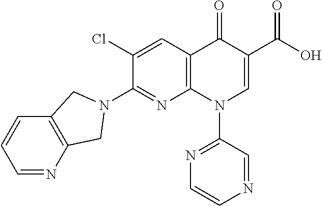 [SMILES: O=C(O)c1cn(-c2cnccn2)c2nc(N3Cc4cccnc4C3)c(Cl)cc2c1=O] | ++++ |
| 3 | 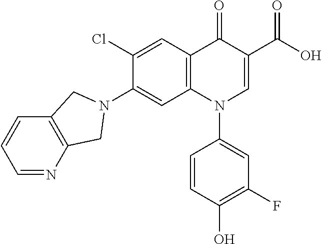 [SMILES: O=C(O)c1cn(-c2ccc(O)c(F)c2)c2cc(N3Cc4cccnc4C3)c(Cl)cc2c1=O] | ++++ |
| 4 | 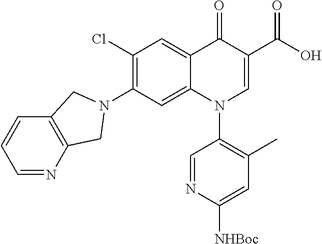 [SMILES: Cc1cc(NC(=O)OC(C)(C)C)ncc1-n1cc(C(=O)O)c(=O)c2cc(Cl)c(N3Cc4cccnc4C3)cc21] | ++++ |
| 5 | 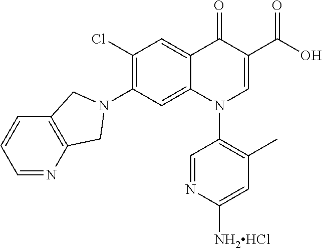 | ++++ |
| 6 | 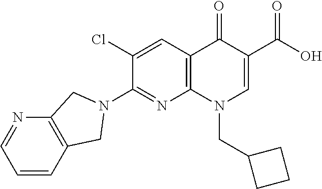 [SMILES: O=C(O)c1cn(CC2CCC2)c2nc(N3Cc4cccnc4C3)c(Cl)cc2c1=O] | ++++ |
| 7 | 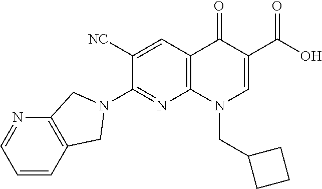 [SMILES: N#Cc1cc2c(=O)c(C(=O)O)cn(CC3CCC3)c2nc1N1Cc2cccnc2C1] | ++++ |
| 8 | 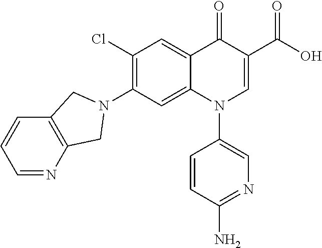 [SMILES: Nc1ccc(-n2cc(C(=O)O)c(=O)c3cc(Cl)c(N4Cc5cccnc5C4)cc32)cn1] | ++++ |
| 9 | 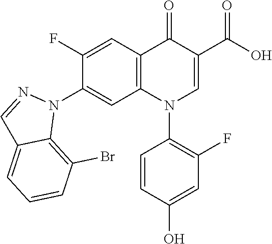 [SMILES: O=C(O)c1cn(-c2ccc(O)cc2F)c2cc(-n3ncc4cccc(Br)c43)c(F)cc2c1=O] | ++++ |
| 10 | 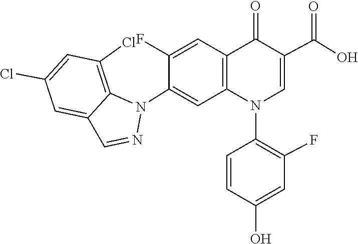 [SMILES: O=C(O)c1cn(-c2ccc(O)cc2F)c2cc(-n3ncc4cc(Cl)cc(Cl)c43)c(F)cc2c1=O] | ++++ |
| 11 | 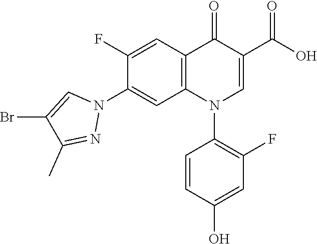 [SMILES: Cc1nn(-c2cc3c(cc2F)c(=O)c(C(=O)O)cn3-c2ccc(O)cc2F)cc1Br] | ++++ |
| 12 | 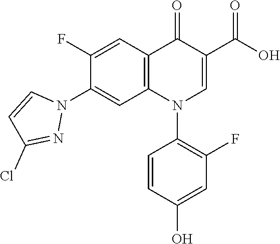 [SMILES: O=C(O)c1cn(-c2ccc(O)cc2F)c2cc(-n3ccc(Cl)n3)c(F)cc2c1=O] | ++++ |
| 13 | 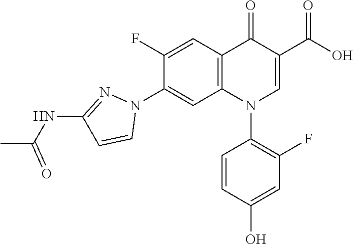 [SMILES: CC(=O)Nc1ccn(-c2cc3c(cc2F)c(=O)c(C(=O)O)cn3-c2ccc(O)cc2F)n1] | ++++ |
| 14 | 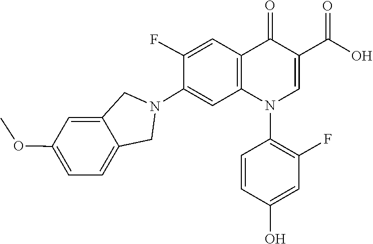 [SMILES: COc1ccc2c(c1)CN(c1cc3c(cc1F)c(=O)c(C(=O)O)cn3-c1ccc(O)cc1F)C2] | ++++ |
| 15 | 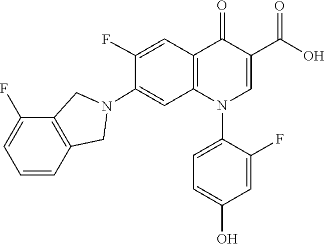 [SMILES: O=C(O)c1cn(-c2ccc(O)cc2F)c2cc(N3Cc4cccc(F)c4C3)c(F)cc2c1=O] | ++++ |
| 16 | 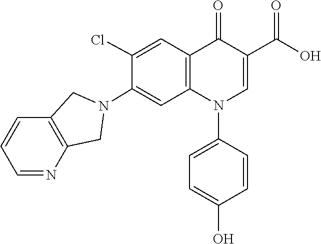 [SMILES: O=C(O)c1cn(-c2ccc(O)cc2)c2cc(N3Cc4cccnc4C3)c(Cl)cc2c1=O] | ++++ |
| 17 | 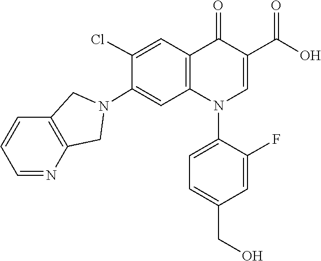 [SMILES: O=C(O)c1cn(-c2ccc(CO)cc2F)c2cc(N3Cc4cccnc4C3)c(Cl)cc2c1=O] | ++++ |
| 18 | 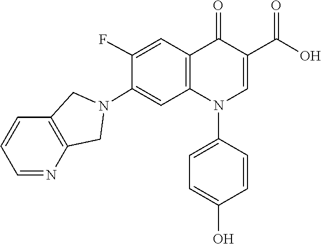 [SMILES: O=C(O)c1cn(-c2ccc(O)cc2)c2cc(N3Cc4cccnc4C3)c(F)cc2c1=O] | ++++ |
| 19 | 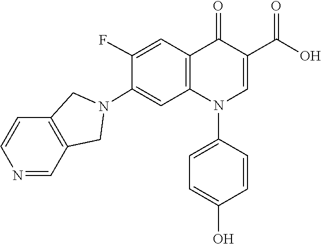 [SMILES: O=C(O)c1cn(-c2ccc(O)cc2)c2cc(N3Cc4ccncc4C3)c(F)cc2c1=O] | ++++ |
| 20 | 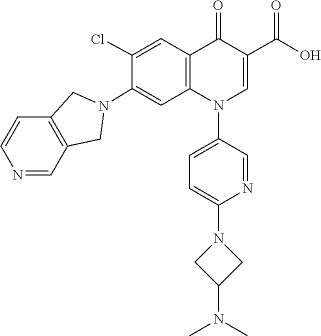 [SMILES: CN(C)C1CN(c2ccc(-n3cc(C(=O)O)c(=O)c4cc(Cl)c(N5Cc6ccncc6C5)cc43)cn2)C1] | ++++ |
| 21 | 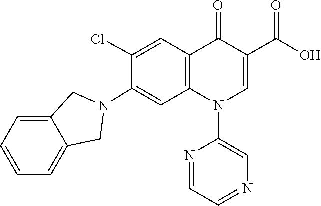 [SMILES: O=C(O)c1cn(-c2cnccn2)c2cc(N3Cc4ccccc4C3)c(Cl)cc2c1=O] | ++++ |
| 22 | 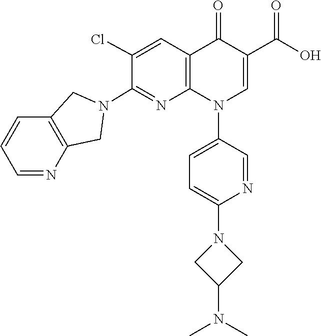 [SMILES: CN(C)C1CN(c2ccc(-n3cc(C(=O)O)c(=O)c4cc(Cl)c(N5Cc6cccnc6C5)nc43)cn2)C1] | ++++ |
| 23 | 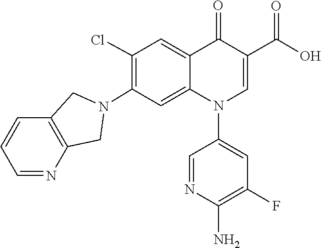 [SMILES: Nc1ncc(-n2cc(C(=O)O)c(=O)c3cc(Cl)c(N4Cc5cccnc5C4)cc32)cc1F] | ++++ |
| 24 | 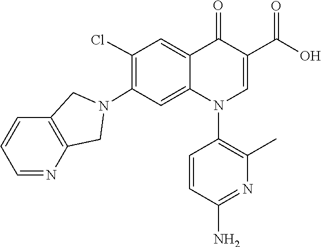 [SMILES: Cc1nc(N)ccc1-n1cc(C(=O)O)c(=O)c2cc(Cl)c(N3Cc4cccnc4C3)cc21] | ++++ |
| 25 | 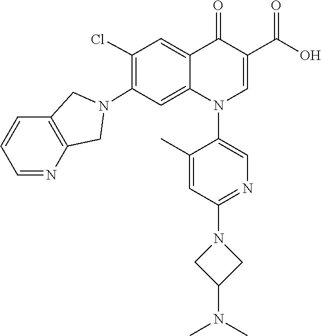 [SMILES: Cc1cc(N2CC(N(C)C)C2)ncc1-n1cc(C(=O)O)c(=O)c2cc(Cl)c(N3Cc4cccnc4C3)cc21] | ++++ |
| 26 | 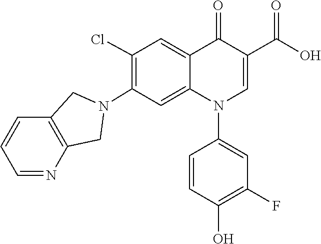 [SMILES: O=C(O)c1cn(-c2ccc(O)c(F)c2)c2cc(N3Cc4cccnc4C3)c(Cl)cc2c1=O] | ++++ |
| 27 | 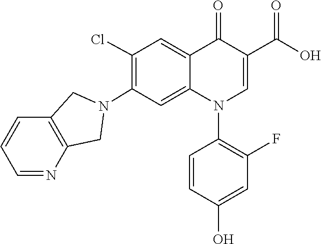 [SMILES: O=C(O)c1cn(-c2ccc(O)cc2F)c2cc(N3Cc4cccnc4C3)c(Cl)cc2c1=O] | ++++ |
| 28 | 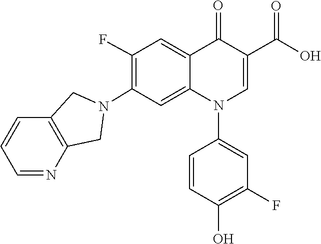 [SMILES: O=C(O)c1cn(-c2ccc(O)c(F)c2)c2cc(N3Cc4cccnc4C3)c(F)cc2c1=O] | ++++ |
| 29 | 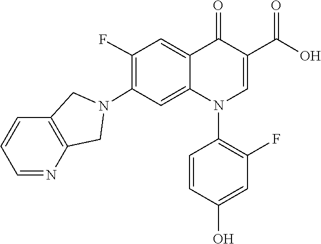 [SMILES: O=C(O)c1cn(-c2ccc(O)cc2F)c2cc(N3Cc4cccnc4C3)c(F)cc2c1=O] | ++++ |
| 30 | 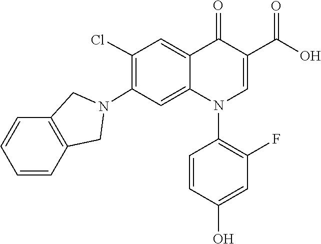 [SMILES: O=C(O)c1cn(-c2ccc(O)cc2F)c2cc(N3Cc4ccccc4C3)c(Cl)cc2c1=O] | ++++ |
| 31 | 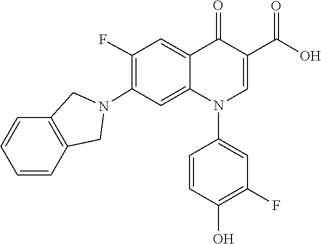 [SMILES: O=C(O)c1cn(-c2ccc(O)c(F)c2)c2cc(N3Cc4ccccc4C3)c(F)cc2c1=O] | ++++ |
| 32 | 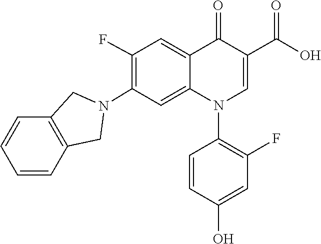 [SMILES: O=C(O)c1cn(-c2ccc(O)cc2F)c2cc(N3Cc4ccccc4C3)c(F)cc2c1=O] | ++++ |
| 33 | 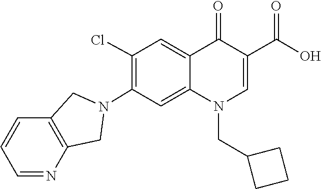 [SMILES: O=C(O)c1cn(CC2CCC2)c2cc(N3Cc4cccnc4C3)c(Cl)cc2c1=O] | ++++ |
| 34 | 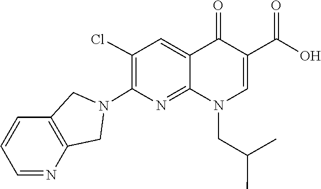 [SMILES: CC(C)Cn1cc(C(=O)O)c(=O)c2cc(Cl)c(N3Cc4cccnc4C3)nc21] | ++++ |
| 35 | 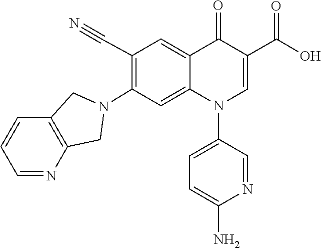 [SMILES: N#Cc1cc2c(=O)c(C(=O)O)cn(-c3ccc(N)nc3)c2cc1N1Cc2cccnc2C1] | ++++ |
| 36 | 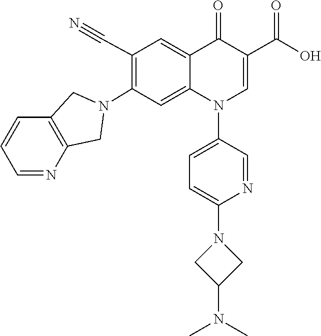 [SMILES: CN(C)C1CN(c2ccc(-n3cc(C(=O)O)c(=O)c4cc(C#N)c(N5Cc6cccnc6C5)cc43)cn2)C1] | ++++ |
| 37 | 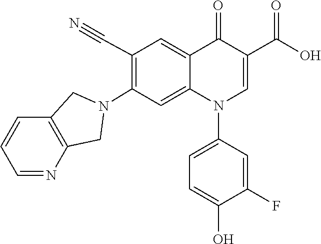 [SMILES: N#Cc1cc2c(=O)c(C(=O)O)cn(-c3ccc(O)c(F)c3)c2cc1N1Cc2cccnc2C1] | ++++ |
| 38 | 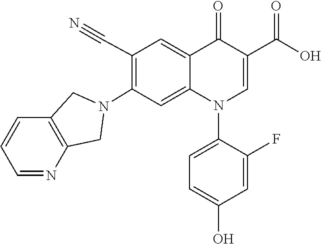 [SMILES: N#Cc1cc2c(=O)c(C(=O)O)cn(-c3ccc(O)cc3F)c2cc1N1Cc2cccnc2C1] | ++++ |
| 39 | 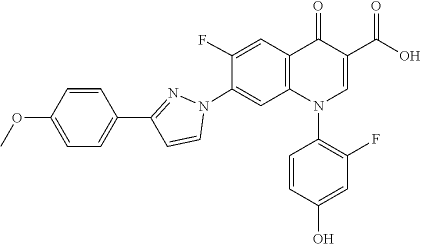 [SMILES: COc1ccc(-c2ccn(-c3cc4c(cc3F)c(=O)c(C(=O)O)cn4-c3ccc(O)cc3F)n2)cc1] | ++++ |
| 40 | 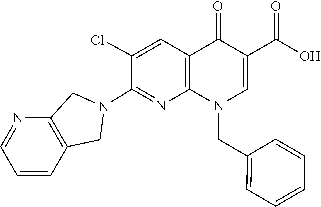 [SMILES: O=C(O)c1cn(Cc2ccccc2)c2nc(N3Cc4cccnc4C3)c(Cl)cc2c1=O] | ++++ |
| 41 | 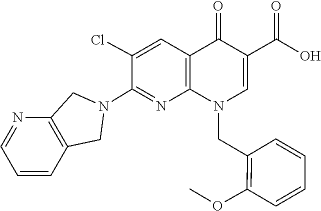 [SMILES: COc1ccccc1Cn1cc(C(=O)O)c(=O)c2cc(Cl)c(N3Cc4cccnc4C3)nc21] | ++++ |
| 42 | 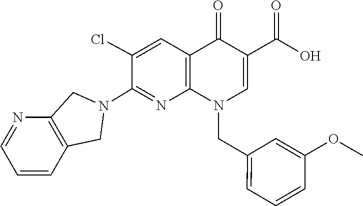 [SMILES: COc1cccc(Cn2cc(C(=O)O)c(=O)c3cc(Cl)c(N4Cc5cccnc5C4)nc32)c1] | ++++ |
| 43 | 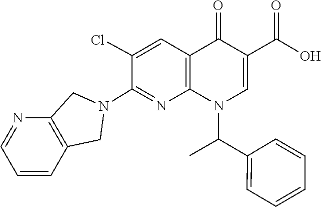 [SMILES: CC(c1ccccc1)n1cc(C(=O)O)c(=O)c2cc(Cl)c(N3Cc4cccnc4C3)nc21] | ++++ |
| 44 | 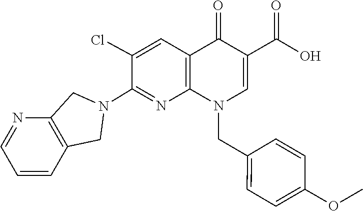 [SMILES: COc1ccc(Cn2cc(C(=O)O)c(=O)c3cc(Cl)c(N4Cc5cccnc5C4)nc32)cc1] | ++++ |
| 45 | 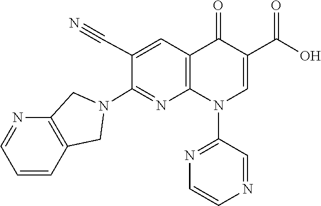 [SMILES: N#Cc1cc2c(=O)c(C(=O)O)cn(-c3cnccn3)c2nc1N1Cc2cccnc2C1] | ++++ |
| 46 | 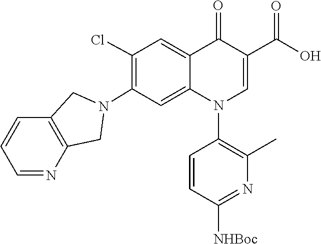 [SMILES: Cc1nc(NC(=O)OC(C)(C)C)ccc1-n1cc(C(=O)O)c(=O)c2cc(Cl)c(N3Cc4cccnc4C3)cc21] | ++++ |
| 47 | 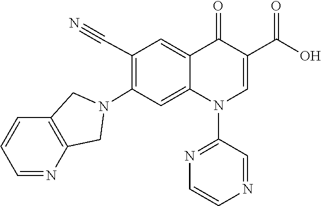 [SMILES: N#Cc1cc2c(=O)c(C(=O)O)cn(-c3cnccn3)c2cc1N1Cc2cccnc2C1] | ++++ |
| 48 | 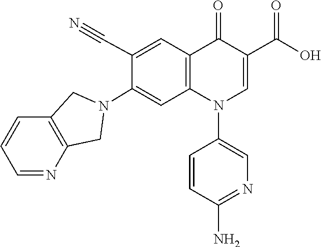 [SMILES: N#Cc1cc2c(=O)c(C(=O)O)cn(-c3ccc(N)nc3)c2cc1N1Cc2cccnc2C1] | ++++ |
| 49 | 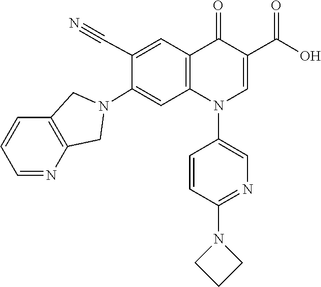 [SMILES: N#Cc1cc2c(=O)c(C(=O)O)cn(-c3ccc(N4CCC4)nc3)c2cc1N1Cc2cccnc2C1] | ++++ |
| 50 | 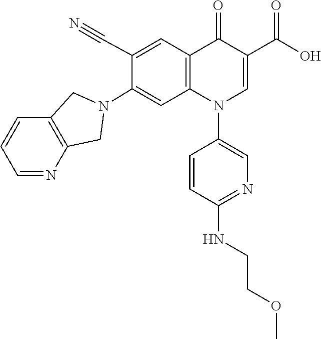 [SMILES: COCCNc1ccc(-n2cc(C(=O)O)c(=O)c3cc(C#N)c(N4Cc5cccnc5C4)cc32)cn1] | ++++ |
| 51 | 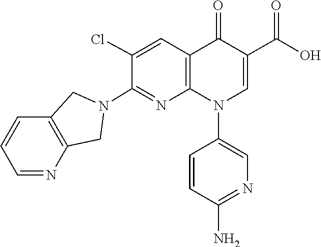 [SMILES: Nc1ccc(-n2cc(C(=O)O)c(=O)c3cc(Cl)c(N4Cc5cccnc5C4)nc32)cn1] | ++++ |
| 52 | 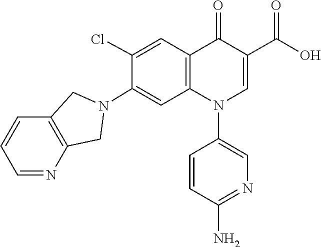 [SMILES: Nc1ccc(-n2cc(C(=O)O)c(=O)c3cc(Cl)c(N4Cc5cccnc5C4)cc32)cn1] | ++++ |
| 53 | 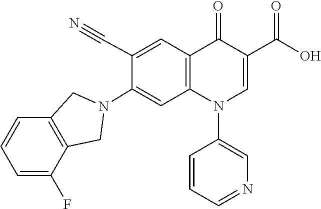 [SMILES: N#Cc1cc2c(=O)c(C(=O)O)cn(-c3cccnc3)c2cc1N1Cc2cccc(F)c2C1] | ++++ |
| 54 | 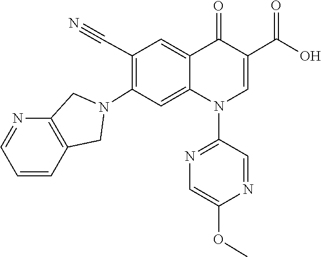 [SMILES: COc1cnc(-n2cc(C(=O)O)c(=O)c3cc(C#N)c(N4Cc5cccnc5C4)cc32)cn1] | ++++ |
| 55 | 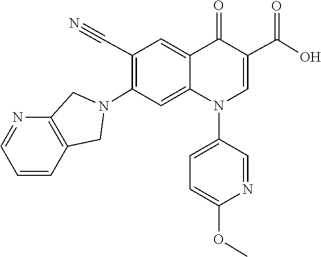 [SMILES: COc1ccc(-n2cc(C(=O)O)c(=O)c3cc(C#N)c(N4Cc5cccnc5C4)cc32)cn1] | ++++ |
| 56 | 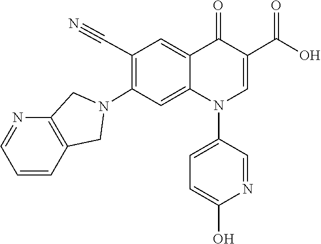 [SMILES: N#Cc1cc2c(=O)c(C(=O)O)cn(-c3ccc(O)nc3)c2cc1N1Cc2cccnc2C1] | ++++ |
| 57 | 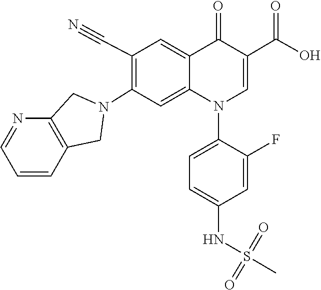 [SMILES: CS(=O)(=O)Nc1ccc(-n2cc(C(=O)O)c(=O)c3cc(C#N)c(N4Cc5cccnc5C4)cc32)c(F)c1] | ++++ |
| 58 | 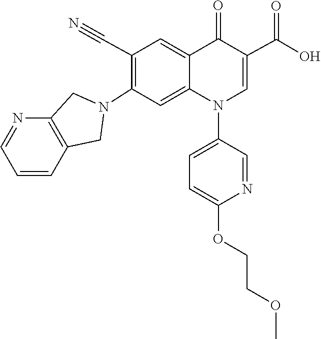 [SMILES: COCCOc1ccc(-n2cc(C(=O)O)c(=O)c3cc(C#N)c(N4Cc5cccnc5C4)cc32)cn1] | ++++ |
| 59 | 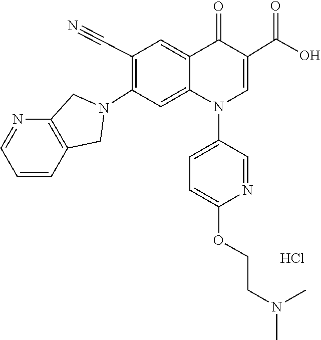 [SMILES: CN(C)CCOc1ccc(-n2cc(C(=O)O)c(=O)c3cc(C#N)c(N4Cc5cccnc5C4)cc32)cn1 ; Cl] | ++++ |
| 60 | 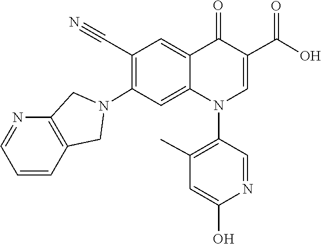 [SMILES: Cc1cc(O)ncc1-n1cc(C(=O)O)c(=O)c2cc(C#N)c(N3Cc4cccnc4C3)cc21] | ++++ |
| 61 | 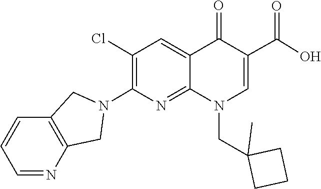 [SMILES: CC1(Cn2cc(C(=O)O)c(=O)c3cc(Cl)c(N4Cc5cccnc5C4)nc32)CCC1] | ++++ |
| 62 | 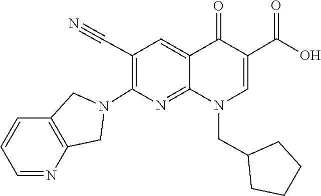 [SMILES: N#Cc1cc2c(=O)c(C(=O)O)cn(CC3CCCC3)c2nc1N1Cc2cccnc2C1] | ++++ |
| 63 | 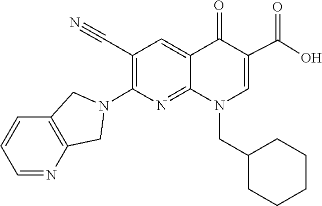 [SMILES: N#Cc1cc2c(=O)c(C(=O)O)cn(CC3CCCCC3)c2nc1N1Cc2cccnc2C1] | ++++ |
| 64 | 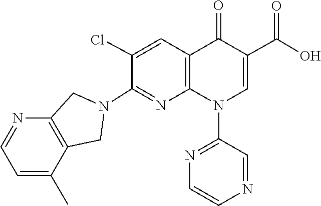 [SMILES: Cc1ccnc2c1CN(c1nc3c(cc1Cl)c(=O)c(C(=O)O)cn3-c1cnccn1)C2] | ++++ |
| 65 | 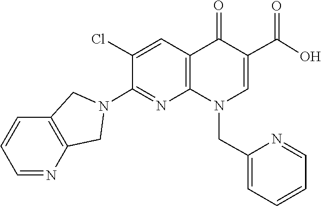 [SMILES: O=C(O)c1cn(Cc2ccccn2)c2nc(N3Cc4cccnc4C3)c(Cl)cc2c1=O] | ++++ |
| 66 | 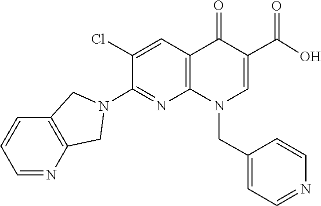 [SMILES: O=C(O)c1cn(Cc2ccncc2)c2nc(N3Cc4cccnc4C3)c(Cl)cc2c1=O] | ++++ |
| 67 | 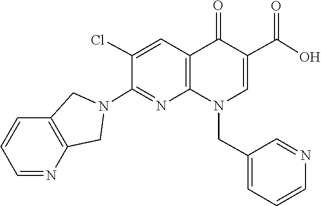 [SMILES: O=C(O)c1cn(Cc2cccnc2)c2nc(N3Cc4cccnc4C3)c(Cl)cc2c1=O] | ++++ |
| 68 | 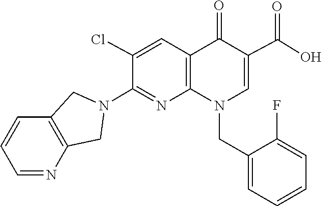 [SMILES: O=C(O)c1cn(Cc2ccccc2F)c2nc(N3Cc4cccnc4C3)c(Cl)cc2c1=O] | ++++ |
| 69 | 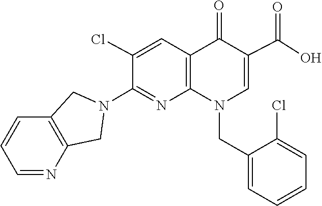 [SMILES: O=C(O)c1cn(Cc2ccccc2Cl)c2nc(N3Cc4cccnc4C3)c(Cl)cc2c1=O] | ++++ |
| 70 | 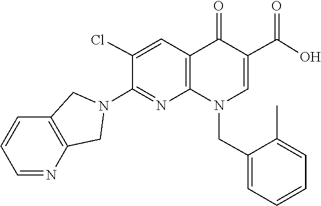 [SMILES: Cc1ccccc1Cn1cc(C(=O)O)c(=O)c2cc(Cl)c(N3Cc4cccnc4C3)nc21] | ++++ |
| 71 | 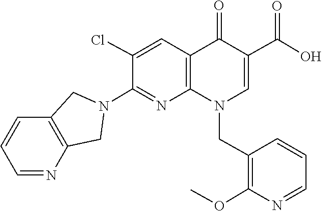 [SMILES: COc1ncccc1Cn1cc(C(=O)O)c(=O)c2cc(Cl)c(N3Cc4cccnc4C3)nc21] | ++++ |
| 72 | 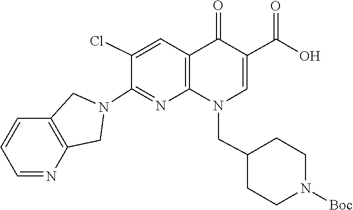 [SMILES: CC(C)(C)OC(=O)N1CCC(Cn2cc(C(=O)O)c(=O)c3cc(Cl)c(N4Cc5cccnc5C4)nc32)CC1] | ++++ |
| 73 | 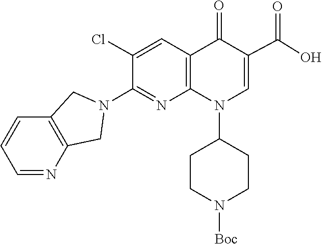 [SMILES: CC(C)(C)OC(=O)N1CCC(n2cc(C(=O)O)c(=O)c3cc(Cl)c(N4Cc5cccnc5C4)nc32)CC1] | ++++ |
| 74 | 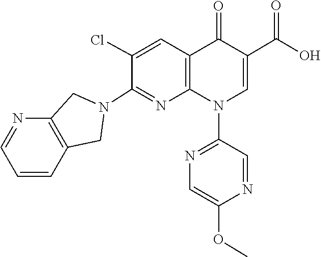 [SMILES: COc1cnc(-n2cc(C(=O)O)c(=O)c3cc(Cl)c(N4Cc5cccnc5C4)nc32)cn1] | ++++ |
| 75 | 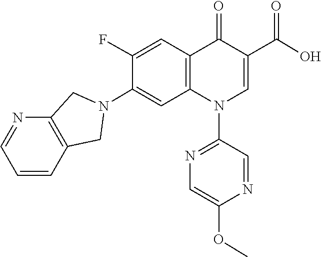 [SMILES: COc1cnc(-n2cc(C(=O)O)c(=O)c3cc(F)c(N4Cc5cccnc5C4)cc32)cn1] | ++++ |
| 76 | 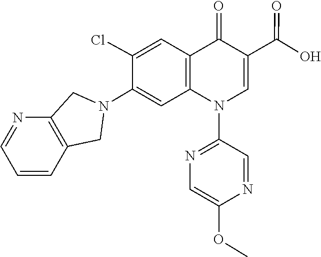 [SMILES: COc1cnc(-n2cc(C(=O)O)c(=O)c3cc(Cl)c(N4Cc5cccnc5C4)cc32)cn1] | ++++ |
| 77 | 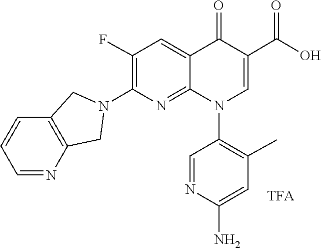 [SMILES: Cc1cc(N)ncc1-n1cc(C(=O)O)c(=O)c2cc(F)c(N3Cc4cccnc4C3)nc21] | ++++ |
| 78 | 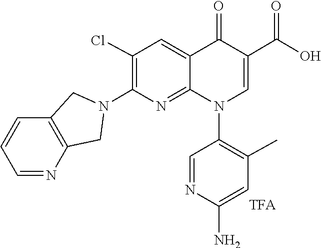 [SMILES: Cc1cc(N)ncc1-n1cc(C(=O)O)c(=O)c2cc(Cl)c(N3Cc4cccnc4C3)nc21] | ++++ |
| 79 | 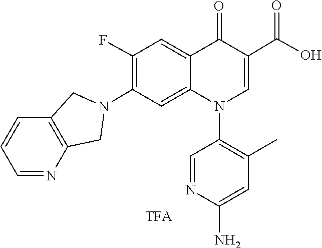 [SMILES: Cc1cc(N)ncc1-n1cc(C(=O)O)c(=O)c2cc(F)c(N3Cc4cccnc4C3)cc21] | ++++ |
| 80 | 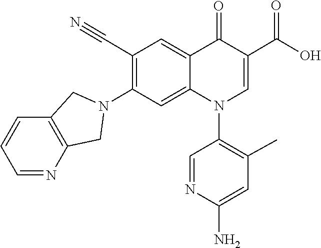 [SMILES: Cc1cc(N)ncc1-n1cc(C(=O)O)c(=O)c2cc(C#N)c(N3Cc4cccnc4C3)cc21] | ++++ |
| 81 | 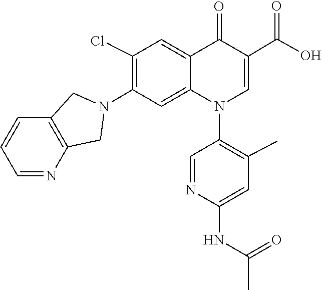 [SMILES: CC(=O)Nc1cc(C)c(-n2cc(C(=O)O)c(=O)c3cc(Cl)c(N4Cc5cccnc5C4)cc32)cn1] | ++++ |
| 82 | 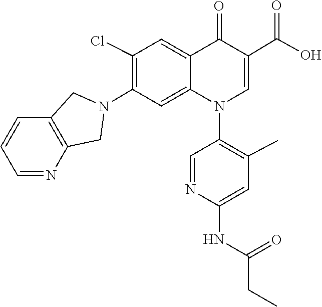 [SMILES: CCC(=O)Nc1cc(C)c(-n2cc(C(=O)O)c(=O)c3cc(Cl)c(N4Cc5cccnc5C4)cc32)cn1] | ++++ |
| 83 | 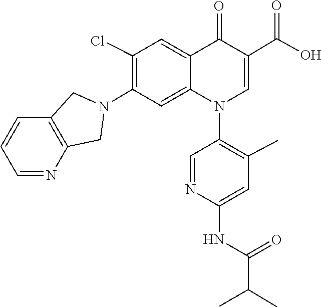 [SMILES: Cc1cc(NC(=O)C(C)C)ncc1-n1cc(C(=O)O)c(=O)c2cc(Cl)c(N3Cc4cccnc4C3)cc21] | ++++ |
| 84 | 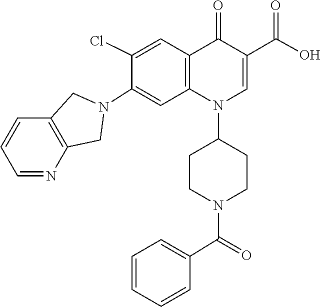 [SMILES: O=C(O)c1cn(C2CCN(C(=O)c3ccccc3)CC2)c2cc(N3Cc4cccnc4C3)c(Cl)cc2c1=O] | ++++ |
| 85 | 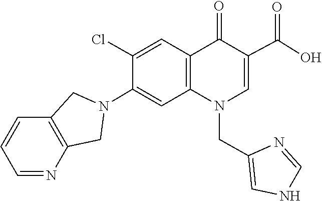 [SMILES: O=C(O)c1cn(Cc2c[nH]cn2)c2cc(N3Cc4cccnc4C3)c(Cl)cc2c1=O] | ++++ |
| 86 | 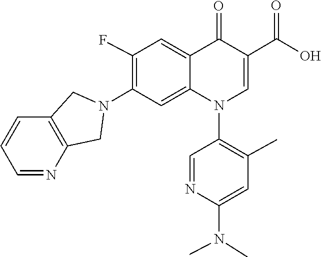 [SMILES: Cc1cc(N(C)C)ncc1-n1cc(C(=O)O)c(=O)c2cc(F)c(N3Cc4cccnc4C3)cc21] | ++++ |
| 87 | 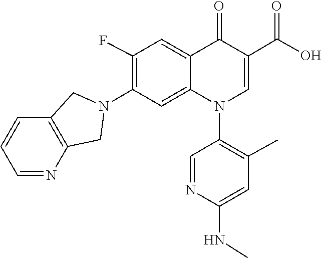 [SMILES: CNc1cc(C)c(-n2cc(C(=O)O)c(=O)c3cc(F)c(N4Cc5cccnc5C4)cc32)cn1] | ++++ |
| 88 | 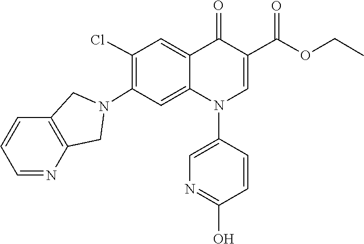 [SMILES: CCOC(=O)c1cn(-c2ccc(O)nc2)c2cc(N3Cc4cccnc4C3)c(Cl)cc2c1=O] | ++++ |
| 89 | 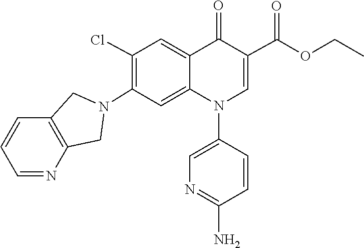 [SMILES: CCOC(=O)c1cn(-c2ccc(N)nc2)c2cc(N3Cc4cccnc4C3)c(Cl)cc2c1=O] | ++++ |
| 90 | 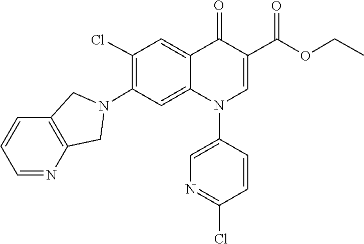 [SMILES: CCOC(=O)c1cn(-c2ccc(Cl)nc2)c2cc(N3Cc4cccnc4C3)c(Cl)cc2c1=O] | ++++ |
| 91 | 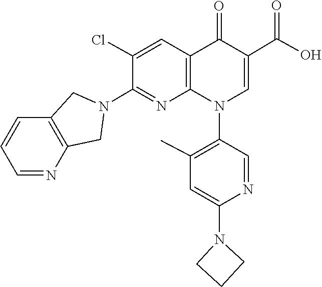 [SMILES: Cc1cc(N2CCC2)ncc1-n1cc(C(=O)O)c(=O)c2cc(Cl)c(N3Cc4cccnc4C3)nc21] | ++++ |
| 92 | 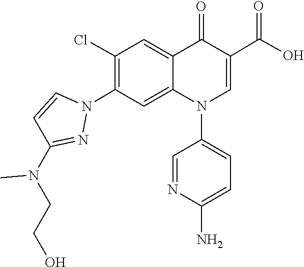 [SMILES: CN(CCO)c1ccn(-c2cc3c(cc2Cl)c(=O)c(C(=O)O)cn3-c2ccc(N)nc2)n1] | ++++ |
| 93 | 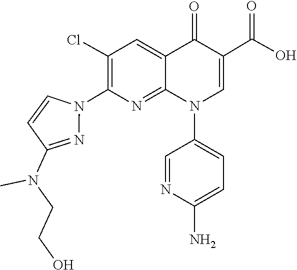 [SMILES: CN(CCO)c1ccn(-c2nc3c(cc2Cl)c(=O)c(C(=O)O)cn3-c2ccc(N)nc2)n1] | ++++ |
| 94 | 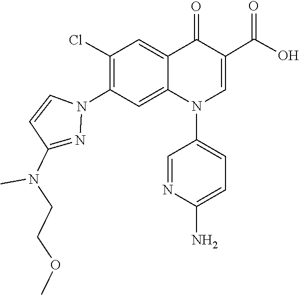 [SMILES: COCCN(C)c1ccn(-c2cc3c(cc2Cl)c(=O)c(C(=O)O)cn3-c2ccc(N)nc2)n1] | ++++ |
| 95 | 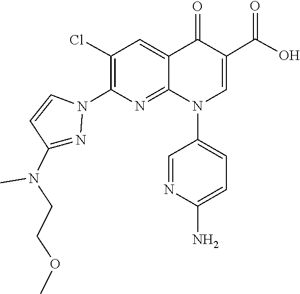 [SMILES: COCCN(C)c1ccn(-c2nc3c(cc2Cl)c(=O)c(C(=O)O)cn3-c2ccc(N)nc2)n1] | ++++ |
| 96 | 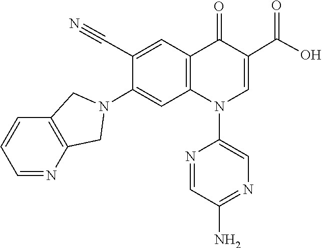 [SMILES: N#Cc1cc2c(=O)c(C(=O)O)cn(-c3cnc(N)cn3)c2cc1N1Cc2cccnc2C1] | ++++ |
| 97 | 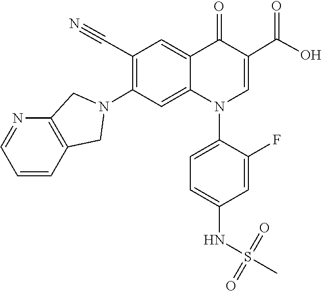 [SMILES: CS(=O)(=O)Nc1ccc(-n2cc(C(=O)O)c(=O)c3cc(C#N)c(N4Cc5cccnc5C4)cc32)c(F)c1] | ++++ |
| 98 | 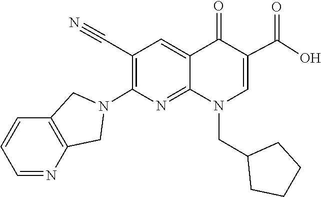 [SMILES: N#Cc1cc2c(=O)c(C(=O)O)cn(CC3CCCC3)c2nc1N1Cc2cccnc2C1] | ++++ |
| 99 | 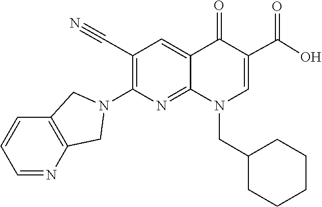 [SMILES: N#Cc1cc2c(=O)c(C(=O)O)cn(CC3CCCCC3)c2nc1N1Cc2cccnc2C1] | ++++ |
| 100 | 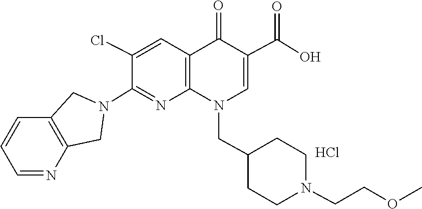 [SMILES: COCCN1CCC(Cn2cc(C(=O)O)c(=O)c3cc(Cl)c(N4Cc5cccnc5C4)nc32)CC1 ; Cl] | ++++ |
| 101 | 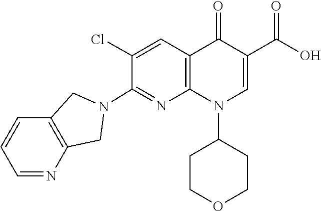 [SMILES: O=C(O)c1cn(C2CCOCC2)c2nc(N3Cc4cccnc4C3)c(Cl)cc2c1=O] | ++++ |
| 102 | 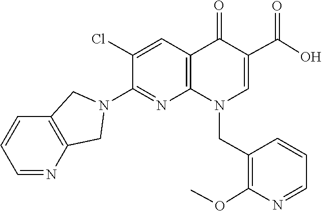 [SMILES: COc1ncccc1Cn1cc(C(=O)O)c(=O)c2cc(Cl)c(N3Cc4cccnc4C3)nc21] | ++++ |
| 103 | 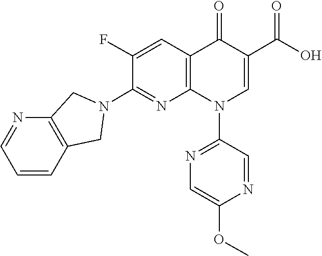 [SMILES: COc1cnc(-n2cc(C(=O)O)c(=O)c3cc(F)c(N4Cc5cccnc5C4)nc32)cn1] | ++++ |
| 104 | 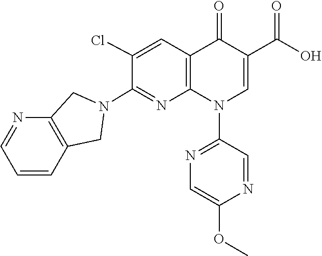 [SMILES: COc1cnc(-n2cc(C(=O)O)c(=O)c3cc(Cl)c(N4Cc5cccnc5C4)nc32)cn1] | ++++ |
| 105 | 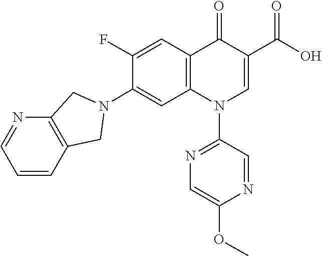 [SMILES: COc1cnc(-n2cc(C(=O)O)c(=O)c3cc(F)c(N4Cc5cccnc5C4)cc32)cn1] | ++++ |
| 106 | 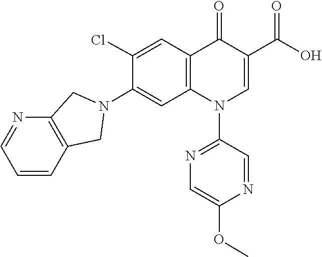 [SMILES: COc1cnc(-n2cc(C(=O)O)c(=O)c3cc(Cl)c(N4Cc5cccnc5C4)cc32)cn1] | ++++ |
| 107 | 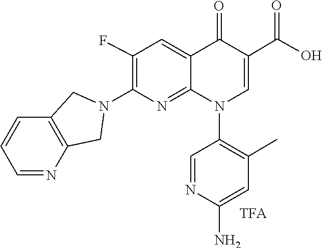 [SMILES: Cc1cc(N)ncc1-n1cc(C(=O)O)c(=O)c2cc(F)c(N3Cc4cccnc4C3)nc21] | ++++ |
| 108 | 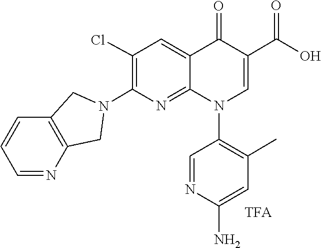 [SMILES: Cc1cc(N)ncc1-n1cc(C(=O)O)c(=O)c2cc(Cl)c(N3Cc4cccnc4C3)nc21] | ++++ |
| 109 | 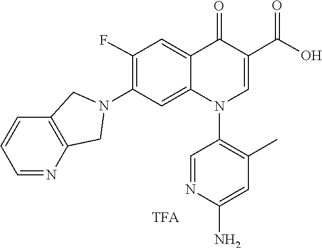 [SMILES: Cc1cc(N)ncc1-n1cc(C(=O)O)c(=O)c2cc(F)c(N3Cc4cccnc4C3)cc21] | ++++ |
| 110 | 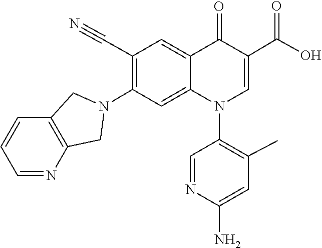 [SMILES: Cc1cc(N)ncc1-n1cc(C(=O)O)c(=O)c2cc(C#N)c(N3Cc4cccnc4C3)cc21] | ++++ |
| 111 | 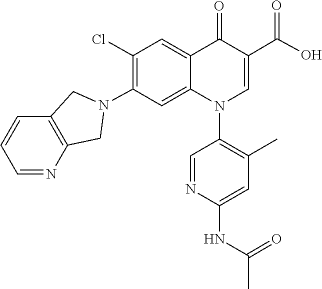 [SMILES: CC(=O)Nc1cc(C)c(-n2cc(C(=O)O)c(=O)c3cc(Cl)c(N4Cc5cccnc5C4)cc32)cn1] | ++++ |
| 112 | 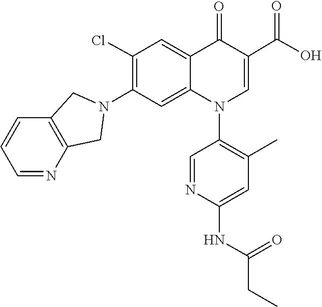 [SMILES: CCC(=O)Nc1cc(C)c(-n2cc(C(=O)O)c(=O)c3cc(Cl)c(N4Cc5cccnc5C4)cc32)cn1] | ++++ |
| 113 | 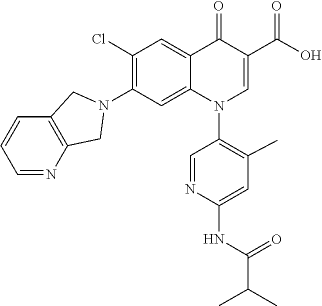 [SMILES: Cc1cc(NC(=O)C(C)C)ncc1-n1cc(C(=O)O)c(=O)c2cc(Cl)c(N3Cc4cccnc4C3)cc21] | ++++ |
| 114 | 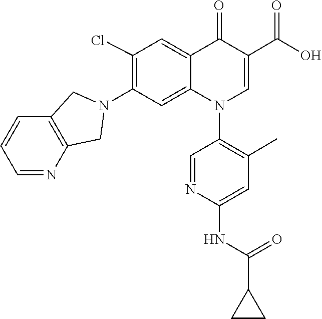 [SMILES: Cc1cc(NC(=O)C2CC2)ncc1-n1cc(C(=O)O)c(=O)c2cc(Cl)c(N3Cc4cccnc4C3)cc21] | ++++ |
| 115 | 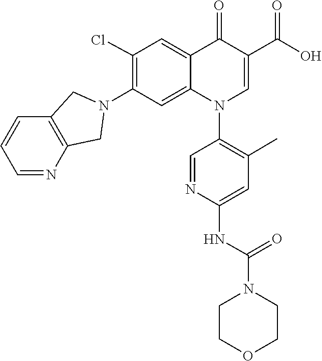 [SMILES: Cc1cc(NC(=O)N2CCOCC2)ncc1-n1cc(C(=O)O)c(=O)c2cc(Cl)c(N3Cc4cccnc4C3)cc21] | ++++ |
| 116 | 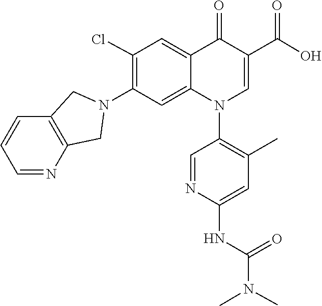 [SMILES: Cc1cc(NC(=O)N(C)C)ncc1-n1cc(C(=O)O)c(=O)c2cc(Cl)c(N3Cc4cccnc4C3)cc21] | ++++ |
| 117 | 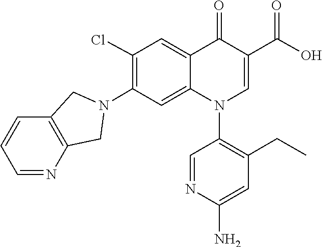 [SMILES: CCc1cc(N)ncc1-n1cc(C(=O)O)c(=O)c2cc(Cl)c(N3Cc4cccnc4C3)cc21] | ++++ |
| 118 | 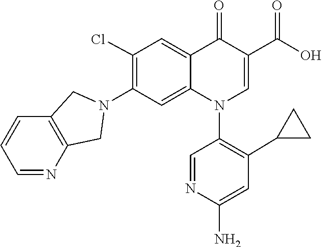 [SMILES: Nc1cc(C2CC2)c(-n2cc(C(=O)O)c(=O)c3cc(Cl)c(N4Cc5cccnc5C4)cc32)cn1] | ++++ |
| 119 | 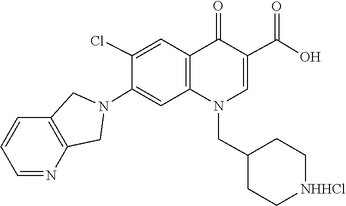 | ++++ |
| 120 | 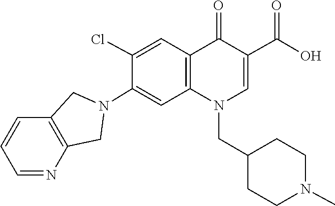 [SMILES: CN1CCC(Cn2cc(C(=O)O)c(=O)c3cc(Cl)c(N4Cc5cccnc5C4)cc32)CC1] | ++++ |
| 121 | 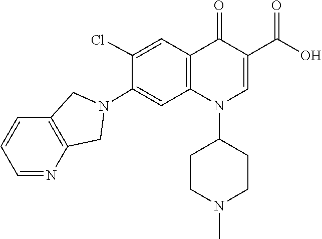 [SMILES: CN1CCC(n2cc(C(=O)O)c(=O)c3cc(Cl)c(N4Cc5cccnc5C4)cc32)CC1] | ++++ |
| 122 | 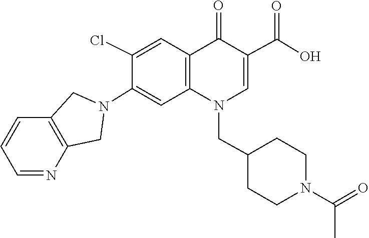 [SMILES: CC(=O)N1CCC(Cn2cc(C(=O)O)c(=O)c3cc(Cl)c(N4Cc5cccnc5C4)cc32)CC1] | ++++ |
| 123 | 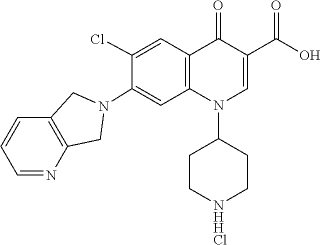 | ++++ |
| 124 | 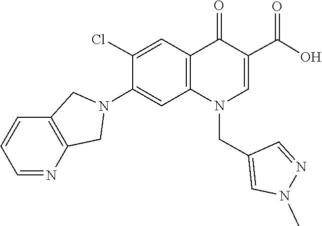 [SMILES: Cn1cc(Cn2cc(C(=O)O)c(=O)c3cc(Cl)c(N4Cc5cccnc5C4)cc32)cn1] | ++++ |
| 125 | 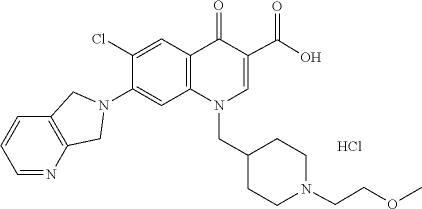 [SMILES: COCCN1CCC(Cn2cc(C(=O)O)c(=O)c3cc(Cl)c(N4Cc5cccnc5C4)cc32)CC1 ; Cl] | ++++ |
| 126 | 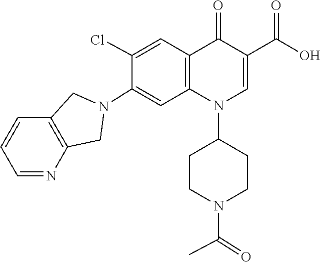 [SMILES: CC(=O)N1CCC(n2cc(C(=O)O)c(=O)c3cc(Cl)c(N4Cc5cccnc5C4)cc32)CC1] | ++++ |
| 127 | 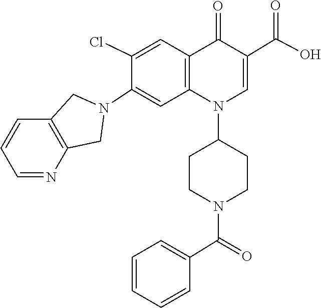 [SMILES: O=C(O)c1cn(C2CCN(C(=O)c3ccccc3)CC2)c2cc(N3Cc4cccnc4C3)c(Cl)cc2c1=O] | ++++ |
| 128 | 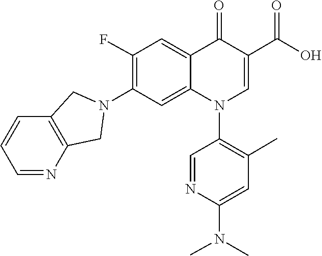 [SMILES: Cc1cc(N(C)C)ncc1-n1cc(C(=O)O)c(=O)c2cc(F)c(N3Cc4cccnc4C3)cc21] | ++++ |
| 129 | 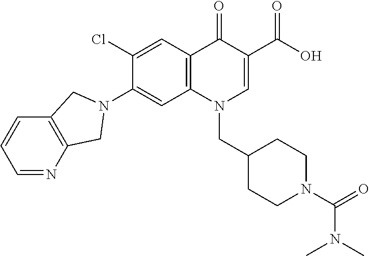 [SMILES: CN(C)C(=O)N1CCC(Cn2cc(C(=O)O)c(=O)c3cc(Cl)c(N4Cc5cccnc5C4)cc32)CC1] | ++++ |
| 130 | 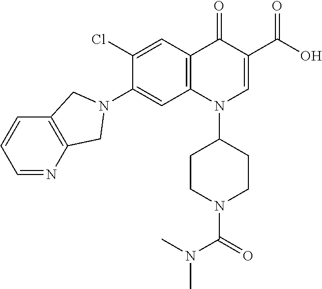 [SMILES: CN(C)C(=O)N1CCC(n2cc(C(=O)O)c(=O)c3cc(Cl)c(N4Cc5cccnc5C4)cc32)CC1] | ++++ |
| 131 | 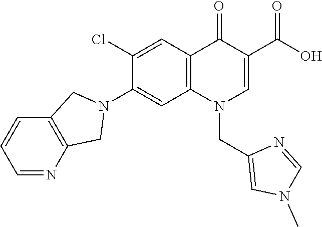 [SMILES: Cn1cnc(Cn2cc(C(=O)O)c(=O)c3cc(Cl)c(N4Cc5cccnc5C4)cc32)c1] | ++++ |
| 132 | 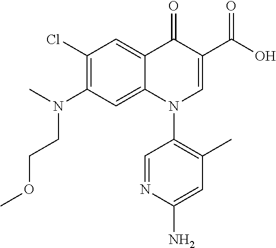 [SMILES: COCCN(C)c1cc2c(cc1Cl)c(=O)c(C(=O)O)cn2-c1cnc(N)cc1C] | ++++ |
| 133 | 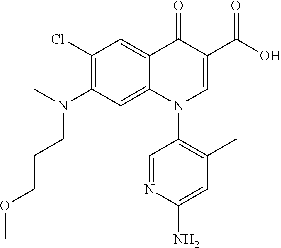 [SMILES: COCCCN(C)c1cc2c(cc1Cl)c(=O)c(C(=O)O)cn2-c1cnc(N)cc1C] | ++++ |
| 134 | 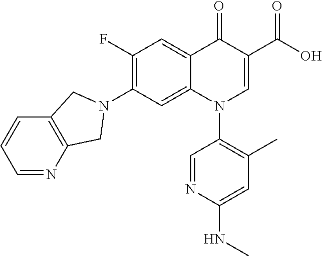 [SMILES: CNc1cc(C)c(-n2cc(C(=O)O)c(=O)c3cc(F)c(N4Cc5cccnc5C4)cc32)cn1] | ++++ |
| 135 | 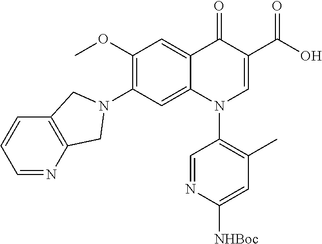 [SMILES: COc1cc2c(=O)c(C(=O)O)cn(-c3cnc(NC(=O)OC(C)(C)C)cc3C)c2cc1N1Cc2cccnc2C1] | ++++ |
| 136 | 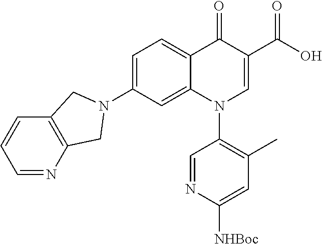 [SMILES: Cc1cc(NC(=O)OC(C)(C)C)ncc1-n1cc(C(=O)O)c(=O)c2ccc(N3Cc4cccnc4C3)cc21] | ++++ |
| 137 | 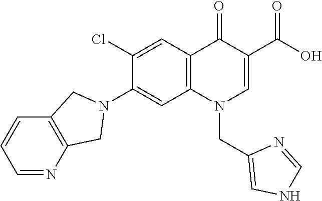 [SMILES: O=C(O)c1cn(Cc2c[nH]cn2)c2cc(N3Cc4cccnc4C3)c(Cl)cc2c1=O] | ++++ |
| 138 | 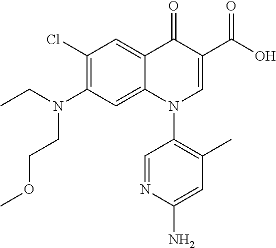 [SMILES: CCN(CCOC)c1cc2c(cc1Cl)c(=O)c(C(=O)O)cn2-c1cnc(N)cc1C] | ++++ |
| 139 | 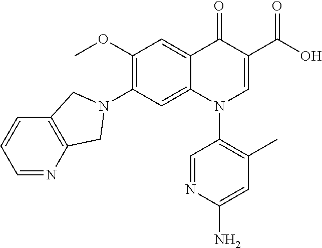 [SMILES: COc1cc2c(=O)c(C(=O)O)cn(-c3cnc(N)cc3C)c2cc1N1Cc2cccnc2C1] | ++++ |
| 140 | 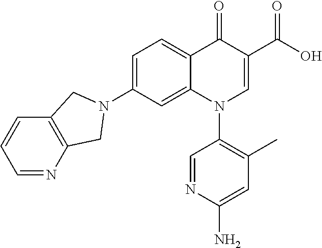 [SMILES: Cc1cc(N)ncc1-n1cc(C(=O)O)c(=O)c2ccc(N3Cc4cccnc4C3)cc21] | ++++ |
| 141 | 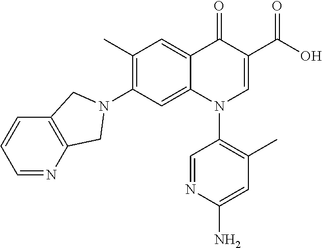 [SMILES: Cc1cc2c(=O)c(C(=O)O)cn(-c3cnc(N)cc3C)c2cc1N1Cc2cccnc2C1] | ++++ |
| 142 | 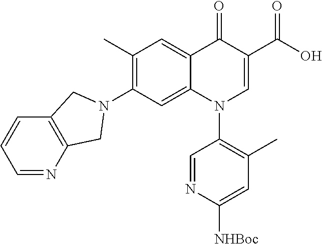 [SMILES: Cc1cc2c(=O)c(C(=O)O)cn(-c3cnc(NC(=O)OC(C)(C)C)cc3C)c2cc1N1Cc2cccnc2C1] | ++++ |
| 143 | 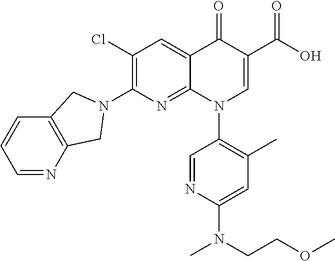 [SMILES: COCCN(C)c1cc(C)c(-n2cc(C(=O)O)c(=O)c3cc(Cl)c(N4Cc5cccnc5C4)nc32)cn1] | ++++ |
| 144 | 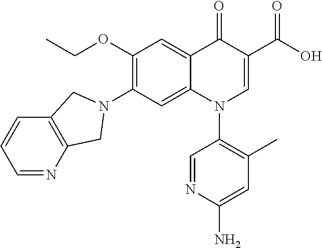 [SMILES: CCOc1cc2c(=O)c(C(=O)O)cn(-c3cnc(N)cc3C)c2cc1N1Cc2cccnc2C1] | ++++ |
| 145 | 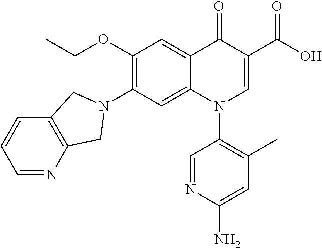 [SMILES: CCOc1cc2c(=O)c(C(=O)O)cn(-c3cnc(N)cc3C)c2cc1N1Cc2cccnc2C1] | ++++ |
| 146 | 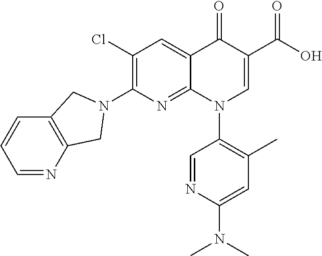 [SMILES: Cc1cc(N(C)C)ncc1-n1cc(C(=O)O)c(=O)c2cc(Cl)c(N3Cc4cccnc4C3)nc21] | ++++ |
| 147 | 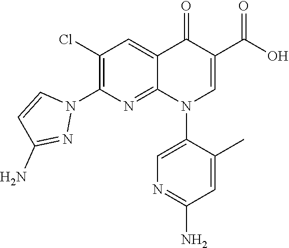 [SMILES: Cc1cc(N)ncc1-n1cc(C(=O)O)c(=O)c2cc(Cl)c(-n3ccc(N)n3)nc21] | ++++ |
| 148 | 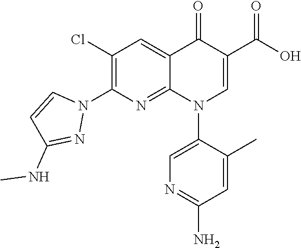 [SMILES: CNc1ccn(-c2nc3c(cc2Cl)c(=O)c(C(=O)O)cn3-c2cnc(N)cc2C)n1] | ++++ |
| 149 | 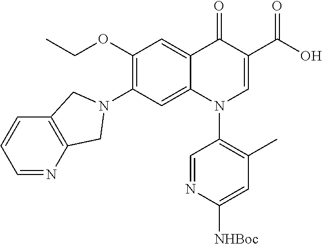 [SMILES: CCOc1cc2c(=O)c(C(=O)O)cn(-c3cnc(NC(=O)OC(C)(C)C)cc3C)c2cc1N1Cc2cccnc2C1] | ++++ |
| 150 | 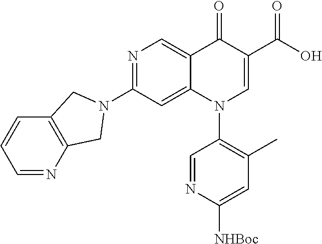 [SMILES: Cc1cc(NC(=O)OC(C)(C)C)ncc1-n1cc(C(=O)O)c(=O)c2cnc(N3Cc4cccnc4C3)cc21] | ++++ |
| 151 | 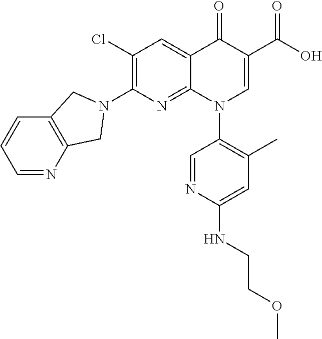 [SMILES: COCCNc1cc(C)c(-n2cc(C(=O)O)c(=O)c3cc(Cl)c(N4Cc5cccnc5C4)nc32)cn1] | ++++ |
| 152 | 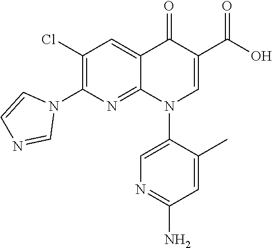 [SMILES: Cc1cc(N)ncc1-n1cc(C(=O)O)c(=O)c2cc(Cl)c(-n3ccnc3)nc21] | ++++ |
| 153 | 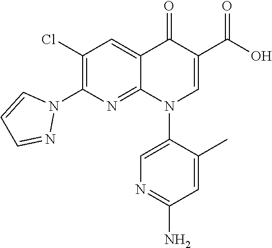 [SMILES: Cc1cc(N)ncc1-n1cc(C(=O)O)c(=O)c2cc(Cl)c(-n3cccn3)nc21] | ++++ |
| 154 | 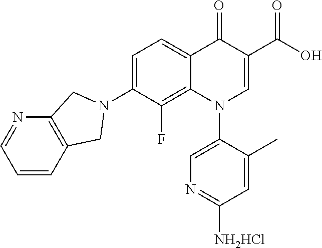 | ++++ |
| 155 | 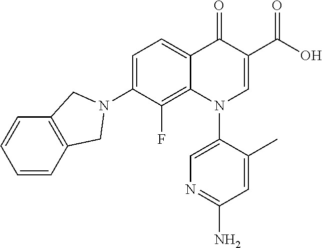 [SMILES: Cc1cc(N)ncc1-n1cc(C(=O)O)c(=O)c2ccc(N3Cc4ccccc4C3)c(F)c21] | ++++ |
| 156 | 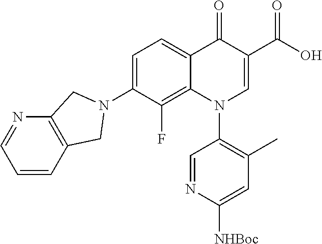 [SMILES: Cc1cc(NC(=O)OC(C)(C)C)ncc1-n1cc(C(=O)O)c(=O)c2ccc(N3Cc4cccnc4C3)c(F)c21] | ++++ |
| 157 | 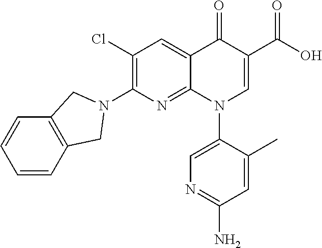 [SMILES: Cc1cc(N)ncc1-n1cc(C(=O)O)c(=O)c2cc(Cl)c(N3Cc4ccccc4C3)nc21] | ++++ |
| 158 | 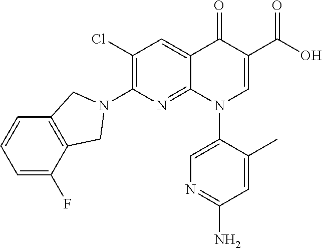 [SMILES: Cc1cc(N)ncc1-n1cc(C(=O)O)c(=O)c2cc(Cl)c(N3Cc4cccc(F)c4C3)nc21] | ++++ |
| 159 | 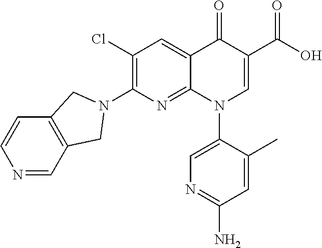 [SMILES: Cc1cc(N)ncc1-n1cc(C(=O)O)c(=O)c2cc(Cl)c(N3Cc4ccncc4C3)nc21] | ++++ |
| 160 | 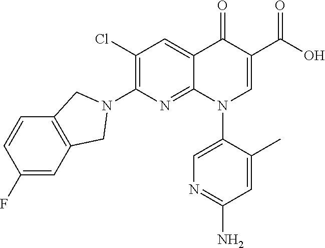 [SMILES: Cc1cc(N)ncc1-n1cc(C(=O)O)c(=O)c2cc(Cl)c(N3Cc4ccc(F)cc4C3)nc21] | ++++ |
| 161 | 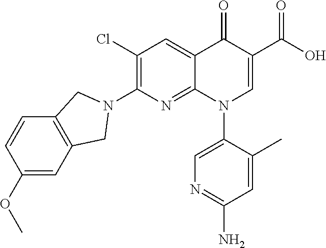 [SMILES: COc1ccc2c(c1)CN(c1nc3c(cc1Cl)c(=O)c(C(=O)O)cn3-c1cnc(N)cc1C)C2] | ++++ |
| 162 | 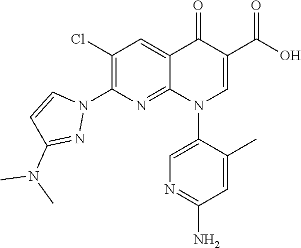 [SMILES: Cc1cc(N)ncc1-n1cc(C(=O)O)c(=O)c2cc(Cl)c(-n3ccc(N(C)C)n3)nc21] | ++++ |
| 163 | 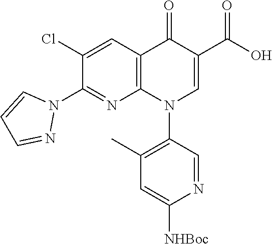 [SMILES: Cc1cc(NC(=O)OC(C)(C)C)ncc1-n1cc(C(=O)O)c(=O)c2cc(Cl)c(-n3cccn3)nc21] | ++++ |
| 164 | 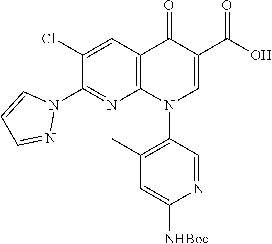 [SMILES: Cc1cc(NC(=O)OC(C)(C)C)ncc1-n1cc(C(=O)O)c(=O)c2cc(Cl)c(-n3cccn3)nc21] | ++++ |
| 165 | 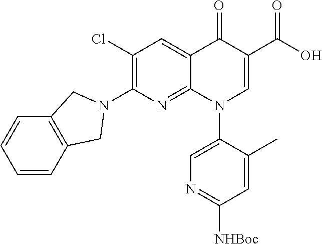 [SMILES: Cc1cc(NC(=O)OC(C)(C)C)ncc1-n1cc(C(=O)O)c(=O)c2cc(Cl)c(N3Cc4ccccc4C3)nc21] | ++++ |
| 166 | 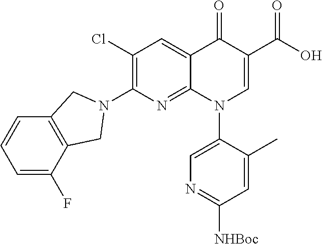 [SMILES: Cc1cc(NC(=O)OC(C)(C)C)ncc1-n1cc(C(=O)O)c(=O)c2cc(Cl)c(N3Cc4cccc(F)c4C3)nc21] | ++++ |
| 167 | 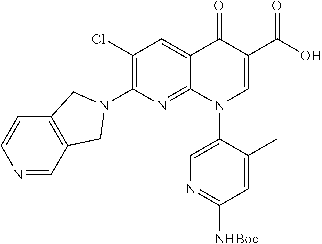 [SMILES: Cc1cc(NC(=O)OC(C)(C)C)ncc1-n1cc(C(=O)O)c(=O)c2cc(Cl)c(N3Cc4ccncc4C3)nc21] | ++++ |
| 168 | 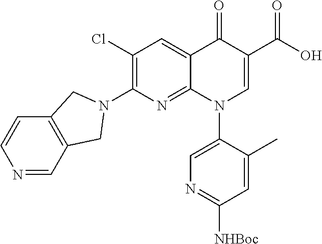 [SMILES: Cc1cc(NC(=O)OC(C)(C)C)ncc1-n1cc(C(=O)O)c(=O)c2cc(Cl)c(N3Cc4ccncc4C3)nc21] | ++++ |
| 169 | 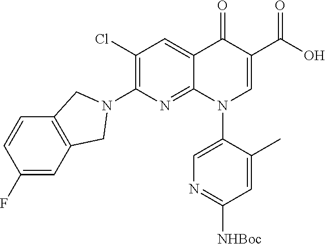 [SMILES: Cc1cc(NC(=O)OC(C)(C)C)ncc1-n1cc(C(=O)O)c(=O)c2cc(Cl)c(N3Cc4ccc(F)cc4C3)nc21] | ++++ |
| 170 | 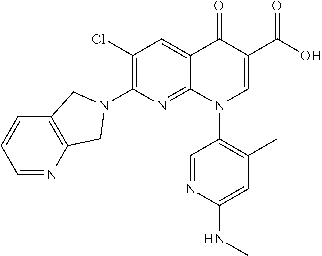 [SMILES: CNc1cc(C)c(-n2cc(C(=O)O)c(=O)c3cc(Cl)c(N4Cc5cccnc5C4)nc32)cn1] | ++++ |
| 171 | 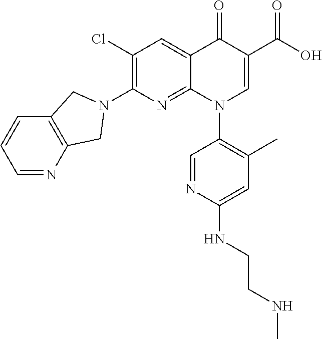 [SMILES: CNCCNc1cc(C)c(-n2cc(C(=O)O)c(=O)c3cc(Cl)c(N4Cc5cccnc5C4)nc32)cn1] | ++++ |
| 172 | 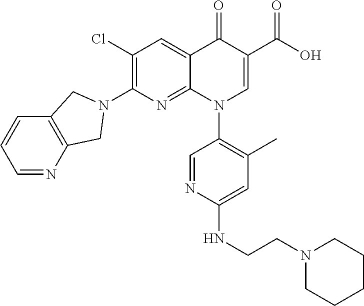 [SMILES: Cc1cc(NCCN2CCCCC2)ncc1-n1cc(C(=O)O)c(=O)c2cc(Cl)c(N3Cc4cccnc4C3)nc21] | ++++ |
| 173 | 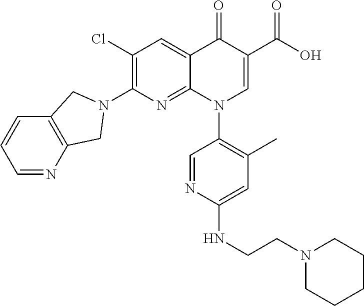 [SMILES: Cc1cc(NCCN2CCCCC2)ncc1-n1cc(C(=O)O)c(=O)c2cc(Cl)c(N3Cc4cccnc4C3)nc21] | ++++ |
| 174 | 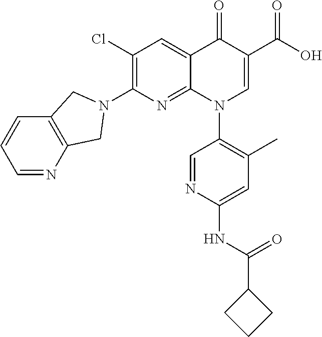 [SMILES: Cc1cc(NC(=O)C2CCC2)ncc1-n1cc(C(=O)O)c(=O)c2cc(Cl)c(N3Cc4cccnc4C3)nc21] | ++++ |
| 175 | 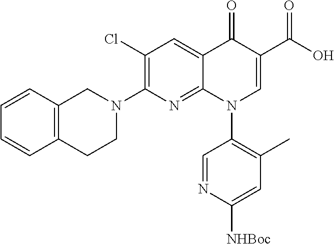 [SMILES: Cc1cc(NC(=O)OC(C)(C)C)ncc1-n1cc(C(=O)O)c(=O)c2cc(Cl)c(N3CCc4ccccc4C3)nc21] | ++++ |
| 176 | 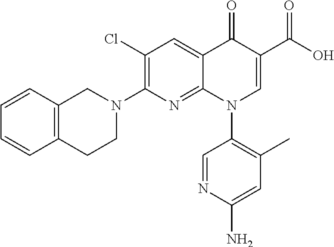 [SMILES: Cc1cc(N)ncc1-n1cc(C(=O)O)c(=O)c2cc(Cl)c(N3CCc4ccccc4C3)nc21] | ++++ |
| 177 | 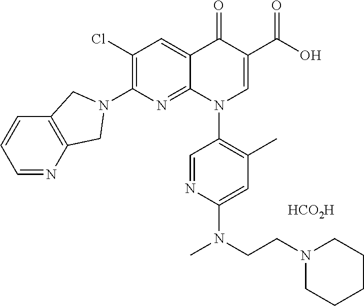 [SMILES: Cc1cc(N(C)CCN2CCCCC2)ncc1-n1cc(C(=O)O)c(=O)c2cc(Cl)c(N3Cc4cccnc4C3)nc21 ; "HCO2H"] | ++++ |
| 178 | 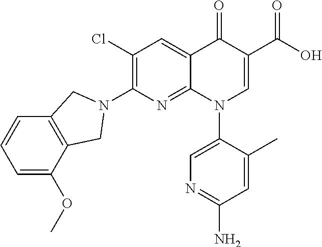 [SMILES: COc1cccc2c1CN(c1nc3c(cc1Cl)c(=O)c(C(=O)O)cn3-c1cnc(N)cc1C)C2] | ++++ |
| 179 | 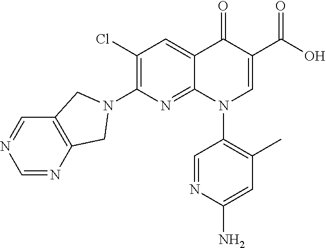 [SMILES: Cc1cc(N)ncc1-n1cc(C(=O)O)c(=O)c2cc(Cl)c(N3Cc4cncnc4C3)nc21] | ++++ |
| 180 | 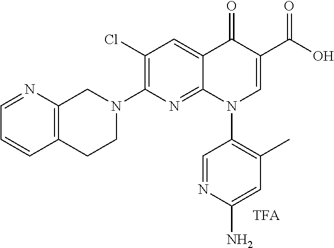 [SMILES: Cc1cc(N)ncc1-n1cc(C(=O)O)c(=O)c2cc(Cl)c(N3CCc4cccnc4C3)nc21] | ++++ |
| 181 | 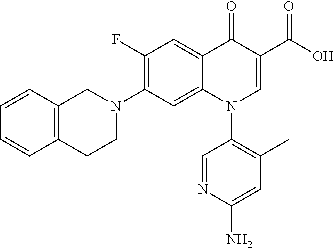 [SMILES: Cc1cc(N)ncc1-n1cc(C(=O)O)c(=O)c2cc(F)c(N3CCc4ccccc4C3)cc21] | ++++ |
| 182 | 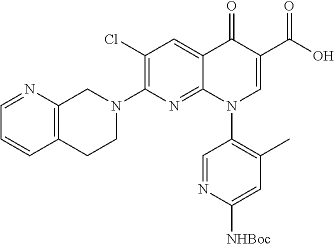 [SMILES: Cc1cc(NC(=O)OC(C)(C)C)ncc1-n1cc(C(=O)O)c(=O)c2cc(Cl)c(N3CCc4cccnc4C3)nc21] | ++++ |
| 183 | 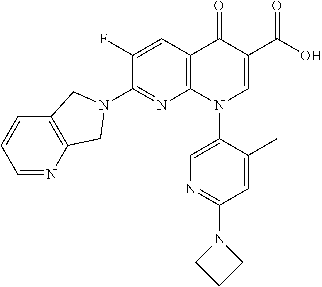 [SMILES: Cc1cc(N2CCC2)ncc1-n1cc(C(=O)O)c(=O)c2cc(F)c(N3Cc4cccnc4C3)nc21] | ++++ |
| 184 | 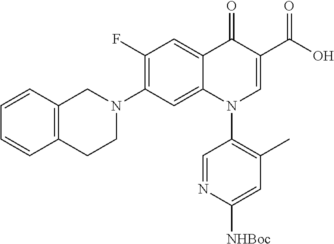 [SMILES: Cc1cc(NC(=O)OC(C)(C)C)ncc1-n1cc(C(=O)O)c(=O)c2cc(F)c(N3CCc4ccccc4C3)cc21] | ++++ |
| 185 | 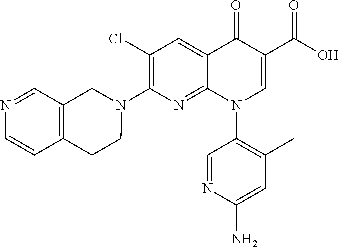 [SMILES: Cc1cc(N)ncc1-n1cc(C(=O)O)c(=O)c2cc(Cl)c(N3CCc4ccncc4C3)nc21] | ++++ |
| 186 | 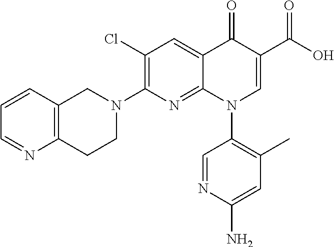 [SMILES: Cc1cc(N)ncc1-n1cc(C(=O)O)c(=O)c2cc(Cl)c(N3CCc4ncccc4C3)nc21] | ++++ |
| 187 | 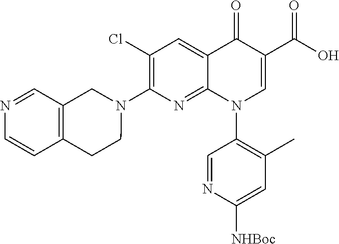 [SMILES: Cc1cc(NC(=O)OC(C)(C)C)ncc1-n1cc(C(=O)O)c(=O)c2cc(Cl)c(N3CCc4ccncc4C3)nc21] | ++++ |
| 188 | 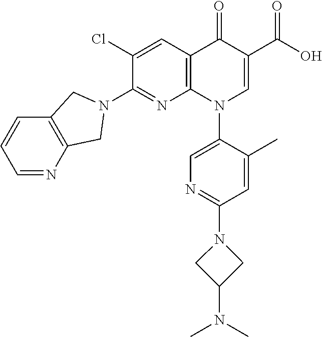 [SMILES: Cc1cc(N2CC(N(C)C)C2)ncc1-n1cc(C(=O)O)c(=O)c2cc(Cl)c(N3Cc4cccnc4C3)nc21] | ++++ |
| 189 | 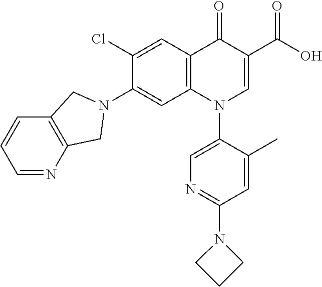 [SMILES: Cc1cc(N2CCC2)ncc1-n1cc(C(=O)O)c(=O)c2cc(Cl)c(N3Cc4cccnc4C3)cc21] | ++++ |
| 190 | 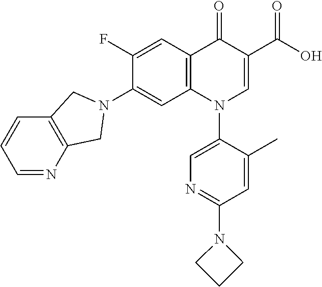 [SMILES: Cc1cc(N2CCC2)ncc1-n1cc(C(=O)O)c(=O)c2cc(F)c(N3Cc4cccnc4C3)cc21] | ++++ |
| 191 | 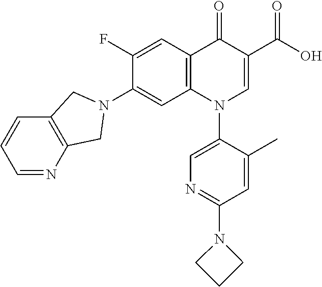 [SMILES: Cc1cc(N2CCC2)ncc1-n1cc(C(=O)O)c(=O)c2cc(F)c(N3Cc4cccnc4C3)cc21] | ++++ |
| 192 | 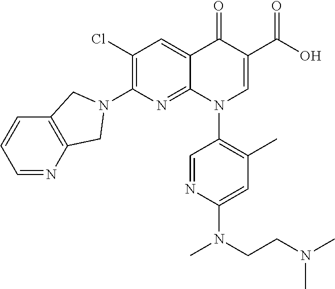 [SMILES: Cc1cc(N(C)CCN(C)C)ncc1-n1cc(C(=O)O)c(=O)c2cc(Cl)c(N3Cc4cccnc4C3)nc21] | ++++ |
| 193 | 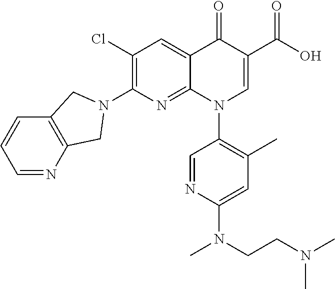 [SMILES: Cc1cc(N(C)CCN(C)C)ncc1-n1cc(C(=O)O)c(=O)c2cc(Cl)c(N3Cc4cccnc4C3)nc21] | ++++ |
| 194 | 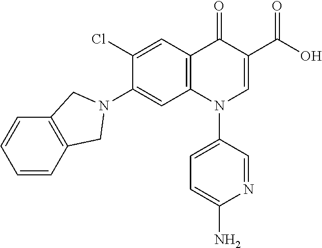 [SMILES: Nc1ccc(-n2cc(C(=O)O)c(=O)c3cc(Cl)c(N4Cc5ccccc5C4)cc32)cn1] | ++++ |
| 195 | 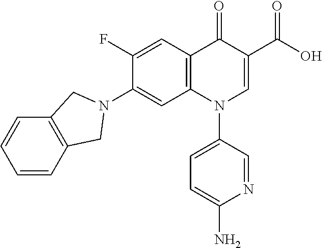 [SMILES: Nc1ccc(-n2cc(C(=O)O)c(=O)c3cc(F)c(N4Cc5ccccc5C4)cc32)cn1] | ++++ |
| 196 | 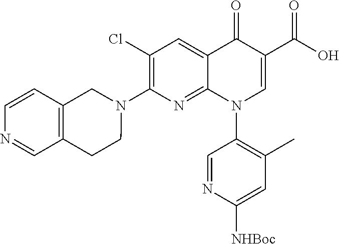 [SMILES: Cc1cc(NC(=O)OC(C)(C)C)ncc1-n1cc(C(=O)O)c(=O)c2cc(Cl)c(N3CCc4cnccc4C3)nc21] | ++++ |
| 197 | 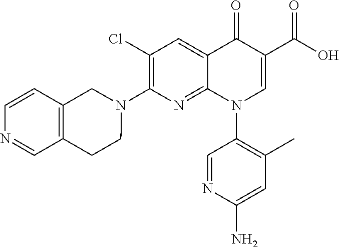 [SMILES: Cc1cc(N)ncc1-n1cc(C(=O)O)c(=O)c2cc(Cl)c(N3CCc4cnccc4C3)nc21] | ++++ |
| 198 | 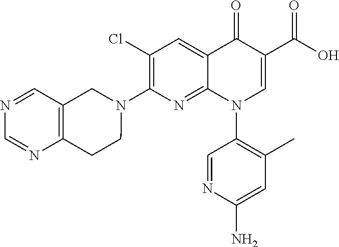 [SMILES: Cc1cc(N)ncc1-n1cc(C(=O)O)c(=O)c2cc(Cl)c(N3CCc4ncncc4C3)nc21] | ++++ |
| 199 | 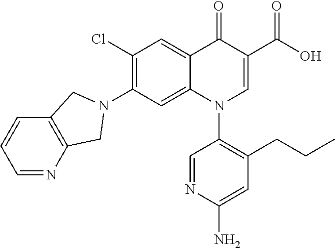 [SMILES: CCCc1cc(N)ncc1-n1cc(C(=O)O)c(=O)c2cc(Cl)c(N3Cc4cccnc4C3)cc21] | ++++ |
| 200 | 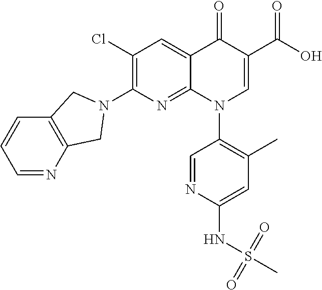 [SMILES: Cc1cc(NS(C)(=O)=O)ncc1-n1cc(C(=O)O)c(=O)c2cc(Cl)c(N3Cc4cccnc4C3)nc21] | ++++ |
| 201 | 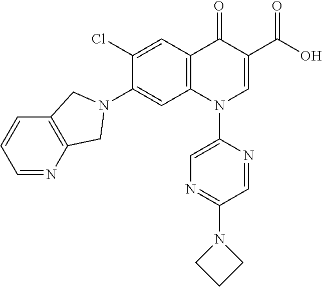 [SMILES: O=C(O)c1cn(-c2cnc(N3CCC3)cn2)c2cc(N3Cc4cccnc4C3)c(Cl)cc2c1=O] | ++++ |
| 202 | 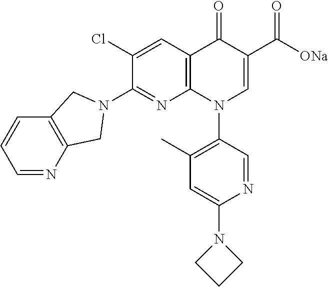 | ++++ |
| 203 | 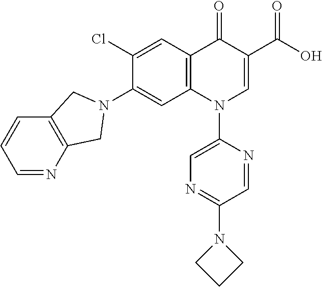 [SMILES: O=C(O)c1cn(-c2cnc(N3CCC3)cn2)c2cc(N3Cc4cccnc4C3)c(Cl)cc2c1=O] | ++++ |

What is claimed is:

1. A compound represented by Formula (I): 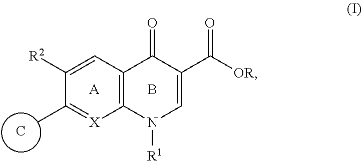
or a pharmaceutically acceptable salt thereof, wherein:
R¹ is C₁₋₆ alkyl, C₃₋₇ cycloaliphatic, phenyl, 5 to 6-membered heteroaryl or 5 to 12-membered heterocyclyl, each optionally substituted with one or more R¹⁰;
each R¹⁰ is independently halo, —OR^{1a}, —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, —SO₂N(R^{1a})₂, C₁₋₄ alkyl, C₃₋₆ cycloaliphatic, phenyl, 5 to 6-membered heteroaryl or 3 to 6-membered heterocyclyl, wherein the C₁₋₄ alkyl, C₃₋₆ cycloaliphatic, phenyl, 5 to 6-membered heteroaryl and 3 to 6-membered heterocyclyl are each optionally substituted by one or more R¹⁵;
each R¹⁵ is independently halo, —OR^{1a}, —N(R^{1a})₂, —N(R^{1a})C(O)OR^{1a}, —C(O)R^{1a}, —N(R^{1a})C(O)OR^{1a}, —N(R^{1a})C(O)N(R^{1a})₂, —N(R^{1a})SO₂R^{1a}, —C(O)N(R^{1a})₂, C₁₋₄ alkyl or C₁₋₄ haloalkyl, wherein the C₁₋₄ alkyl is optionally substituted by OR^{1a};
each R^{1a} is independently H, C₁₋₄ alkyl, phenyl, C₃₋₇ cycloaliphatic or 6 to 7-membered heterocyclyl, wherein the C₁₋₄ alkyl, phenyl, C₃₋₇ cycloaliphatic and 6 to 7-membered heterocyclyl are each optionally substituted with one or more R^{1b} or two R^{1a} taken together with the nitrogen atom to which they are bonded form a 5-6 membered heterocyclyl optionally substituted with one or more R¹⁵;
each R^{1b} is independently halo, —OH, —OCH₃, halomethoxy, methyl, halomethyl, —NH₂, —N(H)CH₃, —N(CH₃)₂, phenyl or 4 to 6-membered hetercyclyl;
R² is H, halo, CN, —OR^{2a} or C₁₋₆ alkyl optionally substituted with one or more selected from halo, —OR^{2a}, —NHR^{2a} or —N(R^{2a})₂;
each R^{2a} is independently H or C₁₋₆ alkyl optionally substituted with one or more R^{2b};
each R^{2b} is independently halo, —OH, —O—C₁₋₄alkyl, —O—C₁₋₄haloalkyl, —NH₂, —N(H)—C₁₋₄alkyl or —N(C₁₋₄alkyl)₂;
ring C is indazolyl, isoindolinyl, pyrazolyl, dihydro-pyrrolopyrazinyl, dihydro-pyrrolopyridinyl, dihydro-pyrrolopyridazinyl, tetrahydronaphthyridinyl, tetrahydroisoquinolinyl, tetrahydropyrido[4,3-d]pyrimidinyl, or dihydro-pyrrolopyrimidinyl, each optionally substituted with one or more R³;
each R³ is independently H, halo, —OR^{3a}, —N(R^{3a})₂, —N(R^{3a})C(O)R^{3a}, —N(R^{3a})C(O)OR^{3a}, —N(R^{3a})C(O)N(R^{3a})₂, —N(R^{3a})SO₂R^{3a}, —C(O)R^{3a}, —C(O)N(R^{3a})₂, oxo, C₁₋₄ alkyl, phenyl or 5 to 6-membered heteroaryl, wherein the C₁₋₄ alkyl, phenyl and 5 to 6-membered heteroaryl are each optionally substituted by one or more R³⁰;
each R³⁰ is independently halo, —OR^{3a}, C₁₋₄ alkyl, C₁₋₄ haloalkyl, phenyl or 5 to 6-membered heteroaryl;
each R^{3a} is independently H, C₁₋₄ alkyl, phenyl or 5 to 6-membered heteroaryl, wherein the C₁₋₄ alkyl, phenyl and 5 to 6-membered heteroaryl are each optionally substituted with one or more R^{3b};
each R^{3b} is independently Br, Cl, F, —OH, —OCH₃, —OCH₂F, —OCH₂CH₃, —OCH₂CF₃, —OCH₂CH₂F, —NH₂, —N(H)CH₃, —N(CH₃)₂, —CH₃, —CH₂CH₃, —CH₂F, —CHF₂, —CF₃, phenyl or 4 to 6-membered heterocyclyl; and
X is N or CR⁴;
R is H or C₁-C₄ alkyl;
R⁴ is H or halo, and
with the provisos that:
when R² is halo and ring C is an isoindolinyl, then R¹ is: i) C₁₋₆ alkyl substituted with C₃₋₆ cycloaliphatic, 5 to 6-membered heteroaryl or 3 to 6-membered heterocyclyl, wherein the C₃₋₆ cycloaliphatic, 5 to 6-membered heteroaryl and 3 to 6-membered heterocyclyl are each optionally substituted by one or more R¹⁵; ii) 5 to 6-membered heteroaryl optionally substituted with one or more R¹⁰; or iii) 5 to 12-membered heterocyclyl optionally substituted with one or more R¹⁰;
when R² is halo and ring C is a dihydro-pyrrolopyridinyl, dihydro-pyrrolopyrimidinyl or dihydro-pyrrolopyrazinyl, then R¹ is: i) C₁₋₆ alkyl substituted with C₃₋₆ cycloaliphatic, phenyl, 5 to 6-membered heteroaryl or 3 to 6-membered heterocyclyl, wherein the C₃₋₆ cycloaliphatic, phenyl, 5 to 6-membered heteroaryl and 3 to 6-membered heterocyclyl are each optionally substituted by one or more R¹⁵; ii) 5 to 6-membered heteroaryl optionally substituted with one or more R¹⁰; iii) 5 to 12-membered heterocyclyl optionally substituted with one or more R¹⁰; or iv) phenyl substituted with one or more R¹⁰ provided that at least one R¹⁰ is other than F;
when ring C is pyrazolyl, and R² is halo, then i) R¹ is: C₁₋₆ alkyl substituted with C₃₋₆ cycloaliphatic, phenyl, 5 to 6-membered heteroaryl or 3 to 6-membered heterocyclyl, wherein the C₃₋₆ cycloaliphatic, phenyl, 5 to 6-membered heteroaryl and 3 to 6-membered heterocyclyl are each optionally substituted by one or more R¹⁰; or ii) R¹ is C₄₋₇ cycloaliphatic, phenyl, 5 to 6-membered heteroaryl or 5 to 12-membered heterocyclyl, each optionally substituted with one or more R¹⁰, provided that when R¹ is phenyl substituted by one or more R¹⁰, then R³ is other than pyridyl and at least one R¹⁰ is —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, C₁₋₄ alkyl, C₃₋₆ cycloaliphatic, phenyl, 5 to 6-membered heteroaryl or 3 to 6-membered heterocyclyl, wherein the C₁₋₄ alkyl, C₃₋₆ cycloaliphatic, phenyl, 5 to 6-membered heteroaryl and 3 to 6-membered heterocyclyl are each optionally substituted by one or more R¹⁵; and
when ring C is indazolyl, R¹ is phenyl optionally substituted with one or more R¹⁰ and R² is halo, then the indazolyl is optionally substituted with one or more R³ wherein each R³ is independently H, halo, —N(R^{3a})₂, —N(R^{3a})C(O)R^{3a}, —N(R^{3a})C(O)OR^{3a}, —N(R^{3a})C(O)N(R^{3a})₂, —N(R^{3a})SO₂R^{3a}, —C(O)R^{3a}, —C(O)N(R^{3a})₂, oxo, C₁₋₄ alkyl, phenyl or 5 to 6-membered heteroaryl, wherein the C₁₋₄ alkyl, phenyl and 5 to 6-membered heteroaryl are each optionally substituted by one or more R³⁰.

2. The compound of claim 1, or a pharmaceutically acceptable salt thereof, wherein:
R is H;
each R¹⁰ is independently halo, —OR^{1a}, —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, C₁₋₄ alkyl, C₃₋₆ cycloaliphatic, phenyl, 5 to 6-membered heteroaryl or 3 to 6-membered heterocyclyl, wherein the C₁₋₄ alkyl, C₃₋₆ cycloaliphatic, phenyl, 5 to 6-membered heteroaryl and 3 to 6-membered heterocyclyl are each optionally substituted by one or more R¹⁵;
each R¹⁵ is independently halo, —OR^{1a}, —N(R^{1a})₂, —N(R^{1a})C(O)OR^{1a}, —C(O)R^{1a}, —N(R^{1a})C(O)OR^{1a}, —N(R^{1a})C(O)N(R^{1a})₂, —N(R^{1a})SO₂R^{1a}, —C(O)N(R^{1a})₂, C₁₋₄ alkyl or C₁₋₄ haloalkyl;
each R^{1a} is independently H, C₁₋₄ alkyl, phenyl, C₃₋₇ cycloaliphatic or 6 to 7-membered heterocyclyl, wherein the C₁₋₄ alkyl, phenyl, C₃₋₇ cycloaliphatic and 6 to 7-membered heterocyclyl are each optionally substituted with one or more R^{1b}; and
ring C is indazolyl, isoindolinyl, pyrazolyl, dihydro-pyrrolopyrazinyl, dihydro-pyrrolopyridinyl, dihydro-pyrrolopyridazinyl or dihydro-pyrrolopyrimidinyl, each optionally substituted with one or more R³.

3. The compound of claim 1 or 2, or a pharmaceutically acceptable salt thereof, wherein:
R¹ is C₁₋₄ alkyl, C₃₋₆ cycloaliphatic, phenyl, 6-membered heteroaryl or 6-membered heterocyclyl, each optionally substituted with one or more R¹⁰;
each R¹⁰ is independently halo, —OR^{1a}, —N(R^{1a})₂, —N(R^{1a})C(O)R^{1a}, —C(O)R^{1a}, —N(R^{1a})C(O)OR^{1a}, —N(R^{1a})C(O)N(R^{1a})₂, —N(R^{1a})SO₂R^{1a}, —C(O)N(R^{1a})₂, C₁₋₃ alkyl, C₃₋₆ cycloaliphatic, phenyl, 5 to 6-membered heteroaryl or 4 to 6-membered heterocyclyl, wherein the C₁₋₃ alkyl, C₃₋₆ cycloaliphatic, phenyl, 5 to 6-membered heteroaryl and 4- to 6-membered heterocyclyl are each optionally substituted by one or more R⁵;
each R¹⁵ is independently halo, —OR^{1a}, —N(R^{1a})₂, —N(R^{1a})C(O)OR^{1a}, —C(O)R^{1a}, —C(O)N(R^{1a})₂ or —CH₃;
each R^{1a} is independently H, C₁₋₄ alkyl, phenyl, cyclopropyl or 6-membered heterocyclyl, wherein the C₁₋₄ alkyl, phenyl, cyclopropyl and 6-membered heterocyclyl are each optionally substituted with one or more R^{1b};
R² is H, halo, CN, OR^{2a} or C₁₋₆ alkyl;
R^{2a} is H or C₁₋₆ alkyl;
ring C is indazolyl, isoindolinyl, pyrazolyl, 6,7-dihydro-5H-pyrrolo[3,4-b]pyrazinyl, 1,3-dihydro-2H-pyrrolo[3,4-c]pyridin-2-yl, 5,7-dihydro-6H-pyrrolo[3,4-b]pyridin-6-yl, 6,7-dihydro-5H-pyrrolo[3,4-c]pyridazinyl or 6,7-dihydro-5H-pyrrolo[3,4-d]pyrimidinyl, each optionally substituted with one or more R³;
each R³ is independently H, halo, —OR^{3a}, —N(R^{3a})₂, —N(R^{3a})C(O)R^{3a}, —C(O)N(R₃ₐ)₂, —CH₃, phenyl or 6-membered heteroaryl, wherein the —CH₃, phenyl and 6-membered heteroaryl are each optionally substituted by one or more R³⁰;
each R³⁰ is independently halo, —OR^{3a}, —CH₃ or phenyl;
each R^{3a} is independently H, —CH₃ or 6-membered heteroaryl, wherein the —CH₃ and 6-membered heteroaryl are each optionally substituted with one or more R^{3b}; and
each R^{3b} is independently Cl, —OH, —OCH₃ or —CH₃.

4. The compound of any one of claims 1-3, wherein the compound is represented by Formula (II): 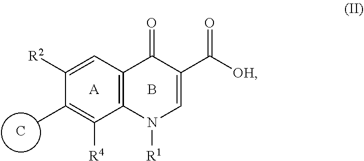
or a pharmaceutically acceptable salt thereof.

5. The compound of any one of claims 1-3, wherein the compound is represented by Formula (III): 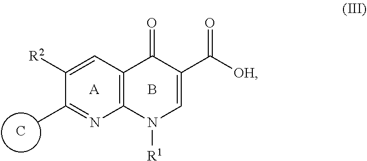
or a pharmaceutically acceptable salt thereof.

6. The compound of claim 4, wherein the compound is represented by Formula (IV), (V) or (VI): 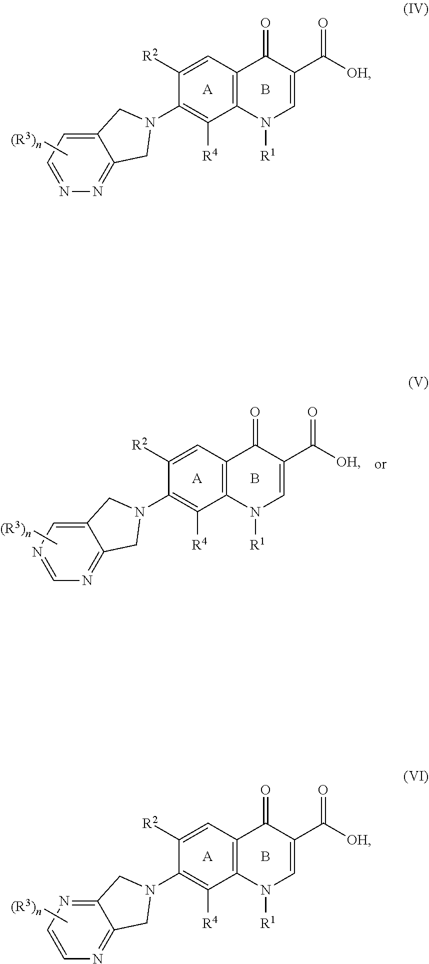
or a pharmaceutically acceptable salt thereof, wherein n is 0, 1, 2, 3, 4 or 5.

7. The compound of claim 5, wherein the compound is represented by Formula (VII), (VIII)
or (IX): 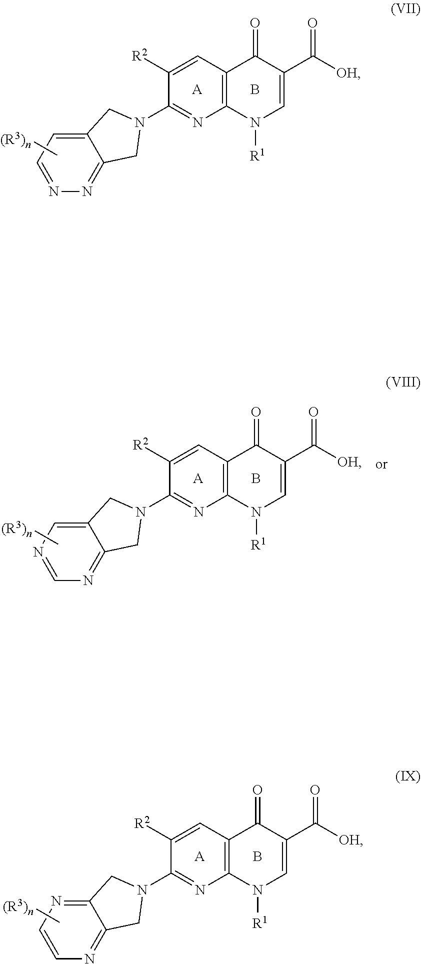
or a pharmaceutically acceptable salt thereof, wherein n is 0, 1, 2, 3, 4 or 5

8. The compound of claim 4, wherein the compound is represented by Formula (X), (XI), (XII) or (XIII): 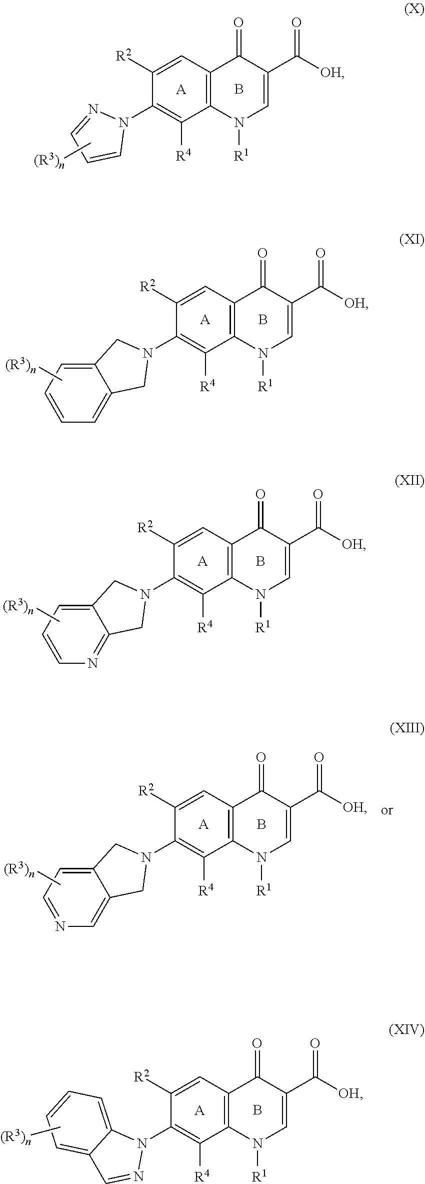
or a pharmaceutically acceptable salt thereof, wherein n is 0, 1, 2, 3, 4 or 5.

9. The compound of claim 5, wherein the compound is represented by Formula (XV), (XVI), (XVII), (XVIII) or (XIX): 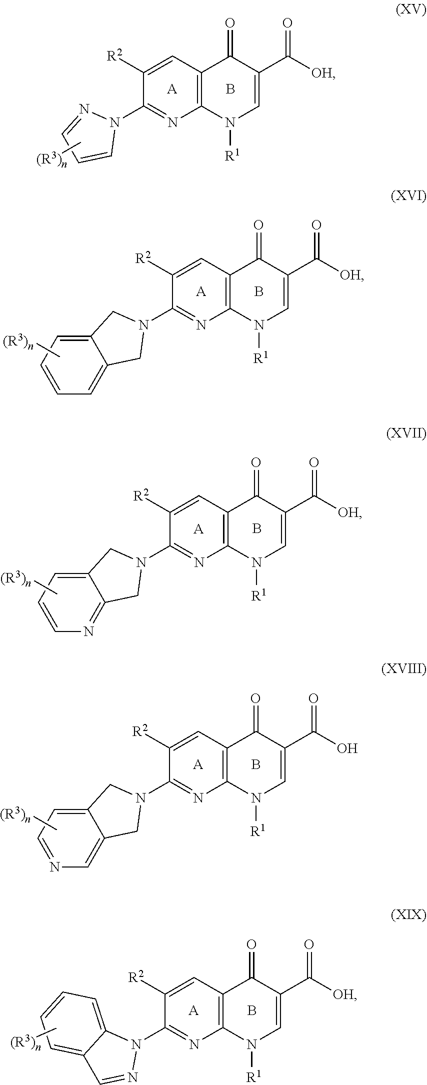
or a pharmaceutically acceptable salt thereof, wherein n is 0, 1, 2, 3, 4 or 5.

10. The compound of claim 8 or 9, or a pharmaceutically acceptable salt thereof, wherein R¹ is —CH₃, —CH₂CH₃, —CH(CH₃)₂, cyclohexyl, cyclopropyl, phenyl, piperidinyl, pyridinyl, pyrazinyl, pyrimidinyl or tetrahydropyranyl, each optionally substituted with one or more R¹⁰, for example R¹ is —CH₃, —CH₂CH₃, —CH(CH₃)₂, cyclohexyl, cyclopropyl, phenyl, piperidinyl, pyridinyl, pyrazinyl or tetrahydropyranyl, each optionally substituted with one or more R¹⁰.

11. The compound of claim 8 or 9, or a pharmaceutically acceptable salt thereof, wherein R¹ is a group of Formula (a) through (g): 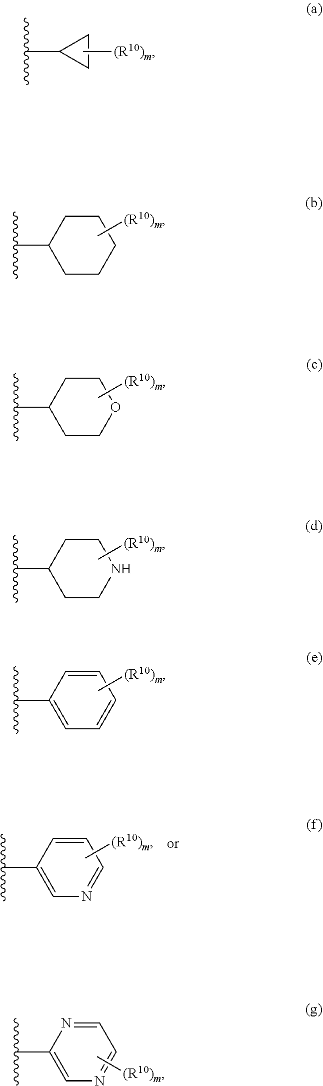
wherein m is 0, 1, 2, 3, 4 or 5, and 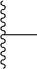
represents a bond to ring B.

12. The compound of claim 8 or 9, or a pharmaceutically acceptable salt thereof, wherein R¹ is a group of Formula (h) through (x): 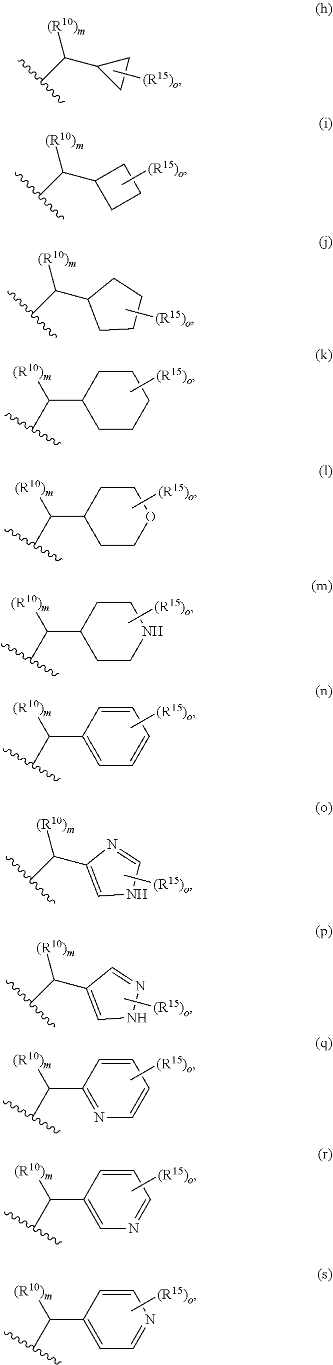 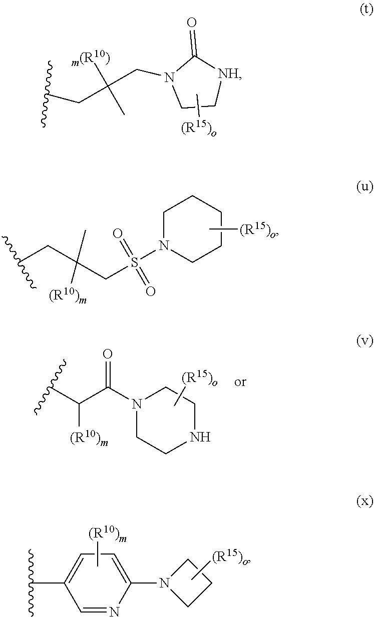
wherein:
R¹⁰ is —CH₃, —CH₂CH₃ or —CH(CH₃)₂;
m is 0, 1 or 2;
o is 0, 1, 2, 3, 4 or 5; and 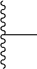
represents a bond to ring B.

13. The compound of any one of claims 8-11, or a pharmaceutically acceptable salt thereof, wherein each R¹⁰ is independently halo, —OR^{1a}, —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, —CH₃, —CH₂CH₃, —CH(CH₃)₂, —CH₂CH₂CH₃, azetidinyl, cyclobutyl, cyclopentyl, cyclopropyl, cyclohexyl, imidazolyl, 2-oxoimidazolidin-1-yl, phenyl, piperidinyl, pyranyl or pyrazolyl, wherein each of the —CH₃, —CH₂CH₃, —CH₂CH₂CH₃, —CH(CH₃)₂, azetinyl, cyclobutyl, cyclopentyl, cyclopropyl, cyclohexyl, imidazolyl, 2-oxoimidazolidin-1-yl, phenyl, piperidinyl, pyranyl and pyrazolyl are optionally substituted by one or more R¹⁵, for example each R¹⁰ is independently halo, —OR^{1a}, —N(R^{1a})₂, —N(R^{1a})C(O)R^{1a}, —N(R^{1a})C(O)OR^{1a}, —N(R^{1a})C(O)N(R^{1a})₂, —N(R^{1a})SO₂R^{1a}, —C(O)R^{1a}, —C(O)N(R^{1a})₂, —CH₃, —CH₂CH₃, —CH(CH₃)₂, azetidinyl, cyclobutyl, cyclopentyl, cyclopropyl, cyclohexyl, imidazolyl, 2-oxoimidazolidin-1-yl, phenyl, piperidinyl, pyranyl or pyrazolyl, wherein each of the —CH₃, —CH₂CH₃, —CH(CH₃)₂, azetinyl, cyclobutyl, cyclopentyl, cyclopropyl, cyclohexyl, imidazolyl, 2-oxoimidazolidin-1-yl, phenyl, piperidinyl, pyranyl and pyrazolyl are optionally substituted by one or more R¹⁵.

14. The compound of any one of claims 8-11, or a pharmaceutically acceptable salt thereof, wherein each R¹⁰ is independently a group of Formula (i) through (xvi): 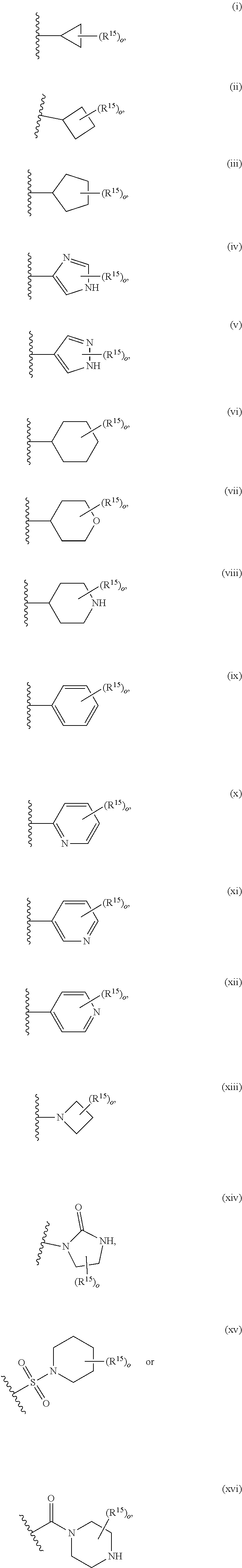
wherein o is 0, 1, 2, 3, 4 or 5, and 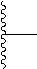
represents a bond to R¹.

15. The compound of any one of claims 8-14, or a pharmaceutically acceptable salt thereof, wherein each R^{1a} is independently H, —CH₃, —CH₂CH₃, —CH(CH₃)₂, —CF₃, tert-butyl, phenyl, cyclopropyl, morpholinyl, piperidin-1-yl or piperazin-1-yl, wherein the —CH₃, —CH₂CH₃, —CH(CH₃)₂, tert-butyl, phenyl, cyclopropyl, morpholinyl, piperidin-1-yl and piperazin-1-yl are each optionally substituted by one or more R^{1b}.

16. The compound of any one of claims 8-15, or a pharmaceutically acceptable salt thereof, wherein each R^{1b} is independently piperidinyl, morpholinyl, —OCH₃, —N(H)CH₃ or —N(CH₃)₂, for example each R^{1b} is independently —OCH₃, —N(H)CH₃ or —N(CH₃)₂.

17. The compound of any one of claims 8-14, or a pharmaceutically acceptable salt thereof, wherein each R^{1a} is independently H, —CH₃, —CH₂CH₃, —CH₂CH₂OCH₃, —CH₂CH₂N(CH₃)₂, —CH₂CH₂NH(CH₃), —C(CH₃)₂, —CF₃, tert-butyl, phenyl, cyclopropyl, morpholinyl, piperidin-1-yl or piperazin-1-yl.

18. The compound of any one of claims 8-14, or a pharmaceutically acceptable salt thereof, wherein each R¹⁵ is independently Cl, F, —OH, —OCH₃, —N(CH₃)₂, —NHC(O)Ot-Bu, —C(O)CH₃, —C(O)N(CH₃)₂, —CH₃ or —CH₂CH₂OCH₃.

19. The compound of any one of claims 8-12, or a pharmaceutically acceptable salt thereof, wherein each R¹⁰ is independently selected from the group consisting of F, —OH, —OCH₃, —NH₂, —CH₂OH, —OCH₂CH₂OMe, —OCH₂CH₂N(H)CH₃, —OCH₂CH₂N(CH₃)₂, —N(H)CH₃, —N(CH₃)₂, —NHCH₂CH₂OCH₃, —N(CH₃)CH₂CH₂N(CH₃)₂, —N(H)C(O)CH₃, —N(H)C(O)CH₂CH₃, —N(H)C(O)CH(CH₃)₂, —N(H)C(O)cyclopropyl, (2-(piperidin-1-yl)ethyl)amino, 1-(dimethyl carbamoyl)piperidin-4-yl, methyl(2-(piperidin-1-yl)ethyl)amino, (2-morpholinoethyl)amino, methyl(2-morpholino ethyl)amino, —NHC(O)Ot-Bu, —N(H)C(O)N(CH₃)₂, —N(H)C(O)(N-morpholine), —N(H)SO₂CH₃, —N(H)SO₂CF₃, —C(O)CH₃, —C(O)phenyl, —C(O)N(CH₃)₂, —CH₃, —CH(CH₃)₂, azetidin-1-yl, 3-(dimethylamino)azetidin-1-yl, benzoyl, 1H-imidazol-4-yl, 1-methyl-1H-imidazol-4-yl, 2-oxoimidazolidin-1-yl, pyridin-2-yl, pyridin-3-yl, pyridin-4-yl, 3-(dimethyl amino)azetidin-1-yl)pyridin-3-yl, cyclobutyl, cyclopentyl, cyclopropyl, cyclohenzyl, phenyl, 2-chlorophenyl, 2-fluorophenyl, 4-methoxyphenyl, 2-methylphenyl, 1-methylpiperidin-4-yl, piperidin-1-ylsulfonyl, tetrahydro-2H-pyran-4-yl, pyrazolyl and 1-methyl-1H-pyrazol-4-yl.

20. The compound of claim 8 or 9, or a pharmaceutically acceptable salt thereof, wherein R¹ is selected from the group consisting of isobutyl, benzyl, 2-chlorobenzyl, 2-fluorobenzyl, 2-methoxybenzyl, 3-methoxybenzyl, 4-methoxybenzyl, 2-methylbenzyl, 1-phenylethyl, (1-methylpiperidin-4-yl)methyl, 1-(dimethylcarbamoyl)piperidin-4-yl, 2-oxo-2-(piperazin-1-yl)ethyl, 2-(methylsulfonamido)ethyl, 2-(piperidin-1-ylsulfonyl)ethyl, 2-(2-oxoimidazolidin-1-yl)ethyl, cyclobutylmethyl, 1-cyclobutylethyl, 2-cyclobutylpropan-2-yl, cyclopropylmethyl, isobutyl, cyclohexylmethyl, 4-hydroxycyclohexyl, cyclopentylmethyl, 1-methylcyclopropyl, pyridin-2-ylmethyl, pyridin-3-ylmethyl, pyridin-4-ylmethyl, tetrahydro-2H-pyran-4-yl, (tetrahydro-2H-pyran-4-yl)methyl, 4-hydroxyphenyl, 2-fluoro-4-hydroxyphenyl, 3-fluoro-4-hydroxyphenyl, 2-fluoro-4-(methylsulfonamido)phenyl, 2-fluoro-4-(hydroxymethyl) phenyl, piperidin-4-yl, piperidin-4-ylmethyl, 1-acetylpiperidin-4-yl, 1-methylpiperidin-4-yl, 1-methylpiperidin-4-yl)methyl, 1-(2-methoxyethyl)piperidin-4-yl, 1-benzoylpiperidin-4-yl), (1-(tert-butoxycarbonyl)piperidin-4-yl)methyl, (1-acetylpiperidin-4-yl)methyl, (2-methoxypyridin-3-yl)methyl, 1-(dimethylcarbamoyl)piperidin-4-yl)methyl, (1H-pyrazol-4-yl)methyl, (1-methyl-1H-pyrazol-4-yl)methyl, (1H-imidazol-4-yl)methyl, (1-methyl-1H-imidazol-4-yl)methyl, pyrazin-2-yl, pyridin-2-yl, pyridin-3-yl, 6-hydroxypyridin-3-yl, 6-hydroxy-4-methylpyridin-3-yl, 4-methyl-6-(methylamino)pyridin-3-yl, 4-methyl-6-((2-(methylamino)ethyl)amino)pyridin-3-yl, 4-methyl-6-(methylsulfonamido)pyridin-3-yl, 4-methyl-6-((trifluoromethyl)sulfonamido)pyridin-3-yl, 5-aminopyrazin-2-yl, 5-amino-3-methylpyrazin-2-yl, 5-methoxy-3-methylpyrazin-2-yl, 6-methylpyrazin-2-yl, 5-methoxypyrazin-2-yl, 6-hydroxy-2-methylpyridin-3-yl, 6-methoxypyridin-3-yl, 6-(dimethylamino)pyridin-3-yl, 6-(dimethylamino)-4-methylpyridin-3-yl, 4-methyl-6-(methyl(2-(piperidin-1-yl)ethyl)amino)pyridin-3-yl, 4-methyl-6-((2-(piperidin-1-yl)ethyl)amino) pyridin-3-yl, 6-((tert-butoxycarbonyl)amino)-4-methylpyridin-yl, 6-aminopyridin-3-yl, 6-amino-5-fluoropyridin-3-yl, 6-amino-2-methylpyridin-3-yl, 6-amino-4-methylpyridin-3-yl, 6-(azetidin-1-yl)-4-methylpyridin-3-yl, 6-amino-2,4-dimethylpyridin-3-yl, 6-acetamidopyridin-3-yl, 6-amino-4-ethylpyridin-3-yl, 6-((2-(dimethylamino)ethyl)(methyl)amino)-4-methylpyridin-3-yl, 6-amino-4-cyclopropylpyridin-3-yl, 6-acetamido-4-methylpyridin-3-yl, 4-methyl-6-propionamidopyridin-3-yl, 6-isobutyramido-4-methylpyridin-3-yl, 6-(cyclopropane carboxamido)-4-methylpyridin-3-yl, 6-((2-methoxyethyl)amino)-4-methylpyridin-3-yl, 6-(azetidin-1-yl)pyridin-3-yl, 6-(3-(dimethylamino)azetidin-1-yl)pyridin-3-yl, 6-(3-(dimethyl amino)azetidin-1-yl)-4-methylpyridin-3-yl, 6-((tert-butoxycarbonyl)amino)-2-methylpyridin-3-yl, 6-((tert-butoxycarbonyl)amino)pyridin-3-yl, 6-(2-methoxyethoxy)pyridin-3-yl, 6-(2-(dimethyl amino)ethoxy)pyridin-3-yl, 6-(3-(dimethylamino)azetidin-1-yl)-4-methylpyridin-3-yl, 6-((2-methoxyethyl)amino)pyridin-3-yl, 4-methyl-6-(morpholine-4-carboxamido)pyridin-3-yl, 4-methyl-6-(methyl(2-morpholinoethyl)amino)pyridin-3-yl, and 6-(3,3-dimethylureido)-4-methylpyridin-3-yl.

21. The compound of any one of claims 8-20, or a pharmaceutically acceptable salt thereof, wherein R² is H, Cl, F, —CN, —OCH₃, —OCH₂CH₃ or —CH₃.

22. The compound of any one of claims 8-21, or a pharmaceutically acceptable salt thereof, wherein each R³ is independently H, halo, —OR^{3a}, —N(R^{3a})₂, —N(R^{3a})C(O)R^{3a}, —C(O)N(R₃a)₂, —CH₃, —CH₂F, —CF₃, phenyl or 6-membered heteroaryl, wherein the methyl, phenyl and 6-membered heteroaryl are each optionally substituted by one or more R³⁰.

23. The compound of any one of claims 8-21, or a pharmaceutically acceptable salt thereof, wherein R³ is independently H, Br, Cl, F, —OH, —OCH₃, —NH₂, —N(H)CH₃, —N(H)CH₂CH₃, —N(CH₃)CH₂CH₃, —N(H)CH₂CH₂OH, —N(H)CH₂CH₂OCH₃, —N(CH₃)CH₂CH₂OCH₃, —N(CH₃)₂, —N(CH₃)CH₂CH₂OH, —N(H)C(O)CH₃, —C(O)NH₂, —C(O)N(H)CH₃, —CH₃, —CF₃, benzyl, phenyl, pyrazin-2-yl, pyridin-2-yl, pyridin-3-yl or pyridin-4-yl, wherein the —CH₃, benzyl, phenyl, pyrazin-2-yl, pyridin-2-yl, pyridin-3-yl and pyridin-4-yl are each optionally substituted by one or
more R³⁰.

24. The compound of any one of claims 8-23, or a pharmaceutically acceptable salt thereof, wherein each R³⁰ is independently halo, —OR^{3a}, —CH₃ or phenyl.

25. The compound of any one of claims 8-24, or a pharmaceutically acceptable salt thereof, wherein each R^{3a} is independently H, —CH₃, —CH₂CH₃ or 6-membered heteroaryl, wherein the —CH₃, —CH₂CH₃ and 6-membered heteroaryl are each optionally substituted with one or more R^{3b}, for example each R^{3a} is independently H, —CH₃ or 6-membered heteroaryl, wherein the —CH₃, and 6-membered heteroaryl are each optionally substituted with one or more R^{3b}.

26. The compound of any one of claims 8-25, or a pharmaceutically acceptable salt thereof, wherein each R^{3b} is independently Cl, —OH, —OCH₃ or —CH₃.

27. The compound of any one of claims 8-24, or a pharmaceutically acceptable salt thereof, wherein each R^{3a} is independently H, —CH₃, —CH₂CH₃, —CH₂CH₂OH, —CH₂CH₂OCH₃, pyridin-2-yl, 3-methylpyridin-2-yl or 3-chloro-6-methoxypyridin-2-yl.

28. The compound of any one of claims 8-23, or a pharmaceutically acceptable salt thereof, wherein each R³⁰ is independently Cl, F, —OCH₃, pyridin-2-yloxy, (3-chloro-6-methoxypyridin-2-yl)oxy, —CH₃ or phenyl.

29. The compound of any one of claims 8-21, or a pharmaceutically acceptable salt thereof, wherein each R³ is independently H, Br, Cl, F, —OH, —OCH₃, —NH₂, —N(CH₃)₂, —N(H)CH₃, —C(O)NH₂, —N(H)CH₂CH₂OH, —N(H)CH₂CH₂OCH₃, —N(CH₃)CH₂CH₂OCH₃, —N(H)C(O)CH₃, —N(CH₃)CH₂CH₂OH, —C(O)N(H)CH₃, —CH₃, —CF₃, benzyl, phenyl, 3-chlorophenyl, 3-fluoro phenyl, 3-methylphenyl, 3-methoxyphenyl, 4-methoxyphenyl, pyrazin-2-yl, pyridin-2-yl, pyridin-3-yl, pyridin-4-yl, 3-chloropyridin-4-yl, 3-methylpyridin-4-yl, (pyridin-2-yloxy)methyl, 3-(((3-methylpyridin-2-yl)oxy)methyl or ((3-chloro-6-methoxypyridin-2-yl)oxy)methyl.

30. The compound of any one of claims 8-21, or a pharmaceutically acceptable salt thereof, wherein ring C is selected from the group consisting of isoindolin-2-yl, 4-chloroindolin-1-yl, 4-fluoroisoindolin-2-yl, 4-methoxyisoindolin-2-yl, 5-fluoroisoindolin-2-yl, 5-methoxyisoindolin-2-yl, 7-bromo-1H-indazol-1-yl, 5,7-dichloro-1H-indazol-1-yl, 6-fluoro-1H-indazol-1-yl, 5,6-difluoro-1H-indazol-1-yl, 7-methyl-1H-indazol-1-yl, 1H-pyrazol-1-yl, 3-carbamoyl-1H-pyrazol-1-yl, 4-carbamoyl-1H-pyrazol-1-yl, 3-(3-chloropyridin-4-yl)-1H-pyrazol-1-yl, 4-(methyl carbamoyl)-1H-pyrazol-1-yl, 3-fluoro-1H-pyrazol-1-yl, 4-fluoro-1H-pyrazol-1-yl, 3-(trifluoro methyl)-1H-pyrazol-1-yl, 3-amino-1H-pyrazol-1-yl, 3-(methylamino)-1H-pyrazol-1-yl, 3-bromo-1H-pyrazol-1-yl, 3-chloro-1H-pyrazol-1-yl, 5-methyl-3-(pyridin-2-yl)-1H-pyrazol-1-yl, 3-(methylamino)-1H-pyrazol-1-yl, 3-(dimethylamino)-1H-pyrazol-1-yl, 4-bromo-3-methyl-1H-pyrazol-1-yl, 3-acetamido-1H-pyrazol-1-yl, 4-acetamido-1H-pyrazol-1-yl, 3-(4-methoxyphenyl)-1H-pyrazol-1-yl, 3-((2-hydroxyethyl)amino)-1H-pyrazol-1-yl, 3-((2-hydroxyethyl)(methyl) amino)-1H-pyrazol-1-yl, 3-((2-methoxyethyl)amino)-1H-pyrazol-1-yl, 3-(3-methylpyridin-4-yl)-1H-pyrazol-1-yl, 3-(((3-methylpyridin-2-yl)oxy)methyl)-1H-pyrazol-1-yl, 3-((2-methoxyethyl) (methyl)amino)-1H-pyrazol-1-yl, 3-(pyrazin-2-yl)-1H-pyrazol-1-yl, 3-((pyridin-2-yloxy)methyl)-1H-pyrazol-1-yl, 3-phenyl-1H-pyrazol-1-yl, 3-(pyridin-3-yl)-1H-pyrazol-1-yl, 3-(pyridin-4-yl)-1H-pyrazol-1-yl, 5,7-dihydro-6H-pyrrolo[3,4-b]pyridin-6-yl, 4-fluoro-5,7-dihydro-6H-pyrrolo [3,4-b]pyridin-6-yl, 3-fluoro-5,7-dihydro-6H-pyrrolo[3,4-b]pyridin-6-yl, 4-methyl-5,7-dihydro-6H-pyrrolo[3,4-b]pyridin-6-yl, 3-methyl-5,7-dihydro-6H-pyrrolo[3,4-b]pyridin-6-yl, 2-methyl-5,7-dihydro-6H-pyrrolo[3,4-b]pyridin-6-yl, 3-methyl-5,7-dihydro-6H-pyrrolo[3,4-b]pyridin-6-yl, 1,3-dihydro-2H-pyrrolo[3,4-c]pyridin-2-yl, 5-oxo-5,7-dihydro-6H-pyrrolo[3,4-b]pyridin-6-yl, 7-methyl-5,7-dihydro-6H-pyrrolo[3,4-b]pyridin-6-yl, 5-methyl-5,7-dihydro-6H-pyrrolo[3,4-b] pyridin-6-yl, 4-methyl-4,5,6,7-tetrahydro-2H-pyrazolo[3,4-b]pyrazin-2-yl, 7-methyl-4,5,6,7-tetrahydro-2H-pyrazolo[3,4-b]pyrazin-2-yl and 7-methyl-6,7-dihydropyrazolo[4,3-b][1,4]oxazin-2(5H)-yl.

31. The compound of any one of claims 8 and 10-30, or a pharmaceutically acceptable salt thereof, wherein R⁴ is H or F.

32. The compound of claim 1, wherein the compound is represented by Formula (XX), (XXI), (XXII), (XXIII), (XXIV) or (XXV): 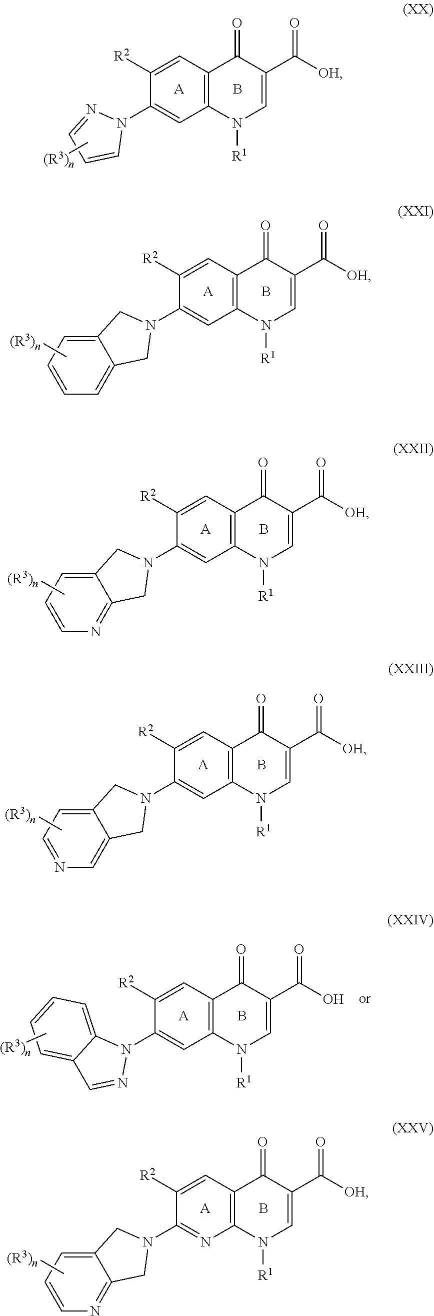
or a pharmaceutically acceptable salt thereof, wherein:
R¹ is —CH₃, —CH₂CH(CH₃)₂, phenyl, piperidin-4-yl, pyridin-3-yl or pyrazin-2-yl, wherein the —CH₃, phenyl, piperidin-4-yl, pyridin-3-yl and pyrazin-2-yl are each each optionally substituted with one or more R¹⁰;
R¹⁰ is independently F, —OH, —OCH₃, —OCH₂CH₂OCH₃, —NH₂, —NH(CH₃), —N(CH₃)₂, —OCH₂CH₂N(CH₃)₂, —NHCH₂CH₂OCH₃, —NHC(O)CH₃, —NHC(O)CH₂CH₃, —NHSO₂CH₃, —NHC(O)CH(CH₃)₂, —NHC(O)OC(CH₃)₃, —CH₃, —C(O)OC(CH₃)₃, benzoyl, —CH₂OH, phenyl, cyclobutyl, cyclohexyl, cyclopentyl, azetidin-1-yl, imidazol-4-yl, piperidin-4-yl, pyridin-2-yl, pyridin-3-yl or pyridin-4-yl, wherein the —CH₃, cyclobutyl, azetidin-1-yl, piperidin-4-yl, phenyl and pyridin-3-yl are each optionally substituted by one or more R⁵;
R¹⁵ is independently Cl, F, —OH, —OCH₃, —N(CH₃)₂, —C(O)OC(CH₃)₃ or —CH₃;
R² is Cl, F or —CN;
R³ is independently Br, Cl, F, —OCH₃, —NHCOCH₃, —CH₃ or 4-methoxyphenyl;
and
n is 0, 1 or 2.

33. The compound of claim 1, wherein the compound is represented by Formula (XXVI) or (XXVII): 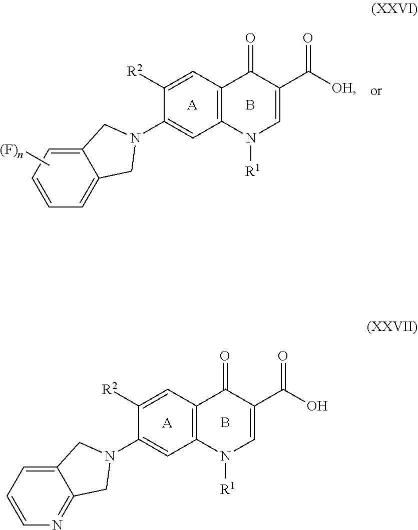
or a pharmaceutically acceptable salt thereof, wherein:
R¹ is —CH₃, phenyl, pyridin-3-yl or pyrazin-2-yl, each optionally substituted with F, —OH, —OCH₃, —NH₂, —NHC(O)OC(CH₃)₃, —NHSO₂CH₃, —CH₃ or 2-chlorophenyl;
R² is Cl, F or —CN; and
n is 0 or 1.

34. A pharmaceutical composition, comprising a compound of any one of claims 1-33 and a pharmaceutically acceptable carrier.

35. A method of treating a telomere disease or disorder associated with telomer dysfunction in a subject, the method comprising administering to the subject in need thereof a therapeutically effective amount of a compound of any one of claims 1-33, or a pharmaceutically acceptable salt thereof, or a pharmaceutical composition of claim 34, except that the provisos of claim 1 do not apply.

36. A method of treating a telomere disease or disorder associated with telomer dysfunction in a subject, the method comprising administering to the subject a therapeutically effective amount of a compound of any one of claims 1-33, or a pharmaceutically acceptable salt thereof, or a pharmaceutical composition of claim 34.

37. The method of claim 35 or 36, wherein the compound is a PAPD5 inhibitor.

38. The method of claim 35 or 36, wherein the compound is a PAPD7 inhibitor.

39. The method of any one of claims 35-37, wherein the telomere disease or disorder associated with telomer dysfunction is selected from the group consisting of a hemotological disease, an immunodeficiency disease, a pulmonary disease, a hepatic disease, a dermatology disease, a mucosal disease, an osteopathic disease, a cardiovascular disease, an endocrine disease, a gastrointestinal disease, a neurological disease and an opthalmic disease.

40. The method of claim 39, wherein:
the hematological disease is selected from the group consisting of dyskeratosis congenita (DC), Revesz syndrome, Hoyeraal-Hreidarrson syndrome, Coats plus syndrome, aplastic anemia, myelodysplastic syndrome and diabetes;
the immunodeficiency disease is selected from the group consisting of primary immunodeficiency and inflammatory bowel disease;
the pulmonary disease is selected from the group consisting of dyskeratosis congenita (DC), Revesz syndrome, Hoyeraal-Hreidarrson syndrome, Coats plus syndrome, familial pulmonary fibrosis and idiophathic pulmonary fibrosis;
the hepatic disease is selected from the group consisting of dyskeratosis congenita (DC), Revesz syndrome, Hoyeraal-Hreidarrson syndrome, Coats plus syndrome, hepatic fibrosis, chronic liver disease, non-alcoholic steatohepatitis, hepatic cirrhosis, nodular regenerative hyperplasia, chronic liver disease, non-alcoholic steatohepatitis and hepatic cirrhosis;
the dermatology disease is selected from the group consisting of dyskeratosis congenita (DC), Revesz syndrome, Hoyeraal-Hreidarrson syndrome and Coats plus syndrome;
the mucosal disease is dyskeratosis congenita (DC);
the osteopathic disease is selected from the group consisting of dyskeratosis congenita (DC), Revesz syndrome, Hoyeraal-Hreidarrson syndrome, Coats plus syndrome, myelodysplastic syndrome, osteoporosis, osteonecrosis, bone marrow failure, osteoarthritis, rheumatoid arthritis and sarcopenia;
the cardiovascular disease is selected from the group consisting of dyskeratosis congenita (DC), Revesz syndrome, Hoyeraal-Hreidarrson syndrome, Coats plus syndrome, vascular malformations, atherosclerosis, hypertension, coronary artery disease, ischaemic heart disease and congestive heart failure;
the endocrine disease is selected from the group consisting of endogenous hypercortisolism (Cushings's disease) and acromegaly;
the gastrointestinal disease is selected from the group consisting of dyskeratosis congenita (DC), Revesz syndrome, Hoyeraal-Hreidarrson syndrome, Telomere Syndrome and Coats plus syndrome;
the neurological disease is selected from the group consisting of dyskeratosis congenita (DC), Revesz syndrome, Hoyeraal-Hreidarrson syndrome, Coats plus syndrome, cerebral hypoplasia, microcephaly, MotorNeuron Disease, Creutzfeldt-Jakob disease, Machado-Joseph disease, Spino-cerebellar ataxia, multiple sclerosis (MS), Parkinson's disease, Huntington's disease, epilepsy, schizophrenia, bipolar disorder, depression, dementia, Pick's Disease, central nervous system hypoxia and cerebral senility; and
the ophthalmic disease is selected from the group consisting of dyskeratosis congenita (DC), Revesz syndrome, Hoyeraal-Hreidarrson syndrome, Coats plus syndrome, glaucoma, cataracts and macular degeneration.

41. The method of claim 39, wherein the telomere disease or disorder associated with telomer dysfunction is selected from the group consisting of dyskeratosis congenita (DC), aplastic anemia, pulmonary fibrosis, hepatic cirrhosis, bone marrow failure and Hoyeraal-Hreidarrson syndrome, except that the provisos of claim 1 do not apply.

42. The method of claim 39, wherein the telomere disease or disorder associated with telomer dysfunction is selected from the group consisting of dyskeratosis congenita (DC), aplastic anemia, pulmonary fibrosis, hepatic cirrhosis, bone marrow failure and Hoyeraal-Hreidarrson syndrome.

43. A method of treating a subject with cancer, the method comprising administering to the subject a therapeutically effective amount of a compound of any one of claims 1-33, or a pharmaceutically acceptable salt thereof, or a pharmaceutical composition of claim 34.

44. A method of treating a subject with hepatitis B, the method comprising administering to the subject a therapeutically effective amount of a compound of any one of claims 1-33, or a pharmaceutically acceptable salt thereof, or a pharmaceutical composition of claim 34, except that the provisos of claim 1 do not apply.

45. A method of treating a subject with hepatitis B, the method comprising administering to the subject a therapeutically effective amount of a compound of any one of claims 1-33, or a pharmaceutically acceptable salt thereof, or a pharmaceutical composition of claim 34.
